(19) United States
(12) Patent Application Publication
(10) Pub. No.: US 2004/0018513 A1
(43) Pub. Date: Jan. 29, 2004

(54) Classification and prognosis prediction of acute lymphoblastic leukemia by gene expression profiling

(76) Inventors: James R. Downing, Cordova, TN (US); Eng-Juh Yeoh, Singapore (SG); Dawn E. Wilkins, Oxford, MS (US); Limsoon Wong, Singapore (SG)

Correspondence Address: ALSTON AND BIRD LLP, ST. JUDE CHILDREN'S RESEARCH HOSPITAL, BANK OF AMERICA PLAZA, 101 SOUTH TRYON STREET, SUITE 4000, CHARLOTTE, NC, 28280-4000, US

(21) Appl. No.: 10/391,271

(22) Filed: Mar. 18, 2003

Related U.S. Application Data
(60) Provisional application No. 60/367,144, filed on Mar. 22, 2002.

(51) Int. Cl. C12Q 1/68

(52) U.S. Cl. 435/6

(57) ABSTRACT

The present invention provides methods and compositions useful for diagnosing and choosing treatment for leukemia patients. The claimed methods include methods of assigning a subject affected by leukemia to a leukemia risk group, methods of predicting whether a subject affected by leukemia has an increased risk of relapse, methods of predicting whether a subject affected by leukemia has an increased risk of developing secondary acute myeloid leukemia, methods to aid in the determination of a prognosis for a subject affected by leukemia, methods of choosing a therapy for a subject affected by leukemia, and methods of monitoring the disease state in a subject undergoing one or more therapies for leukemia. The claimed compositions include arrays having capture probes for the differentially-expressed genes of the invention, computer readable media having digitally-encoded expression profiles associated with leukemia risk groups, and kits for diagnosing and choosing therapy for leukemia patients.

### CROSS-REFERENCE TO RELATED APPLICATIONS

This application claims the benefit of U.S. Provisional Application No. 60/367,144 filed Mar. 22, 2002, which is hereby incorporated in its entirety by reference herein.

### FEDERALLY SPONSORED RESEARCH OR DEVELOPMENT

This research underlying this invention was supported in part with funds from National Institutes of Health grants P01 CA71907-06, CA51001, CA36401, CA78224, Cancer Center CORE Grant CA-21765, and National Science Foundation grant EIA-0074869. The United States Government may have an interest in the subject matter of the invention.

### BACKGROUND OF THE INVENTION

Pediatric acute lymphoblastic leukemia (ALL) is one of the great success stories of modern cancer therapy, with contemporary treatment protocols achieving overall long-term event free survival rates approaching 80% (Schrappe et al. (2000) Blood 95:3310-22; Silverman et al.(2001) Blood 97:1211-18; and Pui and Evans (1998) N. Eng. J. Med. 339:605-15). This success has been achieved in part by using risk-adapted therapy that involves tailoring the intensity of treatment to each patient's risk of relapse. This approach was developed following the realization that pediatric ALL is a heterogeneous disease consisting of various leukemia subtypes that differ markedly in their response to chemotherapy (reviewed in Pui and Evans (1998) N. Eng. J. Med. 339:605-15). By tailoring the intensity of treatment to a patient's relative risk of relapse, patients are neither under-treated or over-treated, and are thus afforded the highest chance for a cure.

Critical to the success of this approach has been the accurate assignment of individual patients to specific risk groups. Although risk assignment is influenced by a variety of clinical and laboratory parameters, the genetic alterations that underlie the pathogenesis of individual leukemia subtypes figure prominently in most classification schemes (Silverman L B et al. (2001) Blood 97:1211-18; and Pui and Evans (1998) N. Engl. J. Med. 339:605-15). Through systematic immunophenotyping and cytogenetic analysis, and the subsequent molecular cloning of the genes targeted by the identified chromosomal rearrangements, a number of genetically distinct leukemia subtypes have been defined. These include B-lineage leukemias that contain t(9;22)[BCR-ABL], t(1;19)[E2A-PBX1], t(12;21)[TEL-AML1], rearrangements in the MLL gene on chromosome 11, band q23, or a hyperdiploid karyotype (i.e., >50 chromosomes), and T-lineage leukemias (T-ALL) (Silverman et al.(2001) Blood 97:1211-18; and Pui and Evans (1998) N. Eng. J. Med. 339:605-15). The underlying genetic lesions in these leukemia subtypes influence the response to cytotoxic drugs. For example, leukemias that express the E2A-PBX1 fusion protein respond poorly to conventional antimetabolite-based treatment, but have cure rates approaching 80% when treated with more intensive therapies (Raimondi et al. (1990) J. Clin. Oncol. 8:1380-88; and Hunger (1996) Blood 87:1211-1224). Similarly, BCR-ABL expressing ALLs, or infants with MLL rearrangements have exceedingly poor cure rates with conventional chemotherapy, and allogeneic hematopoietic stem cell transplantation with HLA matched sibling donor has already been shown to improve outcome for patients with the former leukemia subtype (Pui et al. (1991) Blood 77:440-46; Heerema et al. (1999) Leukemia 13:679-86; Arico et al. (2000) N. Engl. J. Med. 342:998-1006; and Biondi et al. (2000) Blood 96:24-33).

Unfortunately, the accurate assignment of patients to specific risk groups is a difficult and expensive process, requiring intensive laboratory studies including immunophenotyping, cytogenetics, and molecular diagnostics (Pui and Evans (1998) N. Eng. J. Med. 339:605-15; and Pui et al. (2001) Lancet Oncology 2:597-607). Moreover, these diagnostic approaches require the collective expertise of a number of professionals, and although this expertise is available at most major medical centers, it is generally unavailable in developing countries. Accordingly, there remains a need for rapid, less expensive methods of assigning patients affected by ALL into known leukemia risk groups and identifying patients for whom there is a high risk that conventional therapeutic approaches will fail.

### BRIEF SUMMARY OF THE INVENTION

The present invention provides methods and compositions useful for diagnosing and choosing treatment for subjects affected by leukemia. The claimed methods include methods of assigning a subject affected by leukemia to a leukemia risk group, methods of predicting whether a subject affected by leukemia has an increased risk of relapse, methods of predicting whether a subject affected by leukemia has an increased risk of developing secondary acute myeloid leukemia (AML), methods to aid in the determination of a prognosis for a subject affected by leukemia, methods of choosing a therapy for a subject affected by leukemia, and methods of monitoring the disease state in a subject undergoing one or more therapies for leukemia. Methods of screening test compounds to identify therapeutic compounds useful for the treatment of leukemia and molecular targets for these therapeutic compounds are also provided.

The claimed methods comprise providing an expression profile of a sample from a subject affected by leukemia and comparing this subject expression profile to one or more reference expression profiles. In one embodiment, the reference profiles are associated with leukemia risk groups, and the subject expression profile is compared to one or more of these risk group reference profiles to thereby assign the subject affected by leukemia to a leukemia risk group. In another embodiment, one or more reference profiles are associated with relapse of leukemia and the subject expression profile is compared to one or more of these relapse reference profiles to determine if the subject has an increased risk of relapse. In yet another embodiment, one or more reference profiles are associated with secondary AML, and the subject expression profile is compared to one or more of these reference profiles to determine whether the subject has an increased risk of developing secondary AML.

The present invention also provides compositions useful for diagnosing and choosing a therapy for subjects affected by leukemia. These compositions include arrays comprising a plurality of capture probes that can bind specifically to nucleic acid molecules that are differentially expressed in leukemia risk groups, in leukemia subjects who have relapsed, or in leukemia subjects who have developed secondary AML. Also provided is a computer-readable medium comprising digitally-encoded expression profiles comprising values representing the expression levels of genes that are differentially expressed in leukemia risk groups, in leukemia subjects who have relapsed, or in leukemia subjects who have developed secondary AML. Additional compositions of the invention include kits comprising an array of capture probes that can bind specifically to nucleic acid molecules that are differentially expressed in leukemia risk groups, in leukemia subjects who have relapsed, or in leukemia subjects who have developed secondary AML, and a computer-readable medium having digitally encoded expression profiles with values representing the expression level of a nucleic acid molecule detected by the array.

### DETAILED DESCRIPTION OF THE INVENTION

The present invention provides a single platform, expression analysis, that can accurately identify each of the known prognostically and therapeutically relevant subgroups of leukemia and predict the risk of relapse and the risk of secondary (therapy-induced) AML in patients having leukemia. The methods and compositions of the invention provide tools useful in choosing a therapy for leukemia patients, including methods for assigning a leukemia patient to a leukemia risk group, methods of predicting whether a leukemia patient has an increased risk of relapse, methods of predicting whether a leukemia patient has an increased risk of developing secondary (therapy-induced) AML, methods of choosing a therapy for a leukemia patient, methods of determining the efficacy of a therapy in a leukemia patient, and methods of determining the prognosis for a leukemia patient.

The methods of the invention comprise the steps of providing an expression profile from a sample from a subject affected by leukemia and comparing this subject expression profile to one or more reference profiles that are associated with a particular physiologic condition, such as a leukemia risk group, the occurrence of relapse, or the development of secondary AML. By identifying the leukemia risk group reference profile that is most similar to the subject expression profile, the subject can be assigned to a leukemia risk group. Similarly, the risk that a subject affected by leukemia will relapse or develop secondary AML can be predicted by determining whether the expression profile from the subject is sufficiently similar to a reference profile associated with relapse or a reference profile associated with the development of secondary AML. In another embodiment, the subject expression profile is from a subject affected by leukemia who is undergoing a therapy to treat the leukemia. The subject expression profile is compared to one or more reference expression profiles of the invention to monitor the efficacy of the therapy.

Expression Profiles

As used herein, an “expression profile” comprises one or more values corresponding to a measurement of the relative abundance of a gene expression product. Such values may include measurements of RNA levels or protein abundance. Thus, the expression profile can comprise values representing the measurement of the transcriptional state or the translational state of the gene. See, U.S. Pat. Nos. 6,040,138, 5,800,992, 6,020135, 6,344,316, and 6,033,860, which are hereby incorporated by reference in their entireties.

The transcriptional state of a sample includes the identities and relative abundance of the RNA species, especially mRNAs present in the sample. Preferably, a substantial fraction of all constituent RNA species in the sample are measured, but at least a sufficient fraction to characterize the transcriptional state of the sample is measured. The transcriptional state can be conveniently determined by measuring transcript abundance by any of several existing gene expression technologies.

Translational state includes the identities and relative abundance of the constituent protein species in the sample. As is known to those of skill in the art, the transcriptional state and translational state are related.

In some embodiments, the expression profiles of the present invention are generated from samples from subjects affected by leukemia, including subjects having leukemia, subjects suspected of having leukemia, subjects having a propensity to develop leukemia, or subjects who have previously had leukemia, or subjects undergoing therapy for leukemia. The samples from the subject used to generate the expression profiles of the present invention can be derived from a variety of sources including, but not limited to, single cells, a collection of cells, tissue, cell culture, bone marrow, blood, or other bodily fluids. The tissue or cell source may include a tissue biopsy sample, a cell sorted population, cell culture, or a single cell. Sources for the sample of the present invention include cells from peripheral blood or bone marrow, such as blast cells from peripheral blood or bone marrow.

In selecting a sample, the percentage of the sample that constitutes cells having differential gene expression in leukemia risk groups, relapse, or secondary AML should be considered. Samples may comprise at least 20%, at least 30%, at least 40%, at least 50%, at least 55%, at least 60%, at least 70%, at least 75%, at least 80%, at least 85%, at least 90%, or at least 95% cells having differential expression in leukemia risk groups, relapse, or secondary AML, with a preference for samples having a higher percentage of such cells. In some embodiments, these cells are blast cells, such as leukemic cells. The percentage of a sample that constitutes blast cells may be determined by methods well known in the art; see, for example, the methods described elsewhere herein.

In some embodiments of the present invention, the expression profiles comprise values representing the expression levels of genes that are differentially expressed in leukemia risk groups, in subjects affected by leukemia who have relapsed, or in subjects affected by leukemia who have developed secondary AML. The term “differentially expressed” as used herein means that the measurement of a cellular constituent varies in two or more samples. The cellular constituent may be upregulated in a sample from a subject having one physiologic condition in comparison with a sample from a subject having a different physiologic condition, or down regulated in a sample from a subject having one physiologic condition in comparison with a sample from a subject having a different physiologic condition. For example, in one embodiment, the differentially expressed genes of the present invention may be expressed at different levels in different leukemia risk groups. In another embodiment, the differentially expressed genes are expressed in different levels in subjects affected by leukemia who will relapse after conventional treatment in comparison with subjects affected by leukemia who will not relapse and thus will remain in continuous complete remission. In yet another embodiment, the differentially expressed genes are expressed in different levels in subjects affected by leukemia who will develop secondary AML in comparison with subjects affected by leukemia who will not develop secondary AML.

The present invention provides groups of genes that are differentially expressed in diagnostic leukemia samples of patients in different risk groups, or in patients that go on to develop a relapse or a therapy induced (secondary) AML. Some of these genes were identified based on gene expression levels for 12,600 probes in 360 leukemia samples. Values representing the expression levels of the nucleic acid molecules detected by the probes were analyzed using five different statistical metrics to identify genes that were differentially expressed in leukemia risk groups. The methods used to analyze the expression level values to identify differentially expressed genes were the Chi-square statistics method, the Correlation-based Feature Selection method, the T-statistics method, the Wilkins' method, and the self-organizing map and discriminant analysis with variance metric. Although different methods of analysis resulted in the selection of different groups of differentially expressed genes, the genes selected by each method could be used to create an expression profile that could accurately determine whether a leukemia patient should be assigned to a risk group, with an overall diagnostic accuracy of about 96%. See, the Experimental section.

Additional genes that are differentially expressed in diagnostic leukemia samples were identified based on gene expression levels for 26,825 probes in a subset of 132 leukemia samples selected from the 360 leukemia samples described above. A chi-squared metric followed by permutation test was used to identify discriminating genes for the T-ALL, E2A-PBX1, TEL-AML1, BCR-ABL, MLL rearrangement, and Hyperdiploid >50 chromosomes. Genes whose expression is limited to a single B-cell lineage were also identified, and are provided in Tables 70-74.

Thus, distinct sets of differentially expressed genes that can be used to distinguish the T-lineage, hyperdiploid >50 chromosomes, BCR-ABL, E2A-PBX1, TEL-AML1, and MLL gene rearrangement risk groups are provided. Examples of genes that are differentially expressed in the T-ALL risk group are shown in Tables 7, 14, 21, 28, 35, 59, and 67. Examples of genes that are differentially expressed in the E2A-PBX1 risk group are shown in Tables 3, 10, 17, 24, 31, 55, 64, and 71. Examples of genes that are differentially expressed in the TEL-AML1 risk group are shown in Tables 8, 15, 22, 29, 36, 60, 68, and 74. Examples of genes that are differentially expressed in the BCR-ABL risk group are shown in Tables 2, 9, 16, 23, 30, 54, 63, and 70. Examples of genes that are differentially expressed in the MLL risk group are shown in Tables 5, 12, 19, 26, 33, 57, 66, and 73. Examples of genes that are differentially expressed in the Hyperdiploid>50 risk group are shown in Tables 4, 11, 18, 25, 32, 56, 65, and 72.

The present invention further provides a seventh leukemia risk group, herein termed “Novel,” that can be distinguished from the previously-described leukemia risk groups based on expression profiling. The expression profiles from subjects in the Novel risk group are distinguishable from those of the T-ALL, E2A-PBX1, TEL-AML1, BCR-ABL, MLL, and Hyperdiploid >50 risk groups. Subjects assigned to the Novel risk group have similar expression profiles. Examples of genes that are differentially expressed in the Novel leukemia risk group are shown in Tables 4, 11, 18, 25, 32, and 58.

Similarly, sets of differentially expressed genes associated with leukemia patients in the T-ALL, Hyperdiploid >50, TEL-AML1, MLL, and Other (i.e. not the T-ALL, hyperdiploid >50, TEL-AML1, MLL, E2A-PBX1, or BCR-ABL) risk groups who have undergone relapse were identified. Examples of differentially expressed genes associated with relapse in subjects in the T-ALL risk group are shown in Table 44. Examples of differentially expressed genes associated with relapse in subjects in the hyperdiploid >50 risk group are shown in Table 45. Examples of differentially expressed genes associated with relapse in subjects in the TEL-AML1 risk group are shown in Table 46. Examples of differentially expressed genes associated with relapse in subjects in the MLL risk group are shown in Table 47. Examples of differentially expressed genes associated with relapse in subjects in the E2A-PBX1, BCR-ABL, and Novel risk group are shown in Table 48.

The invention also provides genes that are differentially expressed in subjects affected by TEL-AML1 who have developed secondary (treatment-induced) AML. Examples of such genes are shown in Table 52.

The present invention also reveals genes with a high differential level of expression in leukemic compared to normal cells. These highly differentially expressed genes are selected from the genes shown in Tables 2-36 and 44-48, 63-68, and 70-74. These genes and their expression products are useful as markers to detect the presence of minimal residual disease (MRD) in a patient. Antibodies or other reagents or tools may be used to detect the presence of these telltale markers of MRD.

The expression profiles of the invention comprise one or more values representing the expression level of a gene having differential expression in a leukemia risk group, in subjects affected by leukemia who will relapse after conventional therapy, or in subjects affected by leukemia who will develop secondary AML after conventional therapy. Each expression profile contains a sufficient number of values such that the profile can be used to distinguish one leukemia risk group from another, or to distinguish subjects who will relapse after conventional therapy from those who will not relapse, or to distinguish subjects who will develop secondary AML after conventional therapy from those who will not develop secondary AML. In some embodiments, the expression profiles comprise only one value. For example, it can be determined whether a subject affected by leukemia is in the T-ALL risk group based only on the expression level of the CD3D antigen (NCBI Accession No. AA919102; see Table 14). Similarly, it can be determined whether a subject affected by leukemia is in the E2A-PBX1 risk group based only on the expression level of the cDNA of NCBI Accession No. AL049381 (see Table 10). In other embodiments, the expression profile comprises more than one value corresponding to a differentially expressed gene, for example at least 2 values, at least 3 values, at least 4 values, at least 5 values, at least 6 values, at least 7 values, at least 8 values, at least 9 values, at least 10 values, at least 11 values, at least 12 values, at least 13 values, at least 14 values, at least 15 values, at least 16 values, at least 17 values, at least 18 values, at least 19 values, at least 20 values, at least 22 values, at least 25 values, at least 27 values, at least 30 values, at least 35 values, at least 40 values, at least 45 values, at least 50 values, at least 75 values, at least 100 values, at least 125 values, at least 150 values, at least 175 values, at least 200 values, at least 250 values, at least 300 values, at least 400 values, at least 500 values, at least 600 values, at least 700 values, at least 800 values, at least 900 values, at least 1000 values, at least 1200 values, at least 1500 values, or at least 2000 or more values.

It is recognized that the diagnostic accuracy of assigning a subject to a leukemia risk group, determining whether a subject has an increased risk for relapse, or determining whether a subject has an increased risk of developing secondary AML will vary based on the number of values contained in the expression profile. Generally, the number of values contained in the expression profile is selected such that the diagnostic accuracy is at least 85%, at least 87%, at least 90%, at least 91%, at least 92%, at least 93%, at least 94%, at least 95%, at least 96%, at least 97%, at least 98%, or at least 99%, as calculated using methods described elsewhere herein, with an obvious preference for higher percentages of diagnostic accuracy.

It is recognized that the diagnostic accuracy of assigning a subject to a leukemia risk group, determining whether a subject has an increased risk for relapse, or determining whether a subject has an increased risk of developing secondary AML will vary based on the strength of the correlation between the expression levels of the differentially expressed genes and the associated physiologic condition. When the values in the expression profiles represent the expression levels of genes whose expression is strongly correlated with the physiologic condition, it may be possible to use fewer number of values in the expression profile and still obtain an acceptable level of diagnostic or prognostic accuracy.

The strength of the correlation between the expression level of a differentially expressed gene and the presence or absence of a particular physiologic state may be determined by a statistical test of significance. For example, the chi square test used to select genes in some embodiments of the present invention assigns a chi square value to each differentially expressed gene, indicating the strength of the correlation of the expression of that gene and the presence or absence of the associated physiologic condition. Similarly, the T-statistics metric and the Wilkins' metric both provide a value or score indicative of the strength of the correlation between the expression of the gene and the absence or presence of the associated physiologic conditions. These scores may be used to select the genes whose expression levels have the greatest correlation with a particular physiologic state in order to increase the diagnostic or prognostic accuracy of the methods of the invention, or in order to reduce the number of values contained in the expression profile while maintaining the diagnostic or prognostic accuracy of the expression profile.

For example, in one embodiment the chi square test is used to determine the significance of the differentially expressed genes whose expression levels are included in the array, and only those genes having a chi square value of more than 20, more than 25, more than 30, more than 35, more than 40, more than 45, more than 50, more than 55, more than 60, more than 65, more than 70, more than 75, more than 80, more than 90, more than 100, more than 120, more than 140, more than 160, more than 180, or more than 200 are selected.

In another embodiment, the T-statistics metric is used to determine the significance of the differentially expressed genes whose expression levels are included in the array, and only those genes with a score having an absolute value of greater than 4, greater than 5, greater than 6, greater than 7, greater than 8, greater than 9, greater than 10, greater than 12, greater than 25, greater than 27, greater than 30, or greater than 35 are selected.

In yet another embodiment, the Wilkins' metric is used to determine the significance of the differentially expressed genes whose expression levels are included in the array, and only those genes having a score of greater than 0.55, greater than 0.57, greater than 0.59, greater than 0.61, greater than 0.63, greater than 0.65, greater than 0.67, greater than 0.69, greater than 0.71, greater than 0.73, greater than 0.75, greater than 0.77, greater than 0.79, greater than 0.81, greater than 0.83, or greater than 0.85 are selected.

Each value in the expression profiles of the invention is a measurement representing the absolute or the relative expression level of a differentially expressed genes. The expression levels of these genes may be determined by any method known in the art for assessing the expression level of an RNA or protein molecule in a sample. For example, expression levels of RNA may be monitored using a membrane blot (such as used in hybridization analysis such as Northern, Southern, dot, and the like), or microwells, sample tubes, gels, beads or fibers (or any solid support comprising bound nucleic acids). See U.S. Pat. Nos. 5,770,722, 5,874,219, 5,744,305, 5,677,195 and 5,445,934, which are expressly incorporated herein by reference. The gene expression monitoring system may also comprise nucleic acid probes in solution.

In one embodiment of the invention, microarrays are used to measure the values to be included in the expression profiles. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, the Experimental section. See also, U.S. Pat. Nos. 6,040,138, 5,800,992 and 6,020,135, 6,033,860, and 6,344,316, which are incorporated herein by reference. High-density oligonucleotide arrays are particularly useful for determining the gene expression profile for a large number of RNA's in a sample.

In one approach, total mRNA isolated from the sample is converted to labeled cRNA and then hybridized to an oligonucleotide array. Each sample is hybridized to a separate array. Relative transcript levels are calculated by reference to appropriate controls present on the array and in the sample. See, for example, the Experimental section.

In another embodiment, the values in the expression profile are obtained by measuring the abundance of the protein products of the differentially-expressed genes. The abundance of these protein products can be determined, for example, using antibodies specific for the protein products of the differentially-expressed genes. The term “antibody” as used herein refers to an immunoglobulin molecule or immunologically active portion thereof, i.e., an antigen-binding portion. Examples of immunologically active portions of immunoglobulin molecules include F(ab) and F(ab′)₂ fragments which can be generated by treating the antibody with an enzyme such as pepsin.

The antibody can be a polyclonal, monoclonal, recombinant, e.g., a chimeric or humanized, fully human, non-human, e.g., murine, or single chain antibody. In a preferred embodiment it has effector function and can fix complement. The antibody can be coupled to a toxin or imaging agent.

A full-length protein product from a differentially-expressed gene, or an antigenic peptide fragment of the protein product can be used as an immunogen. Preferred epitopes encompassed by the antigenic peptide are regions of the protein product of the differentially expressed gene that are located on the surface of the protein, e.g., hydrophilic regions, as well as regions with high antigenicity. The antibody can be used to detect the protein product of the differentially expressed gene in order to evaluate the abundance and pattern of expression of the protein. These antibodies can also be used diagnostically to monitor protein levels in tissue as part of a clinical testing procedure, e.g., to, for example, determine the efficacy of a given therapy. Detection can be facilitated by coupling (i.e., physically linking) the antibody to a detectable substance (i.e., antibody labeling). Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Once the values comprised in the subject expression profile and the reference expression profile or expression profiles are established, the subject profile is compared to the reference profile to determine whether the subject expression profile is sufficiently similar to the reference profile. Alternatively, the subject expression profile is compared to a plurality of reference expression profiles to select the reference expression profile that is most similar to the subject expression profile.

Any method known in the art for comparing two or more data sets to detect similarity between them may be used to compare the subject expression profile to the reference expression profiles. In some embodiments, the subject expression profile and the reference profile are compared using a supervised learning algorithm such as the support vector machine (SVM) algorithm, prediction by collective likelihood of emerging patterns (PCL) algorithm, the k-nearest neighbor algorithm, or the Artificial Neural Network algorithm. Each of these algorithms is described in the Experimental section of the application. To determine whether a subject expression profile shows “statistically significant similarity” or “sufficient similarity” to a reference profile, statistical tests may be performed to determine whether the similarity between the subject expression profile and the reference expression profile is likely to have been achieved by a random event. An example of such a statistical test is the permutation test described in the Experimental section; however, any statistical test that can calculate the likelihood that the similarity between the subject expression profile and the reference profile results from a random event can be used. The accuracy of assigning a subject to a risk group based on similarity between an expression profile for the subject and an expression profile for the risk group depends in part on the degree of similarity between the two profiles. Therefore, when more accurate diagnoses are required, the stringency with which the similarity between the subject expression profile and the reference profile is evaluated should be increased. For example, in various embodiments, the p-value obtained when comparing the subject expression profile to a reference profile that shares sufficient similarity with the subject expression profile is less than 0.20, less than 0.15, less than 0.10, less than 0.09, less than 0.08, less than 0.07, less than 0.06, less than 0.05, less than 0.04, less than 0.03, less than 0.02, or less than 0.01.

In some embodiments, the assignment of a subject affected by leukemia to a leukemia risk group, the prediction of whether a subject affected by leukemia has an increased risk of relapse, or the prediction of whether a subject by affected by leukemia has an increased risk of developing secondary AML is used in a method of choosing a therapy for the subject affected by leukemia. A therapy, as used herein, refers to a course of treatment intended to reduce or eliminate the affects or symptoms of a disease, in this case leukemia. A therapy regiment will typically comprise, but is not limited to, a prescribed dosage of one or more drugs or hematopoietic stem cell transplantation. Therapies, ideally, will be beneficial and reduce the disease state but in many instances the effect of a therapy will have non-desirable effects as well. Thus, the methods of the invention are useful for monitoring the effectiveness of a therapy even when non-desirable side-effects are observed.

Arrays, Computer-Readable Medium, and Kits

The present invention provides compositions that are useful in determining the gene expression profile for a subject affected by leukemia and selecting a reference profile that is similar to the subject expression profile. These compositions include arrays comprising a substrate having a capture probes that can bind specifically to nucleic acid molecules that are differentially expressed in leukemia risk groups, subjects affected by leukemia who will relapse after conventional therapy, or subjects affected by leukemia who will develop secondary AML after conventional therapy. Also provided is a computer-readable medium having digitally encoded reference profiles useful in the methods of the claimed invention. The invention also encompasses kits comprising an array of the invention and a computer-readable medium having digitally-encoded reference profiles with values representing the expression of nucleic acid molecules detected by the arrays. These kits are useful for assigning a subject affected by leukemia to a leukemia risk group, predicting whether a subject affected by leukemia has an increased risk of relapse, and predicting whether a subject affected by leukemia has an increased risk of developing secondary AML.

The present invention provides arrays comprising capture probes for detecting the differentially expressed genes of the invention. By “array” is intended a solid support or substrate with peptide or nucleic acid probes attached to said support or substrate. Arrays typically comprise a plurality of different nucleic acid or peptide capture probes that are coupled to a surface of a substrate in different, known locations. These arrays, also described as “microarrays” or colloquially “chips” have been generally described in the art, for example, in U.S. Pat. Nos. 5,143,854, 5,445,934, 5,744,305, 5,677,195, 6,040,193, 5,424,186, 6,329,143, and 6,309,831 and Fodor et al. (1991) Science 251:767-77, each of which is incorporated by reference in its entirety. These arrays may generally be produced using mechanical synthesis methods or light directed synthesis methods which incorporate a combination of photolithographic methods and solid phase synthesis methods.

Techniques for the synthesis of these arrays using mechanical synthesis methods are described in, e.g., U.S. Pat. No. 5,384,261, incorporated herein by reference in its entirety for all purposes. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992, each of which is hereby incorporated in its entirety for all purposes. Arrays may be packaged in such a manner as to allow for diagnostics or other manipulation of an all-inclusive device. See, for example, U.S. Pat. Nos. 5,856,174 and 5,922,591 herein incorporated by reference.

The arrays provided by the present invention comprise capture probes that can specifically bind a nucleic acid molecule that is differentially expressed in leukemia risk groups, a nucleic acid molecule that is differentially expressed in subjects affected by leukemia who will relapse after conventional therapy, or a nucleic acid molecule that is differentially expressed in subjects affected by leukemia who will develop secondary AML after conventional therapy. These arrays can be used to measure the expression levels of nucleic acid molecules to thereby create an expression profile for use in methods of determining the diagnosis and prognosis for leukemia patients, and for monitoring the efficacy of a therapy in these patients as described elsewhere herein.

In some embodiments, each capture probe in the array detects a nucleic acid molecule selected from the nucleic acid molecules designated in Tables 2-36, 44-49, 52, 54-60, 63-68, and 70-74. The designated nucleic acid molecules include those differentially expressed in leukemia risk groups selected from the T-ALL risk group (Tables 7, 14, 21, 28, 35, 59, and 67); E2A-PBX1 risk group (Tables 3, 10, 17, 24, 31, 55, 64, and 71), TEL-AML1 risk group (Tables 8, 15, 22, 29, 36, and 60, 68, and 74), BCR-ABL risk group (Tables 2, 9, 16, 23, 30, 54, 63, and 70), MLL risk group (Tables 5, 12, 19, 26, 33, 57, 66, and 73), Hyperdiploid >50 risk group (Tables 4, 11, 18, 25, 32, 56, 65, and 72), and Novel risk group (Tables 6, 13, 20, 27, 34, and 58), those differentially expressed in subjects affected by leukemia who will relapse after conventional therapy (Tables 44-48), and those differentially expressed in subjects affected by TEL-AML1 who will develop secondary AML after conventional therapy (Table 52).

The arrays of the invention comprise a substrate have a plurality of addresses, where each addresses has a capture probe that can specifically bind a target nucleic acid molecule. The number of addresses on the substrate varies with the purpose for which the array is intended. The arrays may be low-density arrays or high-density arrays and may contain 4 or more, 8 or more, 12 or more, 16 or more, 20 or more, 24 or more, 32 or more, 48 or more, 64 or more, 72 or more 80 or more, 96, or more addresses, or 192 or more, 288 or more, 384 or more, 768 or more, 1536 or more, 3072 or more, 6144 or more, 9216 or more, 12288 or more, 15360 or more, or 18432 or more addresses. In some embodiments, the substrate has no more than 12, 24, 48, 96, or 192, or 384 addresses, no more than 500, 600, 700, 800, or 900 addresses, or no more than 1000, 1200, 1600, 2400, or 3600 addressees.

The invention also provides a computer-readable medium comprising one or more digitally-encoded expression profiles, where each profile has one or more values representing the expression of a gene that is differentially expressed in a leukemia risk group, the expression level of a gene that is differentially expressed in subjects affected by leukemia who will relapse after conventional therapy, or the expression level of a gene that is differentially expressed in subjects affected by leukemia who will develop secondary AML after conventional therapy. Such profiles are described elsewhere herein. In some embodiments, the digitally-encoded expression profiles are comprised in a database. See, for example, U.S. Pat. No. 6,308,170.

The present invention also provides kits useful for diagnosing, treating, and monitoring the disease state in subjects affected by leukemia. These kits comprise an array and a computer readable medium. The array comprises a substrate having addresses, where each address has a capture probe that can specifically bind a nucleic acid molecule that is differentially expressed in at least one leukemia risk group, in a subject affected by leukemia who will relapse after conventional therapy, or in a subject affected by leukemia who will develop secondary AML after conventional therapy. The results are converted into a computer-readable medium that has digitally-encoded expression profiles containing values representing the expression level of a nucleic acid molecule detected by the array.

Methods of Screening and Therapeutic Targets

The methods and compositions of the invention may be used to screen test compounds to identify therapeutic compounds useful for the treatment of leukemia. In one embodiment, the test compounds are screened in a sample comprising primary cells or a cell line representative of a particular leukemia risk group. After treatment with the test compound, the expression levels in the sample of one or more of the differentially-expressed genes of the invention are measured using methods described elsewhere herein. Values representing the expression levels of the differentially-expressed genes are used to generate a subject expression profile. This subject expression profile is then compared to a reference profile associated with the leukemia risk group represented by the sample to determine the similarity between the subject expression profile and the reference expression profile. Differences between the subject expression profile and the reference expression profile may be used to determine whether the test compound has anti-leukemogenic activity.

The test compounds of the present invention can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to polypeptide libraries, while the other four approaches are applicable to polypeptide, non-peptide oligomer or small molecule libraries of compounds (Lam (1997) Anticancer Drug Des. 12:145).

Examples of methods for the synthesis of molecular libraries can be found in the art, for example in DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233. Libraries of compounds may be presented in solution (e.g., Houghten (1992) Biotechniques 13:412-421), or on beads (Lam (1991) Nature 354:82-84), chips (Fodor (1993) Nature 364:555-556), bacteria (U.S. Pat. No. 5,223,409), spores (U.S. Pat. No. 5,223,409), plasmids (Cull et al. (1992) Proc. Natl. Acad. Sci. USA 89:1865-1869) or on phage (Scott and Smith (1990) Science 249:386-390); (Devlin (1990) Science 249:404-406); (Cwirla et al. (1990) Proc. Natl. Acad. Sci. U.S.A. 97:6378-6382); (Felici (1991) J. Mol. Biol. 222:301-310).

Candidate compounds include, for example, 1) peptides such as soluble peptides, including Ig-tailed fusion peptides and members of random peptide libraries (see, e.g., Lam et al. (1991) Nature 354:82-84; Houghten et al. (1991) Nature 354:84-86) and combinatorial chemistry-derived molecular libraries made of D- and/or L-configuration amino acids; 2) phosphopeptides (e.g., members of random and partially degenerate, directed phosphopeptide libraries, see, e.g., Songyang et al. (1993) Cell 72:767-778); 3) antibodies (e.g., polyclonal, monoclonal, humanized, anti-idiotypic, chimeric, and single chain antibodies as well as Fab, F(ab′)₂, Fab expression library fragments, and epitope-binding fragments of antibodies); 4) small organic and inorganic molecules (e.g., molecules obtained from combinatorial and natural product libraries; 5) zinc analogs; 6) leukotriene A₄ and derivatives; 7) classical aminopeptidase inhibitors and derivatives of such inhibitors, such as bestatin and arphamenine A and B and derivatives; 8) and artificial peptide substrates and other substrates, such as those disclosed herein above and derivatives thereof.

The present invention discloses a number of genes that are differentially expressed in leukemia risk groups, in subjects affected by leukemia who will relapse after conventional therapy, or in subjects affected by leukemia who will develop secondary AML after conventional therapy. These differentially-expressed genes are shown in Tables 2-36 and 44-48, and 52. Because the expression of these genes is associated with leukemia risk factors, these genes may play a role in leukemogenesis. Accordingly, these genes and their gene products are potential therapeutic targets that are useful in methods of screening test compounds to identify therapeutic compounds for the treatment of leukemia.

The differentially-expressed genes of the invention may be used in cell-based screening assays involving recombinant host cells expressing the differentially-expressed gene product. The recombinant host cells are then screened to identify compounds that can activate the product of the differentially-expressed gene (i.e. agonists) or inactivate the product of the differentially-expressed gene (i.e. antagonists).

Any of the leukemogenic functions mediated by the product of the differentially expressed gene may be used as an endpoint in the screening assay for identifying therapeutic compounds for the treatment of leukemia. Such endpoint assays include assays for cell proliferation, assays for modulation of the cell cycle, assays for the expression of markers indicative of leukemia, and assays for the expression level of genes differentially expressed in leukemia risk groups as described above.

Modulators of the activity of a product of a differentially-expressed gene identified according to these drug screening assays provided above can be used to treat a subject with leukemia. These methods of treatment include the steps of administering the modulators of the activity of a product of a differentially-expressed gene in a pharmaceutical composition as described herein, to a subject in need of such treatment.

The following examples are offered by way of illustration and not by way of limitation.

### EXAMPLES

### Example 1

To determine if gene expression profiling of leukemic cells could identify known biologic ALL subgroups, 327 diagnostic bone marrow (BM) samples were analyzed with AFFYMETRIX® oligonucleotide microarrays (Affymetrix Inc., Santa Clara, Calif.) containing 12,600 probe sets.

In an initial analysis of the gene expression data set (12,600 probe sets in 327 leukemia samples; greater than 4×10⁶ data elements), an unsupervised two-dimensional hierarchical clustering algorithm was used to group leukemia samples with similar gene expression patterns against clusters of similarly expressed genes. This analysis clearly identified 6 major leukemia subtypes that corresponded to T-ALL, hyperdiploid with >50 chromosomes, BCR-ABL, E2A-PBX1, TEL-AML1, and MLL gene rearrangement. Moreover, within the heterogeneous collection of leukemias that were not assigned to one of these subtypes, a novel subgroup of 14 cases was identified that had a distinct gene expression profile. The separation of these seven leukemia subgroups was also seen using the multidimensional scaling procedure of discriminant analysis with variance (DAV), in which the data are reduced into component dimensions consisting of linear combinations of discriminating genes. For example, using the three component dimensions that accounted for 72.8% of the variance of gene expression among the subgroups, it was possible to distinguish T-ALL (43 cases), E2A-PBX1 (27 cases), TEL-AML1 (79 cases) and hyperdiploid >50 (64 cases) from the remaining ALL subtypes (114 cases). Similarly, using three different components that account for an additional 16.1% of the variance in gene expression mad it possible to discriminate cases with BCR-ABL (15 cases), MLL gene rearrangement (20 cases) and the novel subgroup of ALL (14 cases).

Statistical methods were used to identify those genes that best define the individual groups. Expression profiles were obtained using the top 40 genes per subgroup as selected by a Chi square metric. Distinct groups of genes distinguish cases defined by E2A-PBX1, MLL, T-ALL, hyperdiploid >50, BCR-ABL, the novel subgroup, and TEL-AML1. In addition to these specific subgroups, 65 cases (20% of the total) were identified that did not cluster into any of the leukemia subtypes. The expression profiles of these latter cases varied markedly, suggesting that they represent a heterogeneous group of leukemias. Nearly identical results were obtained when the hierarchical clustering was performed with genes selected by other statistical metrics.

For T-ALL, two gene clusters that discriminated this subtype from B-lineage cases were identified. One cluster was expressed at high and one cluster was expressed at low levels. In contrast the top ranked discriminating genes for each of the other leukemia subtypes consisted primarily of genes that were overexpressed within the specific leukemia subtype. With the exception of T-ALL, the identified expression profiles do not represent a specific differentiation stage of the leukemic blasts. For example, although E2A-PBX1 is almost exclusively found in ALLs with a pre-B cell immunophenotype (Hunger (1996) Blood 87:1211-24), the identified expression profile was specific for the E2A-PBX1 genetic lesion and not the pre-B immunophenotype.

To confirm that the microarray analysis provided an accurate reflection of actual gene expression levels, the microarray data was compared with results for RNA levels obtained by real-time RT-PCR (5 genes). In addition, the corresponding protein levels were assessed by immunophenotype analysis performed by flow cytometry using nine specific cell surface antigens). A very high degree of correlation was observed between the levels of RNA expression detected by quantitative RT-PCR and microarray analysis. Similarly, in agreement with results from immunophenotying, T-lineage restricted RNA expression was observed for CD2, CD3, and CD8, whereas B-lineage restricted expression was observed for CD19, and CD22. In addition, the level of CD10 RNA expression closely correlated with protein levels, with high expression detected in TEL-AML1 leukemias, intermediate levels in E2A-PBX1 and low to undetectable expression in cases with rearrangements of MLL. Thus, microarray analysis provides an accurate reflection of expression levels for most genes, and can be used to accurately detect the expression of the more common surface antigens used in the diagnostic evaluation of pediatric ALL patients.

The majority of the leukemia subtype specific genes identified through this study were not previously known to have a restricted pattern of expression. In addition to their use as diagnostic and subclassification markers, these genes provide unique insights into the underlying biology of the different leukemia subtypes. For example, E2A-PBX1 leukemias were characterized by high expression of the c-Mer receptor tyrosine kinase (MERTK), a known transforming gene (Graham et al. (1994) Cell Growth Differ. 5:647-657); and Georgescu et al. (1999) Mol. Cell. Biol. 19:1171-81), suggesting that C-MER may be involved in the abnormal growth of these cells. Similarly, HOXA9 and MEIS1 were exclusively expressed in cases having MLL rearrangements, indicating that they may be directly involved in MLL mediated alterations in the growth of the leukemic cells. Interestingly, high expression of MTG16, a homologue of ETO (Gamou et al. (1998) Blood 91:4028-4037), was found in TEL-AML1 cases. Alteration of ETO family members in both t(8;21) acute myeloid leukemia (by translocation) (Downing (1999) Br. J. Hematol. 106:296-308) and TEL-AML1 (by altered expression) suggests that alteration in the biologic function of ETO genes is mechanistically involved in these leukemias. Little is known about the underlying molecular pathogenesis of hyperdiploid ALL >50 chromosomes, which clinically is distinct from hyperdiploid cases having 47-50 chromosomes. This distinction is supported by the marked differences in gene expression profiles between these two subgroups. Although hyperdiploid >50 ALLs have an excellent prognosis, the specific genetic lesions responsible for the aberrant proliferation in these cases remains poorly understood. Interestingly, almost 70% of the genes that define this subgroup are localized to either chromosome X or 21. Moreover, the class defining genes on chromosome X were overexpressed in the hyperdiploid >50 chromosomes ALLs irrespective of whether the leukemic blasts had a trisomy of this chromosome (data not shown). Detailed analysis will be required to determine the specific signaling pathways that are disrupted as a result of the altered expression of these genes. Lastly, the novel subgroup of ALL was defined by high expression of a group of genes, including the receptor phosphatase PTPRM, and LHFPL2, a gene that is a part of the LHFP-like gene family, the founding member of which was identified as the target of a lipoma-associated chromosomal translocation (Petit et al. (1999) Genomics 57:438-41).

Expression Profiling as a Diagnostic Tool

A major goal of this study was to develop a single platform of expression profiling to accurately identify the known, prognostically important leukemia subtypes. To this end, computer-assisted learning algorithms were used to develop an expression-based leukemia classification. Through a reiterative process of error minimization, these algorithms learn to recognize the optimal gene expression patterns for a leukemia subtype. Classification was approached using a decision tree format, in which the first decision was T-ALL versus B-lineage (non-T-ALL), and then within the B-lineage subset, cases were sequentially classified into the known risk groups characterized by the presence of E2A-PBX1, TEL-AML1, BCR-ABL, MLL chimeric genes, and lastly hyperdiploid with >50 chromosomes. Cases not assigned to one of these classes were left unassigned. Classification was performed using a Support Vector Machine (SVM) algorithm with a set of discriminating genes selected by a correlation-based feature selection (CFS), or if this method selected greater than 20 genes for a particular class, by using the top 20 ranked genes selected by a chi-square metric, or one of the other metrics detailed in the Experimental Procedures section. This approach resulted in an accurate class prediction in a randomly selected training set that consisted of two-thirds of the total cases (215 cases). When this classification model was then applied to a blind test set consisting of the remaining 112 samples, an overall accuracy of 96% was achieved for class assignment. The number of genes required for optimal class assignment varied between classes. A single gene was sufficient to give 100% accuracy for both T-ALL and E2A-PBX1, whereas 7-20 genes were required for prediction of the other classes. Only slight differences were observed in the prediction accuracy of individual classes when the process was repeated using genes selected by a number of other metrics, including T-statistics, a novel metric referred to as Wilkins', or genes selected by a combination of self organizing maps (SOM) and DAV. Moreover, nearly identical results were obtained when the various sets of selected genes were used in a number of different supervised learning algorithms, including κ-Nearest Neighbor (κ-NN), Artificial Neural Network (ANN), and prediction by collective likelihood of emerging patterns (PCL).

Four cases initially appeared to be misclassified as TEL-AML1 by gene expression analysis since they lacked a detectable chimeric transcript by RT-PCR. Upon further analysis by FISH, however, one of these cases was shown to have a TEL-AML1 fusion, presumably, a variant rearrangement that could not be detected with the amplification primers used for the TEL-AML1 RT-PCR assay. In each of the three remaining cases, re-examination of the karyotypes revealed translocations involving the p arm of chromosome 12. FISH analysis demonstrated that two of these cases had deletion of one TEL allele, whereas the remaining case had a partial deletion of one TEL allele. Thus, the identified expression profiles appear to reflect an abnormality of the TEL transcription factor, and may in fact provide a more accurate means of identifying a specific leukemia subtype defined by its underlying biology. Collectively, these data demonstrate that the single platform of gene expression profiling can accurately identify the known prognostic subtypes of ALL.

Use of Expression Profiles to Identify Patients at High Risk of Treatment Failure

Relapse and the development of therapy-induced acute myeloid leukemia (AML) are the major causes of treatment failure in pediatric ALL. To determine if expression profiling might further enhance the ability to identify patients who are likely to relapse, the expression profiles of the four groups of leukemic samples were compared. The groups of samples used for this comparison were: 1)diagnostic samples of patients that developed hematological relapses (n=32); (ii) diagnostic samples from patients who remained in continuous complete remission (CCR) (n=201); (iii) diagnostic samples from patients who developed therapy-induced AML (n=16); and (iv) leukemic samples collected at the time of ALL relapse (n=25). Using DAV, distinct gene expression profiles were identified for each of these groups.

To further assess the predictive power of the different gene expression profiles, supervised learning algorithms were used. Because of the overwhelming differences in the expression profiles of the different leukemia subtypes, it was not possible to identify a single expression signature that would predict relapse irrespective of the genetic subtype. However, within individual leukemic subtypes, distinct expression profiles could be defined that predicted relapse. Class assignment was performed using a SVM supervised learning algorithm with discriminating genes selected by CFS, or if this method returned >20 genes, the top 20 genes selected by T-statistics. For both the T-lineage and hyperdiploid >50 subgroups, expression profiles identified those cases that went on to relapse with an accuracy of 97% and 100%, respectively, as assessed by cross validation. Moreover, the predictive accuracy was statistically significant when compared to results from an analysis of 1000 random permutations of the specific patient data set. Similarly, expression profiles predictive of relapse were identified for TEL-AML, MLL, or cases that lacked any of the known genetic risk features. Although the predictive accuracy of these latter expression profiles was very high as assessed by cross validation, it did not reach statistical significance when compared to results from an analysis of 1000 random permutations of the same patient data set, likely secondary to the limited number of cases. The patterns of expression for a combination of genes, rather than expression levels of a single gene were found to have the greatest predictive accuracy. Since few known risk-stratifying biologic features have been previously identified for either T-ALL or hyperdiploid >50 ALL, the results suggest that the identified expression profiles provide independent risk stratifying information.

A distinct expression profile was identified in the ALL blasts from patients who developed therapy-induced AML. Because secondary AML is thought to arise from a hematopoietic stem cell that is distinct from that giving rise to the primary leukemia, it is difficult to understand how the biology of the original ALL blasts could predict the risk of developing a therapy-induced complication. However, when the accuracy of expression profiling was evaluated in within the TEL-AML1 subgroup, a distinct expression signature consisting of 20 genes was defined. This profile identified, with 100% accuracy in cross validation, all patients who developed secondary AML, with a p value of 0.031 as assessed by comparison to results from an analysis of 1000 random permutations of the patient data set. Genes within this signature included RSU1, a suppressor of the Ras signaling pathway, and Msh3, a mismatch repair enzyme.

Overview of Experimental Procedures

A. Tumor Samples

The diagnosis of ALL was based on the morphologic evaluation of the bone marrow and on the pattern of reactivity of the leukemic blasts with a panel of monoclonal antibodies directed against lineage-associated antigens. A total of 389 pediatric acute leukemia samples were analyzed in this study, from which high quality gene expression data was obtained on 360 (93%). The successfully-analyzed samples included 332 diagnostic BM, 3 diagnostic peripheral bloods (PB), and 25 relapsed ALL samples from BM or PB. 264 (79%) of the diagnostic ALL BM samples and all relapse samples were from patients enrolled on St. Jude Children's Research Hospital Total Therapy Studies XIIIA or XIIIB and corresponded to 64% of the patients treated on these protocols. The details of these protocols have been previously published (Pui et al. (2000) Leukemia 14:2286-94). The remaining samples were obtained from patients treated on St. Jude Total Therapy Studies XI, XII, XIV, XV, or by best clinical management. All protocols and consent forms were approved by the hospital's institutional review board, and informed consent was obtained from parents, guardians, or patients (as appropriate). The composition of the data sets used for the identification of gene expression profiles predictive of specific genetic subtypes, hematological relapse, and risk of developing secondary AML are described below.

B. Gene Expression Profiling

RNA was extracted from cryopreserved mononuclear cell suspensions from diagnostic BM aspirates or PB samples using TRIZOL® (Invitrogen Corp., Carlsbad, Calif.) according to the manufacturer's instructions, and the RNA integrity was assessed by using an Agilent 2100 Bioanalyzer (Agilent Technologies, Palo Alto, Calif.). cDNA was synthesized using a T-7 linked oligo-dT primer and cRNA was then synthesized with biotinylated UTP and CTP. The labeled RNA was then fragmented and hybridized to HG_U95Av2 oligonucleotide arrays (Affymetrix Incorporated, Santa Clara, Calif.) according to the manufacturer's instructions.

Arrays were scanned using a laser confocal scanner (Agilent) and the expression value for each gene was calculated using AFFYMETRIX® Microarray Software version 4.0. The average intensity difference (AID) values were normalized across the sample set and minimum quality control standards were established for including a sample's hybridization data in the study. 10% of samples were run in duplicate to ensure consistency of data acquisition throughout the study. A high level of reproducibility was observed between replicate samples, with fewer than 1% of genes showing a variation in average intensity difference of greater than 2-fold.

C. Statistical Analysis

Unsupervised hierarchical clustering, principal component analysis (PCA), discriminant analysis with variance (DAV), and self organizing maps (SOM) were performed using GeneMaths software (version 1.5, Applied Maths, Belgium). Data reduction to define the genes most useful in class distinction was performed using a variety of metrics as detailed below. Genes selected by the various metrics were used in supervised learning algorithms to build classifiers that could identify the specific genetic or prognostic subgroups. The algorithms used included k-Nearest Neighbors (k-NN), Support Vector Machine (SVM), prediction by collective likelihood of emerging patterns (PCL), an artificial neural network (ANN), and weighted voting. Performance of each model was initially assessed by leave-one-out cross validation on a randomly selected stratified training set consisting of two-thirds of the total cases. True error rates of the best performing classifiers were then determined using the remaining third of the samples as a blinded test group. Details of the individual metrics and supervised learning algorithms are described below.

Detailed Experimental Procedures

A. RNA Extraction, Labeling, Hybridization, and Data analysis

Mononuclear cell suspensions from diagnostic BM aspirates or peripheral blood (PB) samples were prepared from each patient and an aliquot was cryopreserved. RNA was extracted using TRIZOL® following the manufacture's recommended protocol as described above. RNA integrity was assessed by electrophoresis on the Agilent 2100 Bioanalyzer (Agilent, Palo Alto, Calif.).

First and second strand cDNA were synthesized from 5-15 μg of total RNA using the SuperScript Double-Stranded cDNA Synthesis Kit ((Invitrogen Corp., Carlsbad, Calif.) and an oligo-dT₂₄-T7 (5′-GGC CAG TGA ATT GTA ATA CGA CTC ACT ATA GGG AGG CGG-3′; SEQ ID NO: 1) primer according to the manufacturer's instructions. cRNA was synthesized and labeled with biotinylated UTP and CTP by in vitro transcription using the T7 promoter coupled double stranded cDNA as template and the T7 RNA Transcript Labeling Kit according the manufacturer's instructions (Enzo Diagnostics Inc., Farmingdale N.Y.). Briefly, double stranded cDNA synthesized from the previous steps was washed twice with 70% ethanol and resuspended in 22 μl RNase-free water. The cDNA was incubated with 4 μl of 10× each reaction buffer, 1 μl of biotin labeled ribonucleotides, 2 μl of DTT, 1 μl of RNase inhibitor mix and 2 μl 20× T7 RNA polymerase for 5 hours at 37° C. The labeled cRNA was separated from unincorporated ribonucleotides by passing through a CHROMA SPIN-100 column (Clontech, Palo Alto, Calif.) and precipitated at −20° C. for 1 hr to overnight.

The cRNA pellet was resuspended in 10 μl Rnase-free H₂O and 10.0 μg was fragmented by heat and ion-mediated hydrolysis at 95° C. for 35 minutes in 200 mM Tris-acetate, pH 8.1, 500 mM KOAc, 150 mM MgOAc. The fragmented cRNA was hybridized for 16 hr at 45° C. to HG_U95Av2 AFFYMETRIX® oligonucleotide arrays (Affymetrix, Santa Clara, Calif.) containing 12,600 probe sets from full-length annotated genes together with additional probe sets designed to represent EST sequences. Arrays were washed at 25° C. with 6×SSPE (0.9M NaCl, 60 mM NaH₂PO₄, 6 mM EDTA, 0.01% Tween 20) followed by a stringent wash at 50° C. with 100 mM MES, 0.1M NaCl₂, 0.01% Tween 20. The arrays were then stained with phycoerythrin conjugated streptavidin (Molecular Probes, Eugene, Oreg.).

Arrays were scanned using a laser confocal scanner (Agilent, Palo Alto, Calif.) and the expression value for each gene was calculated using AFFYMETRIX® Microarray software (MAS 4.0). The signal intensity for each gene was calculated as the average intensity difference (AID), represented by [Σ(PM−MM)/(number of probe pairs)], where PM and MM denote perfect-match and mismatch probes, respectively. Expression values were normalized across the sample set by scaling the average of the fluorescent intensities of all genes on an array to a constant target intensity of 2500, then any AID over 45,000 was capped to a value of 45,000. All AID's less than 100, including negative values and absent calls were converted to a value of 1. In addition, a variation filter was used to eliminate any probe set in which fewer than 1% of the samples had a present call, or if the Max AID−Min AID across the sample set was less than 100. The average intensity differences for each of the remaining genes were analyzed. For some metrics the data was log transformed prior to analysis. The minimum quality control values required for inclusion of a sample's hybridization data in the study were 10% or greater present calls, a GAPDH/Actin 3′/5′ ratio<5, and use of a scaling factor that was within 3 standard deviations from the mean of the scaling values of all chips analyzed.

The average percent present calls for theoverall data set was 29.7%, and for each of the genetic subgroups was BCR-ABL (31.1%), E2A-PBX1 (28.9%), Hyper >50 (31%), MLL (29.8%), T-ALL (29.1%), TEL-AML1 (28.5%), Novel (30.2%), others (31.1%). In addition, each sample had >75% blasts. The average percentage blasts for the overall data set used to define the genetic subtypes was 93%, and for each genetic subtype was BCR-ABL (92%), E2A-PBX1 (96%), Hyper >50 (93%), MLL (93%), T-ALL (91%), TEL-AML1 (92%), Novel (95%), and others (94%).

B Reproducibility of Microarray Data

The reproducibility of the AFFYMETRIX® microarray system was assessed by comparing the gene expression profiles of RNA extracted from duplicate cryopreserved diagnostic leukemic samples from 23 patients with single RNA samples from 13 patients analyzed on two separate arrays. The mean number of probe sets that displayed a ≧2-fold difference in expression between separately extracted but paired RNA samples was 144, and for single RNA samples analyzed on two separate occasions was 133. Moreover, very few probe sets were found to have a ≧3-fold difference in expression levels between replicate samples. The observed number of probe sets showing a difference in expression values represents less than 2% of the total number of probe sets on the microarray, and thus these data suggest that the AFFYMETRIX® microarray system has a very high degree of reproducibility.

C. Comparison of Expression Profiles from PB and BM leukemia samples

Matched BM and PB samples that contained ≧80% leukemic blasts were obtained from 10 patients and the RNA was extracted and assessed by microarray analysis. A very high level of correlation was observed between the expression profiles of BM and PB, with only 189 probe sets having a greater than a 2-fold difference in expression. No genes were found to be consistently over- or under-expressed in one sample type. These data demonstrate that there are minimal differences in the gene expression profiles of leukemic blasts obtained from BM or PB, and that diagnostic gene expression profiling is possible on samples obtained from the PB.

D. RT-PCR Results

Real-time TAQMAN® RT-PCR assays (Applied Biosystems, Foster City, Calif.) were performed to independently determine the level of mRNA for five genes that were found by microarray analysis to be predictive of either T-lineage ALL (CD3δ, CD3D antigen delta polypeptide TiT3 complex; MAL, mal T-Cell differentiation protein; and PRKCQ, protein kinase C theta) or E2A-PBX1 expressing ALL (MERTK, c-Mer proto-oncogene tyrosine kinase and KIAA802). The RNA samples analyzed included four samples each of E2A-PBX1 and T-ALL, and two samples each from the remaining subtypes (BCR-ABL, MLL, TEL-AML1, Hyperdiploid >50, Hyperdiploid 47-50, Hypodiploid, Pseudodiploid, and normal). Whenever possible, the forward and reverse primers were designed in different exons so that DNA contamination would not be a concern. In the case of MAL where this was not clear, the RNA was treated for 15 minutes at room temperature with 1.0 unit of DNase I (Invitrogen Corp., Carlsbad, Calif.) using the Invitrogen protocol to remove any contaminating DNA.

Thirty-three ng of RNA from each sample was reverse transcribed using random hexamers and Multiscribe Reverse Transcriptase (Applied Biosystems, Foster City, Calif.) in a total volume of 10 μl. Real time PCR was performed on a Applied Biosystems PRISM® 7700 Sequence Detection System (Applied Biosystems). All probes were labeled at the 5′ end with FAM (6-carboxy-fluroescein) and at the 3′ end with TAMRA (6-carboxy-tetramethyl-rhodamine).

The PCR reactions were performed in a total volume of 50 μl containing 10 μl of the reverse transcriptase product, 300 nM each of the forward and reverse primers, 100 nM of probe, 1× master mix and 1 μl of AMPLITAQ GOLD® DNA polymerase (Applied Biosystems). Following a 10 minute incubation at 95° C. to activate the polymerase, samples were denatured at 95° C. for 15 seconds, then annealed and extended at 60° C. for 1 minute, for a total of 40 cycles. The RNA from each sample was also amplified using primers and probes to RNase P (Applied Biosystems) for use in normalization according to the manufacturer's instructions. Negative controls were included in each run. Standard curves were generated for T-cell markers and RNase P using MOLT4 RNA, a T-cell leukemia cell line, and for the E2A-PBX1 markers and RNase P using a leukemia cell line, 697, that contains an E2A-PBX1 fusion.

The expression level of the predictive genes and RNase P were determined in each of the 24 ALL samples. A ratio was then calculated by taking the expression value for the specific gene and dividing it by the expression level of RNase P in the sample. These ratios were then compared to the values obtained from the AFFYMETRIX® chip data from the same RNA sample. The raw AFFYMETRIX® chip data were scaled as described and then normalized using the 3′GAPDH value for each sample, yielding a normalized ratio. The TAQMAN® results and AFFMETRIX® chip ratios were then log transformed and compared. Since the markers selected for TAQMAN® analysis were predictors for either E2A-PBX1 or T-ALLs, each gene was expected to have four RNA samples with high and 20 samples with low expression. For each gene evaluated, an average expression value for both the TAQMAN® results and AFFYMETRIX® data was calculated for all samples in the up-regulated group, and similarly, for the samples in the down-regulated group.

E. Comparison of Real-time RT-PCR Data and AFFYMETRIX® Chip Data

The normalized gene expression ratios for the TAQMAN® data (gene/RNase P) and for the AFFYMETRIX® microarray data (AID for a gene/AID for GAPDH) were log transformed and then the average expression values for each gene was calculated in the four samples in which its expression was expected to be up-regulated and separately in the 20 samples in which its expression was expected to be down-regulated. For example, for genes that were expected to be up-regulated in T-ALL (CD3δ, MAL, and PRKCQ), the log expression ratios in the T-ALL samples were averaged to give the up regulated values and the log expression ratios of each gene in the non-T-ALL cases were averaged to give the down regulated value.

In both the TAQMAN® and the microchip array analysis, MERTK and KIAA802, were very highly expressed in the diagnostic samples containing E2A-PBX1, and expressed at low levels in all of the other samples. Likewise, PRKCQ, CD3δ, and MAL, showed high levels of expression in T cells by both methodologies in comparison with non T-cells. The normalized ratios from the TAQMAN® assay were plotted against the normalized ratios from the microchip array for both the up-regulated and down-regulated genes. The correlation between TAQMAN® results and the microchip array results was 70%, indicating that the same pattern of gene expression was seen in both analyses. The MERTK was extremely high in two of the E2A-PBX1 patient samples by TAQMAN® analysis. Removal of the MERTK gene from the analysis resulted in a correlation of 91% between the TAQMAN® results and the microchip array results.

F. Comparison of AFFYMETRIX® Microarray Chip Results and Immunophenotype Results

Leukemic blasts at the time of diagnosis were analyzed for expression of lineage restricted cell surface antigens using phycoerythrin- or fluorescein isothiocyanate-conjugated monoclonal antibodies against CD2, CD3ε, CD4, CD5, CD7, CD8, CD10, CD19, and CD22 (Becton Dickinson Immunocytometry Systems, San Jose, Calif., USA). Data were obtained using a COULTER® EPICS XL™ (Beckman Coulter, Miami, Fla.), a COULTER® ELITE™ (Beckman Coulter), or a BD FACSCalibur™ flow cytometer (Becton Dickinson, San Jose, Calif.) . The expression patterns for these antigens were then compared to gene expression patterns for the AFFYMETRIX® chip sites specified for CD2 (1 probe set, 40738_at), CD3δ(1 probe set, 38319_at), CD3ε(1 probe set, 36277_at), CD3ζ(1 probe set, 37078_at), CD3γ(1 probe set, 39226_at), CD4 (5 probe sets, 856_at, 1146_at, 35517_{‘at,} 34003_at, and 37942_at), CD5 (1 probe set, 32953_at), CD7 (1 probe set, 771_s_at), CD8α(1 probe set, 40699_at), CD8β(1 probe set, 39239_at), CD10 (1 probe set, 1389_at), CD19 (2 probe sets, 1096_g_at and 1116_at), and CD22 (2 probe sets, 38521_at and 38522_s_at). As a control, the performance of the AFFYMETRIX® microarray probe sets were also assessed using RNA isolated from flow sorted single positive CD4+ and CD8+ thymocytes, and CD10+/CD19+ bone marrow cells. High RNA expression was observed in T-ALL for the T-lineage restricted genes CD2, CD3δ, ε, and ζ, CD8α, and CD7, and in B-lineage ALLs for the B-cell restricted genes CD19, and CD22. A similar high level of correlation was observed between RNA and protein expression for CD10. The observed low expression levels of T-cell restricted genes in B-cell cases, and B-cell restricted genes in T-ALLs, is consistent with the low level of normal contaminating lymphocytes present in the diagnostic marrow samples analyzed.

G. Patient Data Set

A total of 389 Pediatric acute leukemia samples were analyzed in this study, from which high quality gene expression data were obtained on 360 (93%). The successfully analyzed samples included: 332 diagnostic bone marrows (BM), 3 diagnostic peripheral blood samples (PB), and 25 relapse ALL samples from BM or PB. 264 (79%) of the diagnostic ALL BM samples and all relapse samples were from patients treated on St. Jude Children's Research Hospital Total Therapy Studies XIIIA or XIIIB and correspond to 64% of the patients treated on these protocols. The details of these protocols are described in Pui et al., “Risk-adapted treatment for acute lymphoblastic leukemia: findings from St. Jude Children's Research Hospital,” Haematology and Blood Transfusions, 1997, pp 629-37, Springer-Verlag, Berlin and in Pui et al. (2000) Leukemia 14:2286-94. Study XIIIA ran from Dec. 20, 1991 to Aug. 23, 1994 and enrolled 165 patients, whereas Study XIIIB ran from Aug. 24, 94 to Jul. 27, 1998 and enrolled 247 patients. No patients were lost to follow-up during treatment. When the databases were frozen for analysis, 100% and 93% of event-free survivors in studies XIIIA and XIIIB, respectively, had been seen within 12 months. The median (minimum, maximum) follow-up of the event-free survivors was 8.09 (6.59, 9.94) and 4.52 (2.37, 7.06) years for XIIIA and XIIIB, respectively. All other samples were obtained from patients treated on St. Jude Total Therapy Studies XI, XII, XIV, XV, or by best clinical management.

For the identification of gene expression profiles that predict specific genetic subtypes of ALL, 327 diagnostic BM samples were used. The criteria for inclusion in this data set were the availability of a cryopreserved diagnostic BM sample containing ≧75% blasts, and complete data from each of the following diagnostic studies: morphology, immunophenotype, cytogenetics, DNA ploidy, Southern blot for MLL gene rearrangements, and RT-PCR analysis for MLL-AF4, MLL-AF9, E2A-PBX1, TEL-AML1, and BCR-ABL. This final data set includes diagnostic BM samples from XV (38), XIV (4), XIIIA (100), XIIIB (161), or from patients treated on one of our older protocols or by best clinical management (24).

The data sets used to identify expression profiles predicative of hematologic relapse and the development of therapy-induced AML are described in Table 1.

TABLE 1
Patient Database
Diagnostic samples used for subtype classification (n = 327)

| | | |
|---|---|---|
| Label^{@} | Protocol^{#} | Outcome^{%} |

BCR-ABL subgroup (n = 15)

| | | |
|---|---|---|
| BCR-ABL-C1 | T13B | CCR |
| BCR-ABL-R1 | T13A | Heme Relapse |
| BCR-ABL-R2 | T13A | Heme Relapse |
| BCR-ABL-R3 | T13B | Heme Relapse |
| BCR-ABL- | T13B | Heme Relapse |
| Hyperdip-R5 | | |
| BCR-ABL-#1 | T13A | Censored |
| BCR-ABL-#2 | T13B | Censored |
| BCR-ABL-#3 | T13B | Censored |
| BCR-ABL-#4 | T11 | NA |
| BCR-ABL-#5 | T12 | NA |
| BCR-ABL-#6 | T12 | NA |
| BCR-ABL-#7 | T12 | NA |
| BCR-ABL-#8 | T14 | NA |
| BCR-ABL-#9 | T15 | NA |
| BCR-ABL-Hyperdip-#10 | T12 | NA |

E2A-PBX1 subgroup (n = 27)

| | | |
|---|---|---|
| E2A-PBX1-C1 | T13A | CCR |
| E2A-PBX1-C2 | T13A | CCR |
| E2A-PBX1-C3 | T13A | CCR |
| E2A-PBX1-C4 | T13A | CCR |
| E2A-PBX1-C5 | T13A | CCR |
| E2A-PBX1-C6 | T13B | CCR |
| E2A-PBX1-C7 | T13B | CCR |
| E2A-PBX1-C8 | T13B | CCR |
| E2A-PBX1-C9 | T13B | CCR |
| E2A-PBX1-C10 | T13B | CCR |
| E2A-PBX1-C11 | T13B | CCR |
| E2A-PBX1-C12 | T13B | CCR |
| E2A-PBX1-R1 | T13B | Heme Relapse |
| E2A-PBX1-2M#1 | T13B | 2nd AML |
| E2A-PBX1-#1 | Others | NA |
| E2A-PBX1-#2 | Others | NA |
| E2A-PBX1-#3 | Others | NA |
| E2A-PBX1-#4 | Others | NA |
| E2A-PBX1-#5 | Others | NA |
| E2A-PBX1-#6 | Others | NA |
| E2A-PBX1-#7 | T11 | NA |
| E2A-PBX1-#8 | T11 | NA |
| E2A-PBX1-#9 | T12 | NA |
| E2A-PBX1-#10 | T12 | NA |
| E2A-PBX1-#11 | T14 | NA |
| E2A-PBX1-#12 | T15 | NA |
| E2A-PBX1-#13 | T15 | NA |

Hyperdip >50 subgroup (n = 64)

| | | |
|---|---|---|
| Hyperdip >50-C1 | T13A | CCR |
| Hyperdip >50-C2 | T13A | CCR |
| Hyperdip >50-C3 | T13A | CCR |
| Hyperdip >50-C4 | T13A | CCR |
| Hyperdip >50-C5 | T13A | CCR |
| Hyperdip >50-C6 | T13A | CCR |
| Hyperdip >50-C7 | T13A | CCR |
| Hyperdip >50-C8 | T13A | CCR |
| Hyperdip >50-C9 | T13A | CCR |
| Hyperdip >50-C10 | T13A | CCR |
| Hyperdip >50-C11 | T13A | CCR |
| Hyperdip >50-C12 | T13A | CCR |
| | | Relapse |
| Hyperdip >50-C13 | T13A | CCR |
| | | Relapse |
| Hyperdip >50-C14 | T13A | CCR |
| | | Relapse |
| Hyperdip >50-C15 | T13B | CCR |
| | | Relapse |
| Hyperdip >50-C16 | T13B | CCR |
| | | Relapse |
| Hyperdip >50-C17 | T13B | CCR |
| Hyperdip >50-C18 | T13B | CCR |
| Hyperdip >50-C19 | T13B | CCR |
| Hyperdip >50-C20 | T13B | CCR |
| Hyperdip >50-C21 | T13B | CCR |
| Hyperdip >50-C22 | T13B | CCR |
| Hyperdip >50-C23 | T13B | CCR |
| Hyperdip >50-C24 | T13B | CCR |
| Hyperdip >50-C25 | T13B | CCR |
| Hyperdip >50-C26 | T13B | CCR |
| Hyperdip >50- | T13B | CCR |
| C27-N | | |
| Hyperdip >50-C28 | T13B | CCR |
| Hyperdip >50-C29 | T13B | CCR |
| Hyperdip >50-C30 | T13B | CCR |
| Hyperdip >50-C31 | T13B | CCR |
| Hyperdip >50-C32 | T13B | CCR |
| Hyperdip >50-C33 | T13B | CCR |
| Hyperdip >50-C34 | T13B | CCR |
| Hyperdip >50-C35 | T13B | CCR |
| Hyperdip >50-C36 | T13B | CCR |
| Hyperdip >50-C37 | T13B | CCR |
| Hyperdip >50-C38 | T13B | CCR |
| Hyperdip >50-C39 | T13B | CCR |
| Hyperdip >50-C40 | T13B | CCR |
| Hyperdip >50-C41 | T13B | CCR |
| Hyperdip >50-C42 | T13B | CCR |
| Hyperdip >50-C43 | T13B | CCR |
| Hyperdip >50-R1 | T13A | Heme |
| Hyperdip >50-R2 | T13A | Heme |
| Hyperdip >50-R3 | T13A | Heme |
| Hyperdip >50-R4 | T13B | Heme |
| Hyperdip >50-R5 | T13B | Heme |
| Hyperdip >50-2M#1 | T13A | 2nd AML |
| Hyperdip >50-2M#2 | T13B | 2nd AML |
| Hyperdip >50-#1 | T13A | Censored |
| Hyperdip >50-#2 | T13B | Censored |
| Hyperdip >50-#3 | Others | NA |
| Hyperdip >50-#4 | Others | NA |
| Hyperdip >50-#5 | T12 | NA |
| Hyperdip >50-#6 | T15 | NA |
| Hyperdip >50-#7 | T15 | NA |
| Hyperdip >50-#8 | T15 | NA |
| Hyperdip >50-#9 | T15 | NA |
| Hyperdip >50-#10 | T15 | NA |
| Hyperdip >50-#11 | T15 | NA |
| Hyperdip >50-#12 | T15 | NA |
| Hyperdip >50-#13 | T15 | NA |
| Hyperdip >50-#14 | T15 | NA |

Hyperdip 47-50 subgroup (n = 23)

| | | |
|---|---|---|
| Hyperdip 47-50- | 13A | CCR |
| C1 | | |
| Hyperdip 47-50- | T13A | CCR |
| C2 | | |
| Hyperdip 47-50- | T13A | CCR |
| C3-N | | |
| Hyperdip 47-50- | T13A | CCR |
| C4 | | |
| Hyperdip 47-50- | T13A | CCR |
| C5 | | |
| Hyperdip 47-50- | T13B | CCR |
| C6 | | |
| Hyperdip 47-50- | T13B | CCR |
| C7 | | |
| Hyperdip 47-50- | T13B | CCR |
| C8 | | |
| Hyperdip 47-50- | T13B | CCR |
| C9 | | |
| Hyperdip 47-50- | T13B | CCR |
| C10 | | |
| Hyperdip 47-50- | T13B | CCR |
| C11 | | |
| Hyperdip 47-50- | T13B | CCR |
| C12 | | |
| Hyperdip 47-50-C13 | T13B | CCR |
| Hyperdip 47-50-C14-N | T13B | CCR |
| Hyperdip 47-50-C15 | T13B | CCR |
| Hyperdip 47-50-C16 | T13B | CCR |
| Hyperdip 47-50-C17 | T13B | CCR |
| Hyperdip 47-50-C18 | T13B | CCR |
| Hyperdip 47-50-C19 | T13B | CCR |
| Hyperdip 47-50-2M#1 | T13A | 2nd AML |
| Hyperdip 47-50-#1 | T15 | NA |
| Hyperdip 47-50-#2 | T15 | NA |
| Hyperdip 47-50-#3 | T15 | NA |

Hypodip subgroup (n = 9)

| | | |
|---|---|---|
| Hypodip-C1 | T13A | CCR |
| Hypodip-C2 | T13A | CCR |
| Hypodip-C3 | T13B | CCR |
| Hypodip-C4 | T13B | CCR |
| Hypodip-C5 | T13B | CCR |
| Hypodip-C6 | T13B | CCR |
| Hypodip-2M#1 | T13A | 2nd AML |
| Hypodip-#1 | T15 | NA |
| Hypodip-#2 | T15 | NA |

MLL subgroup (n = 20)

| | | |
|---|---|---|
| MLL-C1 | T13A | CCR |
| MLL-C2 | T13B | CCR |
| MLL-C3 | T13B | CCR |
| MLL-C4 | T13B | CCR |
| MLL-C5 | T13B | CCR |
| MLL-C6 | T13B | CCR |
| MLL-R1 | T13A | Heme Relapse |
| MLL-R2 | T13A | Heme Relapse |
| MLL-R3 | T13B | Heme Relapse |
| MLL-R4 | T13B | Heme Relapse |
| MLL-2M#1 | T13A | 2nd AML |
| MLL-2M#2 | T13A | 2nd AML |
| MLL-#1 | T13B | Censored |
| MLL-#2 | T13B | Censored |
| MLL-#3 | Others | NA |
| MLL-#4 | Others | NA |
| MLL-#5 | Others | NA |
| MLL-#6 | T12 | NA |
| MLL-#7 | T14 | NA |
| MLL-#8 | T14 | NA |

Normal subgroup (n = 18)

| | | |
|---|---|---|
| Normal-C1-N | T13A | CCR |
| Normal-C2-N | T13A | CCR |
| Normal-C3-N | T13A | CCR |
| Normal-C4-N | T13B | CCR |
| Normal-C5 | T13B | CCR |
| Normal-C6 | T13B | CCR |
| Normal-C7-N | T13B | CCR |
| Normal-C8 | T13B | CCR |
| Normal-C9 | T13B | CCR |
| Normal-C10 | T13B | CCR |
| Normal-C11-N | T13B | CCR |
| Normal-C12 | T13B | CCR |
| Normal-R1 | T13A | Heme |
| | | Relapse |
| Normal-R2-N | T13B | Heme |
| | | Relapse |
| Normal-R3 | T13B | Heme |
| | | Relapse |
| Normal-#1 | T13A | Censored |
| Normal-#2 | T13B | Censored |
| Normal-#3 | T13B | Censored |

Pseudodip subgroup (n = 29)

| | | |
|---|---|---|
| Pseudodip-C1 | T13A | CCR |
| Pseudodip-C2-N | T13A | CCR |
| Pseudodip-C3 | T13A | CCR |
| Pseudodip-C4 | T13A | CCR |
| Pseudodip-C5 | T13A | CCR |
| Pseudodip-C6 | T13A | CCR |
| Pseudodip-C7 | T13A | CCR |
| Pseudodip-C8 | T13A | CCR |
| Pseudodip-C9 | T13A | CCR |
| Pseudodip-C10 | T13B | CCR |
| Pseudodip-C11 | T13B | CCR |
| Pseudodip-C12 | T13B | CCR |
| Pseudodip-C13 | T13B | CCR |
| Pseudodip-C14 | T13B | CCR |
| Pseudodip-C15 | T13B | CCR |
| Pseudodip-C16-N | T13B | CCR |
| Pseudodip-C17 | T13B | CCR |
| Pseudodip-C18 | T13B | CCR |
| Pseudodip-C19 | T13B | CCR |
| Pseudodip-R1-N | T13A | Heme |
| | | Relapse |
| Pseudodip-#1 | T13B | Other |
| | | Relapse |
| Pseudodip-#2 | T13B | Censored |
| Pseudodip-#3 | Others | NA |
| Pseudodip-#4 | Others | NA |
| Pseudodip-#5 | T15 | NA |
| Pseudodip-#6 | T15 | NA |
| Pseudodip-#7 | T15 | NA |
| Pseudodip-#8-N | T15 | NA |
| Pseudodip-#9 | T15 | NA |

T-ALL subgroup (n = 43)

| | | |
|---|---|---|
| T-ALL-C1 | T13A | CCR |
| T-ALL-C2 | T13A | CCR |
| T-ALL-C3 | T13A | CCR |
| T-ALL-C4 | T13A | CCR |
| T-ALL-C5 | T13A | CCR |
| T-ALL-C6 | T13A | CCR |
| T-ALL-C7 | T13A | CCR |
| T-ALL-C8 | T13A | CCR |
| T-ALL-C9 | T13B | CCR |
| T-ALL-C10 | T13B | CCR |
| T-ALL-C11 | T13B | CCR |
| T-ALL-C12 | T13B | CCR |
| T-ALL-C13 | T13B | CCR |
| T-ALL-C14 | T13B | CCR |
| T-ALL-C15 | T13B | CCR |
| T-ALL-C16 | T13B | CCR |
| T-ALL-C17 | T13B | CCR |
| T-ALL-C18 | T13B | CCR |
| T-ALL-C19 | T13B | CCR |
| T-ALL-C20 | T13B | CCR |
| T-ALL-C21 | T13B | CCR |
| T-ALL-C22 | T13B | CCR |
| T-ALL-C23 | T13B | CCR |
| T-ALL-C24 | T13B | CCR |
| T-ALL-C25 | T13B | CCR |
| T-ALL-C26 | T13B | CCR |
| T-ALL-R1 | T13A | Heme |
| | | Relapse |
| T-ALL-R2 | T13B | Heme |
| | | Relapse |
| T-ALL-R3 | T13B | Heme |
| | | Relapse |
| T-ALL-R4 | T13B | Heme |
| | | Relapse |
| T-ALL-R5 | T13B | Heme |
| | | Relapse |
| T-ALL-R6 | T13B | Heme |
| | | Relapse |
| T-ALL-2M#1 | T13B | 2nd AML |
| T-ALL-#1 | T13B | Other |
| | | Relapse |
| T-ALL-#2 | T13B | Other |
| | | Relapse |
| T-ALL-#4 | T13B | Censored |
| T-ALL-#5 | T13B | Censored |
| T-ALL-#6 | T15 | NA |
| T-ALL-#7 | T15 | NA |
| T-ALL-#8 | T15 | NA |
| T-ALL-#9 | T15 | NA |
| T-ALL-#10 | T15 | NA |
| T-ALL-#11 | T15 | NA |

TEL-AML1 subgroup (n = 79)

| | | |
|---|---|---|
| TEL-AML1-C1 | T13A | CCR |
| TEL-AML1-C2 | T13A | CCR |
| TEL-AML1-C3 | T13A | CCR |
| TEL-AML1-C4 | T13A | CCR |
| TEL-AML1-C5 | T13A | CCR |
| TEL-AML1-C6 | T13A | CCR |
| TEL-AML1-C7 | T13A | CCR |
| TEL-AML1-C8 | T13A | CCR |
| TEL-AML1-C9 | T13A | CCR |
| TEL-AML1-C10 | T13A | CCR |
| TEL-AML1-C11 | T13A | CCR |
| TEL-AML1-C12 | T13A | CCR |
| TEL-AML1-C13 | T13A | CCR |
| TEL-AML1-C14 | T13A | CCR |
| TEL-AML1-C15 | T13A | CCR |
| TEL-AML1-C16 | T13A | CCR |
| TEL-AML1-C17 | T13A | CCR |
| TEL-AML1-C18 | T13A | CCR |
| TEL-AML1-C19 | T13A | CCR |
| TEL-AML1-C20 | T13A | CCR |
| TEL-AML1-C21 | T13A | CCR |
| TEL-AML1-C22 | T13A | CCR |
| TEL-AML1-C23 | T13A | CCR |
| TEL-AML1-C24 | T13A | CCR |
| TEL-AML1-C25 | T13A | CCR |
| TEL-AML1-C26 | T13A | CCR |
| TEL-AML1-C27 | T13A | CCR |
| TEL-AML1-C28 | T13A | CCR |
| TEL-AML1-C29 | T13B | CCR |
| TEL-AML1-C30 | T13B | CCR |
| TEL-AML1-C31 | T13B | CCR |
| TEL-AML1-C32 | T13B | CCR |
| TEL-AML1-C33 | T13B | CCR |
| TEL-AML1-C34 | T13B | CCR |
| TEL-AML1-C35 | T13B | CCR |
| TEL-AML1-C36 | T13B | CCR |
| TEL-AML1-C37 | T13B | CCR |
| TEL-AML1-C38 | T13B | CCR |
| TEL-AML1-C39 | T13B | CCR |
| TEL-AML1-C40 | T13B | CCR |
| TEL-AML1-C41 | T13B | CCR |
| TEL-AML1-C42 | T13B | CCR |
| TEL-AML1-C43 | T13B | CCR |
| TEL-AML1-C44 | T13B | CCR |
| TEL-AML1-C45 | T13B | CCR |
| TEL-AML1-C46 | T13B | CCR |
| TEL-AML1-C47 | T13B | CCR |
| TEL-AML1-C48 | T13B | CCR |
| TEL-AML1-C49 | T13B | CCR |
| TEL-AML1-C50 | T13B | CCR |
| TEL-AML1-C51 | T13B | CCR |
| TEL-AML1-C52 | T13B | CCR |
| TEL-AML1-C53 | T13B | CCR |
| TEL-AML1-C54 | T13B | CCR |
| TEL-AML1-C55 | T13B | CCR |
| TEL-AML1-C56 | T13B | CCR |
| TEL-AML1-C57 | T13B | CCR |
| TEL-AML1-R1 | T13A | Heme |
| | | Relapse |
| TEL-AML1-R2 | T13A | Heme |
| | | Relapse |
| TEL-AML1-R3 | T13B | Heme |
| | | Relapse |
| TEL-AML1-2M#1 | T13A | 2nd AML |
| TEL-AML1-2M#2 | T13A | 2nd AML |
| TEL-AML1-2M#3 | T13A | 2nd AML |
| TEL-AML1-2M#4 | T13B | 2nd AML |
| TEL-AML1-2M#5 | T13B | 2nd AML |
| TEL-AML1-#1 | T13B | Other |
| | | Relapse |
| TEL-AML1-#2 | T13A | Censored |
| TEL-AML1-#3 | T13A | Censored |
| TEL-AML1-#4 | T13B | Censored |
| TEL-AML1-#5 | T15 | NA |
| TEL-AML1-#6 | T15 | NA |
| TEL-AML1-#7 | T15 | NA |
| TEL-AML1-#8 | T15 | NA |
| TEL-AML1-#9 | T15 | NA |
| TEL-AML1-#10 | T15 | NA |
| TEL-AML1-#11 | T15 | NA |
| TEL-AML1-#12 | T15 | NA |
| TEL-AML1-#13 | T15 | NA |
| TEL-AML1-#14 | T15 | NA |

| | |
|---|---|
| ^{@}Label key- | |
| Subtype Name-C# | Dx Sample of patient in CCR |
| Subtype Name-R# | Dx Sample of patient who developed |
| | a hematologic relapse |
| Subtype Name-# | Dx Sample used for subgroup classification only |
| Subtype Name-2M# | Dx Sample of patient who later developed 2^{nd} AML |
| Subtype Name-N | Dx Sample in novel group |

^{#}Protocol-Protocol that patient was treated on

| | |
|---|---|
| ^{%}Outcome- | |
| CCR | Continuous complete remission |
| Heme Relapse | Hematologic relapse |
| Other Relapse | Extramedullary relapse |
| 2nd AML | Diagnostic samples of patients who later |
| | developed 2^{nd AML} |
| Censored | Censored due to BM transplant, |
| | treated off protocol, or died in CR |
| NA | Not applicable, primarily because the patient |
| | was not treated |
| | on Total 13, and thus is excluded |
| | from the analysis used to |
| | identify gene expression profiles |
| | predictive of outcome |

H. Diagnostic Samples Used for Prediction of Prognosis

In addition to the 201 CCR and 27 Heme Relapse cases listed in Table 1, five additional relapse cases were also included in the prognostic analysis, giving a total of 233 cases for this analysis. These additional cases were not included in the subgroup prediction data set because they did not meet the established criteria for the reasons listed below.

| Label | Protocol | Comment |
|---|---|---|
| BCR-ABL-R4 | T13B | Did not meet QC criteria because contained |
| | | 70% blasts |
| MLL-R5 | T13A | Peripheral Blood Sample (90% blasts) |
| Normal-R4- | T13B | Molecular studies not performed |
| T-ALL-R7 | T13A | Peripheral Blood Sample (90% blasts) |
| T-ALL-R8 | T13B | Peripheral Blood Sample (90% blasts) |

I. Diagnostic Samples Used for Prediction of Secondary AML

In addition to the 201 CCR and 13 secondary AML cases listed in Table 1, three additional diagnostic marrow samples from patients who developed secondary AML were also included in the prognostic analysis. This gives a total of 217 cases used for this analysis. These additional cases were not included in the diagnostic data set because they did not meet the established criteria for the reasons listed below.

| Label | Protocol | Comment |
|---|---|---|
| Hyperdip > 50-2M#3 | T12 | Non Total 13 diagnostic sample |
| Hypodip-2M#2 | T13B | No molecular studies performed |
| Hypodip-2M#3 | T12 | Non Total 13 diagnostic sample |

Relapsed Samples (n=25) p Twenty-five relapse samples were analyzed, 17 samples which were paired to the diagnostic samples listed above (Subtype Name-2M#), and 8 additional non-paired relapse samples.

Detailed Analysis

A. Hierarchical Cluster Analysis of Diagnostic Cases Using All Genes that Passed the Variation Filter

Two-dimensional hierarchical clustering was performed using Pearson correlation coefficient and an unweighted pair group method using arithmetic averages (GeneMaths, version 1.5). The results of hierarchical clustering of the 327 diagnostic samples using the 10,991 probe sets that passed the variation filter can be viewed at our web site, www.stjuderesearch.org/ALL1.

B. Methods for Gene Selection

Discriminating genes for the various leukemia subtypes were selected using a variety of statistical metrics. The individual metrics used and the list of selected probe sets and corresponding genes are given below.

1. Chi-Square

The Chi square method evaluates each gene individually by measuring the Chi square statistics with respect to the classes. The method first discretizes the observed expression values of the gene into several intervals using an entropy-based discretization methodⁱ. The Chi square statistics of a gene is then calculated as X²=ΣΣ(Aᵢⱼ−Eᵢⱼ)²/Eᵢⱼ, summing over intervals i=1..m and classes j =1..k. Aᵢⱼ is the number of samples in the i^{th} interval that are of the j^{th} class. Eᵢⱼ is the expected frequency of Aᵢⱼ and is calculated as Eᵢⱼ=Rᵢ* Cᵢ/N, where Rᵢ is the number of samples in the i^{th} interval, Cⱼ is the number of samples in the j^{th} class, and N is the total number of samples. The genes are then sorted according to their Chi square statistics: the larger the Chi square statistics, the more important the gene. The 40 genes with the highest Chi square statistics in each subtype are listed in Tables 2-8. Generally, using anywhere from the top 20 to 40 genes did not result in significant differences in subtype prediction accuracy. Therefore, only the top 20 genes in subtype prediction were used, unless noted otherwise.

TABLE 2
Genes selected by Chi square: BCR-ABL

| | | | | | Chi | Above/ |
|---|---|---|---|---|---|---|
| | Affymetrix | | | Reference | square | Below |
| | number | Gene Name | GeneSymbol | number | value | Mean |
| 1 | 1637_at | mitogen-activated protein kinase- | MAPKAPK3 | U09578 | 62.75 | Above |
| | | activated protein kinase 3 | | | | |
| 2 | 36650_at | cyclin D2 | CCND2 | D13639 | 59.79 | Above |
| 3 | 40196_at | HYA22 protein | HYA22 | D88153 | 54.79 | Above |
| 4 | 1635_at | proto-oncogene tyrosine-protein | ABL | U07563 | 54.77 | Above |
| | | kinase ABL gene | | | | |
| 5 | 33775_s_at | caspase 8 apoptosis-related | CASP8 | X98176 | 49.70 | Above |
| | | cysteine protease | | | | |
| 6 | 1636_g_at | proto-oncogene tyrosine-protein | ABL | U07563 | 48.29 | Above |
| | | kinase ABL gene | | | | |
| 7 | 41295_at | GTT1 protein | GTT1 | AL041780 | 42.60 | Above |
| 8 | 37600_at | extracellular matrix protein 1 | ECM1 | U68186 | 42.60 | Above |
| 9 | 37012_at | capping protein actin filament | CAPZB | U03271 | 38.46 | Above |
| | | muscle Z-line beta | | | | |
| 10 | 39225_at | alkylglycerone phosphate synthase | AGPS | Y09443 | 38.46 | Above |
| 11 | 1326_at | caspase 10 apoptosis-related | CASP10 | U60519 | 37.83 | Above |
| | | cysteine protease | | | | |
| 12 | 34362_at | solute carrier family 2 facilitated | SLC2A5 | M55531 | 37.54 | Above |
| | | glucose transporter member 5 | | | | |
| 13 | 33150_at | disrupter of silencing 10 | SAS10 | AI126004 | 36.95 | Above |
| 14 | 40051_at | TRAM-like protein | KIAA0057 | D31762 | 36.95 | Above |
| 15 | 39061_at | bone marrow stromal cell antigen 2 | BST2 | D28137 | 36.95 | Above |
| 16 | 33172_at | hypothetical protein FLJ10849 | FLJ10849 | T75292 | 36.95 | Above |
| 17 | 37399_at | aldo-keto reductase family 1 | AKR1C3 | D17793 | 36.95 | Above |
| | | member C3 3-alpha | | | | |
| | | hydroxysteroid dehydrogenase | | | | |
| | | type II | | | | |
| 18 | 317_at | protease cysteine 1 legumain | PRSC1 | D55696 | 36.95 | Above |
| 19 | 40953_at | calponin 3 acidic | CNN3 | S80562 | 33.94 | Above |
| 20 | 330_s_at | tubulin, alpha 1, isoform 44 | TUBA1 | HG2259- | 33.32 | Above |
| | | | | HT2348 | | |
| 21 | 40504_at | paraoxonase 2 | PON2 | AF001601 | 31.46 | Above |
| 22 | 38578_at | tumor necrosis factor receptor | TNFRSF7 | M63928 | 30.47 | Above |
| | | superfamily member 7 | | | | |
| 23 | 39044_s_at | diacylglycerol kinase delta 130 kD | DGKD | D73409 | 29.59 | Below |
| 24 | 36634_at | BTG family member 2 | BTG2 | U72649 | 29.16 | Below |
| 25 | 38119_at | glycophorin C Gerbich blood | GYPC | X12496 | 29.16 | Above |
| | | group | | | | |
| 26 | 32562_at | endoglin Osler-Rendu-Weber | ENG | X72012 | 27.96 | Above |
| | | syndrome 1 | | | | |
| 27 | 33228_g_at | interleukin 10 receptor beta | IL10RB | AI984234 | 27.70 | Below |
| 28 | 37006_at | step II splicing factor SLU7 | SLU7 | AI660656 | 27.15 | Above |
| 29 | 38641_at | Homo sapiens mRNA for TSC-22- | | AJ133115 | 27.15 | Above |
| | | like protein | | | | |
| 30 | 38220_at | dihydropyrimidine dehydrogenase | DPYD | U20938 | 27.15 | Above |
| 31 | 1211_s_at | CASP2 and RIPK1 domain | CRADD | U84388 | 26.46 | Above |
| | | containing adaptor with death | | | | |
| | | domain | | | | |
| 32 | 39730_at | v-abl Abelson murine leukemia | ABL1 | X16416 | 25.90 | Above |
| | | viral oncogene homolog 1 | | | | |
| 33 | 36591_at | tubulin alpha 1 testis specific | TUBA1 | X06956 | 25.90 | Above |
| 34 | 36035_at | anchor attachment protein 1 Gaa1p | GPAA1 | AB002135 | 25.34 | Above |
| | | yeast homolog | | | | |
| 35 | 980_at | Niemann-Pick disease type C1 | NPC1 | AF002020 | 25.29 | Above |
| 36 | 671_at | secreted protein acidic cysteine- | SPARC | J03040 | 25.29 | Above |
| | | rich osteonectin | | | | |
| 37 | 40698_at | C-type calcium dependent | CLECSF2 | X96719 | 23.80 | Above |
| | | carbohydrate-recognition domain | | | | |
| | | lectin superfamily member 2 | | | | |
| | | activation-induced | | | | |
| 38 | 39330_s_at | actinin alpha 1 | ACTN1 | M95178 | 23.70 | Above |
| 39 | 1983_at | cyclin D2 | CCND2 | X68452 | 23.70 | Above |
| 40 | 2001_g_at | ataxia telangiectasia mutated | ATM | U26455 | 22.60 | Above |

TABLE 3
Genes selected by Chi Square for E2A-PBX1

| | | | | | Chi | Above/ |
|---|---|---|---|---|---|---|
| | Affymetrix | | | Reference | square | Below |
| | number | Gene Name | GeneSymbol | number | value | Mean |
| 1 | 41146_at | ADP-ribosyltransferase NAD poly | ADPRT | J03473 | 187.00 | Above |
| | | ADP-ribose polymerase | | | | |
| 2 | 1287_at | ADP-ribosyltransferase NAD poly | ADPRT | J03473 | 187.00 | Above |
| | | ADP-ribose polymerase | | | | |
| 3 | 32063_at | pre-B-cell leukemia transcription | PBX1 | M86546 | 187.00 | Above |
| | | factor 1 | | | | |
| 4 | 33355_at | Homo sapiens cDNA FLJ12900 | PBX1 | AL049381 | 187.00 | Above |
| | | fis clone NT2RP2004321 (by | | | | |
| | | CELERA serach of target | | | | |
| | | sequence = PBX1) | | | | |
| 5 | 430_at | nucleoside phosphorylase | NP | X00737 | 187.00 | Above |
| 6 | 40454_at | FAT tumor suppressor Drosophila | FAT | X87241 | 176.11 | Above |
| | | homolog | | | | |
| 7 | 753_at | nidogen 2 | NID2 | D86425 | 164.28 | Above |
| 8 | 33821_at | Human DNA sequence from clone | HELO1 | AL034374 | 155.00 | Above |
| | | RP3-483K16 on chromosome | | | | |
| | | 6p12.1-21.1 | | | | |
| 9 | 39614_at | KIAA0802 protein | KIAA0802 | AB018345 | 153.46 | Above |
| 10 | 38340_at | huntingtin interacting protein-1- | KIAA0655 | AB014555 | 143.85 | Above |
| | | related | | | | |
| 11 | 1786_at | c-mer proto-oncogene tyrosine | MERTK | U08023 | 142.34 | Above |
| | | kinase | | | | |
| 12 | 39929_at | KIAA0922 protein | KIAA0922 | AB023139 | 139.97 | Above |
| 13 | 39379_at | Homo sapiens mRNA cDNA | | AL049397 | 139.49 | Above |
| | | DKFZp586C1019 from clone | | | | |
| | | DKFZp586C1019 | | | | |
| 14 | 717_at | GS3955 protein | GS3955 | D87119 | 135.24 | Above |
| 15 | 362_at | protein kinase C zeta | PRKCZ | Z15108 | 131.36 | Above |
| 16 | 33513_at | signaling lymphocytic activation | SLAM | U33017 | 131.36 | Above |
| | | molecule | | | | |
| 17 | 37225_at | KIAA0172 protein | KIAA0172 | D79994 | 131.36 | Above |
| 18 | 854_at | B lymphoid tyrosine kinase | BLK | S76617 | 130.95 | Above |
| 19 | 35974_at | lymphoid-restricted membrane | LRMP | U10485 | 123.33 | Above |
| | | protein | | | | |
| 20 | 36452_at | synaptopodin | KIAA1029 | AB028952 | 123.33 | Above |
| 21 | 40648_at | c-mer proto-oncogene tyrosine | MERTK | U08023 | 120.51 | Above |
| | | kinase | | | | |
| 22 | 38393_at | KIAA0247 gene product | KIAA0247 | D87434 | 120.51 | Above |
| 23 | 38994_at | STAT induced STAT inhibitor-2 | STATI2 | AF037989 | 118.58 | Below |
| 24 | 34861_at | golgi autoantigen golgin subfamily | GOLGA3 | D63997 | 116.80 | Above |
| | | a 3 | | | | |
| 25 | 38748_at | adenosine deaminase RNA- | ADARB1 | U76421 | 114.13 | Above |
| | | specific B1 homolog of rat RED1 | | | | |
| 26 | 40113_at | GS3955 protein | GS3955 | D87119 | 114.13 | Above |
| 27 | 36179_at | mitogen-activated protein kinase- | MAPKAPK2 | U12779 | 113.43 | Above |
| | | activated protein kinase 2 | | | | |
| 28 | 37493_at | colony stimulating factor 2 | CSF2RB | H04668 | 113.04 | Above |
| | | receptor beta low-affinity | | | | |
| | | granulocyte-macrophage | | | | |
| 29 | 578_at | Human recombination acitivating | RAG2 | M94633 | 111.32 | Above |
| | | protein (RAG2) gene | | | | |
| 30 | 41017_at | myosin-binding protein H | MYBPH | U27266 | 109.73 | Above |
| 31 | 37625_at | interferon regulatory factor 4 | IRF4 | U52682 | 108.51 | Above |
| 32 | 38679_g_at | small nuclear ribonucleoprotein | SNRPE | AA733050 | 106.02 | Above |
| | | polypeptide E | | | | |
| 33 | 1389_at | membrane metallo-endopeptidase | MME | J03779 | 105.65 | Below |
| | | neutral endopeptidase | | | | |
| | | enkephalinase CALLA CD10 | | | | |
| 34 | 34783_s_at | BUB3 budding uninhibited by | BUB3 | AF047473 | 103.87 | Above |
| | | benzimidazoles 3 yeast homolog | | | | |
| 35 | 36959_at | ubiquitin-conjugating enzyme E2 | UBE2V1 | U49278 | 103.87 | Above |
| | | variant 1 | | | | |
| 36 | 39864_at | cold inducible RNA-binding | CIRBP | D78134 | 99.76 | Below |
| | | protein | | | | |
| 37 | 41862_at | KIAA0056 protein | KIAA0056 | D29954 | 99.76 | Above |
| 38 | 41425_at | Friend leukemia virus integration 1 | FLI1 | M98833 | 96.47 | Above |
| 39 | 37177_at | CD58 antigen lymphocyte | CD58 | Y00636 | 93.84 | Above |
| | | function-associated antigen 3 | | | | |
| 40 | 37485_at | fatty-acid-Coenzyme A ligase very | FACVL1 | D88308 | 93.17 | Above |
| | | long-chain 1 | | | | |

TABLE 4
Genes selected by Chi square for Hyperdiploid >50

| | | | | | Chi | Above/ |
|---|---|---|---|---|---|---|
| | Affymetrix | | | Reference | square | Below |
| | number | Gene Name | GeneSymbol | number | value | Mean |
| 1 | 36620_at | superoxide dismutase 1 soluble | SOD1 | X02317 | 52.43 | Above |
| | | amyotrophic lateral sclerosis 1 | | | | |
| | | adult | | | | |
| 2 | 37350_at | Human DNA sequence from clone | PSMD10 | AL031177 | 48.71 | Above |
| | | 889N15 on chromosome Xq22.1-22.3. | | | | |
| 3 | 171_at | von Hippel-Lindau binding protein 1 | VBP1 | U56833 | 45.80 | Above |
| 4 | 37677_at | phosphoglycerate kinase 1 | PGK1 | V00572 | 45.80 | Above |
| 5 | 41724_at | accessory proteins BAP31/BAP29 | DXS1357E | X81109 | 45.58 | Above |
| 6 | 32207_at | membrane protein palmitoylated 1 | MPP1 | M64925 | 44.07 | Above |
| | | 55 kD | | | | |
| 7 | 38738_at | SMT3 suppressor of mif two 3 | SMT3H1 | X99584 | 43.57 | Above |
| | | yeast homolog 1 | | | | |
| 8 | 40480_s_at | FYN oncogene related to SRC | FYN | M14333 | 43.57 | Above |
| | | FGR YES | | | | |
| 9 | 38518_at | sex comb on midleg Drosophila | SCML2 | Y18004 | 43.20 | Above |
| | | like 2 | | | | |
| 10 | 41132_r_at | heterogeneous nuclear | HNRPH2 | U01923 | 43.15 | Above |
| | | ribonucleoprotein H2 H | | | | |
| 11 | 31492_at | muscle specific gene | M9 | AB019392 | 43.01 | Below |
| 12 | 38317_at | transcription elongation factor A | TCEAL1 | M99701 | 41.10 | Above |
| | | SII like 1 | | | | |
| 13 | 40998_at | trinucleotide repeat containing 11 | TNRC11 | AF071309 | 40.88 | Above |
| | | THR-associated protein 230 kDa | | | | |
| | | subunit | | | | |
| 14 | 35688_g_at | mature T-cell proliferation 1 | MTCP1 | Z24459 | 40.52 | Above |
| 15 | 40903_at | ATPase H transporting lysosomal | APT6M8-9 | AL049929 | 40.33 | Above |
| | | vacuolar proton pump membrane | | | | |
| | | sector associated protein M8-9 | | | | |
| 16 | 36489_at | phosphoribosyl pyrophosphate | PRPS1 | D00860 | 40.33 | Above |
| | | synthetase 1 | | | | |
| 17 | 1520_s_at | interleukin 1 beta | IL1B | X04500 | 40.29 | Above |
| 18 | 35939_s_at | POU domain class 4 transcription | POU4F1 | L20433 | 38.74 | Above |
| | | factor 1 | | | | |
| 19 | 38604_at | neuropeptide Y | NPY | AI198311 | 38.26 | Above |
| 20 | 31863_at | KIAA0179 protein | KIAA0179 | D80001 | 38.26 | Above |
| 21 | 890_at | ubiquitin-conjugating enzyme | UBE2A | M74524 | 37.99 | Above |
| | | E2A RAD6 homolog | | | | |
| 22 | 39402_at | interleukin 1 beta | IL1B | M15330 | 37.92 | Above |
| 23 | 41490_at | phosphoribosyl pyrophosphate | PRPS2 | Y00971 | 37.72 | Above |
| | | synthetase 2 | | | | |
| 24 | 34753_at | synaptobrevin-like 1 | SYBL1 | X92396 | 37.72 | Above |
| 25 | 40891_f_at | DNA segment on chromosome X | DXS9879E | X92896 | 37.15 | Above |
| | | unique 9879 expressed sequence | | | | |
| 26 | 306_s_at | high-mobility group nonhistone | HMG14 | J02621 | 37.15 | Above |
| | | chromosomal protein 14 | | | | |
| 27 | 37640_at | hypoxanthine | HPRT1 | M31642 | 37.15 | Above |
| | | phosphoribosyltransferase 1 | | | | |
| | | Lesch-Nyhan syndrome | | | | |
| 28 | 34829_at | dyskeratosis congenita 1 dyskerin | DKC1 | U59151 | 36.48 | Above |
| 29 | 36169_at | NADH dehydrogenase ubiquinone | NDUFA1 | N47307 | 36.48 | Above |
| | | 1 alpha subcomplex 1 7.5 kD | | | | |
| | | MWFE | | | | |
| 30 | 38968_at | SH3-domain binding protein 5 | SH3BP5 | AB005047 | 35.95 | Above |
| | | BTK-associated | | | | |
| 31 | 36128_at | transmembrane trafficking protein | TMP21 | L40397 | 35.88 | Above |
| 32 | 37014_at | myxovirus influenza resistance 1 | MX1 | M33882 | 35.65 | Above |
| | | homolog of murine interferon- | | | | |
| | | inducible protein p78 | | | | |
| 33 | 34374_g_at | upstream regulatory element | UREB1 | Z97054 | 35.55 | Above |
| | | binding protein 1 | | | | |
| 34 | 36542_at | solute carrier family 9 | SLC9A6 | AF030409 | 35.55 | Above |
| | | sodium/hydrogen exchanger | | | | |
| | | isoform 6 | | | | |
| 35 | 688_at | proteasome prosome macropain | PSMC1 | L02426 | 35.55 | Above |
| | | 26S subunit ATPase 1 | | | | |
| 36 | 955_at | calmodulin type I | | HG1862- | 35.55 | Above |
| | | | | HT1897 | | |
| 37 | 35816_at | cystatin B stefin B | CSTB | U46692 | 35.27 | Above |
| 38 | 38459_g_at | Human cytochrome b5 (CYB5) | CYB5 | L39945 | 35.18 | Above |
| | | gene | | | | |
| 39 | 41288_at | matrix Gla protein | MGP | AL036744 | 35.18 | Above |
| 40 | 32251_at | hypothetical protein FLJ21174 | FLJ21174 | AA149307 | 35.14 | Above |

TABLE 5
Genes selected by Chi square for MLL

| | | | | | Chi | Above/ |
|---|---|---|---|---|---|---|
| | Affymetrix | | | Reference | square | Below |
| | number | Gene Name | GeneSymbol | number | value | Mean |
| 1 | 34306_at | muscleblind Drosophila like | MBNL | AB007888 | 64.07 | Above |
| 2 | 40797_at | a disintegrin and | ADAM10 | AF009615 | 62.85 | Above |
| | | metalloproteinase domain 10 | | | | |
| 3 | 33412_at | LGALS1 Lectin, galactoside- | LGALS1 | AI535946 | 57.97 | Above |
| | | binding, soluble, 1 | | | | |
| 4 | 39338_at | S100 calcium-binding protein | S100A10 | AI201310 | 57.97 | Above |
| | | A10 annexin II ligand calpactin | | | | |
| | | I light polypeptide p11 | | | | |
| 5 | 2062_at | insulin-like growth factor | IGFBP7 | L19182 | 55.22 | Above |
| | | binding protein 7 | | | | |
| 6 | 32193_at | plexin C1 | PLXNC1 | AF030339 | 53.59 | Above |
| 7 | 40518_at | protein tyrosine phosphatase | PTPRC | Y00062 | 53.40 | Above |
| | | receptor type C | | | | |
| 8 | 36777_at | DNA segment on chromosome | D12S2489E | AJ001687 | 51.47 | Above |
| | | 12 unique 2489 expressed | | | | |
| | | sequence | | | | |
| 9 | 32207_at | membrane protein palmitoylated | MPP1 | M64925 | 50.73 | Below |
| | | 1 55 kD | | | | |
| 10 | 33859_at | sin3-associated polypeptide | SAP18 | U96915 | 50.48 | Above |
| | | 18 kD | | | | |
| 11 | 38391_at | capping protein actin filament | CAPG | M94345 | 50.26 | Above |
| | | gelsolin-like | | | | |
| 12 | 40763_at | Meis1 mouse homolog | MEIS1 | U85707 | 50.26 | Above |
| 13 | 1126_s_at | cell surface glycoprotein CD44 | CD44 | L05424 | 50.17 | Above |
| | | gene | | | | |
| 14 | 34721_at | FK506-binding protein 5 | FKBP5 | U42031 | 50.17 | Above |
| 15 | 37809_at | homeo box A9 | HOXA9 | U41813 | 50.17 | Above |
| 16 | 34861_at | golgi autoantigen golgin | GOLGA3 | D63997 | 47.58 | Below |
| | | subfamily a 3 | | | | |
| 17 | 38194_s_at | immunoglobulin kappa constant | IGKC | M63438 | 46.18 | Below |
| 18 | 657_at | protocadherin gamma subfamily | PCDHGC3 | L11373 | 46.05 | Above |
| | | C 3 | | | | |
| 19 | 36918_at | guanylate cyclase 1 soluble | GUCY1A3 | Y15723 | 43.90 | Above |
| | | alpha 3 | | | | |
| 20 | 32215_i_at | KIAA0878 protein | KIAA0878 | AB020685 | 43.90 | Above |
| 21 | 38160_at | lymphocyte antigen 75 | LY75 | AF011333 | 43.90 | Above |
| 22 | 38413_at | defender against cell death 1 | DAD1 | D15057 | 43.90 | Above |
| 23 | 1389_at | membrane metallo- | MME | J03779 | 43.82 | Below |
| | | endopeptidase neutral | | | | |
| | | endopeptidase enkephalinase | | | | |
| | | CALLA CD10 | | | | |
| 24 | 34168_at | deoxynucleotidyltransferase | DNTT | M11722 | 43.82 | Below |
| | | terminal | | | | |
| 25 | 2036_s_at | CD44 antigen homing function | CD44 | M59040 | 42.55 | Above |
| | | and Indian blood group system | | | | |
| 26 | 40522_at | glutamate-ammonia ligase | GLUL | X59834 | 42.55 | Above |
| | | glutamine synthase | | | | |
| 27 | 854_at | B lymphoid tyrosine kinase | BLK | S76617 | 42.34 | Above |
| 28 | 40067_at | E74-like factor 1 ets domain | ELF1 | M82882 | 40.85 | Above |
| | | transcription factor | | | | |
| 29 | 39756_g_at | X-box binding protein 1 | XBP1 | Z93930 | 39.95 | Below |
| 30 | 36940_at | TGFB1-induced anti-apoptotic | TIAF1 | D86970 | 39.82 | Below |
| | | factor 1 | | | | |
| 31 | 36935_at | RAS p21 protein activator | RASA1 | M23379 | 38.77 | Above |
| | | GTPase activating protein 1 | | | | |
| 32 | 32134_at | testin | DKFZP586 | AL050162 | 38.77 | Above |
| | | | B2022 | | | |
| 33 | 39379_at | Homo sapiens mRNA cDNA | | AL049397 | 38.77 | Above |
| | | DKFZp586C1019 from clone | | | | |
| | | DKFZp586C1019 | | | | |
| 34 | 40493_at | Human cell surface glycoprotein | CD44 | L05424 | 38.44 | Above |
| | | CD44 | | | | |
| 35 | 769_s_at | annexin A2 | ANXA2 | D00017 | 37.61 | Above |
| 36 | 40415_at | acetyl-Coenzyme A | ACAA1 | X14813 | 37.55 | Above |
| | | acyltransferase 1 peroxisomal 3- | | | | |
| | | oxoacyl-Coenzyme A thiolase | | | | |
| 37 | 35983_at | hypothetical protein R32184_1 | R32184_1 | AC004528 | 37.55 | Above |
| 38 | 40519_at | protein tyrosine phosphatase | PTPRC | Y00638 | 36.56 | Above |
| | | receptor type C | | | | |
| 39 | 794_at | protein tyrosine phosphatase | PTPN6 | X62055 | 36.56 | Above |
| | | non-receptor type 6 | | | | |
| 40 | 41234_at | DnaJ Hsp40 homolog subfamily | DNAJB6 | AI540318 | 36.56 | Above |
| | | B member 6 | | | | |

TABLE 6
Genes selected by Chi square for Novel risk group

| | | | | | Chi | Above/ |
|---|---|---|---|---|---|---|
| | Affymetrix | | | Reference | square | Below |
| | number | Gene Name | GeneSymbol | number | value | Mean |
| 1 | 37960_at | carbohydrate chondroitin | CHST2 | AB014679 | 175.82 | Above |
| | | 6/keratan sulfotransferase 2 | | | | |
| 2 | 31892_at | protein tyrosine phosphatase | PTPRM | X58288 | 172.85 | Above |
| | | receptor type M | | | | |
| 3 | 994_at | protein tyrosine phosphatase | PTPRM | X58288 | 172.85 | Above |
| | | receptor type M | | | | |
| 4 | 995_g_at | protein tyrosine phosphatase | PTPRM | X58288 | 172.85 | Above |
| | | receptor type M | | | | |
| 5 | 41074_at | G protein-coupled receptor 49 | GPR49 | AF062006 | 139.36 | Above |
| 6 | 41073_at | G protein-coupled receptor 49 | GPR49 | AI743745 | 139.36 | Above |
| 7 | 34676_at | KIAA1099 protein | KIAA1099 | AB029022 | 137.71 | Above |
| 8 | 36139_at | DKFZP586G0522 protein | DKFZP586G0522 | AL050289 | 127.05 | Above |
| 9 | 37542_at | lipoma HMGIC fusion partner- | LHFPL2 | D86961 | 120.79 | Above |
| | | like 2 | | | | |
| 10 | 41159_at | clathrin heavy polypeptide Hc | CLTC | D21260 | 115.15 | Above |
| 11 | 40081_at | phospholipid transfer protein | PLTP | L26232 | 108.33 | Above |
| 12 | 32800_at | Human retinoid X receptor | RXR | U66306 | 107.39 | Above |
| | | alpha mRNA, 3′ UTR, partial | | | | |
| | | sequence | | | | |
| 13 | 36906_at | cannabinoid receptor 1 brain | CNR1 | U73304 | 107.39 | Above |
| 14 | 39878_at | protocadherin 9 | PCDH9 | AI524125 | 99.20 | Above |
| 15 | 41747_s_at | Human myocyte-specific | MEF2A | U49020 | 99.20 | Above |
| | | enhancer factor 2A (MEF2A) | | | | |
| | | gene, last coding exon, and | | | | |
| | | complete cds. | | | | |
| 16 | 33410_at | integrin alpha 6 | ITGA6 | S66213 | 96.17 | Above |
| 17 | 34947_at | phorbolin-like protein MDS019 | MDS019 | AA442560 | 93.59 | Above |
| 18 | 36029_at | chromosome 11 open reading | C11ORF8 | U57911 | 93.59 | Above |
| | | frame 8 | | | | |
| 19 | 41708_at | KIAA1034 protein | KIAA1034 | AB028957 | 92.60 | Above |
| 20 | 1664_at | insulin-like growth factor 2 | IGF2 | HG3543- | 92.60 | Above |
| | | | | HT3739 | | |
| 21 | 32736_at | HSPC022 protein | HSPC022 | W68830 | 91.62 | Below |
| 22 | 41266_at | integrin alpha 6 | ITGA6 | X53586 | 86.95 | Above |
| 23 | 36566_at | cystinosis nephropathic | CTNS | AJ222967 | 82.89 | Above |
| 24 | 1825_at | IQ motif containing GTPase | IQGAP1 | L33075 | 81.20 | Below |
| | | activating protein 1 | | | | |
| 25 | 1731_at | platelet-derived growth factor | PDGFRA | M21574 | 78.22 | Above |
| | | receptor alpha polypeptide | | | | |
| 26 | 37023_at | lymphocyte cytosolic protein 1 | LCP1 | J02923 | 78.22 | Below |
| | | L-plastin | | | | |
| 27 | 33037_at | carbohydrate N- | CHST7 | AL022165 | 76.00 | Above |
| | | acetylglucosamine 6-O | | | | |
| | | sulfotransferase 7 | | | | |
| 28 | 33411_g_at | integrin alpha 6 | ITGA6 | S66213 | 75.47 | Above |
| 29 | 538_at | CD34 antigen | CD34 | S53911 | 74.86 | Above |
| 30 | 39108_at | lanosterol synthase 2 3- | LSS | U22526 | 71.90 | Above |
| | | oxidosqualene-lanosterol | | | | |
| | | cyclase | | | | |
| 31 | 38364_at | BCE-1 protein | BCE-1 | AF068197 | 71.90 | Above |
| 32 | 40423_at | KIAA0903 protein | KIAA0903 | AB020710 | 71.29 | Above |
| 33 | 35192_at | glycine dehydrogenase | GLDC | D90239 | 71.29 | Above |
| | | decarboxylating glycine | | | | |
| | | decarboxylase glycine cleavage | | | | |
| | | system protein P | | | | |
| 34 | 39037_at | myeloid/lymphoid or mixed- | MLLT2 | L13773 | 71.29 | Above |
| | | lineage leukemia trithorax | | | | |
| | | Drosophila homolog | | | | |
| | | translocated to 2 | | | | |
| 35 | 38747_at | Human CD34 gene, exon 8. | CD34 | M81945 | 69.45 | Above |
| 36 | 37687_i_at | Fc fragment of IgG low affinity | FCGR2A | M31932 | 67.75 | Above |
| | | IIa receptor for CD32 | | | | |
| 37 | 1857_at | MAD mothers against | MADH7 | AF010193 | 66.28 | Above |
| | | decapentaplegic Drosophila | | | | |
| | | homolog 7 | | | | |
| 38 | 38618_at | Human PAC clone RP3-515N1 | LIMK2 | AC002073 | 64.03 | Above |
| | | from 22q11.2-q22 | | | | |
| 39 | 31782_at | prostaglandin D2 receptor DP | PTGDR | U31099 | 61.92 | Above |
| 40 | 32842_at | B-cell CLL/lymphoma 7A | BCL7A | X89984 | 61.57 | Above |

TABLE 7
Genes selected for Chi square for T-ALL

| | | | | | Chi | Above/ |
|---|---|---|---|---|---|---|
| | Affymetrix | | | Reference | square | Below |
| | number | Gene Name | GeneSymbol | number | value | Mean |
| 1 | 38319_at | CD3D antigen delta polypeptide | CD3D | AA919102 | 215.00 | Above |
| | | TiT3 complex | | | | |
| 2 | 1096_g_at | CD19 antigen | CD19 | M28170 | 206.48 | Below |
| 3 | 38242_at | B cell linker protein | SLP65 | AF068180 | 198.52 | Below |
| 4 | 32794_g_at | T cell receptor beta locus | TRB | X00437 | 197.71 | Above |
| 5 | 37988_at | CD79B antigen | CD79B | M89957 | 197.71 | Below |
| | | immunoglobulin-associated beta | | | | |
| 6 | 38017_at | CD79A antigen | CD79A | U05259 | 197.53 | Below |
| | | immunoglobulin-associated | | | | |
| | | alpha | | | | |
| 7 | 35016_at | Human Ia-associated invariant | M13560 | M13560 | | Below |
| | | gamma-chain gene, exon 8, | | | | |
| | | clones lambda-y(1,2,3). | | | | |
| 8 | 36277_at | Human membran protein (CD3- | CD3E | M23323 | 197.53 | Above |
| | | epsilon) gene, exon 9. | | | | |
| 9 | 38095_i_at | major histocompatibility | HLA-DPB1 | M83664 | 191.09 | Below |
| | | complex class II DP beta 1 | | | | |
| 10 | 39318_at | T-cell leukemia/lymphoma 1A | TCL1A | X82240 | 189.78 | Below |
| 11 | 38147_at | SH2 domain protein 1A Duncans | SH2D1A | AL023657 | 189.78 | Above |
| | | disease lymphoproliferative | | | | |
| | | syndrome | | | | |
| 12 | 41723_s_at | major histocompatibility | HLA-DRB1 | M32578 | 189.25 | Below |
| | | complex class II DR beta 1 | | | | |
| 13 | 38833_at | Human mRNA for SB classII | | X00457 | 189.03 | Below |
| | | histocompatibility antigen | | | | |
| | | alpha-chain | | | | |
| 14 | 33238_at | Human T-lymphocyte specific | lck | U23852 | 189.03 | Above |
| | | protein tyrosine kinase p56lck | | | | |
| | | (lck) abberant mRNA | | | | |
| 15 | 37039_at | major histocompatibility | HLA-DRA | J00194 | 188.93 | Below |
| | | complex class II DR alpha | | | | |
| 16 | 38051_at | mal T-cell differentiation protein | MAL | X76220 | 188.93 | Above |
| 17 | 37344_at | major histocompatibility | HLA-DMA | X62744 | 187.25 | Below |
| | | complex class II DM alpha | | | | |
| 18 | 38096_f_at | major histocompatibility | HLA-DPB1 | M83664 | 182.38 | Below |
| | | complex class II DP beta 1 | | | | |
| 19 | 2059_s_at | lymphocyte-specific protein | LCK | M36881 | 182.38 | Above |
| | | tyrosine kinase | | | | |
| 20 | 1105_s_at | T cell receptor beta locus | TRB | M12886 | 180.45 | Above |
| 21 | 32649_at | transcription factor 7 T-cell | TCF7 | X59871 | 177.84 | Above |
| | | specific HMG-box | | | | |
| 22 | 38949_at | protein kinase C theta | PRKCQ | L01087 | 172.59 | Below |
| 23 | 39709_at | selenoprotein W 1 | SEPW1 | U67171 | 171.96 | Above |
| 24 | 41165_g_at | immunoglobulin heavy constant | IGHM | X67301 | 171.96 | Below |
| | | mu | | | | |
| 25 | 36473_at | ubiquitin specific protease 20 | USP20 | AB023220 | 167.27 | Above |
| 26 | 266_s_at | CD24 antigen small cell lung | CD24 | L33930 | 165.56 | Below |
| | | carcinoma cluster 4 antigen | | | | |
| 27 | 40570_at | forkhead box O1A | FOXO1A | AF032885 | 165.29 | Below |
| | | rhabdomyosarcoma | | | | |
| 28 | 40775_at | integral membrane protein 2A | ITM2A | AL021786 | 164.14 | Above |
| 29 | 37420_i_at | Human DNA sequence from | | AL022723 | 164.14 | Below |
| | | clone RP3-377H14 on | | | | |
| | | chromosome 6p21.32-22.1. | | | | |
| 30 | 1085_s_at | phospholipase C gamma 2 | PLCG2 | M37238 | 161.30 | Below |
| | | phosphatidylinositol-specific | | | | |
| 31 | 38018_g_at | CD79A antigen | CD79A | U05259 | 160.51 | Below |
| | | immunoglobulin-associated | | | | |
| | | alpha | | | | |
| 32 | 35643_at | nucleobindin 2 | NUCB2 | X76732 | 160.07 | Above |
| 33 | 41166_at | immunoglobulin heavy constant | IGHM | X58529 | 158.50 | Below |
| | | mu | | | | |
| 34 | 38415_at | protein tyrosine phosphatase | PTP4A2 | U14603 | 155.78 | Above |
| | | type IVA member 2 | | | | |
| 35 | 38893_at | neutrophil cytosolic factor 4 | NCF4 | AL008637 | 155.78 | Below |
| | | 40 kD | | | | |
| 36 | 1241_at | protein tyrosine phosphatase | PTP4A2 | U14603 | 155.78 | Above |
| | | type IVA member 2 | | | | |
| 37 | 32793_at | T cell receptor beta locus | TRB | X00437 | 155.43 | Above |
| 38 | 36571_at | topoisomerase DNA II beta | TOP2B | X68060 | 152.16 | Below |
| | | 180 kD | | | | |
| 39 | 37399_at | aldo-keto reductase family 1 | AKR1C3 | D17793 | 151.93 | Above |
| | | member C3 3-alpha | | | | |
| | | hydroxysteroid dehydrogenase | | | | |
| | | type II | | | | |
| 40 | 41097_at | telomeric repeat binding factor 2 | TERF2 | AF002999 | 151.86 | Below |

TABLE 8
Genes selected by Chi square for TEL-AML1

| | | | | | Chi | Above/ |
|---|---|---|---|---|---|---|
| | Affymetrix | | | Reference | square | Below |
| | number | Gene Name | GeneSymbol | number | value | Mean |
| 1 | 38652_at | hypothetical protein FLJ20154 | FLJ20154 | AF070644 | 137.92 | Above |
| 2 | 36239_at | POU domain class 2 associating | POU2AF1 | Z49194 | 131.43 | Above |
| | | factor 1 | | | | |
| 3 | 41442_at | core-binding factor runt domain | CBFA2T3 | AB010419 | 130.17 | Above |
| | | alpha subunit 2 translocated to 3 | | | | |
| 4 | 37780_at | piccolo presynaptic cytomatrix | PCLO | AB011131 | 126.79 | Above |
| | | protein | | | | |
| 5 | 36985_at | isopentenyl-diphosphate delta | IDI1 | X17025 | 125.47 | Above |
| | | isomerase | | | | |
| 6 | 38578_at | tumor necrosis factor receptor | TNFRSF7 | M63928 | 115.72 | Above |
| | | superfamily member 7 | | | | |
| 7 | 38203_at | potassium intermediate/small | KCNN1 | U69883 | 112.87 | Above |
| | | conductance calcium-activated | | | | |
| | | channel subfamily N member 1 | | | | |
| 8 | 35614_at | transcription factor-like 5 basic | TCFL5 | AB012124 | 108.45 | Above |
| | | helix-loop-helix | | | | |
| 9 | 32224_at | KIAA0769 gene product | KIAA0769 | AB018312 | 107.08 | Above |
| 10 | 32730_at | Homo sapiens mRNA for | | AL080059 | 104.93 | Above |
| | | KIAA1750 protein partial cds | | | | |
| 11 | 35665_at | phosphoinositide-3-kinase class 3 | PIK3C3 | Z46973 | 104.83 | Above |
| 12 | 1077_at | recombination activating gene 1 | RAG1 | M29474 | 102.90 | Above |
| 13 | 36524_at | Rho guanine nucleotide | ARHGEF4 | AB029035 | 100.67 | Above |
| | | exchange factor GEF 4 | | | | |
| 14 | 34194_at | Homo sapiens cDNA FLJ21697 | | AL049313 | 98.31 | Above |
| | | fis clone COL09740 | | | | |
| 15 | 36937_s_at | PDZ and LIM domain 1 elfin | PDLIM1 | U90878 | 96.91 | Below |
| 16 | 36008_at | protein tyrosine phosphatase | PTP4A3 | AF041434 | 96.68 | Above |
| | | type IVA member 3 | | | | |
| 17 | 1299_at | telomeric repeat binding factor 2 | TERF2 | X93512 | 93.08 | Above |
| 18 | 41814_at | fucosidase alpha-L-1 tissue | FUCA1 | M29877 | 92.77 | Above |
| 19 | 41200_at | CD36 antigen collagen type I | CD36L1 | Z22555 | 90.86 | Above |
| | | receptor thrombospondin | | | | |
| | | receptor like 1 | | | | |
| 20 | 35238_at | TNF receptor-associated factor 5 | TRAF5 | AB000509 | 90.81 | Above |
| 21 | 880_at | FK506-binding protein 1A 12 kD | FKBP1A | M34539 | 86.69 | Above |
| 22 | 33690_at | Homo sapiens mRNA cDNA | | AL080190 | 86.69 | Above |
| | | DKFZp434A202 from clone | | | | |
| | | DKFZp434A202 | | | | |
| 23 | 40272_at | collapsin response mediator | CRMP1 | D78012 | 85.44 | Above |
| | | protein 1 | | | | |
| 24 | 35362_at | myosin X | MYO10 | AB018342 | 83.60 | Above |
| 25 | 41819_at | FYN-binding protein FYB- | FYB | U93049 | 83.25 | Above |
| | | 120/130 | | | | |
| 26 | 40279_at | KIAA0121 gene product | KIAA0121 | D50911 | 81.66 | Above |
| 27 | 1488_at | protein tyrosine phosphatase | PTPRK | L77886 | 81.66 | Above |
| | | receptor type K | | | | |
| 28 | 1325_at | MAD mothers against | MADH1 | U59423 | 81.17 | Above |
| | | decapentaplegic Drosophila | | | | |
| | | homolog 1 | | | | |
| 29 | 37908_at | guanine nucleotide binding | GNG11 | U31384 | 80.37 | Above |
| | | protein 11 | | | | |
| 30 | 769_s_at | annexin A2 | ANXA2 | D00017 | 78.68 | Below |
| 31 | 33415_at | non-metastatic cells 2 protein | NME2 | X58965 | 77.04 | Below |
| | | NM23B expressed in | | | | |
| 32 | 1980_s_at | non-metastatic cells 2 protein | NME2 | X58965 | 76.35 | Below |
| | | NM23B expressed in | | | | |
| 33 | 32579_at | SWI/SNF related matrix | SMARCA4 | D26156 | 76.35 | Above |
| | | associated actin dependent | | | | |
| | | regulator of chromatin | | | | |
| | | subfamily a member 4 | | | | |
| 34 | 39425_at | thioredoxin reductase 1 | TXNRD1 | X91247 | 75.97 | Above |
| 35 | 755_at | inositol 1 4 5-triphosphate | ITPR1 | D26070 | 75.56 | Above |
| | | receptor type 1 | | | | |
| 36 | 37343_at | inositol 1 4 5-triphosphate | ITPR3 | U01062 | 75.11 | Above |
| | | receptor type 3 | | | | |
| 37 | 1336_s at | protein kinase C beta 1 | PRKCB1 | X06318 | 73.96 | Above |
| 38 | 41097_at | telomeric repeat binding factor 2 | TERF2 | AF002999 | 73.84 | Above |
| 39 | 31786_at | Sam68-like phosphotyrosine | T-STAR | AF051321 | 73.72 | Above |
| | | protein T-STAR | | | | |
| 40 | 160029_at | protein kinase C beta 1 | PRKCB1 | X07109 | 73.66 | Above |

2. Correlation-based Feature Selection (CFS)

The Correlation-based Feature Selection (CFS) is a method that evaluates subsets of genes rather than individual genes. (Hall and Holmes (2000),“Benchmarking Attribute Selection Techniques for Data Mining,” Working Paper 00/10, Department of Computer Science, University of Waikato, New Zealand). The core of the algorithm is a subset evaluation heuristic that takes into account the usefulness of individual features for predicting the class along with the level of intercorrelation among them with the belief that “good feature subsets contain features highly correlated with the class, yet uncorrelated with each other”. The heuristic assigns a score Meritₛ to a subset S containing k genes, defined as Meritₛ=(k*r_{cf})/sqrt(k+k*(k−1)*r_{ff}), where r_{cf} is the average gene-class correlation and r_{ff} is the average gene-gene correlation. Like the Chi square method, CFS first discretizes the gene expressions into intervals and then calculates a matrix of gene-class and gene-gene correlations from the training data for merit calculation. The correlation between two genes or a gene and a class is calculated as r_{xy}=2*[H(X)+H(Y)−H(X,Y)]/[H(X)+H(Y)], where H(X) is the entropy of a gene X. CFS starts from an empty set of genes and uses the best-first search technique with a stopping criterion of 5 consecutive fully expanded non-improving subsets. The subset with the highest merit found during the search is selected. Tables 9-15 list the top gene subsets chosen by CFS for each subtype. For subtype prediction, each gene subset must be used in its entirety, as within each subset, all genes are equally ranked.

TABLE 9
Genes selected by CFS: BCR-ABL

| | | | | | Above/ |
|---|---|---|---|---|---|
| | Affymetrix | | | Reference | Below |
| | number | Gene Name | GeneSymbol | number | Mean |
| 1 | 36650_at | cyclin D2 | CCND2 | D13639 | Above |
| 2 | 40196_at | HYA22 protein | HYA22 | D88153 | Above |
| 3 | 1635_at | proto-oncogene tyrosine-protein | ABL | U07563 | Above |
| | | kinase (ABL) gene | | | |
| 4 | 33775_s_at | caspase 8 apoptosis-related cysteine | CASP8 | X98176 | Above |
| | | protease | | | |
| 5 | 1636_g_at | proto-oncogene tyrosine-protein | ABL | U07563 | Above |
| | | kinase (ABL) gene | | | |
| 6 | 41295_at | GTT1 protein | GTT1 | AL041780 | Above |
| 7 | 1326_at | caspase 10 apoptosis-related cysteine | CASP10 | U60519 | Above |
| | | protease | | | |
| 8 | 33150_at | disrupter of silencing 10 | SAS10 | AI126004 | Above |
| 9 | 40051_at | TRAM-like protein | KIAA0057 | D31762 | Above |
| 10 | 39061_at | bone marrow stromal cell antigen 2 | BST2 | D28137 | Above |
| 11 | 33172_at | hypothetical protein FLJ10849 | FLJ10849 | T75292 | Above |
| 12 | 37399_at | aldo-keto reductase family 1 member | AKR1C3 | D17793 | Above |
| | | C3 3-alpha hydroxysteroid | | | |
| | | dehydrogenase type II | | | |
| 13 | 317_at | protease cysteine 1 legumain | PRSC1 | D55696 | Above |
| 14 | 330_s_at | tubulin, alpha 1, isoform 44 | TUBA1 | HG2259- | Above |
| | | | | HT2348 | |
| 15 | 38578_at | tumor necrosis factor receptor | TNFRSF7 | M63928 | Above |
| | | superfamily member 7 | | | |
| 16 | 39044_s_at | diacylglycerol kinase delta 130 kD | DGKD | D73409 | Below |
| 17 | 32562_at | endoglin Osler-Rendu-Weber | ENG | X72012 | Above |
| | | syndrome 1 | | | |
| 18 | 38641_at | Homo sapiens mRNA for TSC-22- | | AJ133115 | Above |
| | | like protein | | | |
| 19 | 1211_s_at | CASP2 and RIPK1 domain containing | CRADD | U84388 | Above |
| | | adaptor with death domain | | | |
| 20 | 39730_at | v-abl Abelson murine leukemia viral | ABL1 | X16416 | Above |
| | | oncogene homolog 1 | | | |
| 21 | 36591_at | tubulin alpha 1 testis specific | TUBA1 | X06956 | Above |
| 22 | 36035_at | anchor attachment protein 1 Gaa1p | GPAA1 | AB002135 | Above |
| | | yeast homolog | | | |
| 23 | 980_at | Niemann-Pick disease type C1 | NPC1 | AF002020 | Above |
| 24 | 40698_at | C-type calcium dependent | CLECSF2 | X96719 | Above |
| | | carbohydrate-recognition domain | | | |
| | | lectin superfamily member 2 | | | |
| | | activation-induced | | | |
| 25 | 39330_s_at | actinin alpha 1 | ACTN1 | M95178 | Above |
| 26 | 2001_g_at | ataxia telangiectasia mutated includes | ATM | U26455 | Above |
| | | complementation groups A C and D | | | |
| 27 | 39319_at | lymphocyte cytosolic protein 2 SH2 | LCP2 | U20158 | Above |
| | | domain-containing leukocyte protein | | | |
| | | of 76 kD | | | |
| 28 | 37685_at | Clathrin assembly lymphoid-myeloid | CLTH | U45976 | Above |
| | | leukemia gene | | | |
| 29 | 33813_at | tumor necrosis factor receptor | TNFRSF1B | AI813532 | Above |
| | | superfamily member 1B | | | |
| 30 | 33134_at | adenylate cyclase 3 | ADCY3 | AB011083 | Above |
| 31 | 36536_at | schwannomin interacting protein 1 | SCHIP-1 | AF070614 | Above |
| 32 | 36985_at | isopentenyl-diphosphate delta | IDI1 | X17025 | Below |
| | | isomerase | | | |
| 33 | 35991_at | Sm protein F | LSM6 | AA917945 | Above |
| 34 | 33774_at | caspase 8 apoptosis-related cysteine | CASP8 | X98172 | Above |
| | | protease | | | |
| 35 | 37470_at | leukocyte-associated Ig-like receptor 1 | LAIR1 | AF013249 | Above |
| 36 | 39245_at | Human 40871 mRNA partial | | U72507 | Above |
| | | sequence | | | |
| 37 | 40076_at | tumor protein D52-like 2 | TPD52L2 | AF004430 | Below |
| 38 | 39370_at | Microtubule-associated proteins 1A | MAP1ALC3 | W28807 | Below |
| | | and 1B light chain 3 | | | |
| 39 | 41594_at | Janus kinase 1 a protein tyrosine | JAK1 | M64174 | Above |
| | | kinase | | | |
| 40 | 41338_at | amino-terminal enhancer of split | AES | AI969192 | Below |
| 41 | 32319_at | tumor necrosis factor ligand | TNFSF4 | AL022310 | Above |
| | | superfamily member 4 tax- | | | |
| | | transcriptionally activated | | | |
| | | glycoprotein 1 34 kD | | | |
| 42 | 33924_at | KIAA1091 protein | KIAA1091 | AB029014 | Above |
| 43 | 37397_at | platelet/endothelial cell adhesion | PECAM | L34657 | Above |
| | | molecule-1 (PECAM-1) gene | | | |
| 44 | 37190_at | WAS protein family member 1 | WASF1 | D87459 | Below |
| 45 | 39070_at | singed Drosophila like sea urchin | SNL | U03057 | Above |
| | | fascin homolog like | | | |
| 46 | 38994_at | STAT induced STAT inhibitor-2 | STATI2 | AF037989 | Above |
| 47 | 32621_at | down-regulator of transcription 1 | DR1 | M97388 | Above |
| | | TBP-binding negative cofactor 2 | | | |
| 48 | 40108_at | KIAA0005 gene product | KIAA0005 | D13630 | Below |
| 49 | 35238_at | TNF receptor-associated factor 5 | TRAF5 | AB000509 | Above |
| 50 | 1558_g_at | p21/Cdc42/Rac1-activated kinase 1 | PAK1 | U24152 | Above |
| | | yeast Ste20-related | | | |
| 51 | 1373_at | transcription factor 3 E2A | TCF3 | M31523 | Below |
| | | immunoglobulin enhancer binding | | | |
| | | factors E12/E47 | | | |
| 52 | 35731_at | integrin alpha 4 antigen CD49D alpha | ITGA4 | X16983 | Above |
| | | 4 subunit of VLA-4 receptor | | | |
| 53 | 38659_at | suppressor of clear C. elegans | SHOC2 | AB020669 | Below |
| | | homolog of | | | |

TABLE 10
Gene selected by CFS for E2A-PBX1

| | | | | | Above/ |
|---|---|---|---|---|---|
| | Affymetrix | | Gene | Reference | Below |
| | number | Gene Name | Symbol | number | Mean |
| 1 | 33355_at | Homo sapiens | PBX1 | AL049381 | Above |
| | | cDNA FLJ12900 | | | |
| | | fis clone NT2RP | | | |
| | | 2004321 | | | |
| | | (by CELERA | | | |
| | | search of target | | | |
| | | sequence = PBX1) | | | |

TABLE 11
Genes selected by CFS for: Hyperdiploid >50

| | | | | | Above/ |
|---|---|---|---|---|---|
| | Affymetrix | | | Reference | Below |
| | number | Gene Name | GeneSymbol | number | Mean |
| 1 | 36620_at | superoxide dismutase 1 soluble | SOD1 | X02317 | Above |
| | | amyotrophic lateral sclerosis 1 adult | | | |
| 2 | 37350_at | clone 889N15 on chromosome | PSMD10 | AL031177 | Above |
| | | Xq22.1-22.3. Contains part of the | | | |
| | | gene for a novel protein similar to X. | | | |
| | | laevis Cortical Thymocyte Marker | | | |
| | | CTX | | | |
| 3 | 41724_at | accessory proteins BAP31/BAP29 | DXS1357E | X81109 | Above |
| 4 | 38738_at | SMT3 suppressor of mif two 3 yeast | SMT3H1 | X99584 | Above |
| | | homolog 1 | | | |
| 5 | 40480_s_at | FYN oncogene related to SRC FGR | FYN | M14333 | Above |
| | | YES | | | |
| 6 | 38518_at | sex comb on midleg Drosophila like 2 | SCML2 | Y18004 | Above |
| 7 | 31492_at | muscle specific gene | M9 | AB019392 | Below |
| 8 | 35688_g_at | mature T-cell proliferation 1 | MTCP1 | Z24459 | Above |
| 9 | 35939_s_at | POU domain class 4 transcription | POU4F1 | L20433 | Above |
| | | factor 1 | | | |
| 10 | 36128_at | transmembrane trafficking protein | TMP21 | L40397 | Above |
| 11 | 37014_at | myxovirus influenza resistance 1 | MX1 | M33882 | Above |
| | | homolog of murine interferon- | | | |
| | | inducible protein p78 | | | |
| 12 | 34374_g_at | upstream regulatory element binding | UREB1 | Z97054 | Above |
| | | protein 1 | | | |
| 13 | 688_at | proteasome prosome macropain 26S | PSMC1 | L02426 | Above |
| | | subunit ATPase 1 | | | |
| 14 | 39878_at | protocadherin 9 | PCDH9 | AI524125 | Below |
| 15 | 38771_at | histone deacetylase 1 | HDAC1 | D50405 | Below |
| 16 | 865_at | ribosomal protein S6 kinase 90 kD | RPS6KA3 | U08316 | Above |
| | | polypeptide 3 | | | |
| 17 | 41143_at | calmodulin (CALM1) gene | CALM1 | U12022 | Above |
| 18 | 39867_at | Tu translation elongation factor | TUFM | S75463 | Below |
| | | mitochondrial | | | |
| 19 | 41470_at | prominin mouse like 1 | PROML1 | AF027208 | Above |
| 20 | 41503_at | KIAA0854 protein | KIAA0854 | AB020661 | Below |
| 21 | 2039_s_at | FYN oncogene related to SRC FGR | FYN | M14333 | Above |
| | | YES | | | |
| 22 | 36845_at | KIAA0136 protein | KIAA0136 | D50926 | Above |
| 23 | 36940_at | TGFB1-induced anti-apoptotic factor 1 | TIAF1 | D86970 | Above |
| 24 | 32236_at | ubiquitin-conjugating enzyme E2G 2 | UBE2G2 | AF032456 | Above |
| | | homologous to yeast UBC7 | | | |
| 25 | 36885_at | spleen tyrosine kinase | SYK | L28824 | Below |
| 26 | 40200_at | heat shock transcription factor 1 | HSF1 | M64673 | Below |
| 27 | 40842_at | U1 snRNP-specific protein A gene | SNRPA | M60784 | Below |
| 28 | 40514_at | hypothetical 43.2 kD protein | LOC51614 | AF091085 | Below |
| 29 | 41222_at | signal transducer and activator of | STAT6 | AF067575 | Below |
| | | transcription 6 (STAT6) gene | | | |
| 30 | 1294_at | ubiquitin-activating enzyme E1-like | UBE1L | L13852 | Below |
| 31 | 34315_at | AFG3 ATPase family gene 3 yeast | AFG3L2 | Y18314 | Above |
| | | like 2 | | | |
| 32 | 39806_at | DKFZP547E2110 protein | DKFZP547E2110 | AL050261 | Above |
| 33 | 40875_s_at | small nuclear ribonucleoprotein 70 kD | SNRP70 | X06815 | Below |
| | | polypeptide RNP antigen | | | |
| 34 | 38458_at | cytochrome b5 (CYB5) gene | CYB5 | L39945 | Above |
| 35 | 1817_at | prefoldin 5 | PFDN5 | D89667 | Below |
| 36 | 34709_r_at | stromal antigen 2 | STAG2 | Z75331 | Above |
| 37 | 33447_at | myosin light polypeptide regulatory | MLCB | X54304 | Above |
| | | non-sarcomeric 20 kD | | | |
| 38 | 1077_at | recombination activating gene 1 | RAG1 | M29474 | Below |
| 39 | 1915_s_at | v-fos FBJ murine osteosarcoma viral | FOS | V01512 | Above |
| | | oncogene homolog | | | |
| 40 | 38854_at | KIAA0635 gene product | KIAA0635 | AB014535 | Above |
| 41 | 37732_at | RING1 and YY1 binding protein | RYBP | AL049940 | Above |
| 42 | 35940_at | POU domain class 4 transcription | POU4F1 | X64624 | Above |
| | | factor 1 | | | |
| 43 | 34733_at | splicing factor 3a subunit 1 120 kD | SF3A1 | X85237 | Below |
| 44 | 245_at | selectin L lymphocyte adhesion | SELL | M25280 | Below |
| | | molecule 1 | | | |
| 45 | 40146_at | RAP1B member of RAS oncogene | RAP1B | AL080212 | Below |
| | | family | | | |
| 46 | 40104_at | serine/threonine kinase 25 Ste20 yeast | STK25 | D63780 | Below |
| | | homolog | | | |
| 47 | 430_at | nucleoside phosphorylase | NP | X00737 | Above |
| 48 | 36899_at | special AT-rich sequence binding | SATB1 | M97287 | Below |
| | | protein 1 binds to nuclear | | | |
| | | matrix/scaffold-associating DNA s | | | |
| 49 | 35727_at | hypothetical protein FLJ20517 | FLJ20517 | AI249721 | Below |
| 50 | 38649_at | KIAA0970 protein | KIAA0970 | AB023187 | Below |
| 51 | 36107_at | ATP synthase H transporting | ATP5J | AA845575 | Above |
| | | mitochondrial F0 complex subunit F6 | | | |
| 52 | 38789_at | transketolase Wernicke-Korsakoff | TKT | L12711 | Below |
| | | syndrome | | | |
| 53 | 39301_at | calpain 3 p94 | CAPN3 | X85030 | Below |
| 54 | 41278_at | BAF53 | BAF53A | AF041474 | Below |
| 55 | 41162_at | protein phosphatase 1G formerly 2C | PPM1G | Y13936 | Below |
| | | magnesium-dependent gamma | | | |
| | | isoform | | | |
| 56 | 37819_at | hypothetical protein | LOC54104 | AF007130 | Below |
| 57 | 38717_at | DKFZP586A0522 protein | DKFZP586A0522 | AL050159 | Below |
| 58 | 40019_at | ecotropic viral integration site 2B | EVI2B | M60830 | Above |
| 59 | 39489_g_at | protocadherin 9 | PCDH9 | W27720 | Below |
| 60 | 857_at | protein phosphatase 1A formerly 2C | PPM1A | S87759 | Above |
| | | magnesium-dependent alpha isoform | | | |
| 61 | 32804_at | RNA binding motif protein 5 | RBM5 | AF091263 | Below |
| 62 | 37676_at | phosphodiesterase 8A | PDE8A | AF056490 | Below |
| 63 | 1519_at | v-ets avian erythroblastosis virus E26 | ETS2 | J04102 | Above |
| | | oncogene homolog 2 | | | |
| 64 | 37680_at | A kinase PRKA anchor protein gravin | AKAP12 | U81607 | Below |
| | | 12 | | | |
| 65 | 548_s_at | spleen tyrosine kinase | SYK | S80267 | Below |
| 66 | 39797_at | KIAA0349 protein | KIAA0349 | AB002347 | Above |
| 67 | 32789_at | nuclear cap binding protein subunit 2 | NCBP2 | AA149428 | Below |
| | | 20 kD | | | |
| 68 | 38091_at | lectin galactoside-binding soluble 9 | LGALS9 | Z49107 | Below |
| | | galectin 9 | | | |
| 69 | 41223_at | cytochrome c oxidase subunit Va | COX5A | M22760 | Below |
| 70 | 933_f_at | zinc finger protein 91 HPF7 HTF10 | ZNF91 | L11672 | Below |
| 71 | 37012_at | capping protein actin filament muscle | CAPZB | U03271 | Below |
| | | Z-line beta | | | |
| 72 | 35214_at | UDP-glucose dehydrogenase | UGDH | AF061016 | Above |
| 73 | 32434_at | myristoylated alanine-rich protein | MACS | D10522 | Above |
| | | kinase C substrate MARCKS 80K-L | | | |
| 74 | 38345_at | centrosomal protein 1 | CEP1 | AF083322 | Below |
| 75 | 40404_s_at | CDC16 cell division cycle 16 S. | CDC16 | U18291 | Below |
| | | cerevisiae homolog | | | |
| 76 | 39096_at | SON DNA binding protein | SON | AB028942 | Above |
| 77 | 33429_at | DKFZP586M1523 protein | DKFZP586M1523 | AL050225 | Above |
| 78 | 40641_at | TBP-associated factor 172 | TAF-172 | AF038362 | Above |
| 79 | 41381_at | KIAA0308 protein | KIAA0308 | AB002306 | Below |
| 80 | 35135_at | Homo sapiens Similar to CG15084 | | X13956 | Below |
| | | gene product clone MGC 10471 | | | |
| | | mRNA complete cds | | | |
| 81 | 39421_at | runt-related transcription factor 1 | RUNX1 | D43969 | Below |
| | | acute myeloid leukemia 1 aml1 | | | |
| | | oncogene | | | |
| 82 | 195_s_at | caspase 4 apoptosis-related cysteine | CASP4 | U28014 | Below |
| | | protease | | | |
| 83 | 36898_r_at | primase polypeptide 2A 58 kD | PRIM2A | X74331 | Above |
| 84 | 38792_at | spermine synthase | SMS | AD001528 | Above |
| 85 | 32643_at | glucan 1 4-alpha-branching enzyme 1 | GBE1 | L07956 | Below |
| | | glycogen branching enzyme Andersen | | | |
| | | disease glycogen storage disease type | | | |
| | | IV | | | |
| 86 | 38808_at | cell membrane glycoprotein 110000M | GP110 | D64154 | Below |
| | | r surface antigen | | | |
| 87 | 36062_at | Leupaxin | LPXN | AF062075 | Below |
| 88 | 300_f_at | transcription factor BTF3 homolog | | HG4518- | Below |
| | | (GB: M90355) | | HT4921 | |
| 89 | 1979_s_at | nucleolar protein 1 120 kD | NOL1 | X55504 | Below |
| 90 | 32230_at | eukaryotic translation initiation factor | EIF3S2 | U39067 | Below |
| | | 3 subunit 2 beta 36 kD | | | |
| 91 | 39893_at | guanine nucleotide binding protein G | GNG7 | AB010414 | Below |
| | | protein gamma 7 | | | |
| 92 | 34651_at | catechol-O-methyltransferase | COMT | M58525 | Above |
| 93 | 1052_s_at | CCAAT/enhancer binding protein | CEBPD | M83667 | Below |
| | | C/EBP delta | | | |
| 94 | 36272_r_at | peripheral myelin protein 2 | PMP2 | X62167 | Below |
| 95 | 2044_s_at | retinoblastoma 1 including | RB1 | M15400 | Below |
| | | osteosarcoma | | | |
| 96 | 32135_at | sterol regulatory element binding | SREBF1 | U00968 | Below |
| | | transcription factor 1 | | | |

TABLE 12
Genes selected by CFS for MLL

| | | | | | Above/ |
|---|---|---|---|---|---|
| | Affymetrix | | | Reference | Below |
| | number | Gene Name | GeneSymbol | number | Mean |
| 1 | 34306_at | muscleblind Drosophila like | MBNL | AB007888 | Above |
| 2 | 40797_at | a disintegrin and metalloproteinase | ADAM10 | AF009615 | Above |
| | | domain 10 | | | |
| 3 | 33412_at | LGALS1 Lectin, galactoside-binding, | LGALS1 | AI535946 | Above |
| | | soluble, 1 (galectin 1) | | | |
| 4 | 39338_at | S100 calcium-binding protein A10 | S100A10 | AI201310 | Above |
| | | annexin II ligand calpactin I light | | | |
| | | polypeptide p11 | | | |
| 5 | 2062_at | insulin-like growth factor binding | IGFBP7 | L19182 | Above |
| | | protein 7 | | | |
| 6 | 32193_at | plexin C1 | PLXNC1 | AF030339 | Above |
| 7 | 40518_at | protein tyrosine phosphatase receptor | PTPRC | Y00062 | Above |
| | | type C | | | |
| 8 | 36777_at | DNA segment on chromosome 12 | D12S2489E | AJ001687 | Above |
| | | unique 2489 expressed sequence | | | |
| 9 | 38391_at | capping protein actin filament | CAPG | M94345 | Above |
| | | gelsolin-like | | | |
| 10 | 40763_at | Meis1 mouse homolog | MEIS1 | U85707 | Above |
| 11 | 34721_at | FK506-binding protein 5 | FKBP5 | U42031 | Above |
| 12 | 37809_at | homeo box A9 | HOXA9 | U41813 | Above |
| 13 | 32215_i_at | KIAA0878 protein | KIAA0878 | AB020685 | Above |
| 14 | 38160_at | lymphocyte antigen 75 | LY75 | AF011333 | Above |
| 15 | 1389_at | membrane metallo-endopeptidase | MME | J03779 | Below |
| | | neutral endopeptidase enkephalinase | | | |
| | | CALLA CD10 | | | |
| 16 | 34168_at | deoxynucleotidyltransferase terminal | DNTT | M11722 | Below |
| 17 | 40522_at | glutamate-ammonia ligase glutamine | GLUL | X59834 | Above |
| | | synthase | | | |
| 18 | 854_at | B lymphoid tyrosine kinase | BLK | S76617 | Above |
| 19 | 40067_at | E74-like factor 1 ets domain | ELF1 | M82882 | Above |
| | | transcription factor | | | |
| 20 | 39756_g_at | X-box binding protein 1 | XBP1 | Z93930 | Below |
| 21 | 32134_at | Testing | DKFZP586B2022 | AL050162 | Above |
| 22 | 39379_at | Homo sapiens mRNA cDNA | | AL049397 | Above |
| | | DKFZp586C1019 from clone | | | |
| | | DKFZp586C1019 | | | |
| 23 | 40415_at | acetyl-Coenzyme A acyltransferase 1 | ACAA1 | X14813 | Above |
| | | peroxisomal 3-oxoacyl-Coenzyme A | | | |
| | | thiolase | | | |
| 24 | 40519_at | protein tyrosine phosphatase receptor | PTPRC | Y00638 | Above |
| | | type C | | | |
| 25 | 33847_s_at | cyclin-dependent kinase inhibitor 1B | CDKN1B | U10906 | Above |
| | | p27 Kip1 | | | |
| 26 | 32696_at | pre-B-cell leukemia transcription | PBX3 | X59841 | Above |
| | | factor 3 | | | |
| 27 | 40417_at | KIAA0098 protein | | D43950 | Above |
| 28 | 1644_at | eukaryotic translation initiation factor | EIF3S2 | U36764 | Above |
| | | 3 subunit 2 beta 36 kD | | | |
| 29 | 948_s_at | peptidylprolyl isomerase D | PPID | D63861 | Above |
| | | cyclophilin D | | | |
| 30 | 34337_s_at | putative DNA binding protein | M96 | AJ010014 | Below |
| 31 | 41747 s_at | myocyte-specific enhancer factor 2A | MEF2A | U49020 | Above |
| | | (MEF2A) gene | | | |
| 32 | 39516_at | hypothetical protein | HSPC004 | AI827793 | Above |
| 33 | 31820_at | hematopoietic cell-specific Lyn | HCLS1 | X16663 | Above |
| | | substrate 1 | | | |
| 34 | 33305_at | serine or cysteine proteinase inhibitor | SERPINB1 | M93056 | Above |
| | | clade B ovalbumin member 1 | | | |
| 35 | 40520_g_at | protein tyrosine phosphatase receptor | PTPRC | Y00638 | Above |
| | | type C | | | |
| 36 | 41222_at | signal transducer and activator of | STAT6 | AF067575 | Above |
| | | transcription 6 (STAT6) gene | | | |
| 37 | 1718_at | actin related protein 2/3 complex | ARPC2 | U50523 | Above |
| | | subunit 2 34 kD | | | |
| 38 | 38342_at | KIAA0239 protein | KIAA0239 | D87076 | Below |
| 39 | 38805_at | TG-interacting factor TALE family | TGIF | X89750 | Below |
| | | homeobox | | | |
| 40 | 32089_at | sperm associated antigen 6 | SPAG6 | AF079363 | Above |
| 41 | 1950_s_at | Smad 3, exon 1 | | AB004922 | Above |
| 42 | 39410_at | development and differentiation | DDEF2 | AB007860 | Above |
| | | enhancing factor 2 | | | |
| 43 | 37280_at | MAD mothers against | MADH1 | U59912 | Below |
| | | decapentaplegic Drosophila homolog 1 | | | |
| 44 | 32607_at | brain acid-soluble protein 1 | BASP1 | AF039656 | Above |
| 45 | 39389_at | CD9 antigen p24 | CD9 | M38690 | Below |
| 46 | 40913_at | ATPase Ca transporting plasma | ATP2B4 | W28589 | Below |
| | | membrane 4 | | | |
| 47 | 1039_s_at | hypoxia-inducible factor 1 alpha | HIF1A | U22431 | Below |
| | | subunit basic helix-loop-helix | | | |
| | | transcription factor | | | |
| 48 | 35939_s_at | POU domain class 4 transcription | POU4F1 | L20433 | Below |
| | | factor 1 | | | |
| 49 | 963_at | ligase IV DNA ATP-dependent | LIG4 | X83441 | Below |
| 50 | 39628_at | RAB9 member RAS oncogene family | RAB9 | U44103 | Below |
| 51 | 38242_at | B cell linker protein | SLP65 | AF068180 | Below |
| 52 | 37692_at | diazepam binding inhibitor GABA | DBI | AI557240 | Above |
| | | receptor modulator acyl-Coenzyme A | | | |
| | | binding protein | | | |
| 53 | 32166_at | KIAA1027 protein | KIAA1027 | AB028950 | Above |
| 54 | 34800_at | DKFZP586O1624 protein | DKFZP586O1624 | AL039458 | Below |
| 55 | 34386_at | methyl-CpG binding domain protein 4 | MBD4 | AF072250 | Below |
| 56 | 40296_at | hypothetical protein | 753P9 | AL023653 | Below |
| 57 | 40456_at | up-regulated by BCG-CWS | LOC64116 | AL049963 | Above |
| 58 | 33943_at | ferritin heavy polypeptide 1 | FTH1 | L20941 | Below |
| 59 | 39049_at | G18.1a and G18.1b proteins (G18.1a | | AJ243937 | Below |
| | | and G18.1b genes, located in the class | | | |
| | | III region of the major | | | |
| | | histocompatibility complex) | | | |
| 60 | 38075_at | synaptophysin-like protein | SYPL | X68194 | Above |
| 61 | 932_i_at | zinc finger protein 91 HPF7 HTF10 | ZNF91 | L11672 | Below |
| 62 | 1825_at | IQ motif containing GTPase | IQGAP1 | L33075 | Above |
| | | activating protein 1 | | | |
| 63 | 34210_at | CDW52 antigen CAMPATH-1 | CDW52 | N90866 | Below |
| | | antigen | | | |
| 64 | 39778_at | mannosyl alpha-1 3- glycoprotein | MGAT1 | M55621 | Below |
| | | beta-1 2-N- | | | |
| | | acetylglucosaminyltransferase | | | |
| 65 | 34699_at | CD2-associated protein | CD2AP | AL050105 | Below |
| 66 | 40066_at | ubiquitin-activating enzyme E1C | UBE1C | AF046024 | Above |
| | | homologous to yeast UBA3 | | | |
| 67 | 41177_at | hypothetical protein FLJ12443 | FLJ12443 | AW024285 | Above |
| 68 | 32736_at | HSPC022 protein | HSPC022 | W68830 | Above |
| 69 | 1928_s_at | mad protein homolog Smad2 gene | Smad2 | U78733 | Below |
| 70 | 1081_at | ornithine decarboxylase 1 | ODC1 | M33764 | Above |
| 71 | 37345_at | Calumenin | CALU | AF013759 | Above |
| 72 | 34099_f_at | nucleosome assembly protein 1-like 1 | NAP1L1 | W26056 | Above |
| 73 | 933_f_at | zinc finger protein 91 HPF7 HTF10 | ZNF91 | L11672 | Below |
| 74 | 32214_at | thioredoxin-like 32 kD | TXNL | AF003938 | Below |
| 75 | 33501_r_at | SNC73 protein SNC73 mRNA | | S71043 | Below |
| | | complete cds | | | |
| 76 | 950_at | translocation protein 1 | TLOC1 | D87127 | Below |
| 77 | 41161_at | death-associated protein 6 | DAXX | AB015051 | Below |
| 78 | 41381_at | KIAA0308 protein | KIAA0308 | AB002306 | Below |
| 79 | 38705_at | ubiquitin-conjugating enzyme E2D 2 | UBE2D2 | AI310002 | Above |
| | | homologous to yeast UBC4/5 | | | |
| 80 | 38617_at | LIM domain kinase 2 | LIMK2 | D45906 | Below |
| 81 | 34305_at | poly rC binding protein 1 | PCBP1 | Z29505 | Above |
| 82 | 40436_g_at | solute carrier family 25 mitochondrial | SLC25A6 | J03592 | Above |
| | | carrier adenine nucleotide translocator | | | |
| | | member 6 | | | |
| 83 | 1827_s_at | c-myc-P64 mRNA, initiating from | | M13929 | Above |
| | | promoter P0 | | | |
| 84 | 38479_at | acidic protein rich in leucines | SSP29 | Y07969 | Below |
| 85 | 33207_at | DnaJ Hsp40 homolog subfamily C | DNAJC3 | AI095508 | Below |
| | | member 3 | | | |
| 86 | 39039_s_at | CGI-76 protein | LOC51632 | AI557497 | Below |
| 87 | 32157_at | protein phosphatase 1 catalytic | PPP1CA | S57501 | Above |
| | | subunit alpha isoform | | | |
| 88 | 905_at | guanylate kinase 1 | GUK1 | L76200 | Below |
| 89 | 35794_at | KIAA0942 protein | KIAA0942 | AB023159 | Below |
| 90 | 1007_s_at | discoidin domain receptor family | DDR1 | U48705 | Below |
| | | member 1 | | | |
| 91 | 39424_at | tumor necrosis factor receptor | TNFRSF14 | U70321 | Below |
| | | superfamily member 14 herpesvirus | | | |
| | | entry mediator | | | |
| 92 | 36634_at | BTG family member 2 | BTG2 | U72649 | Below |
| 93 | 38760_f_at | butyrophilin subfamily 3 member A2 | BTN3A2 | U90546 | Below |

TABLE 13
Genes selected by CFS for Novel Class

| | | | | | Above/ |
|---|---|---|---|---|---|
| | Affymetrix | | | Reference | Below |
| | number | Gene Name | GeneSymbol | number | Mean |
| 1 | 37960_at | carbohydrate chondroitin 6/keratan | CHST2 | AB014679 | Above |
| | | sulfotransferase 2 | | | |
| 2 | 31892_at | protein tyrosine phosphatase receptor | PTPRM | X58288 | Above |
| | | type M | | | |
| 3 | 994_at | protein tyrosine phosphatase receptor | PTPRM | X58288 | Above |
| | | type M | | | |
| 4 | 995_g_at | protein tyrosine phosphatase receptor | PTPRM | X58288 | Above |
| | | type M | | | |
| 5 | 41074_at | G protein-coupled receptor 49 | GPR49 | AF062006 | Above |
| 6 | 41073_at | G protein-coupled receptor 49 | GPR49 | AI743745 | Above |
| 7 | 34676_at | KIAA1099 protein | KIAA1099 | AB029022 | Above |
| 8 | 36139_at | DKFZP586G0522 protein | DKFZP586G0522 | AL050289 | Above |
| 9 | 37542_at | lipoma HMGIC fusion partner-like 2 | LHFPL2 | D86961 | Above |
| 10 | 41159_at | clathrin heavy polypeptide Hc | CLTC | D21260 | Above |
| 11 | 32800_at | retinoid X receptor alpha mRNA | | U66306 | Above |
| 12 | 1664_at | insulin-like growth factor 2 | IGF2 | HG3543- | Above |
| | | | | HT3739 | |
| 13 | 36566_at | cystinosis nephropathic | CTNS | AJ222967 | Above |

TABLE 14
Gene selected by CFS for T-ALL

| | | | | | Above/ |
|---|---|---|---|---|---|
| | Affymetrix | | | Reference | Below |
| | number | Gene Name | GeneSymbol | number | Mean |
| 1 | 38319_at | CD3D antigen | CD3D | AA919102 | Above |
| | | delta | | | |
| | | polypeptide | | | |
| | | TiT3 complex | | | |

TABLE 15
Genes selected by CFS for TEL-AML1L

| | | | | | Above/ |
|---|---|---|---|---|---|
| | Affymetrix | | | Reference | Below |
| | number | Gene Name | GeneSymbol | number | Mean |
| 1 | 38652_at | hypothetical protein FLJ20154 | FLJ20154 | AF070644 | Above |
| 2 | 36239_at | POU domain class 2 associating | POU2AF1 | Z49194 | Above |
| | | factor 1 | | | |
| 3 | 41442_at | core-binding factor runt domain alpha | CBFA2T3 | AB010419 | Above |
| | | subunit 2 translocated to 3 | | | |
| 4 | 37780_at | piccolo presynaptic cytomatrix | PCLO | AB011131 | Above |
| | | protein | | | |
| 5 | 36985_at | isopentenyl-diphosphate delta | IDI1 | X17025 | Above |
| | | isomerase | | | |
| 6 | 38578_at | tumor necrosis factor receptor | TNFRSF7 | M63928 | Above |
| | | superfamily member 7 | | | |
| 7 | 35614_at | transcription factor-like 5 basic helix- | TCFL5 | AB012124 | Above |
| | | loop-helix | | | |
| 8 | 32224_at | KIAA0769 gene product | KIAA0769 | AB018312 | Above |
| 9 | 32730_at | KIAA1750 protein | | AL080059 | Above |
| 10 | 36937_s_at | PDZ and LIM domain 1 elfin | PDLIM1 | U90878 | Below |
| 11 | 36008_at | protein tyrosine phosphatase type IVA | PTP4A3 | AF041434 | Above |
| | | member 3 | | | |
| 12 | 41200_at | CD36 antigen collagen type I receptor | CD36L1 | Z22555 | Above |
| | | thrombospondin receptor like 1 | | | |
| 13 | 33690_at | DKFZp434A202 from clone | | AL080190 | Above |
| | | DKFZp434A202 | | | |
| 14 | 755_at | inositor 1 4 5-triphosphate receptor | ITPR1 | D26070 | Above |
| | | type 1 | | | |
| 15 | 41097_at | telomeric repeat binding factor 2 | TERF2 | AF002999 | Above |
| 16 | 160029_at | protein kinase C beta 1 | PRKCB1 | X07109 | Above |
| 17 | 34481_at | vav proto-oncogene | Vav | AF030227 | Above |
| 18 | 41498_at | KIAA0911 protein | KIAA0911 | AB020718 | Above |
| 19 | 37280_at | MAD mothers against | MADH1 | U59912 | Above |
| | | decapentaplegic Drosphila homolog | | | |
| | | 1 | | | |
| 20 | 1647_at | IQ motif containing GTPase | IQGAP2 | U51903 | Below |
| | | activating protein 2 | | | |
| 21 | 37724_at | v-myc avian myelocytomatosis viral | MYC | V00568 | Below |
| | | oncogene homolog | | | |
| 22 | 37981_at | drebrin 1 | DBN1 | U00802 | Above |
| 23 | 37326_at | proteolipid protein 2 colonic | PLP2 | U93305 | Below |
| | | epithelium-enriched | | | |
| 24 | 37344_at | major histocompatibility complex | HLA-DMA | X62744 | Above |
| | | class II DM alpha | | | |
| 25 | 38666_at | pleckstrin homology Sec7 and | PSCD1 | M85169 | Below |
| | | coiled/coil domains 1 cytohesin 1 | | | |
| 26 | 39039_s_at | CGI-76 protein | LOC51632 | AI557497 | Below |
| 27 | 34819_at | CD164 antigen sialomucin | CD164 | D14043 | Below |
| 28 | 40729_s_at | nuclear factor of kappa light | NFKBIL1 | Y14768 | Above |
| | | polypeptide gene enhancer in B-cells | | | |
| | | inhibitor-like 1 | | | |
| 29 | 34224_at | fatty acid desaturase 3 | FADS3 | AC004770 | Above |
| 30 | 39827_at | hypothetical protein | FLJ20500 | AA522530 | Below |
| 31 | 32157_at | protein phosphatase 1 catalytic | PPP1CA | S57501 | Below |
| | | subunit alpha isoform | | | |
| 32 | 34183_at | DKFZP434C171 protein | DKFZP434C17 | AL080169 | Below |
| | | | 1 | | |
| 33 | 39329_at | actinin alpha 1 | ACTN1 | X15804 | Below |
| 34 | 38124_at | midkine neurite growth-promoting | MDK | X55110 | Above |
| | | factor 2 | | | |
| 35 | 33304_at | interferon stimulated gene 20 kD | ISG20 | U88964 | Above |
| 36 | 41295_at | GTT1 protein | GTT1 | AL041780 | Below |
| 37 | 40745_at | adaptor-related protein complex 1 | AP1B1 | L13939 | Above |
| | | beta 1 subunit | | | |
| 38 | 38906_at | spectrin alpha erythrocytic 1 | SPTA1 | M61877 | Above |
| | | elliptocytosis 2 | | | |
| 39 | 263_g_at | S-adenosylmethionine decarboxylase | AMD1 | M21154 | Below |
| | | 1 | | | |
| 40 | 41609_at | major histocompatibility complex | HLA-DMB | U15085 | Above |
| | | class II DM beta | | | |
| 41 | 39045_at | hypothetical protein FLJ21432 | FLJ21432 | W26655 | Below |
| 42 | 39421_at | runt-related transcription factor 1 | RUNX1 | D43969 | Above |
| | | acute myeloid leukemia 1 aml1 | | | |
| | | oncogene | | | |
| 43 | 34210_at | CDW52 antigen CAMPATH-1 | CDW52 | N90866 | Above |
| | | antigen | | | |
| 44 | 37276_at | IQ motif containing GTPase | IQGAP2 | U51903 | Below |
| | | activating protein 2 | | | |
| 45 | 38763_at | L-iditol-2 dehydrogenase gene | | L29254 | Below |
| 46 | 40960_at | UDP-Gal betaGlcNAc beta 1 4- | B4GALT1 | D29805 | Below |
| | | galactosyltransferase polypeptide 1 | | | |
| 47 | 1127_at | ribosomal protein S6 kinase 90 kD | RPS6KA1 | L07597 | Below |
| | | polypeptide 1 | | | |
| 48 | 37359_at | KIAA0102 gene product | KIAA0102 | D14658 | Below |
| 49 | 38968_at | SH3-domain binding protein 5 BTK- | SH3BP5 | AB005047 | Below |
| | | associated | | | |
| 50 | 39135_at | KIAA0767 protein | KIAA0767 | AB018310 | Below |
| 51 | 36128_at | transmembrane trafficking protein | TMP21 | L40397 | Below |
| 52 | 1158_s_at | calmodulin 3 phosphorylase kinase | CALM3 | J04046 | Above |
| | | delta | | | |
| 53 | 34782_at | jumonji mouse homolog | JMJ | AL021938 | Below |
| 54 | 37893_at | protein tyrosine phosphatase non- | PTPN2 | AI828880 | Below |
| | | receptor type 2 | | | |
| 55 | 39758_f_at | Lysosomal-associated membrane | LAMP1 | J04182 | Below |
| | | protein 1 | | | |
| 56 | 35151_at | tumor suppressor deleted in oral | DOC-1R | AF089814 | Below |
| | | cancer-related 1 | | | |
| 57 | 38096_f_at | major histocompatibility complex | HLA-DPB1 | M83664 | Above |
| | | class II DP beta 1 | | | |
| 58 | 40467_at | succinate dehydrogenase complex | SDHD | AB006202 | Below |
| | | subunit D integral membrane protein | | | |
| 59 | 39712_at | S100 calcium-binding protein A13 | S100A13 | AI541308 | Below |
| 60 | 41812_s_at | KIAA0906 protein | KIAA0906 | AB020713 | Below |
| 61 | 34336_at | lysyl-tRNA synthetase | KARS | D32053 | Below |
| 62 | 38336_at | KIAA1013 protein | KIAA1013 | AB023230 | Below |
| 63 | 32253_at | arginine-glutamic acid dipeptide RE | RERE | AB007927 | Below |
| | | repeats | | | |
| 64 | 35731_at | integrin alpha 4 antigen CD49D alpha | ITGA4 | X16983 | Below |
| | | 4 subunit of VLA-4 receptor | | | |
| 65 | 40698_at | C-type calcium dependent | CLECSF2 | X96719 | Below |
| | | carbohydrate-recognition domain | | | |
| | | lectin superfamily member 2 | | | |
| | | activation-induced | | | |
| 66 | 840_at | zinc finger protein 220 | ZNF220 | U47742 | Above |
| 67 | 41171_at | proteasome prosome macropain | PSME2 | D45248 | Above |
| | | activator subunit 2 PA28 beta | | | |
| 68 | 34877_at | Janus kinase 1 a protein tyrosine | JAK1 | AL039831 | Above |
| | | kinase | | | |
| 69 | 37190_at | WAS protein family member 1 | WASF1 | D87459 | Below |
| 70 | 31690_at | Glutamate dehydrogenase-2 | GLUD2 | U08997 | Below |
| 71 | 40961_at | SWI/SNF related matrix associated | SMARCA2 | X72889 | Below |
| | | actin dependent regulator of | | | |
| | | chromatin subfamily a member 2 | | | |
| 72 | 38149_at | KIAA0053 gene product | KIAA0053 | D29642 | Above |
| 73 | 2061_at | integrin alpha 4 antigen CD49D alpha | ITGA4 | L12002 | Below |
| | | 4 subunit of VLA-4 receptor | | | |
| 74 | 2012_s_at | protein kinase DNA-activated | PRKDC | U34994 | Below |
| | | catalytic polypeptide | | | |
| 75 | 36878_f_at | major histocompatibility complex | HLA-DQB1 | M60028 | Above |
| | | class II DQ beta 1 | | | |
| 76 | 34821_at | DKFZP586D0623 protein | DKFZP586D06 | AL050197 | Below |
| | | | 23 | | |
| 77 | 36980_at | proline-rich protein with nuclear | B4-2 | U03105 | Below |
| | | targeting signal | | | |
| 78 | 853_at | nuclear factor erythroid-derived 2 like | NFE2L2 | S74017 | Below |
| | | 2 | | | |
| 79 | 39320_at | caspase 1 apoptosis-related cysteine | CASP1 | U13697 | Below |
| | | protease interleukin 1 beta convertase | | | |
| 80 | 32572_at | ubiquitin specific protease 9 X | USP9X | X98296 | Below |
| | | chromosome Drosophila fat facets | | | |
| | | related | | | |
| 81 | 387_at | cyclin-dependent kinase 9 CDC2- | CDK9 | X80230 | Below |
| | | related kinase | | | |
| 82 | 35300_at | glutamyl-prolyl-tRNA synthetase | EPRS | X54326 | Below |
| 83 | 36155_at | KIAA0275 gene product | KIAA0275 | D87465 | Below |
| 84 | 37625_at | Interfuron regulatory factor 4 | IRF4 | U52682 | Below |
| 85 | 35763_at | KIAA0540 protein | KIAA0540 | AB011112 | Below |
| 86 | 39077_at | DR1-associated protein 1 negative | DRAP1 | U41843 | Below |
| | | cofactor 2 alpha | | | |
| 87 | 40132_g_at | Follistatin-like 1 | FSTL1 | D89937 | Below |
| 88 | 32615_at | aspartyl-tRNA synthetase | DARS | J05032 | Below |
| 89 | 38357_at | Homo sapiens mRNA cDNA | | AL049321 | Above |
| | | DKFZp564D156 from clone | | | |
| | | DKFZp564D156 | | | |
| 90 | 34817_s_at | ataxin 2 related protein | A2LP | U70671 | Above |
| 91 | 40856_at | serine or cysteine proteinase inhibitor | SERPINF1 | U29953 | Below |
| | | clade F alpha-2 antiplasmin pigment | | | |
| | | epithelium derived factor member 1 | | | |
| 92 | 39784_at | eukaryotic translation initiation factor | EIF2S1 | U26032 | Below |
| | | 2 subunit 1 alpha 35 kD | | | |
| 93 | 37600_at | extracellular matrix protein 1 | ECM1 | U68186 | Below |
| 94 | 40839_at | ubiquitin-like 3 | UBL3 | AL080177 | Below |
| 95 | 34832_s_at | KIAA0763 gene product | KIAA0763 | AB018306 | Below |
| 96 | 33244_at | chimerin chimaerin 2 | CHN2 | U07223 | Below |
| 97 | 31516_f_at | basic transcription factor 3 like 1 | BTF3L1 | M90354 | Below |
| 98 | 35266_at | bladder cancer associated protein | BLCAP | AL049288 | Above |
| 99 | 253_g_at | (clone GPCR W) G protein-linked | | L42324 | Below |
| | | receptor gene (GPCR) gene | | | |
| 100 | 35227_at | retinoblastoma-binding protein 8 | RBBP8 | U72066 | Below |
| 101 | 41073_at | G protein-coupled receptor 49 | GPR49 | AI743745 | Below |
| 102 | 38084_at | chromobox homolog 3 Drosophila | CBX3 | AI797801 | Below |
| | | HP1 gamma | | | |
| 103 | 39025_at | 6.2 kd protein | LOC54543 | AI557912 | Below |
| 104 | 32085_at | KIAA0981 protein | KIAA0981 | AB023198 | Above |
| 105 | 38902_r_at | Activating transcription factor 2 | ATF2 | X15875 | Below |

3. T-statistics

T-statistics is a classical feature selection approach. The t-statistics of a gene is defined as T=|μ₁−μ₂|/sqrt(σ₁²/n₁+σ₂²/n₂), where μᵢ is the mean expression of that gene in the i^{th} class, σᵢ² is the variance of that gene in the i^{th} class and nᵢ is the size of the i^{th} class. This formula assigns higher value to a gene that has larger mean difference between two classes and has smaller variance within both classes. For BCR-ABL, hyperdiploid >50, MLL, Novel, and TEL-AML1 the top ranked 40 genes are listed in Tables 16, 18, 19, 20, and 22, whereas for E2A-PBX1 and T-ALL only the top 30 and 31 genes are shown. Additional genes that may be used in expression profiles to assign subjects to a leukemia risk group are shown in Tables 54-60. The genes in Tables 54-60 were selected on the basis of having a T-statistic value greater than the T-statistic vlaue for the gene when examined as a disciminator in 999 of 1000 permutations of the data set (p<0.001; this statistical test is described elsewhere herein). Of these genes, only those having a T-statistic absolute values equal to or greater than 8 (representing a nominal p value of ˜<0.0001) are shown in Tables 54-50.

Generally, using the top 20-40 genes did not result in significant changes to subtype prediction accuracy. Accordingly, the top 20 genes were used for subtype prediction, unless noted otherwise.

TABLE 16
Genes Selected by T statistics for BCR-ABL

| | | | | | | Above/ |
|---|---|---|---|---|---|---|
| | Affymetrix | | Gene | Reference | T-stat | Below |
| | number | Gene Name | Symbol | number | value | Mean |
| 1 | 32319_at | tumor necrosis factor ligand | TNFSF4 | AL022310 | 12.0346 | Above |
| | | superfamily member 4 tax- | | | | |
| | | transcriptionally activated | | | | |
| | | glycoprotein 1 34 kD | | | | |
| 2 | 36194_at | low density lipoprotein-related | LRPAP1 | M63959 | −11.3077 | Below |
| | | protein-associated protein 1 alpha- | | | | |
| | | 2-macroglobulin receptor- | | | | |
| | | associated protein 1 | | | | |
| 3 | 1211_s_at | CASP2 and RIPK1 domain | CRADD | U84388 | 10.6627 | Above |
| | | containing adaptor with death | | | | |
| | | domain | | | | |
| 4 | 37397_at | Homo sapiens platelet/endothelial | PECAM | L34657 | 10.2460 | Above |
| | | cell adhesion molecule-1 | | | | |
| | | (PECAM-1) gene, exon 16 and | | | | |
| | | complete cds. | | | | |
| 5 | 330_s_at | tubulin, alpha 1, isoform 44 | TUBA1 | HG2259- | 10.0540 | Above |
| | | | | HT2348 | | |
| 6 | 33774_at | caspase 8 apoptosis-related | CASP8 | X98172 | 9.9147 | Above |
| | | cysteine protease | | | | |
| 7 | 202_at | heat shock transcription factor 2 | HSF2 | M65217 | −9.7639 | Below |
| 8 | 1558_g_at | p21/Cdc42/Rac1-activated kinase | PAK1 | U24152 | 9.6562 | Above |
| | | 1 yeast Ste20-related | | | | |
| 9 | 39691_at | SH3-containing protein SH3GLB1 | SH3GLB1 | AB007960 | 9.5307 | Above |
| 10 | 2045_s_at | hemopoietic cell kinase | HCK | M16592 | −9.3898 | Below |
| 11 | 36591_at | tubulin alpha 1 testis specific | TUBA1 | X06956 | 9.3382 | Above |
| 12 | 1386_at | protein tyrosine phosphatase non- | PTPN9 | M83738 | −9.2414 | Below |
| | | receptor type 9 | | | | |
| 13 | 35991_at | Sm protein F | LSM6 | AA917945 | 9.0298 | Above |
| 14 | 41273_at | FK506 binding protein 12- | FRAP1 | AL046940 | 8.9732 | Above |
| | | rapamycin associated protein 1 | | | | |
| 15 | 35970_g_at | M-phase phosphoprotein 9 | MPHOSPH9 | N23137 | 8.6474 | Above |
| 16 | 38636_at | immunoglobulin superfamily | ISLR | AB003184 | 8.4291 | Above |
| | | containing leucine-rich repeat | | | | |
| 17 | 36683_at | matrix Gla protein | MGP | AI953789 | −8.3872 | Below |
| 18 | 39070_at | singed Drosophila like sea urchin | SNL | U03057 | 8.2583 | Above |
| | | fascin homolog like | | | | |
| 19 | 40798_s_at | a disintegrin and metalloproteinase | ADAM10 | Z48579 | 8.2283 | Above |
| | | domain 10 | | | | |
| 20 | 41649_at | FOXJ2 forkhead factor | LOC55810 | AF038177 | 8.2275 | Above |
| 21 | 38966_at | glycoprotein synaptic 2 | GPSN2 | AF038958 | 8.2080 | Above |
| 22 | 34759_at | Human hbc647 mRNA sequence | | U68494 | 8.1863 | Above |
| 23 | 1434_at | phosphatase and tensin homolog | PTEN | U92436 | 8.1671 | Above |
| | | mutated in multiple advanced | | | | |
| | | cancers 1 | | | | |
| 24 | 40167_s_at | CS box-containing WD protein | LOC55884 | AF038187 | 8.1655 | Above |
| 25 | 40264_g_at | zinc finger protein-like 1 | ZFPL1 | AF001891 | 8.1384 | Above |
| 26 | 36129_at | KIAA0397 gene product | KIAA0397 | AB007857 | 8.0041 | Above |
| 27 | 551_at | E1A binding protein p300 | EP300 | U01877 | −7.7578 | Below |
| 28 | 38345_at | centrosomal protein 1 | CEP1 | AF083322 | −7.7431 | Below |
| 29 | 41137_at | myosin phosphatase target subunit 2 | MYPT2 | AB007972 | −7.7301 | Below |
| 30 | 39068_at | protein phosphatase 2 regulatory | PPP2R5D | L76702 | −7.6161 | Below |
| | | subunit B B56 delta isoform | | | | |
| 31 | 38160_at | lymphocyte antigen 75 | LY75 | AF011333 | 7.5830 | Above |
| 32 | 34314_at | ribonucleotide reductase M1 | RRM1 | X59543 | 7.5778 | Above |
| | | polypeptide | | | | |
| 33 | 39519_at | KIAA0692 protein | KIAA0692 | AB014592 | 7.4662 | Above |
| 34 | 32788_at | RAN binding protein 2 | RANBP2 | D42063 | 7.4114 | Above |
| 35 | 34882_at | nucleolar protein KKE/D repeat | NOP56 | Y12065 | 7.3622 | Above |
| 36 | 2064_g_at | excision repair cross- | ERCC5 | L20046 | 7.3597 | Above |
| | | complementing rodent repair | | | | |
| | | deficiency complementation group 5 | | | | |
| 37 | 41836_at | protein with polyglutamine repeat | ERPROT213-21 | U94836 | 7.3350 | Above |
| | | calcium ca2 homeostasis | | | | |
| | | endoplasmic reticulum protein | | | | |
| 38 | 1563_s_at | tumor necrosis factor receptor | TNFRSF1A | M58286 | 7.3039 | Above |
| | | superfamily member 1A | | | | |
| 39 | 37047_at | Niemann-Pick disease type C1 | NPC1 | AF002020 | 7.2357 | Above |
| 40 | 32724_at | phytanoyl-CoA hydroxylase | PHYH | AF023462 | −7.2252 | Below |
| | | Refsum disease | | | | |

TABLE 17
Genes Selected by T statistics for E2A-PBX1

| | | | | | | Above/ |
|---|---|---|---|---|---|---|
| | Affymetrix | | Gene | Reference | T-stat | Below |
| | number | Gene Name | Symbol | number | value | Mean |
| 1 | 32063_at | pre-B-cell leukemia transcription | PBX1 | M86546 | 126.7442 | Above |
| | | factor 1 | | | | |
| 2 | 33355_at | Homo sapiens cDNA FLJ12900 | PBX1 | AL049381 | 36.6116 | Above |
| | | fis clone NT2RP2004321 (by | | | | |
| | | CELERA search of target | | | | |
| | | sequence = PBX1) | | | | |
| 3 | 40454_at | FAT tumor suppressor Drosophila | FAT | X87241 | 30.7577 | Above |
| | | homolog | | | | |
| 4 | 717_at | GS3955 protein | GS3955 | D87119 | 23.7813 | Above |
| 5 | 39070_at | singed Drosophila like sea urchin | SNL | U03057 | −22.8956 | Below |
| | | fascin homolog like | | | | |
| 6 | 33641_g_at | nuclear factor of kappa light | NFKBIL1 | Y14768 | −20.4637 | Below |
| | | polypeptide gene enhancer in B- | | | | |
| | | cells inhibitor-like 1 | | | | |
| 7 | 36536_at | schwannomin interacting protein 1 | SCHIP-1 | AF070614 | −20.1554 | Below |
| 8 | 854_at | B lymphoid tyrosine kinase | BLK | S76617 | 19.6467 | Above |
| 9 | 37625_at | interferon regulatory factor 4 | IRF4 | U52682 | 18.8419 | Above |
| 10 | 39614_at | KIAA0802 protein | KIAA0802 | AB018345 | 17.8214 | Above |
| 11 | 37099_at | arachidonate 5-lipoxygenase- | ALOX5AP | AI806222 | −17.7944 | Below |
| | | activating protein | | | | |
| 12 | 38994_at | STAT induced STAT inhibitor-2 | STATI2 | AF037989 | −17.6553 | Below |
| 13 | 37641_at | Human gene for hepatitis C- | | D28915 | −17.3074 | Below |
| | | associated microtubular aggregate | | | | |
| | | protein p44, exon 9 and complete | | | | |
| | | cds. | | | | |
| 14 | 40113_at | GS3955 protein | GS3955 | D87119 | 16.7288 | Above |
| 15 | 2031_s_at | cyclin-dependent kinase inhibitor | CDKN1A | U03106 | −14.9826 | Below |
| | | 1A p21 Cip1 | | | | |
| 16 | 330_s_at | tubulin, alpha 1, isoform 44 | TUBA1 | HG2259- | −14.8016 | Below |
| | | | | HT2348 | | |
| 17 | 38340_at | huntingtin interacting protein-1- | KIAA0655 | AB014555 | 14.7180 | Above |
| | | related | | | | |
| 18 | 38510_at | Homo sapiens mRNA cDNA | | AL049435 | −14.4522 | Below |
| | | DKFZp586B0220 | | | | |
| 19 | 268_at | Homo sapiens platelet/endothelial | PECAM | L34657 | −13.7540 | Below |
| | | cell adhesion molecule-1 | | | | |
| | | (PECAM-1) gene, exon 16 and | | | | |
| | | complete cds. | | | | |
| 20 | 2062_at | insulin-like growth factor binding | IGFBP7 | L19182 | 13.6403 | Above |
| | | protein 7 | | | | |
| 21 | 37893_at | protein tyrosine phosphatase non- | PTPN2 | AI828880 | 13.5099 | Above |
| | | receptor type 2 | | | | |
| 22 | 38580_at | guanine nucleotide binding protein | GNAQ | U43083 | −12.8525 | Below |
| | | G protein q polypeptide | | | | |
| 23 | 40049_at | death-associated protein kinase 1 | DAPK1 | X76104 | −12.3837 | Below |
| 24 | 38393_at | KIAA0247 gene product | KIAA0247 | D87434 | 12.3436 | Above |
| 25 | 39379_at | Homo sapiens mRNA cDNA | | AL049397 | 12.2102 | Above |
| | | DKFZp586C1019 | | | | |
| 26 | 430_at | nucleoside phosphorylase | NP | X00737 | 12.1307 | Above |
| 27 | 37975_at | cytochrome b-245 beta | CYBB | X04011 | −12.0743 | Below |
| | | polypeptide chronic | | | | |
| | | granulomatous disease | | | | |
| 28 | 34862_at | CGI-49 protein | LOC51097 | AA005018 | 12.0264 | Above |
| 29 | 39756_g_at | X-box binding protein 1 | XBP1 | Z93930 | −11.9796 | Below |
| 30 | 307_at | arachidonate 5-lipoxygenase | ALOX5 | J03600 | −11.9492 | Below |
| 31 | 37304_at | chromobox homolog 1 Drosophila | CBX1 | U35451 | 11.9422 | Above |
| | | HP1 beta | | | | |
| 32 | 1287_at | ADP-ribosyltransferase NAD poly | ADPRT | J03473 | 11.9051 | Above |
| | | ADP-ribose polymerase | | | | |
| 33 | 1520_s_at | interleukin 1 beta | IL1B | X04500 | 11.7327 | Above |
| 34 | 596_s_at | colony stimulating factor 3 | CSF3R | M59820 | −11.6814 | Below |
| | | receptor granulocyte | | | | |
| 35 | 37493_at | colony stimulating factor 2 | CSF2RB | H04668 | 11.6620 | Above |
| | | receptor beta low-affinity | | | | |
| | | granulocyte-macrophage | | | | |
| 36 | 36452_at | synaptopodin | KIAA1029 | AB028952 | 11.4021 | Above |
| 37 | 1081_at | ornithine decarboxylase 1 | ODC1 | M33764 | 11.2865 | Above |
| 38 | 1563_s_at | tumor necrosis factor receptor | TNFRSF1A | M58286 | −11.1361 | Below |
| | | superfamily member 1A | | | | |
| 39 | 39069_at | AE-binding protein 1 | AEBP1 | AF053944 | 11.0984 | Above |
| 40 | 36203_at | ornithine decarboxylase 1 | ODC1 | X16277 | 10.9475 | Above |

TABLE 18
Genes Selected by T statistics for Hyperdiploid >50

| | | | | | | Above/ |
|---|---|---|---|---|---|---|
| | Affymetrix | | Gene | Reference | T-stat | Below |
| | number | Gene Name | Symbol | number | value | Mean |
| 1 | 36620_at | superoxide dismutase 1 soluble | SOD1 | X02317 | 9.1574 | Above |
| | | amyotrophic lateral sclerosis 1 | | | | |
| | | adult | | | | |
| 2 | 39878_at | protocadherin 9 | PCDH9 | AI524125 | −6.9008 | Below |
| 3 | 37543_at | Rac/Cdc42 guanine exchange | ARHGEF6 | D25304 | 6.8366 | Above |
| | | factor GEF 6 | | | | |
| 4 | 41470_at | prominin mouse like 1 | PROML1 | AF027208 | 6.7290 | Above |
| 5 | 31492_at | muscle specific gene | M9 | AB019392 | −6.6885 | Below |
| 6 | 38968_at | SH3-domain binding protein 5 | SH3BP5 | AB005047 | 6.4051 | Above |
| | | BTK-associated | | | | |
| 7 | 1915_s_at | v-fos FBJ murine osteosarcoma | FOS | V01512 | 6.4008 | Above |
| | | viral oncogene homolog | | | | |
| 8 | 37677_at | phosphoglycerate kinase 1 | PGK1 | V00572 | 6.2865 | Above |
| 9 | 39867_at | Tu translation elongation factor | TUFM | S75463 | −6.2299 | Below |
| | | mitochondrial | | | | |
| 10 | 36795_at | prosaposin variant Gaucher | PSAP | J03077 | 6.1812 | Above |
| | | disease and variant metachromatic | | | | |
| | | leukodystrophy | | | | |
| 11 | 40875_s_at | small nuclear ribonucleoprotein | SNRP70 | X06815 | −6.0877 | Below |
| | | 70 kD polypeptide RNP antigen | | | | |
| 12 | 306_s_at | high-mobility group nonhistone | HMG14 | J02621 | 6.0804 | Above |
| | | chromosomal protein 14 | | | | |
| 13 | 41724_at | accessory proteins BAP31/BAP29 | DXS1357E | X81109 | 6.0244 | Above |
| 14 | 39168_at | Ac-like transposable element | ALTE | AB018328 | 5.9336 | Above |
| 15 | 955_at | calmodulin type I | CALM1 | HG1862- | 5.8650 | Above |
| | | | | HT1897 | | |
| 16 | 38604_at | neuropeptide Y | NPY | AI198311 | 5.8313 | Above |
| 17 | 39147_g_at | alpha thalassemia/mental | ATRX | U72936 | 5.8181 | Above |
| | | retardation syndrome X-linked | | | | |
| | | RAD54 S. cerevisiae homolog | | | | |
| 18 | 39069_at | AE-binding protein 1 | AEBP1 | AF053944 | −5.6901 | Below |
| 19 | 37014_at | myxovirus influenza resistance 1 | MX1 | M33882 | 5.6688 | Above |
| | | homolog of murine interferon- | | | | |
| | | inducible protein p78 | | | | |
| 20 | 1520_s_at | interleukin 1 beta | IL1B | X04500 | 5.6605 | Above |
| 21 | 1488_at | protein tyrosine phosphatase | PTPRK | L77886 | −5.5877 | Below |
| | | receptor type K | | | | |
| 22 | 32553_at | MYC-associated zinc finger | MAZ | M94046 | −5.5000 | Below |
| | | protein purine-binding | | | | |
| | | transcription factor | | | | |
| 23 | 36169_at | NADH dehydrogenase ubiquinone | NDUFA1 | N47307 | 5.4376 | Above |
| | | 1 alpha subcomplex 1 7.5 kD | | | | |
| | | MWFE | | | | |
| 24 | 1817_at | prefoldin 5 | PFDN5 | D89667 | −5.4110 | Below |
| 25 | 578_at | Human recombination acitivating | RAG2 | M94633 | −5.4026 | Below |
| | | protein (RAG2) gene, last exon | | | | |
| 26 | 1556_at | RNA binding motif protein 5 | RBM5 | U23946 | −5.3032 | Below |
| 27 | 40998_at | trinucleotide repeat containing 11 | TNRC11 | AF071309 | 5.2349 | Above |
| | | THR-associated protein 230 kDa | | | | |
| | | subunit | | | | |
| 28 | 37294_at | B-cell translocation gene 1 anti- | BTG1 | X61123 | −5.1877 | Below |
| | | proliferative | | | | |
| 29 | 1447_at | proteasome prosome macropain | PSMB1 | D00761 | 5.1699 | Above |
| | | subunit beta type 1 | | | | |
| 30 | 35940_at | POU domain class 4 transcription | POU4F1 | X64624 | 5.1200 | Above |
| | | factor 1 | | | | |
| 31 | 33307_at | kraken-like | BK126B4.1 | AL022316 | −5.0984 | Below |
| 32 | 1081_at | ornithine decarboxylase 1 | ODC1 | M33764 | −5.0822 | Below |
| 33 | 34336_at | lysyl-tRNA synthetase | KARS | D32053 | −5.0692 | Below |
| 34 | 41143_at | Human calmodulin (CALM1) | CALM1 | U12022 | 5.0543 | Above |
| | | gene, exons 2, 3, 4, 5 and | | | | |
| | | 6, and complete cds | | | | |
| 35 | 32251_at | hypothetical protein FLJ21174 | FLJ21174 | AA149307 | 5.0373 | Above |
| 36 | 35298_at | eukaryotic translation initiation | EIF3S7 | U54558 | −4.9499 | Below |
| | | factor 3 subunit 7 zeta 66/67 kD | | | | |
| 37 | 38649_at | KIAA0970 protein | KIAA0970 | AB023187 | −4.9228 | Below |
| 38 | 36629_at | glucocorticoid-induced leucine | GILZ | AI635895 | 4.8061 | Above |
| | | zipper | | | | |
| 39 | 39721_at | ephrin-B1 | EFNB1 | U09303 | 4.7968 | Above |
| 40 | 2094_s_at | v-fos FBJ murine osteosarcoma | FOS | K00650 | 4.7446 | Above |
| | | viral oncogene homolog | | | | |

TABLE 19
Genes Selected by T statistics for MLL

| | | | | | | Above/ |
|---|---|---|---|---|---|---|
| | Affymetrix | | Gene | Reference | T-stat | Below |
| | number | Gene Name | Symbol | number | value | Mean |
| 1 | 307_at | arachidonate 5-lipoxygenase | ALOX5 | J03600 | −16.8244 | Below |
| 2 | 37280_at | MAD mothers against | MADH1 | U59912 | −15.4460 | Below |
| | | decapentaplegic Drosophila | | | | |
| | | homolog 1 | | | | |
| 3 | 1520_s_at | interleukin 1 beta | IL1B | X04500 | −13.6764 | Below |
| 4 | 36908_at | Human macrophage mannose | MRC1 | M93221 | −11.8629 | Below |
| | | receptor (MRC1) gene, exon 30. | | | | |
| 5 | 33412_at | LGALS1 Lectin, galactoside- | LGALS1 | AI535946 | 11.0223 | Above |
| | | binding, soluble, 1 (galectin 1) | | | | |
| 6 | 2062_at | insulin-like growth factor binding | IGFBP7 | L19182 | 10.4318 | Above |
| | | protein 7 | | | | |
| 7 | 35940_at | POU domain class 4 transcription | POU4F1 | X64624 | −10.1815 | Below |
| | | factor 1 | | | | |
| 8 | 39721_at | ephrin-B1 | EFNB1 | U09303 | −9.6158 | Below |
| 9 | 39402_at | interleukin 1 beta | IL1B | M15330 | −9.5998 | Below |
| 10 | 1737_s_at | insulin-like growth factor-binding | IGFBP4 | M62403 | −9.4119 | Below |
| | | protein 4 | | | | |
| 11 | 37413_at | dipeptidase 1 renal | DPEP1 | J05257 | −9.4101 | Below |
| 12 | 40519_at | protein tyrosine phosphatase | PTPRC | Y00638 | 9.3163 | Above |
| | | receptor type C | | | | |
| 13 | 1971_g_at | fragile histidine triad gene | FHIT | U46922 | −9.2257 | Below |
| 14 | 1983_at | cyclin D2 | CCND2 | X68452 | −9.2213 | Below |
| 15 | 38869_at | KIAA1069 protein | KIAA1069 | AB028992 | −9.1951 | Below |
| 16 | 40520_g_at | protein tyrosine phosphatase | PTPRC | Y00638 | 9.1099 | Above |
| | | receptor type C | | | | |
| 17 | 1718_at | actin related protein 2/3 complex | ARPC2 | U50523 | 9.0435 | Above |
| | | subunit 2 34 kD | | | | |
| 18 | 34237_at | HBS1 S. cerevisiae like | HBS1L | AB028961 | −8.8208 | Below |
| 19 | 1726_at | DNA polymerase, epsilon, | | HG919- | −8.4664 | Below |
| | | catalytic subunit | | HT919 | | |
| 20 | 36643_at | discoidin domain receptor family | DDR1 | L20817 | −8.4627 | Below |
| | | member 1 | | | | |
| 21 | 1325_at | MAD mothers against | MADH1 | U59423 | −8.3762 | Below |
| | | decapentaplegic Drosophila | | | | |
| | | homolog 1 | | | | |
| 22 | 39379_at | Homo sapiens mRNA cDNA | | AL049397 | 8.2974 | Above |
| | | DKFZp586C1019 | | | | |
| 23 | 36536_at | schwannomin interacting protein 1 | SCHIP-1 | AF070614 | −8.1177 | Below |
| 24 | 564_at | guanine nucleotide binding protein | GNA11 | M69013 | −8.1107 | Below |
| | | G protein alpha 11 Gq class | | | | |
| 25 | 39705_at | KIAA0700 protein | KIAA0700 | AB014600 | −7.9334 | Below |
| 26 | 36105_at | Human nonspecific crossreacting | NCA | M18728 | −7.6911 | Below |
| | | antigen mRNA, complete cds. | | | | |
| 27 | 174_s_at | intersectin 2 | ITSN2 | U61167 | 7.5752 | Above |
| 28 | 39114_at | decidual protein induced by | DEPP | AB022718 | −7.4767 | Below |
| | | progesterone | | | | |
| 29 | 40436_g_at | solute carrier family 25 | SLC25A6 | J03592 | 7.3952 | Above |
| | | mitochondrial carrier adenine | | | | |
| | | nucleotide translocator member 6 | | | | |
| 30 | 794_at | protein tyrosine phosphatase non- | PTPN6 | X62055 | 7.2192 | Above |
| | | receptor type 6 | | | | |
| 31 | 38032_at | KIAA0736 gene product | K1AA0736 | AB018279 | −7.0718 | Below |
| 32 | 40518_at | protein tyrosine phosphatase | PTPRC | Y00062 | 6.9829 | Above |
| | | receptor type C | | | | |
| 33 | 41762_at | TIA1 cytotoxic granule-associated | TIAL1 | D64015 | −6.9118 | Below |
| | | RNA-binding protein-like 1 | | | | |
| 34 | 1389_at | membrane metallo-endopeptidase | MME | J03779 | −6.7734 | Below |
| | | neutral endopeptidase | | | | |
| | | enkephalinase CALLA CD10 | | | | |
| 35 | 39967_at | leucine zipper down-regulated in | LDOC1 | AB019527 | −6.7415 | Below |
| | | cancer 1 | | | | |
| 36 | 188_at | ephrin-B1 | EFNB1 | U09303 | −6.5964 | Below |
| 37 | 160033_s_at | X-ray repair complementing | XRCC1 | NM_006297 | −6.5936 | Below |
| | | defective repair in Chinese | | | | |
| | | hamster cells 1 | | | | |
| 38 | 40913_at | ATPase Ca transporting plasma | ATP2B4 | W28589 | −6.5774 | Below |
| | | membrane 4 | | | | |
| 39 | 37398_at | platelet/endothelial cell adhesion | PECAM1 | AA100961 | −6.5675 | Below |
| | | molecule CD31 antigen | | | | |
| 40 | 1488_at | protein tyrosine phosphatase | PTPRK | L77886 | −6.5584 | Below |
| | | receptor type K | | | | |

TABLE 20
Genes Selected by T statistics for Novel Risk Group

| | | | | | | Above/ |
|---|---|---|---|---|---|---|
| | Affymetrix | | Gene | Reference | T-stat | Below |
| | number | Gene Name | Symbol | number | value | Mean |
| 1 | 41734_at | KIAA0870 protein | KIAA0870 | AB020677 | −40.5168 | Below |
| 2 | 31892_at | protein tyrosine phosphatase | PTPRM | X58288 | 33.4654 | Above |
| | | receptor type M | | | | |
| 3 | 995_g_at | protein tyrosine phosphatase | PTPRM | X58288 | 24.7557 | Above |
| | | receptor type M | | | | |
| 4 | 34676_at | KIAA1099 protein | KIAA1099 | AB029022 | 14.0491 | Above |
| 5 | 37908_at | guanine nucleotide binding protein | GNG11 | U31384 | 11.4548 | Above |
| | | 11 | | | | |
| 6 | 37960_at | carbohydrate chondroitin 6/keratan | CHST2 | AB014679 | 10.9971 | Above |
| | | sulfotransferase 2 | | | | |
| 7 | 33410_at | integrin alpha 6 | ITGA6 | S66213 | 10.0370 | Above |
| 8 | 40585_at | adenylate cyclase 7 | ADCY7 | D25538 | −9.5897 | Below |
| 9 | 33284_at | myeloperoxidase | MPO | M19507 | −9.4724 | Below |
| 10 | 41159_at | clathrin heavy polypeptide Hc | CLTC | D21260 | 9.4489 | Above |
| 11 | 36591_at | tubulin alpha 1 testis specific | TUBA1 | X06956 | −9.1387 | Below |
| 12 | 37712_g_at | MADS box transcription enhancer | MEF2C | S57212 | −9.1225 | Below |
| | | factor 2 polypeptide C myocyte | | | | |
| | | enhancer factor 2C | | | | |
| 13 | 38576_at | H2B histone family member B | H2BFB | AJ223353 | −9.0869 | Below |
| 14 | 38408_at | transmembrane 4 superfamily | TM4SF2 | L10373 | −8.7026 | Below |
| | | member 2 | | | | |
| 15 | 33907_at | eukaryotic translation initiation | EIF4G3 | AF012072 | −8.3540 | Below |
| | | factor 4 gamma 3 | | | | |
| 16 | 41273_at | FK506 binding protein 12- | FRAP1 | AL046940 | −8.3212 | Below |
| | | rapamycin associated protein 1 | | | | |
| 17 | 402_s_at | intercellular adhesion molecule 3 | ICAM3 | X69819 | −7.9741 | Below |
| 18 | 35112_at | regulator of G-protein signalling 9 | RGS9 | AF071476 | 7.8348 | Above |
| 19 | 34850_at | ubiquitin-conjugating enzyme E2E | UBE2E3 | AB017644 | 7.8197 | Above |
| | | 3 homologous to yeast UBC4/5 | | | | |
| 20 | 37030_at | KIAA0887 protein | KIAA0887 | AB020694 | −7.6343 | Below |
| 21 | 36322_at | fucosyltransferase 7 alpha 13 | FUT7 | AB012668 | −7.6240 | Below |
| | | fucosyltransferase | | | | |
| 22 | 39509_at | Homo sapiens cDNA FLJ22071 | | AI692348 | −7.6232 | Below |
| 23 | 40091_at | B-cell CLL/lymphoma 6 zinc | BCL6 | U00115 | −7.6171 | Below |
| | | finger protein 51 | | | | |
| 24 | 37280_at | MAD mothers against | MADH1 | U59912 | 7.5991 | Above |
| | | decapentaplegic Drosophila | | | | |
| | | homolog 1 | | | | |
| 25 | 1325_at | MAD mothers against | MADH1 | U59423 | 7.5824 | Above |
| | | decapentaplegic Drosophila | | | | |
| | | homolog 1 | | | | |
| 26 | 831_at | DEAD/H Asp-Glu-Ala-Asp/His | DDX10 | U28042 | 7.4276 | Above |
| | | box polypeptide 10 RNA helicase | | | | |
| 27 | 37600_at | extracellular matrix protein 1 | ECM1 | U68186 | −7.2991 | Below |
| 28 | 41266_at | integrin alpha 6 | ITGA6 | X53586 | 7.2985 | Above |
| 29 | 36958_at | zyxin | ZYX | X95735 | −7.2889 | Below |
| 30 | 36564_at | Human DNA sequence from clone | | W27419 | −7.2848 | Below |
| | | RP5-1174N9 on chromosome | | | | |
| | | 1p134.1-35.3 | | | | |
| 31 | 32174_at | solute carrier family 9 | SLC9A3R1 | AF015926 | −7.2749 | Below |
| | | sodium/hydrogen exchanger | | | | |
| | | isoform 3 regulatory factor 1 | | | | |
| 32 | 619_s_at | membrane-spanning 4-domains | MS4A2 | M27394 | −7.2325 | Below |
| | | subfamily A member 2 Fc | | | | |
| | | fragment of IgE high affinity I | | | | |
| | | receptor for beta polypeptide | | | | |
| 33 | 40749_at | membrane-spanning 4-domains | MS4A2 | X07203 | −7.2063 | Below |
| | | subfamily A member 2 Fc | | | | |
| | | fragment of IgE high affinity I | | | | |
| | | receptor for beta polypeptide | | | | |
| 34 | 31894_at | centromere protein C 1 | CENPC1 | M95724 | 6.9679 | Above |
| 35 | 32319_at | tumor necrosis factor ligand | TNFSF4 | AL022310 | 6.8225 | Above |
| | | superfamily member 4 tax- | | | | |
| | | transcriptionally activated | | | | |
| | | glycoprotein 1 34 kD | | | | |
| 36 | 38259_at | syntaxin binding protein 2 | STXBP2 | AB002559 | −6.6992 | Below |
| 37 | 35629_at | hypothetical protein | DJ1042K10.2 | AL022238 | −6.6968 | Below |
| 38 | 38700_at | cysteine and glycine-rich protein 1 | CSRP1 | M33146 | −6.6962 | Below |
| 39 | 37397_at | Homo sapiens platelet/endothelial | PECAM | L34657 | −6.6934 | Below |
| | | cell adhesion molecule-1 | | | | |
| | | (PECAM-1) gene, exon 16 and | | | | |
| | | complete cds. | | | | |
| 40 | 41127_at | solute carrier family 1 | SLC1A4 | L14595 | −6.6892 | Below |
| | | glutamate/neutral amino acid | | | | |
| | | transporter member 4 | | | | |

TABLE 21
Genes Selected by T statistics for T-ALL

| | | | | | | Above/ |
|---|---|---|---|---|---|---|
| | Affymetrix | | Gene | Reference | T-stat | Below |
| | number | Gene Name | Symbol | number | value | Mean |
| 1 | 38242_at | B cell linker protein | SLP65 | AF068180 | −115.8362 | Below |
| 2 | 38319_at | CD3D antigen delta polypeptide | CD3D | AA919102 | 27.6995 | Above |
| | | TiT3 complex | | | | |
| 3 | 37988_at | CD79B antigen immunoglobulin- | CD79B | M89957 | −23.7294 | Below |
| | | associated beta | | | | |
| 4 | 38147_at | SH2 domain protein 1A Duncan s | SH2D1A | AL023657 | 22.4501 | Above |
| | | disease lymphoproliferative | | | | |
| | | syndrome | | | | |
| 5 | 38522_s_at | CD22 antigen | CD22 | X52785 | −21.2795 | Below |
| 6 | 35350_at | B cell RAG associated protein | BRAG | AB011170 | −19.1460 | Below |
| 7 | 36277_at | Human membran protein (CD3- | CD3E | M23323 | 19.0859 | Above |
| | | epsilon) gene, exon 9. | | | | |
| 8 | 38604_at | neuropeptide Y | NPY | AI198311 | −18.8194 | Below |
| 9 | 33705_at | phosphodiesterase 4B cAMP- | PDE4B | L20971 | −18.6383 | Below |
| | | specific dunce Drosophila | | | | |
| | | homolog phosphodiesterase E4 | | | | |
| 10 | 36878_f_at | major histocompatibility complex | HLA-DQB1 | M60028 | −18.5620 | Below |
| | | class II DQ beta 1 | | | | |
| 11 | 36638_at | connective tissue growth factor | CTGF | X78947 | −18.2772 | Below |
| 12 | 32794_g_at | T cell receptor beta locus | TRB | X00437 | 17.9081 | Above |
| 13 | 32174_at | solute carrier family 9 | SLC9A3R1 | AF015926 | 17.4427 | Above |
| | | sodium/hydrogen exchanger | | | | |
| | | isoform 3 regulatory factor 1 | | | | |
| 14 | 160041_at | protein tyrosine phosphatase non- | PTPN18 | X79568 | −17.3412 | Below |
| | | receptor type 18 brain-derived | | | | |
| 15 | 38521_at | CD22 antigen | CD22 | X59350 | −17.0388 | Below |
| 16 | 38018_g_at | CD79A antigen immunoglobulin- | CD79A | U05259 | −16.7948 | Below |
| | | associated alpha | | | | |
| 17 | 36571_at | topoisomerase DNA II beta 180 kD | TOP2B | X68060 | −16.7508 | Below |
| 18 | 1096_g_at | CD19 antigen | CD19 | M28170 | −16.4583 | Below |
| 19 | 39318_at | T-cell leukemia/lymphoma 1A | TCL1A | X82240 | −16.2017 | Below |
| 20 | 41710_at | hypothetical protein | LOC54103 | AL079277 | −15.9099 | Below |
| 21 | 599_at | H2.0 Drosophila like homeo box 1 | HLX1 | M60721 | −15.5425 | Below |
| 22 | 266_s_at | CD24 antigen small cell lung | CD24 | L33930 | −15.0123 | Below |
| | | carcinoma cluster 4 antigen | | | | |
| 23 | 36502_at | PFTAIRE protein kinase 1 | PFTK1 | AB020641 | −14.9972 | Below |
| 24 | 39114_at | decidual protein induced by | DEPP | AB022718 | −14.9886 | Below |
| | | progesterone | | | | |
| 25 | 37539_at | RalGDS-like gene KIAA0959 | KIAA0959 | AB023176 | −14.6872 | Below |
| | | protein | | | | |
| 26 | 40775_at | integral membrane protein 2A | ITM2A | AL021786 | 14.5666 | Above |
| 27 | 34033_s_at | leukocyte immunoglobulin-like | LILRA2 | AF025531 | −14.3809 | Below |
| | | receptor subfamily A with TM | | | | |
| | | domain member 2 | | | | |
| 28 | 2031_s_at | cyclin-dependent kinase inhibitor | CDKN1A | U03106 | −14.1071 | Below |
| | | 1A p21 Cip1 | | | | |
| 29 | 38051_at | mal T-cell differentiation protein | MAL | X76220 | 14.0743 | Above |
| 30 | 35794_at | KIAA0942 protein | KIAA0942 | AB023159 | −13.9659 | Below |
| 31 | 41156_g_at | catenin cadherin-associated | CTNNA1 | U03100 | −13.8135 | Below |
| | | protein alpha 1 102 kD | | | | |
| 32 | 32979_at | GRB2-associated binding protein 1 | GAB1 | U43885 | −13.5842 | Below |
| 33 | 32562_at | endoglin Osler-Rendu-Weber | ENG | X72012 | −13.4209 | Below |
| | | syndrome 1 | | | | |
| 34 | 36536_at | schwannomin interacting protein 1 | SCHIP-1 | AF070614 | −13.4172 | Below |
| 35 | 36108_at | major histocompatibility complex | HLA-DQB1 | M16276 | −13.3518 | Below |
| | | class II DQ beta 1 | | | | |
| 36 | 41734_at | KIAA0870 protein | KIAA0870 | AB020677 | −13.2672 | Below |
| 37 | 41153_f_at | Homo sapiens alphaE-catenin | CTNNA1 | AF102803 | −12.7927 | Below |
| | | (CTNNA1) gene, exon 18 and | | | | |
| | | complete cds. | | | | |
| 38 | 37710_at | MADS box transcription enhancer | MEF2C | L08895 | −12.7716 | Below |
| | | factor 2 polypeptide C myocyte | | | | |
| | | enhancer factor 2C | | | | |
| 39 | 39893_at | guanine nucleotide binding protein | GNG7 | AB010414 | −12.7696 | Below |
| | | G protein gamma 7 | | | | |
| 40 | 37908_at | guanine nucleotide binding protein | GNG11 | U31384 | −12.7353 | Below |
| | | 11 | | | | |

TABLE 22
Genes Selected by T statistics for TEL-AML1

| | | | | | | Above/ |
|---|---|---|---|---|---|---|
| | Affymetrix | | Gene | Reference | T-stat | Below |
| | number | Gene Name | Symbol | number | value | Mean |
| 1 | 38578_at | tumor necrosis factor receptor | TNFRSF7 | M63928 | 15.2209 | Above |
| | | superfamily member 7 | | | | |
| 2 | 38203_at | potassium intermediate/small | KCNN1 | U69883 | 15.0804 | Above |
| | | conductance calcium-activated | | | | |
| | | channel subfamily N member 1 | | | | |
| 3 | 36524_at | Rho guanine nucleotide exchange | ARHGEF4 | AB029035 | 14.9774 | Above |
| | | factor GEF 4 | | | | |
| 4 | 37780_at | piccolo presynaptic cytomatrix | PCLO | ABO11131 | 14.1405 | Above |
| | | protein | | | | |
| 5 | 35614_at | transcription factor-like 5 basic | TCFL5 | AB012124 | 12.9369 | Above |
| | | helix-loop-helix | | | | |
| 6 | 160029_at | protein kinase C beta 1 | PRKCB1 | X07109 | 12.5429 | Above |
| 7 | 1980_s_at | non-metastatic cells 2 protein | NME2 | X58965 | −12.5035 | Below |
| | | NM23B expressed in | | | | |
| 8 | 1488_at | protein tyrosine phosphatase | PTPRK | L77886 | 12.3871 | Above |
| | | receptor type K | | | | |
| 9 | 34194_at | Homo sapiens cDNA FLJ21697 | | AL049313 | 12.1089 | Above |
| 10 | 37908_at | guanine nucleotide binding protein | GNG11 | U31384 | 11.4322 | Above |
| | | 11 | | | | |
| 11 | 40272_at | collapsin response mediator | CRMP1 | D78012 | 11.0625 | Above |
| | | protein 1 | | | | |
| 12 | 41097_at | telomeric repeat binding factor 2 | TERF2 | AF002999 | 11.0133 | Above |
| 13 | 33690_at | Homo sapiens mRNA cDNA | | AL080190 | 10.8763 | Above |
| | | DKFZp434A202 | | | | |
| 14 | 32730_at | Homo sapiens mRNA for | | AL080059 | 10.7439 | Above |
| | | KIAA1750 | | | | |
| 15 | 1325_at | MAD mothers against | MADH1 | U59423 | 10.5332 | Above |
| | | decapentaplegic Drosophila | | | | |
| | | homolog 1 | | | | |
| 16 | 41819_at | FYN-binding protein FYB- | FYB | U93049 | 10.3692 | Above |
| | | 120/130 | | | | |
| 17 | 1299_at | telomeric repeat binding factor 2 | TERF2 | X93512 | 10.2921 | Above |
| 18 | 35665_at | phosphoinositide-3-kinase class 3 | PIK3C3 | Z46973 | 10.0568 | Above |
| 19 | 36537_at | Rho-specific guanine nucleotide | P114-RHO- | AB011093 | 9.8824 | Above |
| | | exchange factor p114 | GEF | | | |
| 20 | 37280_at | MAD mothers against | MADH1 | U59912 | 9.8662 | Above |
| | | decapentaplegic Drosophila | | | | |
| | | homolog 1 | | | | |
| 21 | 1936_s_at | proto-oncogene c-myc, alt. | | HG3523- | −9.6621 | Below |
| | | transcript 3, ORF 114 | | HT4899 | | |
| 22 | 1077_at | recombination activating gene 1 | RAG1 | M29474 | 9.4563 | Above |
| 23 | 38763_at | Human (clone D21-1) L-iditol-2 | | L29254 | −9.2719 | Below |
| | | dehydrogenase gene, exon 9 and | | | | |
| | | complete cds. | | | | |
| 24 | 41295_at | GTT1 protein | GTT1 | AL041780 | −9.1813 | Below |
| 25 | 36008_at | protein tyrosine phosphatase type | PTP4A3 | AF041434 | 9.1682 | Above |
| | | IVA member 3 | | | | |
| 26 | 38570_at | major histocompatibility complex | HLA-DOB | X03066 | 9.0394 | Above |
| | | class II DO beta | | | | |
| 27 | 32163_f_at | EST | | AA216639 | 9.0392 | Above |
| 28 | 40570_at | forkhead box O1A | FOXO1A | AF032885 | 8.9931 | Above |
| | | rhabdomyosarcoma | | | | |
| 29 | 32724_at | phytanoyl-CoA hydroxylase | PHYH | AF023462 | 8.9571 | Above |
| | | Refsum disease | | | | |
| 30 | 932_i_at | zinc finger protein 91 HPF7 | ZNF91 | L11672 | 8.8075 | Above |
| | | HTF10 | | | | |
| 31 | 37343_at | inositol 1 4 5-triphosphate receptor | ITPR3 | U01062 | 8.7321 | Above |
| | | type 3 | | | | |
| 32 | 33447_at | myosin light polypeptide | MLCB | X54304 | −8.6848 | Below |
| | | regulatory non-sarcomeric 20 kD | | | | |
| 33 | 35362_at | myosin X | MYO10 | AB018342 | 8.6700 | Above |
| 34 | 38906_at | spectrin alpha erythrocytic 1 | SPTA1 | M61877 | 8.5010 | Above |
| | | elliptocytosis 2 | | | | |
| 35 | 324_f_at | basic transcription factor 3 | BTF3 | HG1515- | −8.4705 | Below |
| | | | | HT1515 | | |
| 36 | 39329_at | actinin alpha 1 | ACTN1 | X15804 | −8.3219 | Below |
| 37 | 577_at | midkine neurite growth-promoting | MDK | M94250 | 8.2693 | Above |
| | | factor 2 | | | | |
| 38 | 40729_s_at | nuclear factor of kappa light | NFKBIL1 | Y14768 | 8.2000 | Above |
| | | polypeptide gene enhancer in B- | | | | |
| | | cells inhibitor-like 1 | | | | |
| 39 | 41442_at | core-binding factor runt domain | CBFA2T3 | AB010419 | 8.0604 | Above |
| | | alpha subunit 2 translocated to 3 | | | | |
| 40 | 36275_at | Homo sapiens mRNA from | | AB002438 | 7.8550 | Above |
| | | chromosome 5q21-22 clone | | | | |
| | | FBR89 | | | | |

4. Wilkins'

This method of selecting genes uses the weighted sum of three components to estimate the discriminative value of each gene. The higher the score, the better the gene is at discriminating between the two classes. The input to the scoring method is preprocessed and normalized data. The idea of the metric is that a gene is a good discriminator if: (1) it is expressed in one class and not in the other, or if the gene is expressed in both classes, but significantly more so in one than the other, or (2) the gene is present in most samples, and the data are pure, in the sense that there is a threshold expression value for the gene where the gene generally has expression levels larger than the threshold in one class, and smaller than the threshold in the other class. The components of the metric were quantified as follows. For a gene, assume PR₁ is the ratio of “present” samples to all samples in class 1, where present means that the gene's expression value was not preprocessed to a constant (1). Assume PR₂ is defined similarly for class 2. The first component of the metric, M₁, is estimated as the absolute difference between PR₁ and PR₂. This value is between 0 (when the gene is equally present in both classes) and 1 (when the gene is expressed in one class and not in the other). The second component of the metric, M₂, measures the extent to which the gene is present overall, and is defined as the average of PR₁ and PR₂. The final component, M₃, estimates the “purity”, or existence of a threshold value. The gene expression values for the present samples are sorted into ascending order and a vector of their class labels is built, for example {+, +, +, −, −, −, +, −, −, +, −}. The next step is to find the best place to partition the samples so that the expression values for one class (maybe +) are less than the partition point, and the values from the other class are larger. Let L_{C1} and L_{C2} be the number of class 1 and class 2 samples on the left side of the partition, respectively. Assume R_{C1} and R_{C2} are defined similarly for the right side of the partition. Then the purity is estimated as: max {L_{C1}-L_{C2}+R_{C2}−R_{C1}, L_{C2}−L_{C1}+R_{C1}−R_{C2}}/ number of total present samples. Each possible partition is checked. In the example above, the partition {+, +, +, ∥−, −, −, +, −, −, +, −} is the best partition, with a purity value of M₃=7/11=0.64. The score for the gene is the weighted sum of 0.5*M₁+0.25*M₂+0.25*M₃. The top 50 genes for each subgroup selected by this metric are listed in Tables 23-29. For class prediction all 50 genes were used, unless otherwise stated.

TABLE 23
Genes Selected by Wilkins' for BCR-ABL

| | | | | | | Above/ |
|---|---|---|---|---|---|---|
| | Affymetrix | | Gene | Reference | Train set | Below |
| | number | Gene Name | Symbol | number | score | Mean |
| 1 | 32319_at | tumor necrosis factor ligand | TNFSF4 | AL022310 | 0.6354 | Above |
| | | superfamily member 4 tax- | | | | |
| | | transcriptionally activated | | | | |
| | | glycoprotein 1 34 kD | | | | |
| 2 | 37479_at | CD72 antigen | CD72 | M54992 | 0.6352 | Below |
| 3 | 1211_s_at | CASP2 and RIPK1 domain | CRADD | U84388 | 0.6265 | Above |
| | | containing adaptor with death | | | | |
| | | domain | | | | |
| 4 | 37397_at | platelet/endothelial cell adhesion | PECAM | L34657 | 0.6161 | Above |
| | | molecule-1 (PECAM-1) gene | | | | |
| 5 | 33162_at | insulin receptor | INSR | X02160 | 0.6118 | Below |
| 6 | 39691_at | SH3-containing protein SH3GLB1 | SH3GLB1 | AB007960 | 0.6089 | Above |
| 7 | 1558_g_at | p21/Cdc42/Rac1-activated kinase 1 | PAK1 | U24152 | 0.6087 | Above |
| | | yeast Ste20-related | | | | |
| 8 | 34759_at | Human hbc647 mRNA sequence | | U68494 | 0.6061 | Above |
| 9 | 33774_at | caspase 8 apoptosis-related cysteine | CASP8 | X98172 | 0.6040 | Above |
| | | protease | | | | |
| 10 | 1326_at | caspase 10 apoptosis-related | CASP10 | U60519 | 0.6021 | Above |
| | | cysteine protease | | | | |
| 11 | 38312_at | DKFZp564O222 from clone | | AL050002 | 0.6010 | Above |
| | | DKFZp564O222 | | | | |
| 12 | 35970_g_at | M-phase phosphoprotein 9 | MPHOSPH9 | N23137 | 0.5989 | Above |
| 13 | 41273_at | FK506 binding protein 12- | FRAP1 | AL046940 | 0.5989 | Above |
| | | rapamycin associated protein 1 | | | | |
| 14 | 40798_s_at | a disintegrin and metalloproteinase | ADAM10 | Z48579 | 0.5980 | Above |
| | | domain 10 | | | | |
| 15 | 40953_at | calponin 3 acidic | CNN3 | S80562 | 0.5972 | Above |
| 16 | 1434_at | phosphatase and tensin homolog | PTEN | U92436 | 0.5963 | Below |
| | | mutated in multiple advanced | | | | |
| | | cancers 1 | | | | |
| 17 | 38966_at | glycoprotein synaptic 2 | GPSN2 | AF038958 | 0.5953 | Above |
| 18 | 35991_at | Sm protein F | LSM6 | AA917945 | 0.5938 | Above |
| 19 | 330_s_at | tubulin, alpha 1, isoform 44 | TUBA1 | HG2259- | 0.5938 | Above |
| | | | | HT2348 | | |
| 20 | 38032_at | KIAA0736 gene product | KIAA0736 | AB018279 | 0.5934 | Above |
| 21 | 1983_at | cyclin D2 | CCND2 | X68452 | 0.5927 | Above |
| 22 | 36194_at | low density lipoprotein-related | LRPAP1 | M63959 | 0.5914 | Below |
| | | protein-associated protein 1 alpha- | | | | |
| | | 2-macroglobulin receptor- | | | | |
| | | associated protein 1 | | | | |
| 23 | 34460_at | peripheral benzodiazepine receptor- | PRAX-1 | AB014512 | 0.5911 | Above |
| | | associated protein 1 | | | | |
| 24 | 2001_g_at | ataxia telangiectasia mutated | ATM | U26455 | 0.5910 | Above |
| | | includes complementation groups A | | | | |
| | | C and D | | | | |
| 25 | 31443_at | AML1 | AML1 | S76346 | 0.5896 | Above |
| 26 | 33410_at | integrin alpha 6 | ITGA6 | S66213 | 0.5896 | Above |
| 27 | 37472_at | mannosidase beta A lysosomal | MANBA | U60337 | 0.5887 | Below |
| 28 | 36099_at | splicing factor arginine/serine-rich | SFRS1 | M69040 | 0.5877 | Below |
| | | 1 splicing factor 2 alternate splicing | | | | |
| | | factor | | | | |
| 29 | 38636_at | immunoglobulin superfamily | ISLR | AB003184 | 0.5858 | Above |
| | | containing leucine-rich repeat | | | | |
| 30 | 34314_at | ribonucleotide reductase M1 | RRM1 | X59543 | 0.5858 | Below |
| | | polypeptide | | | | |
| 31 | 36129_at | KIAA0397 gene product | KIAA0397 | AB007857 | 0.5858 | Above |
| 32 | 40264_g_at | zinc finger protein-like 1 | ZFPL1 | AF001891 | 0.5858 | Above |
| 33 | 37399_at | aldo-keto reductase family 1 | AKR1C3 | D17793 | 0.5852 | Above |
| | | member C3 3-alpha hydroxysteroid | | | | |
| | | dehydrogenase type II | | | | |
| 34 | 38160_at | lymphocyte antigen 75 | LY75 | AF011333 | 0.5832 | Above |
| 35 | 41649_at | FOXJ2 forkhead factor | LOC55810 | AF038177 | 0.5832 | Above |
| 36 | 36591_at | tubulin alpha 1 testis specific | TUBA1 | X06956 | 0.5832 | Above |
| 37 | 40167_s_at | CS box-containing WD protein | LOC55884 | AF038187 | 0.5832 | Above |
| 38 | 2064_g_at | excision repair cross- | ERCC5 | L20046 | 0.5832 | Above |
| | | complementing rodent repair | | | | |
| | | deficiency complementation group | | | | |
| 39 | 39729_at | Human natural killer cell enhancing | NKEFB | L19185 | 0.5829 | Below |
| | | factor (NKEFB) mRNA, complete | | | | |
| | | cds. | | | | |
| 40 | 38270_at | poly ADP-ribose glycohydrolase | PARG | AF005043 | 0.5828 | Below |
| 41 | 40613_at | uncharacterized hypothalamus | HT012 | AL031775 | 0.5819 | Below |
| | | protein HT012 | | | | |
| 42 | 39070_at | singed Drosophila like sea urchin | SNL | U03057 | 0.5813 | Above |
| | | fascin homolog like | | | | |
| 43 | 40782_at | short-chain | SDR1 | AF061741 | 0.5813 | Above |
| | | dehydrogenase/reductase 1 | | | | |
| 44 | 34256_at | sialyltransferase 9 CMP-NeuAc | SIAT9 | AB018356 | 0.5797 | Above |
| | | lactosylceramide alpha-2 3- | | | | |
| | | sialyltransferase GM3 synthase | | | | |
| 45 | 41836_at | protein with polyglutamine repeat | ERPROT213- | U94836 | 0.5777 | Above |
| | | calcium ca2 homeostasis | 21 | | | |
| | | endoplasmic reticulum protein | | | | |
| 46 | 35681_r_at | zinc finger homeobox 1B | ZFHX1B | AB011141 | 0.5759 | Below |
| 47 | 37190_at | WAS protein family member 1 | WASF1 | D87459 | 0.5759 | Below |
| 48 | 32788_at | RAN binding protein 92 | RANBP2 | D42063 | 0.5756 | Above |
| 49 | 828_at | prostaglandin E receptor 2 subtype | PTGER2 | U19487 | 0.5740 | Above |
| | | EP2 53 kD | | | | |
| 50 | 38220_at | dihydropyrimidine dehydrogenase | DPYD | U20938 | 0.5737 | Above |

TABLE 24
Genes Selected by Wilkins' for E2A-PBX1

| | | | | | | Above/ |
|---|---|---|---|---|---|---|
| | Affymetrix | | Gene | Reference | Train set | Below |
| | number | Gene Name | Symbol | number | score | Mean |
| 1 | 32063_at | pre-B-cell leukemia transcription | PBX1 | M86546 | 0.8750 | Above |
| | | factor 1 | | | | |
| 2 | 38994_at | STAT induced STAT inhibitor-2 | STATI2 | AF037989 | 0.8252 | Below |
| 3 | 33355_at | Homo sapiens cDNA FLJ12900 fis | PBX1 | AL049381 | 0.8040 | Above |
| | | clone NT2RP2004321 (by | | | | |
| | | CELERA serach of target sequence = | | | | |
| | | PBX1) | | | | |
| 4 | 40454_at | FAT tumor suppressor Drosophila | FAT | X87241 | 0.7899 | Above |
| | | homolog | | | | |
| 5 | 753_at | nidogen 2 | NID2 | D86425 | 0.7368 | Above |
| 6 | 717_at | GS3955 protein | GS3955 | D87119 | 0.7306 | Above |
| 7 | 1786_at | c-mer proto-oncogene tyrosine | MERTK | U08023 | 0.7300 | Above |
| | | kinase | | | | |
| 8 | 39070_at | singed Drosophila like sea urchin | SNL | U03057 | 0.7271 | Below |
| | | fascin homolog like | | | | |
| 9 | 1065_at | fms-related tyrosine kinase 3 | FLT3 | U02687 | 0.7160 | Below |
| 10 | 36650_at | cyclin D2 | CCND2 | D13639 | 0.7151 | Below |
| 11 | 33513_at | signaling lymphocytic activation | SLAM | U33017 | 0.7096 | Above |
| | | molecule | | | | |
| 12 | 33748_at | minor histocompatibility antigen | KIAA0223 | D86976 | 0.7084 | Below |
| | | HA-1 | | | | |
| 13 | 37225_at | KIAA0172 protein | KIAA0172 | D79994 | 0.7033 | Above |
| 14 | 38717_at | DKFZP586A0522 protein | DKFZP586A | AL050159 | 0.7003 | Below |
| | | | 0522 | | | |
| 15 | 854_at | B lymphoid tyrosine kinase | BLK | S76617 | 0.6982 | Above |
| 16 | 33641_g_at | nuclear factor of kappa light | NFKBIL1 | Y14768 | 0.6975 | Below |
| | | polypeptide gene enhancer in B- | | | | |
| | | cells inhibitor-like 1 | | | | |
| 17 | 40468_at | KIAA0554 protein | KIAA0554 | AB011126 | 0.6971 | Below |
| 18 | 41266_at | integrin alpha 6 | ITGA6 | X53586 | 0.6965 | Below |
| 19 | 36536_at | schwannomin interacting protein 1 | SCHIP-1 | AF070614 | 0.6938 | Below |
| 20 | 362_at | protein kinase C zeta | PRKCZ | Z15108 | 0.6904 | Above |
| 21 | 755_at | inositol 1 4 5-triphosphate receptor | ITPR1 | D26070 | 0.6877 | Below |
| | | type 1 | | | | |
| 22 | 307_at | arachidonate 5-lipoxygenase | ALOX5 | J03600 | 0.6875 | Below |
| 23 | 39614_at | KIAA0802 protein | KIAA0802 | AB018345 | 0.6863 | Above |
| 24 | 1563_s_at | tumor necrosis factor receptor | TNFRSF1A | M58286 | 0.6837 | Below |
| | | superfamily member 1A | | | | |
| 25 | 38748_at | adenosine deaminase RNA-specific | ADARB1 | U76421 | 0.6763 | Above |
| | | B1 homolog of rat RED1 | | | | |
| 26 | 41409_at | basement membrane-induced gene | ICB-1 | AF044896 | 0.6757 | Below |
| 27 | 34892_at | tumor necrosis factor receptor | TNFRSF10B | AF016266 | 0.6726 | Below |
| | | superfamily member 10b | | | | |
| 28 | 40648_at | c-mer proto-oncogene tyrosine | MERTK | U08023 | 0.6710 | Above |
| | | kinase | | | | |
| 29 | 38408_at | transmembrane 4 superfamily | TM4SF2 | L10373 | 0.6667 | Below |
| | | member 2 | | | | |
| 30 | 34583_at | fms-related tyrosine kinase 3 | FLT3 | U02687 | 0.6665 | Below |
| 31 | 36900_at | stromal interaction molecule 1 | STIM1 | U52426 | 0.6650 | Below |
| 32 | 37625_at | interferon regulatory factor 4 | IRF4 | U52682 | 0.6636 | Above |
| 33 | 38340_at | huntingtin interacting protein-1- | KIAA0655 | AB014555 | 0.6609 | Above |
| | | related | | | | |
| 34 | 1830_s_at | transforming growth factor beta 1 | TGFB1 | M38449 | 0.6608 | Below |
| 35 | 37099_at | arachidonate 5-lipoxygenase- | ALOX5AP | AI806222 | 0.6605 | Below |
| | | activating protein | | | | |
| 36 | 38254_at | KIAA0882 protein | KIAA0882 | AB020689 | 0.6539 | Below |
| 37 | 37641_at | Human gene for hepatitis C- | | D28915 | 0.6531 | Below |
| | | associated microtubular aggregate | | | | |
| | | protein p44, exon 9 and complete | | | | |
| | | cds. | | | | |
| 38 | 33865_at | adenovirus 5 E1A binding protein | BS69 | AA127624 | 0.6515 | Below |
| 39 | 40729_s_at | nuclear factor of kappa light | NFKBIL1 | Y14768 | 0.6502 | Below |
| | | polypeptide gene enhancer in B- | | | | |
| | | cells inhibitor-like 1 | | | | |
| 40 | 40113_at | GS3955 protein | GS3955 | D87119 | 0.6476 | Above |
| 41 | 32979_at | GRB2-associated binding protein 1 | GAB1 | U43885 | 0.6457 | Below |
| 42 | 36591_at | tubulin alpha 1 testis specific | TUBA1 | X06956 | 0.6427 | Below |
| 43 | 38739_at | v-ets avian erythroblastosis virus | ETS2 | AF017257 | 0.6424 | Below |
| | | E26 oncogene homolog 2 | | | | |
| 44 | 37485_at | fatty-acid-Coenzyme A ligase very | FACVL1 | D88308 | 0.6363 | Above |
| | | long-chain 1 | | | | |
| 45 | 538_at | CD34 antigen | CD34 | S53911 | 0.6326 | Below |
| 46 | 37893_at | protein tyrosine phosphatase non- | PTPN2 | AI828880 | 0.6318 | Above |
| | | receptor type 2 | | | | |
| 47 | 41017_at | myosin-binding protein H | MYBPH | U27266 | 0.6297 | Above |
| 48 | 37967_at | lymphocyte antigen 117 | LY117 | AF000424 | 0.6260 | Below |
| 49 | 37281_at | KIAA0233 gene product | KIAA0233 | D87071 | 0.6250 | Below |
| 50 | 35675_at | vinexin beta SH3-containing | SCAM-1 | AF037261 | 0.6229 | Below |
| | | adaptor molecule-1 | | | | |

TABLE 25
Genes selected for Wilkins for Hyperdiploid >50

| | | | | | | Above/ |
|---|---|---|---|---|---|---|
| | Affymetrix | | Gene | Reference | Train set | Below |
| | number | Gene Name | Symbol | number | score | Mean |
| 1 | 39878_at | protocadherin 9 | PCDH9 | AI524125 | 0.5838 | Below |
| 2 | 41470_at | Prominin mouse like 1 | PROML1 | AF027208 | 0.5616 | Above |
| 3 | 39069_at | AE-binding protein 1 | AEBP1 | AF053944 | 0.5423 | Below |
| 4 | 1520_s_at | interleukin 1 beta | IL1B | X04500 | 0.5399 | Above |
| 5 | 578_at | Human recombination acitivating | RAG2 | M94633 | 0.5208 | Below |
| | | protein (RAG2) gene, last exon | | | | |
| 6 | 32251_at | hypothetical protein FLJ21174 | FLJ21174 | AA149307 | 0.5164 | Above |
| 7 | 40480_s_at | FYN oncogene related to SRC FGR | FYN | M14333 | 0.5090 | Above |
| | | YES | | | | |
| 8 | 38604_at | neuropeptide Y | NPY | AI198311 | 0.5083 | Above |
| 9 | 40903_at | ATPase H transporting lysosomal | APT6M8-9 | AL049929 | 0.5080 | Above |
| | | vacuolar proton pump membrane | | | | |
| | | sector associated protein M8-9 | | | | |
| 10 | 38968_at | SH3-domain binding protein 5 | SH3BP5 | AB005047 | 0.5057 | Above |
| | | BTK-associated | | | | |
| 11 | 37272_at | inositol 1 4 5-trisphosphate 3- | ITPKB | X57206 | 0.5025 | Below |
| | | kinase B | | | | |
| 12 | 35688_g_at | mature T-cell proliferation 1 | MTCP1 | Z24459 | 0.5018 | Above |
| 13 | 1488_at | protein tyrosine phosphatase | PTPRK | L77886 | 0.4977 | Below |
| | | receptor type K | | | | |
| 14 | 36885_at | spleen tyrosine kinase | SYK | L28824 | 0.4964 | Below |
| 15 | 1630_s_at | tyrosine kinase syk | syk | HG3730- | 0.4913 | Below |
| | | | | HT4000 | | |
| 16 | 38317_at | transcription elongation factor A | TCEAL1 | M99701 | 0.4901 | Above |
| | | SII like 1 | | | | |
| 17 | 38649_at | KIAA0970 protein | KIAA0970 | AB023187 | 0.4898 | Below |
| 18 | 39721_at | ephrin-B1 | EFNB1 | U09303 | 0.4895 | Above |
| 19 | 33307_at | kraken-like | BK126B4.1 | AL022316 | 0.4880 | Below |
| 20 | 38518_at | sex comb on midleg Drosophila like 2 | SCML2 | Y18004 | 0.4879 | Above |
| 21 | 39402_at | interleukin 1 beta | IL1B | M15330 | 0.4750 | Above |
| 22 | 36489_at | phosphoribosyl pyrophosphate | PRPS1 | D00860 | 0.4718 | Above |
| | | synthetase 1 | | | | |
| 23 | 37747_at | Human annexin V (ANX5) gene, | (ANX5 | U05770 | 0.4717 | Above |
| | | exon 13. | | | | |
| 24 | 40200_at | heat shock transcription factor 1 | HSF1 | M64673 | 0.4689 | Below |
| 25 | 35940_at | POU domain class 4 transcription | POU4F1 | X64624 | 0.4685 | Above |
| | | factor 1 | | | | |
| 26 | 35727_at | hypothetical protein FLJ20517 | FLJ20517 | AI249721 | 0.4675 | Below |
| 27 | 1357_at | ubiquitin specific protease 4 proto- | USP4 | U20657 | 0.4670 | Below |
| | | oncogene | | | | |
| 28 | 36592_at | prohibitin | PHB | S85655 | 0.4668 | Above |
| 29 | 37014_at | myxovirus influenza resistance 1 | MX1 | M33882 | 0.4635 | Above |
| | | homolog of murine interferon- | | | | |
| | | inducible protein p78 | | | | |
| 30 | 40891_f_at | DNA segment on chromosome X | DXS9879E | X92896 | 0.4608 | Above |
| | | unique 9879 expressed sequence | | | | |
| 31 | 40846_g_at | interleukin enhancer binding factor | ILF3 | U10324 | 0.4605 | Below |
| | | 3 90 Kd | | | | |
| 32 | 41132_r_at | heterogeneous nuclear | HNRPH2 | U01923 | 0.4605 | Above |
| | | ribonucleoprotein H2 H | | | | |
| 33 | 37280_at | MAD mothers against | MADH1 | U59912 | 0.4595 | Below |
| | | decapentaplegic Drosophila | | | | |
| | | homolog 1 | | | | |
| 34 | 35939_s_at | POU domain class 4 transcription | POU4F1 | L20433 | 0.4594 | Above |
| | | factor 1 | | | | |
| 35 | 890_at | ubiquitin-conjugating enzyme E2A | UBE2A | M74524 | 0.4570 | Above |
| | | RAD6 homolog | | | | |
| 36 | 38738_at | SMT3 suppressor of mif two 3 | SMT3H1 | X99584 | 0.4568 | Above |
| | | yeast homolog 1 | | | | |
| 37 | 38458_at | Human cytochrome b5 (CYB5) | CYB5 | L39945 | 0.4552 | Above |
| | | gene, exon 6 and complete cds. | | | | |
| 38 | 38869_at | KIAA1069 protein | KIAA1069 | AB028992 | 0.4549 | Above |
| 39 | 915_at | interferon-induced protein with | IFIT1 | M24594 | 0.4544 | Above |
| | | tetratricopeptide repeats 1 | | | | |
| 40 | 38408_at | transmembrane 4 superfamily | TM4SF2 | L10373 | 0.4535 | Above |
| | | member 2 | | | | |
| 41 | 39301_at | calpain 3 p94 | CAPN3 | X85030 | 0.4533 | Below |
| 42 | 41425_at | Friend leukemia virus integration 1 | FLI1 | M98833 | 0.4519 | Below |
| 43 | 2094_s_at | v-fos FBJ murine osteosarcoma | FOS | K00650 | 0.4514 | Above |
| | | viral oncogene homolog | | | | |
| 44 | 36605_at | transcription factor 4 | TCF4 | M74719 | 0.4497 | Above |
| 45 | 37709_at | DNA segment numerous copies | DXF68S1E | M86934 | 0.4493 | Above |
| | | expressed probes GS1 gene | | | | |
| 46 | 36128_at | transmembrane trafficking protein | TMP21 | L40397 | 0.4488 | Above |
| 47 | 171_at | von Hippel-Lindau binding protein 1 | VBP1 | U56833 | 0.4473 | Above |
| 48 | 41490_at | phosphoribosyl pyrophosphate | PRPS2 | Y00971 | 0.4466 | Above |
| | | synthetase 2 | | | | |
| 49 | 36536_at | schwannomin interacting protein 1 | SCHIP-1 | AF070614 | 0.4448 | Above |
| 50 | 35843_at | Homo sapiens mRNA cDNA | | L40402 | 0.4443 | Above |
| | | DKFZp434D0935 | | | | |

TABLE 26
Genes Selected by Wilkins' for MLL

| | | | | | | Above/ |
|---|---|---|---|---|---|---|
| | Affymetrix | | Gene | Reference | Train set | Below |
| | number | Gene Name | Symbol | number | score | Mean |
| 1 | 39402_at | interleukin 1 beta | IL1B | M15330 | 0.7355 | Below |
| 2 | 307_at | arachidonate 5-lipoxygenase | ALOX5 | J03600 | 0.7221 | Below |
| 3 | 1389_at | membrane metallo-endopeptidase | MME | J03779 | 0.7178 | Below |
| | | neutral endopeptidase | | | | |
| | | enkephalinase CALLA CD10 | | | | |
| 4 | 37280_at | MAD mothers against | MADH1 | U59912 | 0.7021 | Below |
| | | decapentaplegic Drosophila | | | | |
| | | homolog 1 | | | | |
| 5 | 36650_at | cyclin D2 | CCND2 | D13639 | 0.6759 | Below |
| 6 | 37043_at | inhibitor of DNA binding 3 | ID3 | AL021154 | 0.6743 | Below |
| | | dominant negative helix-loop-helix | | | | |
| | | protein | | | | |
| 7 | 1520_s_at | interleukin 1 beta | IL1B | X04500 | 0.6689 | Below |
| 8 | 40913_at | ATPase Ca transporting plasma | ATP2B4 | W28589 | 0.6684 | Below |
| | | membrane 4 | | | | |
| 9 | 36536_at | schwannomin interacting protein 1 | SCHIP-1 | AF070614 | 0.6554 | Below |
| 10 | 37398_at | platelet/endothelial cell adhesion | PECAM1 | AA100961 | 0.6548 | Below |
| | | molecule CD31 antigen | | | | |
| 11 | 39114_at | decidual protein induced by | DEPP | AB022718 | 0.6478 | Below |
| | | progesterone | | | | |
| 12 | 37967_at | lymphocyte antigen 117 | LY117 | AF000424 | 0.6432 | Below |
| 13 | 1325_at | MAD mothers against | MADH1 | U59423 | 0.6421 | Below |
| | | decapentaplegic Drosophila | | | | |
| | | homolog 1 | | | | |
| 14 | 38336_at | KIAA1013 protein | KIAA1013 | AB023230 | 0.6395 | Below |
| 15 | 577_at | midkine neurite growth-promoting | MDK | M94250 | 0.6363 | Below |
| | | factor 2 | | | | |
| 16 | 38671_at | KIAA0620 protein | KIAA0620 | AB014520 | 0.6353 | Below |
| 17 | 33412_at | LGALS1 Lectin, galactoside- | LGALS1 | AI535946 | 0.6351 | Above |
| | | binding, soluble, 1 | | | | |
| 18 | 40451_at | hypothetical protein FLJ21434 | FLJ21434 | AL080203 | 0.6350 | Below |
| 19 | 36908_at | Human macrophage mannose | MRC1 | M93221 | 0.6290 | Below |
| | | receptor (MRC1) gene, exon 30. | | | | |
| 20 | 963_at | ligase IV DNA ATP-dependent | LIG4 | X83441 | 0.6282 | Below |
| 21 | 41346_at | like-glycosyltransferase | LARGE | AJ007583 | 0.6214 | Below |
| 22 | 32207_at | membrane protein palmitoylated 1 | MPP1 | M64925 | 0.6155 | Below |
| | | 55 kD | | | | |
| 23 | 2062_at | insulin-like growth factor binding | IGFBP7 | L19182 | 0.6145 | Above |
| | | protein 7 | | | | |
| 24 | 38408_at | transmembrane 4 superfamily | TM4SF2 | L10373 | 0.6137 | Below |
| | | member 2 | | | | |
| 25 | 854_at | B lymphoid tyrosine kinase | BLK | S76617 | 0.6075 | Above |
| 26 | 32193_at | plexin C1 | PLXNC1 | AF030339 | 0.6065 | Above |
| 27 | 35939_s_at | POU domain class 4 transcription | POU4F1 | L20433 | 0.6046 | Below |
| | | factor 1 | | | | |
| 28 | 33705_at | phosphodiesterase 4B cAMP- | PDE4B | L20971 | 0.5991 | Below |
| | | specific dunce Drosophila homolog | | | | |
| | | phosphodiesterase E4 | | | | |
| 29 | 34168_at | deoxynucleotidyltransferase | DNTT | M11722 | 0.5979 | Below |
| | | terminal | | | | |
| 30 | 36383_at | v-ets avian erythroblastosis virus | ERG | M17254 | 0.5976 | Below |
| | | E26 oncogene related | | | | |
| 31 | 38968_at | SH3-domain binding protein 5 | SH3BP5 | AB005047 | 0.5976 | Below |
| | | BTK-associated | | | | |
| 32 | 39263_at | 2 5 oligoadenylate synthetase 2 | OAS2 | M87434 | 0.5967 | Below |
| 33 | 39329_at | actinin alpha 1 | ACTN1 | X15804 | 0.5953 | Below |
| 34 | 34699_at | CD2-associated protein | CD2AP | AL050105 | 0.5945 | Below |
| 35 | 1267_at | protein kinase C eta | PRKCH | M55284 | 0.5941 | Below |
| 36 | 35172_at | tyrosylprotein sulfotransferase 2 | TPST2 | AF049891 | 0.5937 | Below |
| 37 | 38124_at | midkine neurite growth-promoting | MDK | X55110 | 0.5936 | Below |
| | | factor 2 | | | | |
| 38 | 33813_at | tumor necrosis factor receptor | TNFRSF1B | AI813532 | 0.5934 | Below |
| | | superfamily member 1B | | | | |
| 39 | 34176_at | hypothetical protein from clone 643 | LOC57228 | AF091087 | 0.5930 | Below |
| 40 | 39424_at | tumor necrosis factor receptor | TNFRSF14 | U70321 | 0.5930 | Below |
| | | superfamily member 14 herpesvirus | | | | |
| | | entry mediator | | | | |
| 41 | 40729_s_at | nuclear factor of kappa light | NFKBIL1 | Y14768 | 0.5905 | Below |
| | | polypeptide gene enhancer in B- | | | | |
| | | cells inhibitor-like 1 | | | | |
| 42 | 32607_at | brain acid-soluble protein 1 | BASP1 | AF039656 | 0.5905 | Above |
| 43 | 38342_at | KIAA0239 protein | KIAA0239 | D87076 | 0.5896 | Below |
| 44 | 32533_s_at | vesicle-associated membrane | VAMP5 | AF054825 | 0.5880 | Below |
| | | protein 5 myobrevin | | | | |
| 45 | 39330_s_at | actinin alpha 1 | ACTN1 | M95178 | 0.5867 | Below |
| 46 | 40519_at | protein tyrosine phosphatase | PTPRC | Y00638 | 0.5848 | Above |
| | | receptor type C | | | | |
| 47 | 39338_at | S100 calcium-binding protein A10 | S100A10 | AI201310 | 0.5844 | Above |
| | | annexin II ligand calpactin I light | | | | |
| | | polypeptide p11 | | | | |
| 48 | 35940_at | POU domain class 4 transcription | POU4F1 | X64624 | 0.5824 | Below |
| | | factor 1 | | | | |
| 49 | 39712_at | S100 calcium-binding protein A13 | S100A13 | AI541308 | 0.5818 | Below |
| 50 | 39379_at | Homo sapiens mRNA cDNA | | AL049397 | 0.5811 | Above |
| | | DKFZp586C1019 from clone | | | | |
| | | DKFZp586C1019 | | | | |

TABLE 27
Genes Selected by Wilkins' for Novel Risk Group

| | | | | | | Above/ |
|---|---|---|---|---|---|---|
| | Affymetrix | | Gene | Reference | Train set | Below |
| | number | Gene Name | Symbol | number | score | Mean |
| 1 | 31892_at | protein tyrosine phosphatase | PTPRM | X58288 | 0.8668 | Above |
| | | receptor type M | | | | |
| 2 | 41734_at | KIAA0870 protein | KIAA0870 | AB020677 | 0.8614 | Below |
| 3 | 995_g_at | protein tyrosine phosphatase | PTPRM | X58288 | 0.8505 | Above |
| | | receptor type M | | | | |
| 4 | 994_at | protein tyrosine phosphatase | PTPRM | X58288 | 0.7694 | Above |
| | | receptor type M | | | | |
| 5 | 37967_at | lymphocyte antigen 117 | LY117 | AF000424 | 0.7399 | Below |
| 6 | 34676_at | KIAA1099 protein | KIAA1099 | AB029022 | 0.7298 | Above |
| 7 | 41159_at | Clathrin heavy polypeptide Hc | CLTC | D21260 | 0.7283 | Above |
| 8 | 39728_at | interferon gamma-inducible protein | IFI30 | J03909 | 0.7138 | Below |
| | | 30 | | | | |
| 9 | 37542_at | lipoma HMGIC fusion partner-like 2 | LHFPL2 | D86961 | 0.7069 | Above |
| 10 | 35350_at | B cell RAG associated protein | BRAG | AB011170 | 0.7049 | Below |
| 11 | 41438_at | KIAA1451 protein | KIAA1451 | AL049923 | 0.6999 | Below |
| 12 | 34370_at | Archain 1 | ARCN1 | X81198 | 0.6999 | Below |
| 13 | 36029_at | chromosome 11 open reading frame 8 | C11ORF8 | U57911 | 0.6964 | Above |
| 14 | 37960_at | carbohydrate chondroitin 6/keratan | CHST2 | AB014679 | 0.6947 | Above |
| | | sulfotransferase 2 | | | | |
| 15 | 35869_at | MD-1 RP105-associated | MD-1 | AB020499 | 0.6908 | Below |
| 16 | 36601_at | Vinculin | VCL | M33308 | 0.6908 | Below |
| 17 | 40775_at | Integral membrane protein 2A | ITM2A | AL021786 | 0.6879 | Above |
| 18 | 37281_at | KIAA0233 gene product | KIAA0233 | D87071 | 0.6837 | Below |
| 19 | 957_at | Arrestin, beta 2 | ARRB2 | HG2059- | 0.6744 | Below |
| | | | | HT2114 | | |
| 20 | 33284_at | myeloperoxidase | MPO | M19507 | 0.6712 | Below |
| 21 | 40585_at | adenylate cyclase 7 | ADCY7 | D25538 | 0.6712 | Below |
| 22 | 37908_at | guanine nucleotide binding protein | GNG11 | U31384 | 0.6656 | Above |
| | | 11 | | | | |
| 23 | 40167_s_at | CS box-containing WD protein | LOC55884 | AF038187 | 0.6581 | Below |
| 24 | 38576_at | H2B histone family member B | H2BFB | AJ223353 | 0.6576 | Below |
| 25 | 36591_at | tubulin alpha 1 testis specific | TUBA1 | X06956 | 0.6576 | Below |
| 26 | 37712_g_at | MADS box transcription enhancer | MEF2C | S57212 | 0.6576 | Below |
| | | factor 2 polypeptide C myocyte | | | | |
| | | enhancer factor 2C | | | | |
| 27 | 33924_at | KIAA1091 protein | KIAA1091 | AB029014 | 0.6484 | Below |
| 28 | 32724_at | phytanoyl-CoA hydroxylase | PHYH | AF023462 | 0.6466 | Above |
| | | Refsum disease | | | | |
| 29 | 33358_at | EST (retina) | | W29087 | 0.6457 | Above |
| 30 | 33740_at | chromosome 1 open reading frame 2 | C1ORF2 | AF023268 | 0.6441 | Below |
| 31 | 36588_at | KIAA0810 protein | KIAA0810 | AB018353 | 0.6441 | Below |
| 32 | 38802_at | progesterone binding protein | HPR6.6 | Y12711 | 0.6441 | Below |
| 33 | 38408_at | transmembrane 4 superfamily | TM4SF2 | L10373 | 0.6440 | Below |
| | | member 2 | | | | |
| 34 | 32227_at | proteoglycan 1 secretory granule | PRG1 | X17042 | 0.6409 | Below |
| 35 | 34840_at | Homo sapiens cDNA FLJ22642 fis | | AI700633 | 0.6409 | Below |
| | | clone HSI06970 | | | | |
| 36 | 1131_at | mitogen-activated protein kinase | MAP2K2 | L11285 | 0.6409 | Below |
| | | kinase 2 | | | | |
| 37 | 33410_at | integrin alpha 6 | ITGA6 | S66213 | 0.6391 | Above |
| 38 | 38006_at | CD48 antigen B-cell membrane | CD48 | M37766 | 0.6342 | Below |
| | | protein | | | | |
| 39 | 33907_at | eukaryotic translation initiation | EIF4G3 | AF012072 | 0.6304 | Below |
| | | factor 4 gamma 3 | | | | |
| 40 | 41273_at | FK506 binding protein 12- | FRAP1 | AL046940 | 0.6304 | Below |
| | | rapamycin associated protein 1 | | | | |
| 41 | 39781_at | insulin-like growth factor-binding | IGFBP4 | U20982 | 0.6301 | Below |
| | | protein 4 | | | | |
| 42 | 39893_at | guanine nucleotide binding protein | GNG7 | AB010414 | 0.6301 | Below |
| | | G protein gamma 7 | | | | |
| 43 | 37326_at | proteolipid protein 2 colonic | PLP2 | U93305 | 0.6267 | Below |
| | | epithelium-enriched | | | | |
| 44 | 36687_at | cytochrome c oxidase subunit VIIb | COX7B | N50520 | 0.6266 | Below |
| 45 | 40423_at | KIAA0903 protein | KIAA0903 | AB020710 | 0.6254 | Above |
| 46 | 32542_at | four and a half LIM domains 1 | FHL1 | AF063002 | 0.6236 | Below |
| 47 | 33232_at | cysteine-rich protein 1 intestinal | CRIP1 | AI017574 | 0.6211 | Below |
| 48 | 37280_at | MAD mothers against | MADH1 | U59912 | 0.6208 | Above |
| | | decapentaplegic Drosophila | | | | |
| | | homolog 1 | | | | |
| 49 | 1325_at | MAD mothers against | MADH1 | U59423 | 0.6208 | Above |
| | | decapentaplegic Drosophila | | | | |
| | | homolog 1 | | | | |
| 50 | 40729_s_at | nuclear factor of kappa light | NFKBIL1 | Y14768 | 0.6199 | Below |
| | | polypeptide gene enhancer in B- | | | | |
| | | cells inhibitor-like 1 | | | | |

TABLE 28
Genes selected by Wilkins' for T-ALL

| | | | | | | Above/ |
|---|---|---|---|---|---|---|
| | Affymetrix | | Gene | Reference | Train set | Below |
| | number | Gene Name | Symbol | number | score | Mean |
| 1 | 38242_at | B cell linker protein | SLP65 | AF068180 | 0.8683 | Below |
| 2 | 37988_at | CD79B antigen immunoglobulin- | CD79B | M89957 | 0.8422 | Below |
| | | associated beta | | | | |
| 3 | 1096_g_at | CD19 antigen | CD19 | M28170 | 0.8181 | Below |
| 4 | 39318_at | T-cell leukemia/lymphoma 1A | TCL1A | X82240 | 0.8128 | Below |
| 5 | 38018_g_at | CD79A antigen immunoglobulin- | CD79A | U05259 | 0.8127 | Below |
| | | associated alpha | | | | |
| 6 | 36878_f_at | major histocompatibility complex | HLA-DQB1 | M60028 | 0.8053 | Below |
| | | class II DQ beta 1 | | | | |
| 7 | 38147_at | SH2 domain protein 1A Duncan s | SH2D1A | AL023657 | 0.8016 | Above |
| | | disease lymphoproliferative | | | | |
| | | syndrome | | | | |
| 8 | 35350_at | B cell RAG associated protein | BRAG | AB011170 | 0.7914 | Below |
| 9 | 38051_at | mal T-cell differentiation protein | MAL | X76220 | 0.7900 | Above |
| 10 | 266_s_at | CD24 antigen small cell lung | CD24 | L33930 | 0.7867 | Below |
| | | carcinoma cluster 4 antigen | | | | |
| 11 | 38521_at | CD22 antigen | CD22 | X59350 | 0.7856 | Below |
| 12 | 37344_at | major histocompatibility complex | HLA-DMA | X62744 | 0.7835 | Below |
| | | class II DM alpha | | | | |
| 13 | 34033_s_at | leukocyte immunoglobulin-like | LILRA2 | AF025531 | 0.7761 | Below |
| | | receptor subfamily A with TM | | | | |
| | | domain member 2 | | | | |
| 14 | 36638_at | connective tissue growth factor | CTGF | X78947 | 0.7755 | Below |
| 15 | 38213_at | galactosidase alpha | GLA | U78027 | 0.7701 | Below |
| 16 | 41734_at | KIAA0870 protein | KIAA0870 | AB020677 | 0.7693 | Below |
| 17 | 37711_at | MADS box transcription enhancer | MEF2C | S57212 | 0.7560 | Below |
| | | factor 2 polypeptide C myocyte | | | | |
| | | enhancer factor 2C | | | | |
| 18 | 36239_at | POU domain class 2 associating | POU2AF1 | Z49194 | 0.7440 | Below |
| | | factor 1 | | | | |
| 19 | 38319_at | CD3D antigen delta polypeptide | CD3D | AA919102 | 0.7426 | Above |
| | | TiT3 complex | | | | |
| 20 | 38894_g_at | neutrophil cytosolic factor 4 40 kD | NCF4 | AL008637 | 0.7422 | Below |
| 21 | 33705_at | phosphodiesterase 4B cAMP- | PDE4B | L20971 | 0.7414 | Below |
| | | specific dunce Drosophila homolog | | | | |
| | | phosphodiesterase E4 | | | | |
| 22 | 38017_at | CD79A antigen immunoglobulin- | CD79A | U05259 | 0.7360 | Below |
| | | associated alpha | | | | |
| 23 | 41156_g_at | catenin cadherin-associated protein | CTNNA1 | U03100 | 0.7315 | Below |
| | | alpha 1 102 kD | | | | |
| 24 | 38994_at | STAT induced STAT inhibitor-2 | STATI2 | AF037989 | 0.7292 | Below |
| 25 | 37710_at | MADS box transcription enhancer | MEF2C | L08895 | 0.7283 | Below |
| | | factor 2 polypeptide C myocyte | | | | |
| | | enhancer factor 2C | | | | |
| 26 | 41155_at | catenin cadherin-associated protein | CTNNA1 | U03100 | 0.7278 | Below |
| | | alpha 1 102 kD | | | | |
| 27 | 40570_at | forkhead box O1A | FOXO1A | AF032885 | 0.7258 | Below |
| | | rhabdomyosarcoma | | | | |
| 28 | 34224_at | fatty acid desaturase 3 | FADS3 | AC004770 | 0.7254 | Below |
| 29 | 38604_at | neuropeptide Y | NPY | AI198311 | 0.7212 | Below |
| 30 | 36773_f_at | major histocompatibility complex | HLA-DQB1 | M81141 | 0.7197 | Below |
| | | class II DQ beta 1 | | | | |
| 31 | 32562_at | endoglin Osler-Rendu-Weber | ENG | X72012 | 0.7180 | Below |
| | | syndrome 1 | | | | |
| 32 | 36502_at | PFTAIRE protein kinase 1 | PFTK1 | AB020641 | 0.7179 | Below |
| 33 | 37180_at | phospholipase C gamma 2 | PLCG2 | X14034 | 0.7114 | Below |
| | | phosphatidylinositol-specific | | | | |
| 34 | 38893_at | neutrophil cytosolic factor 4 40 kD | NCF4 | AL008637 | 0.7100 | Below |
| 35 | 387_at | cyclin-dependent kinase 9 CDC2- | CDK9 | X80230 | 0.7024 | Below |
| | | related kinase | | | | |
| 36 | 32035_at | Human MHC class II HLA- | | M16942 | 0.6992 | Below |
| | | DRw53-associated glycoprotein | | | | |
| | | beta-chain mRNA complete cds | | | | |
| 37 | 41153_f_at | Homo sapiens alphaE-catenin | CTNNA1 | AF102803 | 0.6976 | Below |
| | | (CTNNA1) gene | | | | |
| 38 | 40780_at | C-terminal binding protein 2 | CTBP2 | AF016507 | 0.6976 | Below |
| 39 | 40775_at | integral membrane protein 2A | ITM2A | AL021786 | 0.6952 | Above |
| 40 | 39402_at | interleukin 1 beta | IL1B | M15330 | 0.6945 | Below |
| 41 | 38522_s_at | CD22 antigen | CD22 | X52785 | 0.6945 | Below |
| 42 | 41166_at | immunoglobulin heavy constant mu | IGHM | X58529 | 0.6941 | Below |
| 43 | 36937_s_at | PDZ and LIM domain 1 elfin | PDLIM1 | U90878 | 0.6937 | Below |
| 44 | 38833_at | Human mRNA for SB classII | | X00457 | 0.6925 | Below |
| | | histocompatibility antigen alpha- | | | | |
| | | chain | | | | |
| 45 | 2047_s_at | junction plakoglobin | JUP | M23410 | 0.6920 | Below |
| 46 | 36277_at | Human membran protein (CD3- | CD3E | M23323 | 0.6899 | Above |
| | | epsilon) gene, exon 9. | | | | |
| 47 | 40688_at | linker for activation of T cells | LAT | AJ223280 | 0.6898 | Above |
| 48 | 39389_at | CD9 antigen p24 | CD9 | M38690 | 0.6879 | Below |
| 49 | 33162_at | Insulin receptor | INSR | X02160 | 0.6879 | Below |
| 50 | 31891_at | chitinase 3-like 2 | CHI3L2 | U58515 | 0.6872 | Above |

TABLE 29
Genes Selected by Wilkins' for TEL-AML1

| | | | | | | Above/ |
|---|---|---|---|---|---|---|
| | Affymetrix | | Gene | Reference | Train set | Below |
| | number | Gene Name | Symbol | number | score | Mean |
| 1 | 37780_at | Piccolo presynaptic cytomatrix | PCLO | AB011131 | 0.7121 | Above |
| | | protein | | | | |
| 2 | 38203_at | potassium intermediate/small | KCNN1 | U69883 | 0.7086 | Above |
| | | conductance calcium-activated | | | | |
| | | channel subfamily N member 1 | | | | |
| 3 | 36524_at | Rho guanine nucleotide exchange | ARHGEF4 | AB029035 | 0.6782 | Above |
| | | factor GEF 4 | | | | |
| 4 | 38578_at | tumor necrosis factor receptor | TNFRSF7 | M63928 | 0.6718 | Above |
| | | superfamily member 7 | | | | |
| 5 | 32730_at | Homo sapiens mRNA for KIAA1750 | | AL080059 | 0.6616 | Above |
| | | protein partial cds | | | | |
| 6 | 34194_at | Homo sapiens cDNA FLJ21697 fis | | AL049313 | 0.6518 | Above |
| | | clone COL09740 | | | | |
| 7 | 40272_at | collapsin response mediator protein 1 | CRMP1 | D78012 | 0.6160 | Above |
| 8 | 41819_at | FYN-binding protein FYB-120/130 | FYB | U93049 | 0.6058 | Above |
| 9 | 1488_at | protein tyrosine phosphatase receptor | PTPRK | L77886 | 0.6056 | Above |
| | | type K | | | | |
| 10 | 35665_at | phosphoinositide-3-kinase class 3 | PIK3C3 | Z46973 | 0.6022 | Above |
| 11 | 35614_at | transcription factor-like 5 basic helix- | TCFL5 | AB012124 | 0.5983 | Above |
| | | loop-helix | | | | |
| 12 | 36008_at | protein tyrosine phosphatase type IVA | PTP4A3 | AF041434 | 0.5976 | Above |
| | | member 3 | | | | |
| 13 | 35362_at | Myosin X | MYO10 | AB018342 | 0.5964 | Above |
| 14 | 37908_at | guanine nucleotide binding protein 11 | GNG11 | U31384 | 0.5888 | Above |
| 15 | 39329_at | Actinin alpha 1 | ACTN1 | X15804 | 0.5840 | Below |
| 16 | 1936_s_at | proto-oncogene c-myc, alt. transcript | | HG3523- | 0.5761 | Below |
| | | 3, ORF 114 | | HT4899 | | |
| 17 | 33690_at | Homo sapiens mRNA cDNA | DKFZp434A202 | AL080190 | 0.5725 | Above |
| | | DKFZp434A202 | | | | |
| 18 | 39389_at | CD9 antigen p24 | CD9 | M38690 | 0.5684 | Below |
| 19 | 37343_at | inositol 1 4 5-triphosphate receptor | ITPR3 | U01062 | 0.5642 | Above |
| | | type 3 | | | | |
| 20 | 1299_at | telomeric repeat binding factor 2 | TERF2 | X93512 | 0.5585 | Above |
| 21 | 38652_at | hypothetical protein FLJ20154 | FLJ20154 | AF070644 | 0.5563 | Above |
| 22 | 38763_at | (clone D21-1) L-iditol-2 | | L29254 | 0.5535 | Below |
| | | dehydrogenase gene | | | | |
| 23 | 37724_at | v-myc avian myelocytomatosis viral | MYC | V00568 | 0.5506 | Below |
| | | oncogene homolog | | | | |
| 24 | 36937_s_at | PDZ and LIM domain 1 elfin | PDLIM1 | U90878 | 0.5506 | Below |
| 25 | 1325_at | MAD mothers against | MADH1 | U59423 | 0.5482 | Above |
| | | decapentaplegic Drosophila homolog 1 | | | | |
| 26 | 41549_s_at | adaptor-related protein complex 1 | AP1S2 | AF091077 | 0.5474 | Below |
| | | sigma 2 subunit | | | | |
| 27 | 39827_at | hypothetical protein | FLJ20500 | AA522530 | 0.5471 | Below |
| 28 | 32724_at | phytanoyl-CoA hydroxylase Refsum | PHYH | AF023462 | 0.5459 | Above |
| | | disease | | | | |
| 29 | 31786_at | Sam68-like phosphotyrosine protein | T-STAR | AF051321 | 0.5403 | Above |
| | | T-STAR | | | | |
| 30 | 38570_at | major histocompatibility complex | HLA-DOB | X03066 | 0.5384 | Above |
| | | class II DO beta | | | | |
| 31 | 39330_s_at | actinin alpha 1 | ACTN1 | M95178 | 0.5375 | Below |
| 32 | 36493_at | lymphocyte-specific protein 1 | LSP1 | M33552 | 0.5356 | Below |
| 33 | 574_s_at | caspase 1 apoptosis-related cysteine | CASP1 | M87507 | 0.5336 | Below |
| | | protease interleukin 1 beta convertase | | | | |
| 34 | 32224_at | KIAA0769 gene product | KIAA0769 | AB018312 | 0.5326 | Above |
| 35 | 1077_at | recombination activating gene 1 | RAG1 | M29474 | 0.5302 | Above |
| 36 | 37280_at | MAD mothers against | MADH1 | U59912 | 0.5283 | Above |
| | | decapentaplegic Drosophila homolog 1 | | | | |
| 37 | 41200_at | CD36 antigen collagen type I receptor | CD36L1 | Z22555 | 0.5261 | Above |
| | | thrombospondin receptor like 1 | | | | |
| 38 | 36009_at | hypothetical protein | CL683 | AF091092 | 0.5259 | Below |
| 39 | 36933_at | N-myc downstream regulated | NDRG1 | D87953 | 0.5254 | Below |
| 40 | 1126_s_at | Human cell surface glycoprotein | CD44 | L05424 | 0.5232 | Below |
| | | CD44 (CD44) gene, 3′ end of long | | | | |
| | | tailed isoform. | | | | |
| 41 | 39824_at | ESTs | | AI391564 | 0.5231 | Above |
| 42 | 38078_at | filamin B beta actin-binding protein- | FLNB | AF042166 | 0.5208 | Below |
| | | 278 | | | | |
| 43 | 38127_at | syndecan 1 | SDC1 | Z48199 | 0.5199 | Above |
| 44 | 32941_at | interferon consensus sequence | ICSBP1 | M91196 | 0.5195 | Below |
| | | binding protein 1 | | | | |
| 45 | 37276_at | IQ motif containing GTPase | IQGAP2 | U51903 | 0.5191 | Below |
| | | activating protein 2 | | | | |
| 46 | 34768_at | DKFZP564E1962 protein | DKFZP564 | AL080080 | 0.5184 | Below |
| | | | E1962 | | | |
| 47 | 39781_at | insulin-like growth factor-binding | IGFBP4 | U20982 | 0.5173 | Below |
| | | protein 4 | | | | |
| 48 | 37918_at | integrin beta 2 antigen CD18 p95 | ITGB2 | M15395 | 0.5162 | Below |
| | | lymphocyte function-associated | | | | |
| | | antigen 1 macrophage antigen 1 mac- | | | | |
| | | 1 beta subunit | | | | |
| 49 | 41490_at | phosphoribosyl pyrophosphate | PRPS2 | Y00971 | 0.5155 | Below |
| | | synthetase 2 | | | | |
| 50 | 41814_at | fucosidase alpha-L-1 tissue | FUCA1 | M29877 | 0.5101 | Above |

5. SOM/DAV

The 10,991 probe sets that passed the variation filter were used for subsequent selection of discriminating genes using the self-organizing map (SOM) and discriminant analysis with variance (DAV) programs in the GeneMaths software package (version 1.5, Applied Maths, Belgium). The subgroups for which genes were selected included T-lineage ALL, TEL-AML1, E2A-PBX1, MLL rearrangement, BCR-ABL, hyperdiploid ALL (chromosomal number >50) and the novel subgroup described in the text of the paper. The target number of total genes chosen by each algorithm was 500.

The SOM analysis was performed using 30×18 node format to enable an optimal number of genes per node (˜20 genes per node). Nodes that contained genes whose expression varied more than 2-fold from the mean in more than 70% of the samples in a particular subgroup were chosen. A total of 451 genes were chosen using the SOM algorithm and 443 genes using the DAV algorithm. The combined gene sets contained 755 unique genes, of which 185 were present in both subsets. 2-D hierarchical clustering of the genes and samples were performed using Pearson's correlation coefficient as the metric and unweighted pair group method using arithmetic averages (UPGMA). Approximately 10% of the genes that were found to have correlation coefficients less than 0.7 in each branch of the dendrogram were removed and the process was repeated reiteratively until the correlation coefficient for all genes within a branch was >0.7, or until the removal of additional gene resulted in a deterioration of the class distinction as indicated by inappropriate clustering of cases. Through this approach a subset of 215 genes were selected that optimally separated the 7 subgroups. These genes are listed in Tables 30-36. The selection of genes by this approach does not provide for a ranking. For class prediction between 20 and 30 genes were used for each genetic subgroup, unless otherwise stated.

TABLE 30
Genes selected by DAV-SOM for BCR-ABL

| | | | | | Above/ |
|---|---|---|---|---|---|
| | Affymetrix | | | Reference | Below |
| | number | Gene Name | GeneSymbol | number | Mean |
| 1 | 39250_at | nephroblastoma overexpressed gene | NOV | X96584 | Above |
| 2 | 37600_at | extracellular matrix protein 1 | ECM1 | U68186 | Above |
| 3 | 38312_at | DKFZp564O222 from clone | | AL050002 | Above |
| | | DKFZp564O222 | | | |
| 4 | 38342_at | KIAA0239 protein | KIAA0239 | D87076 | Above |
| 5 | 39712_at | S100 calcium-binding protein A13 | S100A13 | AI541308 | Above |
| 6 | 39730_at | v-ab1 Abelson murine leukemia viral | ABL1 | X16416 | Above |
| | | oncogene homolog 1 | | | |
| 7 | 39781_at | Insulin-like growth factor-binding protein 4 | IGFBP4 | U20982 | Above |
| 8 | 40051_at | TRAM-like protein | KIAA0057 | D31762 | Above |
| 9 | 40504_at | paraoxonase 2 | PON2 | AF001601 | Above |
| 10 | 33362_at | Cdc42 effector protein 3 | CEP3 | AF094521 | Above |
| 11 | 33404_at | adenylyl cyclase-associated protein 2 | CAP2 | U02390 | Above |
| 12 | 34362_at | solute carrier family 2 facilitated glucose | SLC2A5 | M55531 | Above |
| | | transporter member 5 | | | |
| 13 | 36591_at | Tubulin alpha 1 testis specific | TUBA1 | X06956 | Above |
| 14 | 38077_at | collagen type VI alpha 3 | COL6A3 | X52022 | Above |
| 15 | 40196_at | HYA22 protein | HYA22 | D88153 | Above |
| 16 | 1911_s_at | Growth arrest and DNA-damage- | GADD45A | M60974 | Above |
| | | inducible alpha | | | |
| 17 | 1702_at | interleukin 2 receptor alpha | IL2RA | X01057 | Above |
| 18 | 1635_at | Human proto-oncogene tyrosine-protein | ABL | U07563 | Above |
| | | kinase (ABL) gene, exon 1a and exons 2-10, | | | |
| | | complete cds. | | | |
| 19 | 1636_g_at | Human proto-oncogene tyrosine-protein | ABL | U07563 | Above |
| | | kinase (ABL) gene, exon 1a and exons 2-10, | | | |
| | | complete cds. | | | |
| 20 | 1326_at | Caspase 10 apoptosis-related cysteine | CASP10 | U60519 | Above |
| | | protease | | | |
| 21 | 330_s_at | Tubulin, alpha 1, isoform 44 | TUBA1 | HG2259- | Above |
| | | | | HT2348 | |

TABLE 31
Genes selected by DAV-SOM for E2A-PBX1

| | | | | | Above/ |
|---|---|---|---|---|---|
| | Affymetrix | | | Reference | Below |
| | number | Gene Name | GeneSymbol | number | Mean |
| 1 | 33513_at | signaling lymphocytic activation molecule | SLAM | U33017 | Above |
| 2 | 37479_at | CD72 antigen | CD72 | M54992 | Above |
| 3 | 37485_at | fatty-acid-Coenzyme A ligase very long- | FACVL1 | D88308 | Above |
| | | chain 1 | | | |
| 4 | 39614_at | KIAA0802 protein | KIAA0802 | AB018345 | Above |
| 5 | 39929_at | KIAA0922 protein | KIAA0922 | AB023139 | Above |
| 6 | 40648_at | c-mer proto-oncogene tyrosine kinase | MERTK | U08023 | Above |
| 7 | 41017_at | Myosin-binding protein H | MYBPH | U27266 | Above |
| 8 | 41425_at | Friend leukemia virus integration 1 | FLI1 | M98833 | Above |
| 9 | 41862_at | KIAA0056 protein | KIAA0056 | D29954 | Above |
| 10 | 32063_at | pre-B-cell leukemia transcription factor 1 | PBX1 | M86546 | Above |
| 11 | 37225_at | KIAA0172 protein | KIAA0172 | D79994 | Above |
| 12 | 38285_at | mu-crystallin gene | | AF039397 | Above |
| 13 | 38286_at | KIAA1071 protein | KIAA1071 | AB028994 | Above |
| 14 | 38340_at | huntingtin interacting protein-1-related | KIAA0655 | AB014555 | Above |
| 15 | 39379_at | cDNA DKFZp586C1019 from clone | | AL049397 | Above |
| | | DKFZp586C1019 | | | |
| 16 | 39402_at | interleukin 1 beta | IL1B | M15330 | Above |
| 17 | 40454_at | FAT tumor suppressor Drosophila homolog | FAT | X87241 | Above |
| 18 | 41139_at | melanoma antigen family D 1 | MAGED1 | W26633 | Above |
| 19 | 41146_at | ADP-ribosyltransferase NAD poly ADP- | ADPRT | J03473 | Above |
| | | ribose polymerase | | | |
| 20 | 33355_at | Homo sapiens cDNA FLJ12900 fis clone | | AL049381 | Above |
| | | NT2RP2004321 | | | |
| 21 | 34783_s_at | BUB3 budding uninhibited by | BUB3 | AF047473 | Above |
| | | benzimidazoles 3 yeast homolog | | | |
| 22 | 36179_at | mitogen-activated protein kinase-activated | MAPKAPK2 | U12779 | Above |
| | | protein kinase 2 | | | |
| 23 | 36589_at | aldo-keto reductase family 1 member B1 | AKR1B1 | X15414 | Above |
| | | aldose reductase | | | |
| 24 | 38393_at | KIAA0247 gene product | KIAA0247 | D87434 | Above |
| 25 | 38438_at | Nuclear factor of kappa light polypeptide | NFKB1 | M58603 | Above |
| | | gene enhancer in B-cells 1 p105 | | | |
| 26 | 1786_at | c-mer proto-oncogene tyrosine kinase | MERTK | U08023 | Above |
| 27 | 1520_s_at | interleukin 1 beta | IL1B | X04500 | Above |
| 28 | 1287_at | ADP-ribosyltransferase NAD poly ADP- | ADPRT | J03473 | Above |
| | | ribose polymerase | | | |
| 29 | 854_at | B lymphoid tyrosine kinase | BLK | S76617 | Above |
| 30 | 753_at | Nidogen 2 | NID2 | D86425 | Above |
| 31 | 430_at | nucleoside phosphorylase | NP | X00737 | Above |
| 32 | 362_at | Protein kinase C zeta | PRKCZ | Z15108 | Above |

TABLE 32
Genes selected by DAV/SOM for Hyperdiploid >50

| | | | | | Above/ |
|---|---|---|---|---|---|
| | Affymetrix | | | Reference | Below |
| | number | Gene Name | GeneSymbol | number | Mean |
| 1 | 36795_at | prosaposin variant Gaucher disease and | PSAP | J03077 | Above |
| | | variant metachromatic leukodystrophy | | | |
| 2 | 38242_at | B cell linker protein | SLP65 | AF068180 | Above |
| 3 | 38518_at | sex comb on midleg Drosophila like 2 | SCML2 | Y18004 | Above |
| 4 | 39628_at | RAB9 member RAS oncogene family | RAB9 | U44103 | Above |
| 5 | 31863_at | KIAA0179 protein | KIAA0179 | D80001 | Above |
| 6 | 33228_g_at | interleukin 10 receptor beta | IL10RB | AI984234 | Above |
| 7 | 33753_at | KIAA0666 protein | KIAA0666 | AB014566 | Above |
| 8 | 37543_at | Rac/Cdc42 guanine exchange factor GEF 6 | ARHGEF6 | D25304 | Above |
| 9 | 38968_at | SH3-domain binding protein 5 BTK- | SH3BP5 | AB005047 | Above |
| | | associated | | | |
| 10 | 39039_s_at | CGI-76 protein | LOC51632 | AI557497 | Above |
| 11 | 39329_at | Actinin alpha 1 | ACTN1 | X15804 | Above |
| 12 | 39389_at | CD9 antigen p24 | CD9 | M38690 | Above |
| 13 | 32207_at | membrane protein palmitoylated 1 55 kD | MPP1 | M64925 | Above |
| 14 | 32236_at | ubiquitin-conjugating enzyme E2G 2 | UBE2G2 | AF032456 | Above |
| | | homologous to yeast UBC7 | | | |
| 15 | 32251_at | hypothetical protein FLJ21174 | FLJ21174 | AA149307 | Above |
| 16 | 35764_at | chromosome X open reading frame 5 | OFD1 | Y15164 | Above |
| 17 | 36620_at | superoxide dismutase 1 soluble | SOD1 | X02317 | Above |
| | | amyotrophic lateral sclerosis 1 adult | | | |
| 18 | 36937_s_at | PDZ and LIM domain 1 elfin | PDLIM1 | U90878 | Above |
| 19 | 37326_at | proteolipid protein 2 colonic epithelium- | PLP2 | U93305 | Above |
| | | enriched | | | |
| 20 | 37350_at | clone 889N15 on chromosome Xq22.1-22.3. | PSMD10 | AL031177 | Above |
| | | Contains part of the gene for a novel | | | |
| | | protein similar to X. laevis Cortical | | | |
| | | Thymocyte Marker CTX | | | |
| 21 | 38738_at | SMT3 suppressor of mif two 3 yeast | SMT3H1 | X99584 | Above |
| | | homolog 1 | | | |
| 22 | 39168_at | Ac-like transposable element | ALTE | AB018328 | Above |
| 23 | 40903_at | ATPase H transporting lysosomal vacuolar | APT6M8-9 | AL049929 | Above |
| | | proton pump membrane sector associated | | | |
| | | protein M8-9 | | | |
| 24 | 32572_at | ubiquitin specific protease 9 X chromosome | USP9X | X98296 | Above |
| | | Drosophila fat facets related | | | |
| 25 | 1065_at | fms-related tyrosine kinase 3 | FLT3 | U02687 | Above |
| 26 | 306_s_at | high-mobility group nonhistone | HMG14 | J02621 | Above |
| | | chromosomal protein 14 | | | |

TABLE 33
Genes selected by DAV/SOM for MLL

| | | | | | Above/ |
|---|---|---|---|---|---|
| | Affymetrix | | | Reference | Below |
| | number | Gene Name | GeneSymbol | number | Mean |
| 1 | 31492_at | Muscle specific gene | M9 | AB019392 | Above |
| 2 | 36777_at | DNA segment on chromosome 12 unique | D12S2489E | AJ001687 | Above |
| | | 2489 expressed sequence | | | |
| 3 | 39301_at | Calpain 3 p94 | CAPN3 | X85030 | Below |
| 4 | 41448_at | Homeo box A4 | HOXA4 | AC004080 | Above |
| 5 | 39424_at | tumor necrosis factor receptor superfamily | TNFRSF14 | U70321 | Below |
| | | member 14 herpesvirus entry mediator | | | |
| 6 | 40076_at | Tumor protein D52-like 2 | TPD52L2 | AF004430 | Above |
| 7 | 40493_at | Human cell surface glycoprotein CD44 | CD44 | L05424 | Above |
| | | (CD44) gene, 3′ end of long tailed isoform. | | | |
| 8 | 40506_s_at | Homo sapiens polyadenylate binding | | U75686 | Above |
| | | protein mRNA, complete cds. | | | |
| 9 | 40514_at | hypothetical 43.2 Kd protein | LOC51614 | AF091085 | Above |
| 10 | 40763_at | Meis1 mouse homolog | MEIS1 | U85707 | Above |
| 11 | 40797_at | a disintegrin and metalloproteinase domain | ADAM10 | AF009615 | Above |
| | | 10 | | | |
| 12 | 40798_s_at | a disintegrin and metalloproteinase domain | ADAM10 | Z48579 | Above |
| | | 10 | | | |
| 13 | 41747_s_at | myocyte-specific enhancer factor 2A | MEF2A | U49020 | Above |
| | | (MEF2A) gene | | | |
| 14 | 32193_at | Plexin C1 | PLXNC1 | AF030339 | Above |
| 15 | 32215_i_at | KIAA0878 protein | KIAA0878 | AB020685 | Above |
| 16 | 33412_at | LGALS1 Lectin, galactoside-binding, | LGALS1 | AI535946 | Above |
| | | soluble, 1 (galectin 1) | | | |
| 17 | 34306_at | muscleblind Drosophila like | MBNL | AB007888 | Above |
| 18 | 34785_at | KIAA1025 protein | KIAA1025 | AB028948 | Above |
| 19 | 35298_at | eukaryotic translation initiation factor 3 | EIF3S7 | U54558 | Above |
| | | subunit 7 zeta 66/67 kD | | | |
| 20 | 36690_at | Nuclear receptor subfamily 3 group C | NR3C1 | M10901 | Above |
| | | member 1 | | | |
| 21 | 37675_at | solute carrier family 25 mitochondrial | SLC25A3 | X60036 | Above |
| | | carrier phosphate carrier member 3 | | | |
| 22 | 38391_at | capping protein actin filament gelsolin-like | CAPG | M94345 | Above |
| 23 | 38413_at | defender against cell death 1 | DAD1 | D15057 | Above |
| 24 | 39110_at | eukaryotic translation initiation factor 4B | EIF4B | X55733 | Above |
| 25 | 39867_at | Tu translation elongation factor | TUFM | S75463 | Above |
| | | mitochondrial | | | |
| 26 | 2062_at | Insulin-like growth factor binding protein 7 | IGFBP7 | L19182 | Above |
| 27 | 2036_s_at | CD44 antigen homing function and Indian | CD44 | M59040 | Above |
| | | blood group system | | | |
| 28 | 1914_at | Cyclin A1 | CCNA1 | U66838 | Above |
| 29 | 1327_s_at | mitogen-activated protein kinase kinase | MAP3K5 | U67156 | Above |
| | | kinase 5 | | | |
| 30 | 1126_s_at | Human cell surface glycoprotein CD44 | CD44 | L05424 | Above |
| | | (CD44) gene, 3′ end of long tailed isoform. | | | |
| 31 | 1102_s_at | Nuclear receptor subfamily 3 group C | NR3C1 | M10901 | Above |
| | | member 1 | | | |
| 32 | 873_at | homeo box A5 | HOXA5 | M26679 | Above |
| 33 | 706_at | Glucocorticoid receptor, beta | | HG4582- | Above |
| | | | | HT4987 | |
| 34 | 657_at | protocadherin gamma subfamily C 3 | PCDHGC3 | L11373 | Above |

TABLE 34
Genes selected by DAV/SOM for Novel Class

| | | | | | Above/ |
|---|---|---|---|---|---|
| | Affymetrix | | | Reference | Below |
| | number | Gene Name | GeneSymbol | number | Mean |
| 1 | 33137_at | latent transforming growth factor beta | LTBP4 | Y13622 | Above |
| | | binding protein 4 | | | |
| 2 | 38081_at | leukotriene A4 hydrolase | LTA4H | J03459 | Above |
| 3 | 38661_at | seb4D | HSRNASEB | X75314 | Above |
| 4 | 39878_at | protocadherin 9 | PCDH9 | AI524125 | Above |
| 5 | 35260_at | KIAA0867 protein | MONDOA | AB020674 | Above |
| 6 | 1373_at | transcription factor 3 E2A immunoglobulin | TCF3 | M31523 | Above |
| | | enhancer binding factors E12/E47 | | | |
| 7 | 35177_at | KIAA0725 protein | KIAA0725 | AB018268 | Above |
| 8 | 38618_at | Human PAC clone RP3-515N1 from | LIMK2 | AC002073 | Above |
| | | 22q11.2-q22 | | | |
| 9 | 34947_at | phorbolin-like protein MDS019 | MDS019 | AA442560 | Above |
| 10 | 40692_at | transducin-like enhancer of split 4 homolog | TLE4 | M99439 | Above |
| | | of Drosophila E sp1 | | | |
| 11 | 38364_at | BCE-1 protein | BCE-1 | AF068197 | Above |
| 12 | 37960_at | carbohydrate chondroitin 6/keratan | CHST2 | AB014679 | Above |
| | | sulfotransferase 2 | | | |
| 13 | 994_at | Protein tyrosine phosphatase receptor type M | PTPRM | X58288 | Above |
| 14 | 31892_at | Protein tyrosine phosphatase receptor type M | PTPRM | X58288 | Above |
| 15 | 995_g_at | Protein tyrosine phosphatase receptor type M | PTPRM | X58288 | Above |
| 16 | 41073_at | G protein-coupled receptor 49 | GPR49 | AI743745 | Above |
| 17 | 41708_at | KIAA1034 protein | KIAA1034 | AB028957 | Above |
| 18 | 34376_at | protein kinase cAMP-dependent catalytic | PKIG | AB019517 | Below |
| | | inhibitor gamma | | | |
| 19 | 37978_at | quinolinate phosphoribosyltransferase | QPRT | D78177 | Below |
| | | nicotinate-nucleotide pyrophosphorylase | | | |
| | | carboxylating | | | |
| 20 | 38717_at | DKFZP586A0522 protein | DKFZP586A0522 | AL050159 | Below |
| 21 | 33999_f_at | Human L2-9 transcript of unrearranged | | X58398 | Above |
| | | immunoglobulin V H 5 pseudogene | | | |
| 22 | 36181_at | LIM and SH3 protein 1 | LASP1 | X82456 | Below |
| 23 | 41202_s_at | conserved gene amplified in osteosarcoma | OS4 | AF000152 | Above |
| 24 | 41138_at | Antigen identified by monoclonal | MIC2 | M16279 | Below |
| | | antibodies 12E7 F21 and O13 | | | |
| 25 | 40771_at | Moesin | MSN | Z98946 | Above |
| 26 | 39070_at | singed Drosophila like sea urchin fascin | SNL | U03057 | Below |
| | | homolog like | | | |
| 27 | 32562_at | endoglin Osler-Rendu-Weber syndrome 1 | ENG | X72012 | Below |
| 28 | 36536_at | schwannomin interacting protein 1 | SCHIP-1 | AF070614 | Below |
| 29 | 36650_at | cyclin D2 | CCND2 | D13639 | Below |
| 30 | 39756_g_at | X-box binding protein 1 | XBP1 | Z93930 | Above |
| 31 | 34168_at | deoxynucleotidyltransferase terminal | DNTT | M11722 | Above |
| 32 | 1389_at | membrane metallo-endopeptidase neutral | MME | J03779 | Below |
| | | endopeptidase enkephalinase CALLA | | | |
| | | CD10 | | | |
| 33 | 41213_at | peroxiredoxin 1 | PRDX1 | X67951 | Above |
| 34 | 36571_at | Topoisomerase DNA II beta 180 kD | TOP2B | X68060 | Above |
| 35 | 253_g_at | clone GPCR W G protein-linked receptor | | L42324 | Below |
| | | gene (GPCR) gene, 5′ end of cds. | | | |
| 36 | 252_at | clone GPCR W G protein-linked receptor | | L42324 | Above |
| | | gene (GPCR) gene, 5′ end of cds. | | | |
| 37 | 2087_s_at | cadherin 11 type 2 OB-cadherin osteoblast | CDH11 | D21254 | Above |
| 38 | 36976_at | cadherin 11 type 2 OB-cadherin osteoblast | CDH11 | D21255 | Above |

TABLE 35
Genes selected by DAV/SOM for T-ALL

| | | | | | Above/ |
|---|---|---|---|---|---|
| | Affymetrix | | | Reference | Below |
| | number | Gene Name | GeneSymbol | number | Mean |
| 1 | 35016_at | Human Ia-associated invariant gamma- | | M13560 | Below |
| | | chain gene, exon 8, clones lambda-y(1, 2, 3). | | | |
| 2 | 36277_at | membrane protein (CD3-epsilon) gene | CD3E | M23323 | Above |
| 3 | 38147_at | SH2 domain protein 1A Duncan s disease | SH2D1A | AL023657 | Above |
| | | lymphoproliferative syndrome | | | |
| 4 | 38949_at | protein kinase C theta | PRKCQ | L01087 | Above |
| 5 | 32649_at | transcription factor 7 T-cell specific HMG- | TCF7 | X59871 | Above |
| | | box | | | |
| 6 | 33238_at | Human T-lymphocyte specific protein | LCK | U23852 | Above |
| | | tyrosine kinase p56lck (LCK) aberrant | | | |
| | | mRNA, complete cds. | | | |
| 7 | 35643_at | nucleobindin 2 | NUCB2 | X76732 | Above |
| 8 | 36473_at | ubiquitin specific protease 20 | USP20 | AB023220 | Above |
| 9 | 38319_at | CD3D antigen delta polypeptide TiT3 | CD3D | AA919102 | Above |
| | | complex | | | |
| 10 | 39709_at | selenoprotein W 1 | SEPW1 | U67171 | Above |
| 11 | 40775_at | integral membrane protein 2A | ITM2A | AL021786 | Above |
| 12 | 32794_g_at | T cell receptor beta locus | TRB | X00437 | Above |
| 13 | 37039_at | major histocompatibility complex class II | HLA-DRA | J00194 | Below |
| | | DR alpha | | | |
| 14 | 38051_at | mal T-cell differentiation protein | MAL | X76220 | Above |
| 15 | 38095_i_at | major histocompatibility complex class II | HLA-DPB1 | M83664 | Below |
| | | DP beta 1 | | | |
| 16 | 38096_f_at | major histocompatibility complex class II | HLA-DPB1 | M83664 | Below |
| | | DP beta 1 | | | |
| 17 | 38415_at | protein tyrosine phosphatase type IVA | PTP4A2 | U14603 | Above |
| | | member 2 | | | |
| 18 | 38833_at | Human mRNA for SB classII | | X00457 | Below |
| | | histocompatibility antigen alpha-chain | | | |
| 19 | 2059_s_at | lymphocyte-specific protein tyrosine kinase | LCK | M36881 | Above |
| 20 | 1241_at | protein tyrosine phosphatase type IVA | PTP4A2 | U14603 | Above |
| | | member 2 | | | |
| 21 | 1105_s_at | T cell receptor beta locus | TRB | M12886 | Above |

TABLE 36
Genes selected by DAV/SOM for TEL-AML1

| | | | | | Above/ |
|---|---|---|---|---|---|
| | Affymetrix | | | Reference | Below |
| | number | Gene Name | GeneSymbol | number | Mean |
| 1 | 31508_at | upregulated by 1, 25-dihydroxyvitamin D-3 | VDUP1 | S73591 | Above |
| 2 | 33690_at | cDNA DKFZp434A202 from clone | | AL080190 | Above |
| | | DKFZp434A202 | | | |
| 3 | 34481_at | vav proto-oncogene, exon 27, and complete | VAV | AF030227 | Above |
| | | cds. | | | |
| 4 | 36239_at | POU domain class 2 associating factor 1 | POU2AF1 | Z49194 | Above |
| 5 | 37470_at | Leukocyte-associated Ig-like receptor 1 | LAIR1 | AF013249 | Above |
| 6 | 38203_at | Potassium intermediate/small conductance | KCNN1 | U69883 | Above |
| | | calcium-activated channel subfamily N | | | |
| | | member 1 | | | |
| 7 | 38570_at | major histocompatibility complex class II | HLA-DOB | X03066 | Above |
| | | DO beta | | | |
| 8 | 38578_at | tumor necrosis factor receptor superfamily | TNFRSF7 | M63928 | Above |
| | | member 7 | | | |
| 9 | 38906_at | spectrin alpha erythrocytic 1 elliptocytosis 2 | SPTA 1 | M61877 | Above |
| 10 | 40729_s_at | nuclear factor of kappa light polypeptide | NFKBIL1 | Y14768 | Above |
| | | gene enhancer in B-cells inhibitor-like 1 | | | |
| 11 | 40745_at | adaptor-related protein complex 1 beta 1 | AP1B1 | L13939 | Above |
| | | subunit | | | |
| 12 | 41097_at | telomeric repeat binding factor 2 | TERF2 | AF002999 | Above |
| 13 | 41381_at | KIAA0308 protein | KIAA0308 | AB002306 | Above |
| 14 | 41442_at | core-binding factor runt domain alpha | CBFA2T3 | AB010419 | Above |
| | | subunit 2 translocated to 3 | | | |
| 15 | 31898_at | KIAA0212 gene product | KIAA0212 | D86967 | Above |
| 16 | 32660_at | KIAA0342 gene product | KIAA0342 | AB002340 | Above |
| 17 | 34194_at | cDNA FLJ21697 fis clone COL09740 | | AL049313 | Above |
| 18 | 35614_at | transcription factor-like 5 basic helix-loop- | TCFL5 | AB012124 | Above |
| | | helix | | | |
| 19 | 35665_at | Phosphoinositide-3-kinase class 3 | PIK3C3 | Z46973 | Above |
| 20 | 36008_at | protein tyrosine phosphatase type IVA | PTP4A3 | AF041434 | Above |
| | | member 3 | | | |
| 21 | 36524_at | Rho guanine nucleotide exchange factor | ARHGEF4 | AB029035 | Above |
| | | GEF 4 | | | |
| 22 | 36537_at | Rho-specific guanine nucleotide exchange | P114-RHO- | AB011093 | Above |
| | | factor p114 | GEF | | |
| 23 | 37280_at | MAD mothers against decapentaplegic | MADH1 | U59912 | Above |
| | | Drosophila homolog 1 | | | |
| 24 | 38652_at | hypothetical protein FLJ20154 | FLJ20154 | AF070644 | Above |
| 25 | 41200_at | CD36 antigen collagen type I receptor | CD36L1 | Z22555 | Above |
| | | thrombospondin receptor like 1 | | | |
| 26 | 32224_at | KIAA0769 gene product | KIAA0769 | AB018312 | Above |
| 27 | 36985_at | isopentenyl-diphosphate delta isomerase | IDI1 | X17025 | Above |
| 28 | 38124_at | midkine neurite growth-promoting factor 2 | MDK | X55110 | Above |
| 29 | 39824_at | ESTs | | AI391564 | Above |
| 30 | 40570_at | forkhead box O1A rhabdomyosarcoma | FOXO1A | AF032885 | Above |
| 31 | 41498_at | KIAA0911 protein | KIAA0911 | AB020718 | Above |
| 32 | 41814_at | fucosidase alpha-L- 1 tissue | FUCA1 | M29877 | Above |
| 33 | 32579_at | SWI/SNF related matrix associated actin | SMARCA4 | D26156 | Above |
| | | dependent regulator of chromatin subfamily | | | |
| | | a member 4 | | | |
| 34 | 33162_at | insulin receptor | INSR | X02160 | Above |
| 35 | 1779_s_at | pim-1 oncogene | PIM1 | M16750 | Above |
| 36 | 1488_at | protein tyrosine phosphatase receptor type K | PTPRK | L77886 | Above |
| 37 | 1325_at | MAD mothers against decapentaplegic | MADH1 | U59423 | Above |
| | | Drosophila homolog 1 | | | |
| 38 | 1336_s_at | protein kinase C beta 1 | PRKCB1 | X06318 | Above |
| 39 | 1299_at | Telomeric repeat binding factor 2 | TERF2 | X93512 | Above |
| 40 | 1217_g_at | protein kinase C beta 1 | PRKCB1 | X07109 | Above |
| 41 | 1077_at | recombination activating gene 1 | RAG1 | M29474 | Above |
| 42 | 932_i_at | zinc finger protein 91 HPF7 HTF10 | ZNF91 | L11672 | Above |
| 43 | 880_at | FK506-binding protein 1A 12 kD | FKBP1A | M34539 | Above |
| 44 | 755_at | inositol 1 4 5-triphosphate receptor type 1 | ITPR1 | D26070 | Above |
| 45 | 577_at | midkine neurite growth-promoting factor 2 | MDK | M94250 | Above |
| 46 | 160029_at | protein kinase C beta 1 | PRKCB1 | X07109 | Above |

C. Comparison of Genes Selected by the Different Metrics

There is a high degree of overlap between the genes chosen by the various metrics, however the top ranked genes for each metric differ. Despite this, the top genes selected by the various metrics are all able to accurately identify the leukemia risk groups as detailed below. As a result, a limited number of genes can be used to accurately identify the genetic subtypes and one can use non-overlapping lists and still achieve high prediction accuracy. Thus, there are many genes that are distinct discriminators of these seven risk groups, and one need only to use a small subset of these in a supervised learning algorithm to accurately identify a case as belonging to the genetic subtype.

D. Decision Tree for the Diagnosis of Genetic Subtypes

Classification was approached using a decision tree format, in which the first decision was T-ALL versus B-lineage (non-T-ALL). Within the B-lineage subset, cases were then sequentially classified into the known risk groups characterized by the presence of E2A-PBX1, TEL-AML1, BCR-ABL, MLL chimeric genes, and lastly hyperdiploid>50 chromosomes. Cases not assigned to one of these classes were left unassigned. Classification was performed using the supervised learning algorithms described below.

E. Description of Supervised Learning Algorithms

An analysis of the profiles was performed using alinear classifier, C4.5, and a variety of different non-linear classifiers. The non-linear classifiers consistently outperformed the linear classifier. Therefore, only the description and data from non-linear classifiers are included below.

1. Support Vector Machine (SVM)

Support vector machine (SVM) selects a small number of critical boundary instances from each class and builds a linear discriminant function that separates them as widely as possible (Witten and Frank, Data Mining: Practical Machine Learning Tools and Techniques with Java Implementation, Morgan Kaufmann, 1999, herein incorporated by reference). In the case where no linear separation is possible, the technique of “kernel” is used to automatically inject the training instances into a higher dimensional space and a separator is learned in that space. The Weka version of SVM developed at the University of Waikato of New Zealand (www.cs.waikato.ac.nz/ml/weka), which implements Platt's sequence minimal optimization algorithm for training a support vector classifier using polynomial kernels was used (Platt, “Fast Training of Support Vector Machines Using Sequential Minimal Optimization,” Advances in Kernel Methods—Support Vector Learning, Schlkpof et al., eds., MIT Press, 1998, herein incorporated by reference).

2. Prediction by Collective Likelihood of Emerging Patterns (PCL)

Emerging patterns (EPs) are a notion used in data mining to discover sharp differences between two classes of data (Dong and Li, “Efficient Mining of Emerging Patterns: Discovering Trends and Differences,” Proc. 5th ACM SIGKDD International Conference on Knowledge Discovery and Data Mining, pp. 43-52 (1999), herein incorporated by reference). An EP is a pattern—the expression level of several genes in our case—whose frequency increases significantly from one class of samples to another class. In particular, the most general patterns that have infinite growth in the sense that their frequency in one class is 0% and in another class is greater than 0% and none of their proper subpatterns are EPs were identified. These EPs can then be combined into reliable rules for subtype prediction. Three earlier methods for classification based on EPs are JEP(Li et al. (2001) Knowledge and Information System 3:131-45, herein incorporated by reference), DeEPs (Li et al., “DeEPs: Instance-based Classification by Emerging Patterns,” Proc. 4th European Conference on Principles and Practice of Knowledge Discovery in Databases, pp. 191-200, 2000, herein incorporated by reference), and CAEP (Dong et al., “CAEP: Classification by Aggregation Emerging Patterns,” Proc. 2nd International Conference on Discovery Science, pages 30-42, 1999, herein incorporated by reference).

In this analysis an original variation in the spirit of JEP but with a different manner of aggregating EPs was used. Given two training data sets Dₚ and Dₙ and a testing sample T, the first phase was to discover EPs from Dₚ and Dₙ. Denote the EPs of Dₚ, in descending order of frequency, as TopEP^{p}₁, . . . , TopEP^{p}ᵢ, and those of Dₙ as TopEPⁿ₁, . . . , TopEPⁿⱼ. Suppose T contains the following EPs of Dₚ: TopEP^{p}ᵢₗ, . . . , TopEP^{p}ᵢₓ, where i1<i2<. . . <ix<=i; and the following EPs of Dₙ: TopEPⁿⱼₗ, . . . , TopEPⁿ_{jy}, where j1<j2<. . . <jy<=j. In the next step, two scores were calculated for T: scoreₚ=Σ[frequency(TopEP^{p}ⱼₘ)/frequency(TopEP^{p}ₘ)] and scoreₙ=Σ[frequency(TopEPⁿⱼₘ)/frequency(TopEPⁿₘ)], summing over m=1 . . . k, where k<<i and k<<j. In this case, k is chosen to be 25. Finally, a prediction is made on T as follows: If scoreₚ>scoreₙ, then T is predicted to be in class Dₚ; otherwise, it is predicted as class Dₙ.

The spirit of this variation is to measure how far the top k EPs contained in T are away from the top k EPs of a class. For example, if k=1, then scoreₚ indicates whether the number-one EP contained in T is far from the most frequent EP of Dₚ. If the score is the maximum value 1, then the “distance” is very close, namely the most common property of Dₚ is also present in this testing sample. With smaller scores, the distance becomes further and the likelihood of T belonging to Dₚ becomes weaker. Using more than one top-ranked EPs in this way leads to very reliable predictions. This variation of EP-based classification method was termed “prediction by collective likelihood of EPs” or PCL for short.

3. k-Nearest Neighbor (k-NN)

k-NN is a typical instance-based learner where the class of a new instance is decided by the majority class of its k closest neighbors (Cover and Hart (1967) IEEE Transactions on Information Theory 13:21-27, herein incorporated by reference). This method was used with the Euclidean distance metric. Conceptually, this is one of the most straightforward methods and is often used as a baseline for comparison purposes. The data were normalized using the z-score method, then the “best” few genes were chosen using one of the statistical gene selection methods. For these experiments, the “top n” genes, where n=1-50, were used. The expression values of the top genes from each diagnostic sample were treated as a vector in n-dimensional space. To classify a new sample, the same top n genes were chosen, and the Euclidean distance was computed between this new vector and each vector in the training data. The prediction was made by a majority vote of the k nearest samples, where k=1 or k=3. In this experiment, k was set to 1.

4. Artificial Neural Network (ANN)

The artificial neural network (ANN) learning models built are all feed-forward, fully connected, and non-recurrent. The input layer of each ANN contains 50 units, which correspond to the 50 input values (the “top 50” scoring genes). Each ANN has one hidden layer with 4 units, and an output layer that contains two units, which represent the two class labels. In a preprocessing step all input data was normalized using the z-score method. The apparent error was estimated using 3-fold cross-validation. That is, for each training procedure, the training samples were randomly shuffled and divided into three groups of approximately equal size. A model was built with two of the groups and the third group was set aside for validation. This step was repeated three times, each time with a different group for validation. This shuffling-training process was repeated ten times, resulting in 30 ANN models. Each test sample was fed into each of the 30 ANN models, and the output was the average of the 30 outputs. The class predicted was the one that was represented by the output unit with the larger average output value.

F. Table of Results Using the Different Algorithms to Predict the Genetic Subgroups

A summary of the true prediction accuracy on the blinded test set of 112 cases are presented in Tables 37-39. Sensitivity was calculated as the number of positive samples predicted/the number of true positives. Specificity was calculated as the number of negative samples predicted/the number of true negatives.

TABLE 37
True Prediction Accuracy Results
on Test Set using SVM and ANN algorithms

| | | SVM | | | | ANN |
|---|---|---|---|---|---|---|
| | | Chi Sq | CFS | T-stats | SOM/DAV | Wilkins' |
| T-ALL | True | 100 | 100 | 100 | 100 | 100 |
| | Accuracy | | | | | |
| | Sensitivity | 100 | 100 | 100 | 100 | 100 |
| | Specificity | 100 | 100 | 100 | 100 | 100 |
| E2A- | True | 100 | 100 | 100 | 100 | 100 |
| PBX1 | Accuracy | | | | | |
| | Sensitivity | 100 | 100 | 100 | 100 | 100 |
| | Specificity | 100 | 100 | 100 | 100 | 100 |
| TEL- | True | 99 | 99 | 98 | 97 | 100 |
| AML1 | Accuracy | | | | | |
| | Sensitivity | 100 | 100 | 100 | 100 | 100 |
| | Specificity | 98 | 98 | 97 | 97 | 100 |
| BCR- | True | 95 | 97 | 94 | 97 | 97 |
| ABL | Accuracy | | | | | |
| | Sensitivity | 50 | 67 | 33 | 83 | 83 |
| | Specificity | 100 | 100 | 100 | 98 | 98 |
| MLL | True | 100 | 98 | 100 | 97 | 100 |
| | Accuracy | | | | | |
| | Sensitivity | 100 | 100 | 100 | 86 | 100 |
| | Specificity | 100 | 98 | 100 | 100 | 100 |
| H>50 | True | 96 | 96 | 96 | 95 | 94 |
| | Accuracy | | | | | |
| | Sensitivity | 100 | 100 | 100 | 95 | 100 |
| | Specificity | 93 | 93 | 93 | 93 | 89 |

TABLE 38
True Prediction Accuracy Results on Test Set using k-NN

| | | k-NN | | | |
|---|---|---|---|---|---|
| | | Chi Sq | CFS | T-stats | Wilkins' |
| T-ALL | True Accuracy | 100 | 100 | 100 | 100 |
| | Sensitivity | 100 | 100 | 100 | 100 |
| | Specificity | 100 | 100 | 100 | 100 |
| E2A-PBX1 | True Accuracy | 100 | 100 | 100 | 100 |
| | Sensitivity | 100 | 100 | 100 | 100 |
| | Specificity | 100 | 100 | 100 | 100 |
| TEL-AML1 | True Accuracy | 98 | 98 | 99 | 100 |
| | Sensitivity | 100 | 96 | 96 | 100 |
| | Specificity | 97 | 98 | 100 | 100 |
| BCR-ABL | True Accuracy | 94 | 97 | 95 | 93 |
| | Sensitivity | 33 | 67 | 50 | 67 |
| | Specificity | 100 | 100 | 100 | 96 |
| MLL | True Accuracy | 100 | 98 | 95 | 100 |
| | Sensitivity | 100 | 83 | 100 | 100 |
| | Specificity | 100 | 100 | 94 | 100 |
| H>50 | True Accuracy | 98 | 96 | 94 | 98 |
| | Sensitivity | 100 | 100 | 95 | 100 |
| | Specificity | 96 | 93 | 93 | 96 |

TABLE 39
True Prediction Accuracy Results on Test Set using PCL

PCL

| | | Chi Sq | CFS |
|---|---|---|---|
| T-ALL | True Accuracy | 100 | 100 |
| | Sensitivity | 100 | 100 |
| | Specificity | 100 | 100 |
| E2A-PBX1 | True Accuracy | ND | 100 |
| | Sensitivity | ND | 100 |
| | Specificity | ND | 100 |
| TEL-AML1 | True Accuracy | 99 | ND |
| | Sensitivity | 96 | ND |
| | Specificity | 100 | ND |
| BCR-ABL | True Accuracy | 97 | ND |
| | Sensitivity | 67 | ND |
| | Specificity | 100 | ND |
| MLL | True Accuracy | 100 | ND |
| | Sensitivity | 100 | ND |
| | Specificity | 100 | ND |
| H > 50 | True Accuracy | 98 | ND |
| | Sensitivity | 100 | ND |
| | Specificity | 96 | ND |

The assignment of a leukemic sample to a specific biologic subgroup is more accurately reflected by its gene expression profile than by the presence or absence of a specific genetic lesion. For example, four patients that had expression profiles classified as TEL-AML1, despite lacking a TEL-AML1 chimeric message by the reverse transcriptase polymerase chain reaction (RT-PCR) were found to have an alteration in TEL, suggesting a common underlying biology. Thus, from a technical viewpoint, gene expression profiling provides a viable alternative to standard diagnostic approaches.

G. Absence of Correlation of Expression Data for Genetic Subtypes with Stage of B-Cell Differentiation

The expression profiles of the different risk groups of B-cell leukemias do notcorrespond to markers of different stages of B-cell differentiation,. The first issue is defining the stage of B-cell differentiation. The defined stages of BM derived B-cells relevant to pediatric ALL are outlined below in Table 40, along with their frequency in pediatric ALL (Campana and Behm (2000) J. Immunologic Methods, 243:59-75). Three stages of differentiation are defined by a limited number of markers. In Table 41 below, the distribution of the leukemia cases into these B-cell differentiation stages is shown. As can be seen, none of the genetic subtypes is specifically associated with one of these three stages of differentiation. Thus, this simple analysis clearly shows that the majority of the chromosomal translocation subgroups in pediatric ALL do not correspond to a specific stage of B-cell differentiation. This is a well-known fact in the field of pediatric ALL and differs from the relationship typically seen between chromosomal translocations and other genetic lesions, and the stage of differentiation seen in B-cell lymphomas.

TABLE 40
Immunophenotyping of acute lymphoblastic leukemias^{a}

| | Leukocyte antigen expression | | | | | |
|---|---|---|---|---|---|---|
| | (% of cases positive) | | | | | Frequency |
| Subtype | CD19 | CD22 | cIgμ | sIgμ | sIg κ or λ | (%) |
| Early Pre-B | 100 | >95 | 0 | 0 | 0 | 60-65 |
| Pre-B | 100 | 100 | 100 | 0 | 0 | 20-25 |
| Transitional | 100 | 100 | 100 | 100 | 0 | 1-3 |

Abbreviations: cIg μ, cytoplasmic immunoglobulin μ chain; sIg μ, surface immunoglobulin μ chain; sIg κor λ, surface immunoglobulin κ or λ chains
^{a}D. Campana and F. G. Behm, “Immunophenotyping of leukemia”, Journal of Immunological Methods 243: 59-75, 2000.

TABLE 41
Distribution of genetic subtypes by immunophenotype^{a}

| | | | TRANSITIONAL |
|---|---|---|---|
| | EARLY PRE-B | PRE-B | PRE B |
| E2A | 0 | 17 | 6 |
| TEL | 55 | 23 | 0 |
| BCR | 11 | 3 | 0 |
| MLL | 12 | 6 | 1 |
| Hyperdip > 50 | 49 | 9 | 5 |
| Novel | 8 | 4 | 1 |
| Total | 172 | 77 | 24 |

^{a}For this analysis, samples with other immunophenotypes (NOS or mature B-cell) were not included

The next goal was to determine whether a set of genes that could accurately identify subjectss by their stage of differentiation, regardless of leukemai risk group. To accomplish this, cases were assigned into one of three classes, early pre-B, pre-B, or transitional pre-B based on their immunophenotype. The top 50 genes that distinguished each group from the other two groups were selected using the Wilkins' metric. These genes were then used in an ANN analysis to assess their performance in correctly classifying the 273 diagnostic B-lineage ALL samples, for which a stage of differentiation could be determined, through a process of cross validation. The results of this analysis are included below.

TABLE 42
Accuracy Results for immunophenotype discrimination using
Wilkins' metric and ANN algorithm

| | Accuracy | Sensitivity | Specificity |
|---|---|---|---|
| Early Pre-B^{a} | 78.39% | 85.47% | 66.34% |
| Pre-B^{b} | 71.79% | 38.96% | 84.69% |
| Transitional Pre-B^{c} | 91.24% | 33.33% | 96.79% |

^{a}Cells with CD19+, CD22+, cytoplasmic Igμ−, surface Igμ− immunophenotype
^{b}Cells with CD19+, CD22+, cytoplasmic Igμ+, surface Igμ− immunophenotype
^{c}Cells with CD19+, CD22+, cytoplasmic Igμ+, surface Igμ+ immunophenotype

The selected genes perform rather poorly in correctly assigning cases to specific B-cell differentiation stages, with accuracies well below those achieved for prediction of the genetic subgroups. When these genes are used in a two-dimensional hierarchical clustering algorithm they failed to cluster cases by immunophenotype, but instead, resulted in the loose clustering of some of the genetic subgroups, including E2A-PBX1, TEL-AML1, BCR-ABL, MLL, and hyperdiploid>50. The analysis was repeated using genes selected by DAV and again, no clustering of the immunophenotypically-defined stages was observed. Thus, it was not possible to identify expression profiles that can accurately identify the immunophenotypically-defined differentiation stages of pediatric B-cell ALL. Moreover, the expression profiles that were defined for the genetic subtypes are not profiles that correspond to specific stages of B-cell differentiation. Although some of the genes that define specific genetic subtypes can be associated with a particular stage of B-cell differentiation, the majority of the discriminating genes show no correlation with differentiation.

H. Results for Relapse Prediction

In the prediction of whether a patient would go into continuous complete remission or would relapse, a subtype-specific approach was adopted. An individual classifier was constructed for each subtype of ALL. Given a sample, the subtype was first predicted, and then the corresponding subtype-specific prognostic classifier was invoked to predict whether the patient would relapse. This subtype-specific approach was required because an expression profile predictive of relapse for the entire group could not be defined.

In the construction of the type-specific classifiers, genes were selected by CFS unless this algorithm returned >20 genes, in which case the top 20 ranked genes by T-statistics were used. When the T-statistics method was used, the selection of how many among the top 20 T-statistics genes were to be used was made by performing cross validation experiments—that is, the top n genes for n=1 . . . 20 were picked the n that gave the best cross validation results was selected. The cross validation results for the optimal ice of genes are summarized in Table 43 below. The genes that were chosen for use subtype-specific relapse predictions are summarized in Table 44.

TABLE 43
Results of relapse prediction on indicated subgroups

| | | | | | | P value |
|---|---|---|---|---|---|---|
| | | | | | | by permutation |
| | Relapse | CCR | # genes | metric | Accuracy | test |
| T-ALL | 8 | 26 | 7 | t-stats | 97 | 0.034 |
| H>50 | 5 | 43 | 13 | t-stats | 100 | 0.018 |
| TEL- | 3 | 56 | 7 | CFS | 100 | 0.145 |
| AML1 | | | | | | |
| MLL | 5 | 7 | 4 | t-stats | 100 | 0.104 |
| Others | 4 | 56 | 20 | t-stats | 98.3 | 0.079 |

TABLE 44
Genes selected by T-statistics/CFS for relapse (T-ALL)

| | | | Above/ |
|---|---|---|---|
| | | Reference | Below |
| Gene Name | GeneSymbol | Number | Mean |
| Human TBXAS1 gene for | TBXAS1 | D34625 | Above |
| thromboxane synthase | | | |
| Homo sapiens mRNA for 41-kDa | | AB007851 | Above |
| phosphoribosylpyrophosphate | | | |
| synthetase- | | | |
| associated protein | | | |
| Human DNA sequence | | Z82206 | Above |
| from PAC 370M22 | | | |
| Human spinal | SMA5 | X83301 | Above |
| muscular atrophy gene | | | |
| Human cell surface | CD44 | L05424 | Above |
| glycoprotein CD44 | | | |
| Human mRNA for KIAA0056 gene | KIAA0056 | D29954 | Above |
| Human BTK region | | U01923 | Above |
| clone ftp-3 mRNA | | | |

TABLE 45
Genes Selected by T statistics/CFS for relapse Hyperdiploid >50

| | | | | | Above/ |
|---|---|---|---|---|---|
| | Affymetrix | | | Reference | Below |
| | number | Gene Name | Gene Symbol | Number | Mean |
| 1 | 37721_at | deoxyhypusine synthase | DHPS | U79262 | Above |
| 2 | 38721_at | KIAA1536 protein | KIAA1536 | W72733 | Above |
| 3 | 40120_at | hydroxyacyl glutathione | HAGH | X90999 | Above |
| | | hydrolase | | | |
| 4 | 41386_i_at | KIAA0346 protein | KIAA0346 | AB002344 | Above |
| 5 | 38677_at | stress 70 protein chaperone | STCH | U04735 | Above |
| | | microsome-associated 60 kD | | | |
| 6 | 37620_at | Human TFIID subunits TAF20 | | U57693 | Above |
| | | and TAF15 mRNA, complete | | | |
| | | cds. | | | |
| 7 | 34703_f_at | EST | | AA151971 | Above |
| 8 | 38355_at | DEAD/H Asp-Glu-Ala-Asp/His | DBY | AF000984 | Above |
| | | box polypeptide Y chromosome | | | |
| 9 | 41214_at | ribosomal protein S4 Y-linked | RPS4Y | M58459 | Above |
| 10 | 34530_at | Homo sapiens cDNA FLJ22448 | | W73822 | Above |
| | | fis clone HRC09541 | | | |
| 11 | 603_at | nuclear receptor subfamily 2 | NR2C1 | M29960 | Above |
| | | group C member 1 | | | |
| 12 | 32697_at | inositol myo 1 or 4 | IMPA1 | AF042729 | Above |
| | | monophosphatase 1 | | | |
| 13 | 41129_at | KIAA0033 protein | KIAA0033 | D26067 | Above |
| 14 | 33333_at | KIAA0403 protein | KIAA0403 | AB007863 | Above |
| 15 | 37078_at | CD3Z antigen zeta polypeptide | CD3Z | J04132 | Above |
| | | TiT3 complex | | | |
| 16 | 38148_at | cryptochrome 1 photolyase-like | CRY1 | D83702 | Above |
| 17 | 39150_at | ring finger protein 11 | RNF11 | U69559 | Above |
| 18 | 33869_at | DKFZp586N1323 from clone | | AL080218 | Above |
| | | DKFZp586N1323 | | | |
| 19 | 41447_at | KIAA0990 protein | KIAA0990 | AB023207 | Above |
| 20 | 39369_at | KIAA0935 protein | KIAA0935 | AB023152 | Above |

TABLE 46
Genes selected by T-statistics/CFS for relapse (TEL-AML1I)

| | | | | | Above/ |
|---|---|---|---|---|---|
| | Affymetrix | | Gene | Reference | Below |
| | number | Gene Name | Symbol | number | Mean |
| 1 | 35797_at | Human | IL-13Ra | Y10659 | Above |
| | | interleukin- | | | |
| | | 13 gene | | | |
| 2 | 37524_at | Human death- | DRAK2 | AB011421 | Above |
| | | associated | | | |
| | | protein kinase | | | |
| 3 | 34243_i_at | Human 1(3)mbt | | U89358 | Above |
| | | protein homolog | | | |
| | | mRNA | | | |
| 4 | 41398_at | Homo sapiens | | AL049305 | Above |
| | | mRNA. CDNA | | | |
| | | DKFZp564A186 | | | |
| 5 | 35195_at | H. sapiens | | Y11651 | Above |
| | | mRNA for | | | |
| | | phosphate cyclase | | | |
| 6 | 32393_s_at | Homo sapiens | | W27466 | Above |
| | | cDNA | | | |
| 7 | 31909_at | Homo sapiens | KIAA0754 | AB018297 | Above |
| | | mRNA for | | | |
| | | KIAA0754 | | | |
| | | protein | | | |

TABLE 47
Genes selected by T-statistics/CFS for relapse (MLL)

| | | | | | Above/ |
|---|---|---|---|---|---|
| | Affymetrix | | Gene | Reference | Below |
| | number | Gene Name | Symbol | number | Mean |
| 1 | 294_s_at | Protein Kinase | | | Below |
| | | Pitslre, Alpha, | | | |
| | | Alt. Splice 1- | | | |
| | | Feb | | | |
| 2 | 38226_at | 23h11 Homo | | W27152 | Below |
| | | sapiens cDNA | | | |
| 3 | 1398_g_at | Human protein | HUMMLK3A | L32976 | Above |
| | | kinase | | | |
| | | (MLK-3) | | | |
| | | mRNA | | | |
| 4 | 409_at | Human mRNA | | X56468 | Below |
| | | for 14.3.3 | | | |
| | | protein, a | | | |
| | | protein | | | |
| | | kinase | | | |
| | | regulator | | | |

TABLE 48
Genes selected by T-statistics/CFS for relapse (Others)

| | | | | | Above/ |
|---|---|---|---|---|---|
| | Affymetrix | | | Reference | Below |
| | number | Gene Name | GeneSymbol | number | Mean |
| 1 | 33782_r_at | nn82f03.s1 Homo sapiens cDNA, 3 end/ | | AA587372 | Above |
| | | clone = IMAGE-1090397 | | | |
| 2 | 33338_at | Human transcription factor ISGF-3 mRNA | | M97936 | Above |
| 3 | 40242_at | Human (clone N5-4) protein p84 mRNA | | L36529 | Above |
| 4 | 37018_at | qd05c04.x1 Homo sapiens cDNA, 3 end/ | | AI189287 | Above |
| | | clone = IMAGE-1722822 | | | |
| 5 | 38337_at | Homo sapiens zinc finger protein mRNA | | U62392 | Above |
| 6 | 41464_at | Human mRNA for KIAA0339 gene | KIAA0339 | AB002337 | Above |
| 7 | 38064_at | H. sapiens lrp mRNA | LRP | X79882 | Above |
| 8 | 33173_g_at | yc89b05.r1 Homo sapiens cDNA, 5 end/ | | T75292 | Below |
| | | clone = IMAGE-23231 | | | |
| 9 | 33365_at | Homo sapiens mRNA for KIAA0945 | KIAA0945 | AB023162 | Above |
| | | protein | | | |
| 10 | 39367_at | ni38e08.s1 Homo sapiens cDNA, 3 end/ | | AA522537 | Above |
| | | clone = IMAGE-979142 | | | |
| 11 | 41108_at | Homo sapiens mRNA for putative GTP- | PGPL | Y14391 | Above |
| | | binding protein | | | |
| 12 | 37304_at | Homo sapiens heterochromatin protein p25 | P25beta | U35451 | Below |
| | | mRNA | | | |
| 13 | 40359_at | Human DNA-binding protein (HRC1) | HRC1 | M91083 | Above |
| | | mRNA | | | |
| 14 | 32792_at | Human DNA sequence from clone 465N24 | | AL031432 | Above |
| | | on chromosome 1p35.1-36.13. Contains | | | |
| | | two novel genes, ESTs, GSSs and CpG | | | |
| | | islands | | | |
| 15 | 34726_at | Human voltage-gated calcium channel beta | | U07139 | Above |
| | | subunit mRNA | | | |
| 16 | 40299_at | Homo sapiens G-protein coupled receptor | | AF091890 | Above |
| | | RE2 mRNA, | | | |
| 17 | 40704_at | H. sapiens mRNA for phosphatidylinositol | | Z29090 | Above |
| | | 3-kinase | | | |
| 18 | 38568_at | Homo sapiens p53 binding protein mRNA | | U82939 | Above |
| 19 | 32038_s_at | wi30c12.x1 Homo sapiens cDNA, 3 end/ | | AI739308 | Above |
| | | clone = IMAGE-2391766 | | | |
| 20 | 39613_at | H. sapiens HUMM9 mRNA | | X74837 | Above |

I. Permutations Test Results

As the number of relapse samples were small, in addition to the usual cross validation experiments, 1000 permutation experiments were performed for each subtype-specific relapse study. In each permutation experiment, the samples were re-partitioned in a manner that preserved class size by randomly swapping the class labels (“relapse” or “continuous complete remission”). The same metric was then employed to pick the same number of genes as in the original partitioning of the samples given by the original class labels. SVM was then used to obtain a prediction accuracy by cross validation for this random partition using these freshly selected genes. The percentage of these 1000 permutation experiments was taken as a p-value that gave an indication on how many random partitions of the original samples could achieve the same accuracy as the original samples. The results of these permutation experiments are summarized in the last column of Table 43 above. These results show that the high accuracy obtained on the predictability of relapse in T-lineage ALL, Hyperdiploid>50, and others are unlikely to be a random event. The higher p-values obtained for the subtypes of TEL-AML1 and MLL are probably due to the small number of relapse samples available for analysis.

TABLE 49
Permutation test results for predictors of T-ALL relapse

| | Affymetrix | | | | |
|---|---|---|---|---|---|
| Rank | number | t-statistic value | Perm 1% | Perm 5% | neighbors |
| 1 | 33777_at | 7.8337 | 7.3774 | 5.4783 | 6 |
| 2 | 41853_at | 6.1727 | 6.5948 | 4.8117 | 16 |
| 3 | 38866_at | 5.9890 | 6.0293 | 4.5611 | 12 |
| 4 | 41643_at | 5.6106 | 5.6815 | 4.3877 | 12 |
| 5 | 1126_s_at | 5.4777 | 5.5162 | 4.2375 | 11 |
| 6 | 41862_at | 5.3734 | 5.3759 | 4.1208 | 11 |
| 7 | 41131_f_at | 4.9134 | 5.2280 | 4.0295 | 17 |

TABLE 50
Permutation test results for predictors of
Hyperdiploid >50 relapse

| | Affymetrix | t-statistics | | | |
|---|---|---|---|---|---|
| Rank | number | value | Perm 1% | Perm 5% | neighbors |
| 1 | 37721_at | 8.7160 | 12.7358 | 9.9506 | 75 |
| 2 | 38721_at | 8.4162 | 10.7256 | 8.8438 | 59 |
| 3 | 40120_at | 7.2736 | 9.9837 | 8.0383 | 73 |
| 4 | 41386_i_at | 6.3436 | 9.0552 | 7.5579 | 88 |
| 5 | 38677_at | 6.2698 | 8.8633 | 7.2466 | 88 |
| 6 | 37620_at | 6.2174 | 8.4154 | 6.9604 | 82 |
| 7 | 34703_f_at | 6.0770 | 8.0982 | 6.8835 | 83 |
| 8 | 38355_at | 5.5120 | 7.8657 | 6.7434 | 92 |
| 9 | 41214_at | 5.4262 | 7.6583 | 6.6094 | 90 |
| 10 | 34530_at | 5.4013 | 7.5991 | 6.5109 | 87 |
| 11 | 603_at | 5.3142 | 7.5903 | 6.4409 | 87 |
| 12 | 32697_at | 5.1785 | 7.5146 | 6.3265 | 90 |
| 13 | 41129_at | 5.1450 | 7.3939 | 6.2121 | 88 |
| 14 | 33333_at | 5.1061 | 7.2601 | 6.1389 | 87 |
| 15 | 37078_at | 5.0738 | 7.1484 | 6.0308 | 86 |
| 16 | 38148_at | 4.9256 | 6.9688 | 5.9230 | 93 |
| 17 | 39150_at | 4.9061 | 6.9273 | 5.9015 | 93 |
| 18 | 33869_at | 4.8256 | 6.8900 | 5.8367 | 93 |
| 19 | 41447_at | 4.7919 | 6.8135 | 5.7621 | 93 |
| 20 | 39369_at | 4.7790 | 6.7731 | 5.7391 | 92 |

Individually, the discriminating genes for relapse in T-ALL are significant at either the 1% or 5% level, while those for hyperdiploid >50 fall at approximaltely the 7% level.

TABLE 51
Results of relapse prediction on indicated subgroups

| | | | | | | P value by |
|---|---|---|---|---|---|---|
| | | | # | | | permutation |
| | Relapse | CCR | genes | metric | Accuracy | test |
| T-ALL | 8 | 26 | 7 | t-stats | 97 | 0.034 |
| H > 50 | 5 | 43 | 13 | t-stats | 100 | 0.018 |
| TEL-AML1 | 3 | 56 | 7 | CFS | 100 | 0.145 |
| MLL | 5 | 7 | 4 | t-stats | 100 | 0.104 |
| Others | 4 | 56 | 20 | t-stats | 98.3 | 0.079 |

As the number of relapse samples were small, in addition to the usual cross validation experiments, 1000 permutation experiments were also performed for each subtype-specific relapse study. In each permutation experiment, the samples were re-partitioned in a manner that preserved class size by randomly swapping the class labels (“relapse” or “continuous complete remission”). The same metric was employed to pick the same number of genes as in the original partitioning of the samples given by the original class labels. SVM was then used to obtain a prediction accuracy by cross validation for this random partition using these freshly selected genes. The percentage of these 1000 permutation experiments was taken as a p-value that gave an indication on how many random partitions of the original samples could achieve the same accuracy as the original samples. The results of these permutation experiments are summarized in the last column of Table 51 above. These results show that the high accuracy obtained on the predictability of relapse in T-lineage ALL, Hyperdiploid>50, and others are unlikely to be a random event. The p-values for the subtypes of TEL-AM1 and MLL are weaker than the other subtypes. However, in the case of TEL-AML1 the number of relapse samples were exceedingly small (3) and in the case of MLL the number of relapse and non-relapse samples were both very small.

J. Results for Secondary AML Prediction

For the secondary AML prediction, the same subtype-specific approach was adopted as described earlier in relapse prediction. This time only the TEL-AML1 subtype had sufficient number of samples for a secondary AML prediction model to be developed. For this model, the MIT score (Golub et al. (1999) Science 286:531-37, herein incorporated by reference) was used to select genes and SVM to perform classification using these genes. The MIT score of a gene is defined as T=|μ₁-μ₂|/(σ₁+σ₂), where μᵢ is the mean expression of that gene in the i^{th} class and σᵢ is the standard deviation of that gene in the i^{th} class. This formula assigns higher value to a gene that has larger mean difference between two classes and has smaller variance within both classes. The 20 genes with the highest MIT scores in TEL-AML1 patients that went into continuous complete remission versus those TEL-AML1 samples that developed secondary AML are listed in Table 52 below. 100% accuracy for secondary AML prediction accuracy was achieved on TEL-AML1 specific subtype samples using these 20 genes. A permutation test was also performed in the same manner as described earlier in the subtype-specific relapse prediction, and obtained a p-value of 0.031 was obtained, demonstrating that the predictability of the development of secondary AML in TEL-AML1-specific patients was unlikely to be a random event.

TABLE 52
Genes selected by MIT score for secondary AML

| | | | | |
|---|---|---|---|---|
| | | | | Above/ |
| Affymetrix | | Gene | Reference | Below |
| Number | Gene Name | Symbol | Number | Mean |

TEL-AML1

| | | | | | |
|---|---|---|---|---|---|
| 1 | 34890_at | ATPase H transporting lysosomal vacuolar | ATP6A1 | L09235 | Above |
| | | proton pump alpha polypeptide 70 kD | | | |
| | | isoform 1 | | | |
| 2 | 40925_at | hypothetical protein FLJ10803 | FLJ10803 | AA554945 | Above |
| 3 | 1719_at | mutS E. coli homolog 3 | MSH3 | U61981 | Above |
| 4 | 32877_i_at | EST IMAGE: 954213 | | AA524802 | Above |
| 5 | 32650_at | neuronal protein | NP25 | Z78388 | Above |
| 6 | 33173_g_at | hypothetical protein FLJ10849 | FLJ10849 | T75292 | Above |
| 7 | 32545_r_at | RSU-1/RSP-1 | RSU-1 | L12535 | Above |
| 8 | 34889_at | ATPase H transporting lysosomal vacuolar | ATP6A1 | AA056747 | Above |
| | | proton pump alpha polypeptide 70 kD | | | |
| | | isoform 1 | | | |
| 9 | 35180_at | cDNA DKFZp586F1323 from clone | | AL050205 | Above |
| | | DKFZp586F1323 | | | |
| 10 | 34274_at | KIAA1116 protein | KIAA1116 | AB029039 | Above |
| 11 | 35727_at | hypothetical protein FLJ20517 | FLJ20517 | AI249721 | Above |
| 12 | 1627_at | tyrosine kinase (GB: Z25437) | | HG2715- | Above |
| | | | | HT2811 | |
| 13 | 1461_at | nuclear factor of kappa light polypeptide | NFKBIA | M69043 | Below |
| | | gene enhancer in B-cells inhibitor alpha | | | |
| 14 | 36023_at | lacrimal proline rich protein | LPRP | AI864120 | Above |
| 15 | 39167_r_at | serine or cysteine proteinase inhibitor | SERPINH2 | D83174 | Above |
| | | clade H heat shock protein 47 member 2 | | | |
| 16 | 39969_at | H4 histone family member G | H4FG | AA255502 | Above |
| 17 | 38692_at | NGFI-A binding protein 1 ERG1 binding | NAB1 | AF045451 | Above |
| | | protein 1 | | | |
| 18 | 1594_at | polymerase RNA II DNA directed | POLR2C | J05448 | Above |
| | | polypeptide C 33 kD | | | |
| 19 | 33234_at | RBP1-like protein | LOC51742 | AA887480 | Above |
| 20 | 34739_at | hypothetical protein FLJ20275 | FLJ20275 | W26023 | Above |

TABLE 53
Permutation test results for secondary AML

| | Affymetrix | t-statistics | Perm | Perm | Perm | |
|---|---|---|---|---|---|---|
| Rank | number | number | 1% | 5% | median | neighbors |
| 1 | 34890_at | 1.2204 | 2.7933 | 2.2138 | 1.4712 | 822 |
| 2 | 40925_at | 1.0712 | 2.0006 | 1.7607 | 1.2884 | 859 |
| 3 | 1719_at | 1.0599 | 1.8536 | 1.6272 | 1.1894 | 767 |
| 4 | 32877_i_at | 1.0364 | 1.7125 | 1.5218 | 1.1200 | 715 |
| 5 | 32650_at | 1.0217 | 1.6580 | 1.4584 | 1.0776 | 646 |
| 6 | 33173_g_at | 1.0126 | 1.5868 | 1.4132 | 1.0416 | 595 |
| 7 | 32545_r_at | 1.0097 | 1.5536 | 1.3630 | 1.0223 | 536 |
| 8 | 34889_at | 0.9959 | 1.5164 | 1.3241 | 1.0009 | 512 |
| 9 | 35180_at | 0.9854 | 1.4838 | 1.2938 | 0.9777 | 477 |
| 10 | 34274_at | 0.9420 | 1.4759 | 1.2721 | 0.9600 | 550 |
| 11 | 35727_at | 0.8493 | 1.4482 | 1.2507 | 0.9415 | 809 |
| 12 | 1627_at | 0.8471 | 1.4207 | 1.2398 | 0.9254 | 782 |
| 13 | 1461_at | 0.8312 | 1.4012 | 1.2260 | 0.9114 | 801 |
| 14 | 36023_at | 0.8177 | 1.3551 | 1.2012 | 0.8995 | 813 |
| 15 | 39167_r_at | 0.8136 | 1.3462 | 1.1806 | 0.8894 | 790 |
| 16 | 39969_at | 0.8122 | 1.3395 | 1.1702 | 0.8785 | 759 |
| 17 | 38692_at | 0.8109 | 1.3333 | 1.1565 | 0.8696 | 729 |
| 18 | 1594_at | 0.8103 | 1.3142 | 1.1503 | 0.8626 | 696 |

TABLE 54
Additional Genes selected by
T statistics for BCR-ABL risk group

| Gene symbol | Accession Number |
|---|---|
| TUBA1 | HG2259-HT2348 |
| TUBA1 | X06956 |
| CRADD | U84388 |
| SLC2A5 | M55531 |
| PHYH | AF023462 |
| ZFPL1 | AF001891 |
| CD34 | S53911 |
| KIAA0015 | D13640 |
| CLECSF2 | X96719 |
| CD34 | M81945 |
| GAB1 | U43885 |
| E2F5 | U31556 |
| CLTB | M20470 |
| ENG | X72012 |
| LOC55884 | AF038187 |
| TNFRSF1A | M58286 |
| TMSNB | D82345 |
| SNL | U03057 |
| KIAA0990 | AB023207 |
| MAP1A | W26631 |
| MYPT2 | AB007972 |
| IFI30 | J03909 |
| ERPROT213-21 | U94836 |
| DKFZP586A0522 | AL050159 |
| LOC51109 | AA126515 |
| | W29087 |
| TSTA3 | U58766 |
| TNFRSF1B | AI813532 |
| GSN | X04412 |
| KIAA0582 | AI761647 |
| STATI2 | AF037989 |
| | AL049313 |
| ITGA4 | X16983 |
| FLJ20500 | AA522530 |
| SDR1 | AF061741 |
| ARHGEF4 | AB029035 |
| C18ORF1 | AF009426 |
| MAPK14 | U19775 |
| FHL1 | AF063002 |
| GATA3 | X58072 |
| KIAA0076 | D38548 |
| KCNN1 | U69883 |
| POM121L1 | D87002 |
| IFI30 | J03909 |
| ABL1 | X16416 |
| NELL2 | D83018 |
| MEST | D78611 |
| S100A4 | W72186 |
| D12S2489E | AJ001687 |
| ATP2B4 | W28589 |
| CTGF | X78947 |
| RGS1 | S59049 |
| CDK9 | X80230 |
| | AI524873 |
| STIM1 | U52426 |
| VEGFB | U48801 |
| PPP2R2A | M64929 |
| CASP2 | U13022 |
| SPS | U34044 |
| HRK | D83699 |
| KIAA0870 | AB020677 |
| ABL | U07563 |
| PKIA | S76965 |
| FLJ12474 | AA306076 |
| CD97 | X94630 |
| HCK | M16591 |
| FYN | M14333 |
| KIR2DL3 | AC006293 |
| DMPK | L08835 |
| N33 | U42360 |
| FLJ13949 | AL041879 |
| PRKCZ | Z15108 |
| IL17R | U58917 |
| FMR2 | U48436 |
| INSR | M10051 |
| AHNAK | M80899 |
| KIAA0878 | AB020685 |
| CD86 | U04343 |
| | U82303 |
| KIAA1043 | AL033538 |
| N33 | U42349 |
| SYN47 | Y17829 |
| ITPR1 | D26070 |
| SFRS9 | AL021546 |
| EPOR | M60459 |
| GAC1 | AF030435 |
| CAMK4 | D30742 |
| KIAA0084 | D42043 |
| LAT | AJ223280 |
| XBP1 | Z93930 |
| FLT3LG | U03858 |
| TESK1 | D50863 |
| | AF070633 |
| KIAA0681 | U89358 |
| FUT8 | Y17979 |

TABLE 55
Additional Genes selected
by T statistics for E2A-PBX1 Risk Group

| Gene symbol | Accession Number |
|---|---|
| PBX1 | M86546 |
| | AL049381 |
| FAT | X87241 |
| BLK | S76617 |
| IRF4 | U52682 |
| GS3955 | D87119 |
| KIAA0802 | AB018345 |
| SCHIP-1 | AF070614 |
| SNL | U03057 |
| KIAA0655 | AB014555 |
| GS3955 | D87119 |
| IGFBP7 | L19182 |
| CDKN1A | U03106 |
| CSF2RB | H04668 |
| STATI2 | AF037989 |
| KIAA1029 | AB028952 |
| KIAA0247 | D87434 |
| | AL049397 |
| NP | X00737 |
| TM4SF2 | L10373 |
| ALOX5 | J03600 |
| LRMP | U10485 |
| PTPN2 | AI828880 |
| ALOX5AP | AI806222 |
| AEBP1 | AF053944 |
| TGFBR2 | D50683 |
| ODC1 | M33764 |
| NID2 | D86425 |
| ODC1 | X16277 |
| CBX1 | U35451 |
| CSF3R | M59820 |
| KIAA0172 | D79994 |
| IL1B | M15330 |
| KIAA0922 | AB023139 |
| LOC51097 | AA005018 |
| TUBA1 | X06956 |
| ITGA6 | S66213 |
| NFKBIL1 | Y14768 |
| ADPRT | J03473 |
| ADPRT | J03473 |
| CSF3R | M59818 |
| EFNB1 | U09303 |
| CD9 | M38690 |
| CDKN2D | U40343 |
| KIAA0442 | AB007902 |
| PRKCZ | Z15108 |
| | AF055029 |
| RECK | D50406 |
| GOLGA3 | D63997 |
| ZAP70 | L05148 |
| FLI1 | M98833 |
| LASP1 | X82456 |
| | AJ001381 |
| TBXA2R | D38081 |
| BHLHB2 | AB004066 |
| ADARB1 | U76421 |
| PTPN6 | X62055 |
| | X58398 |
| TIMP1 | D11139 |
| KIAA0554 | AB011126 |
| SRP14 | AI525652 |
| ATP9A | AB014511 |
| HELO1 | AL034374 |
| GNAQ | U43083 |
| POU4F1 | X64624 |
| MERTK | U08023 |
| KIAA0625 | AB014525 |
| PCLO | AB011131 |
| IL7R | AF043129 |
| ITGA6 | X53586 |
| TUBA1 | HG2259-HT2348 |
| PIR121 | L47738 |
| MAGED1 | W26633 |
| CD48 | M37766 |
| TLR1 | AL050262 |
| NPR1 | X15357 |
| GLUL | X59834 |
| DAPK1 | X76104 |
| | X58398 |
| ARHGEF4 | AB029035 |
| NKEFB | L19185 |
| | AL049435 |
| ITM2A | AL021786 |
| RAG2 | M94633 |
| | L24521 |
| SCGF | AF020044 |
| PRKACB | M34181 |
| KCNN4 | AF022797 |
| KCNN1 | U69883 |
| MAPKAPK2 | U12779 |
| PIN | AI540958 |
| TOP2B | X68060 |
| GATA2 | M68891 |
| IL1B | X04500 |
| PDE3B | U38178 |
| DGKD | D73409 |
| KIAA0993 | AB023210 |
| ADAM10 | AF009615 |
| IGLL1 | M27749 |
| PDLIM1 | U90878 |
| PRKAR1A | M33336 |
| CD34 | S53911 |
| GLA | U78027 |
| BAZ1B | AF072810 |
| EFNA1 | M57730 |
| FADS3 | AC004770 |
| FLT3 | U02687 |
| LOC57228 | AF091087 |
| BCL6 | U00115 |
| BMP2 | M22489 |
| CD22 | X59350 |
| KIAA0429 | AB007889 |
| DKFZP434C171 | AL080169 |
| CTBP2 | AF016507 |
| | M11810 |
| SIAT9 | AB018356 |
| CYBB | X04011 |
| AKR1B1 | X15414 |
| NFKBIL1 | Y14768 |
| UBE2V1 | U49278 |
| DOC-1R | AF089814 |
| BUB3 | AF047473 |
| IL7R | M29696 |
| ACK1 | L13738 |
| ENIGMA | L35240 |
| KIAA1071 | AB028994 |
| IGL | AI932613 |
| MN1 | X82209 |
| KIAA0823 | AB020630 |
| NFKB1 | M58603 |
| CD24 | L33930 |
| YWHAQ | X56468 |
| VDAC1 | L06132 |
| P85SPR | D63476 |
| SYNGR1 | AL022326 |
| NDR | Z35102 |
| JMJ | AL021938 |
| PRSC1 | D55696 |
| MRC1 | M93221 |
| | AI184710 |
| CRIP1 | AI017574 |
| KIAA0056 | D29954 |
| | AF039397 |
| | U79265 |
| SLAM | U33017 |
| LYL1 | AC005546 |
| KIAA0620 | AB014520 |
| VDAC1P | AJ002428 |
| SRP9 | AF070649 |
| PRDX1 | X67951 |
| SLC9A3R1 | AF015926 |
| CD72 | M54992 |
| ECM1 | U68186 |
| PPP2R5A | L42373 |
| HDGF | D16431 |
| MERTK | U08023 |
| | L02326 |
| CD34 | M81945 |
| IL17R | U58917 |
| ARL7 | AB016811 |
| P4HA2 | U90441 |
| BZRP | M36035 |
| F13A1 | M14539 |
| KRAS2 | M54968 |
| BS69 | X86098 |
| ORP150 | U65785 |
| | D28915 |
| LEF1 | AL049409 |
| SH2D1A | AL023657 |
| LY6E | U66711 |
| FACVL1 | D88308 |
| EPB42 | M60298 |
| | AL049471 |
| BMI1 | L13689 |
| KCNJ13 | N36926 |
| N33 | U42349 |
| VIL2 | X51521 |
| CCNG2 | U47414 |
| C18ORF1 | AF009425 |
| NUMA1 | Z11584 |
| DBN1 | U00802 |
| FLT3 | U02687 |
| KIAA0854 | AB020661 |
| MGC4175 | AI656421 |
| KIAA1012 | AB023229 |
| CIRBP | D78134 |
| ST5 | U15131 |
| KIAA0001 | D13626 |
| CCR1 | D10925 |
| CD19 | M28170 |
| SNRPE | AA733050 |
| CR2 | M26004 |
| HEXA | M16424 |
| IFIT4 | AF026939 |
| | W26667 |
| EPOR | M60459 |
| TMSNB | D82345 |
| GCLM | L35546 |
| H41 | H15872 |
| TUBB2 | HG1980-HT2023 |
| TNFAIP2 | M92357 |
| GAB1 | U43885 |
| PTPRK | L77886 |
| BCL7A | X89984 |

TABLE 56
Additional Genes selected by
T statistics for Hyperdiploid > 50
Risk Group

| Gene symbol | Accession Number |
|---|---|
| SH3BP5 | AB005047 |
| FLT3 | U02687 |
| MX1 | M33882 |
| NPY | AI198311 |
| SOD1 | X02317 |
| PTPRK | L77886 |
| IL1B | X04500 |
| CD9 | M38690 |
| FLT3 | U02687 |
| PGK1 | V00572 |
| EFNB1 | U09303 |
| FOS | K00650 |
| IL1B | M15330 |
| MRC1 | M93221 |
| HMG14 | J02621 |
| SNRP70 | X06815 |
| PDLIM1 | U90878 |
| ALOX5 | J03600 |
| RAG2 | M94633 |
| CALM1 | U12022 |
| KIAA1013 | AB023230 |
| NDUFA1 | N47307 |
| FOS | V01512 |
| DXS1357E | X81109 |
| ICSBP1 | M91196 |
| ETS2 | J04102 |
| PCDH9 | AI524125 |
| LILRA2 | AF025531 |
| PSAP | J03077 |
| SCHIP-1 | AF070614 |
| CCND2 | D13639 |
| KCNN1 | U69883 |
| ALTE | AB018328 |
| IGFBP4 | U20982 |
| M9 | AB019392 |
| SCML2 | Y18004 |
| LOC51632 | AI557497 |
| UBE2G2 | AF032456 |
| STATI2 | AF037989 |
| ATRX | U72936 |
| APT6M8-9 | AL049929 |
| PTPRE | X54134 |
| GILZ | AI635895 |
| PECAM1 | AA100961 |
| ARHGEF4 | AB029035 |
| ECM1 | U68186 |

TABLE 57
Additional Genes selected by
T statistics for the MLL Risk Group

| Gene symbol | Accession Number |
|---|---|
| EPOR | M60459 |
| CD44 | L05424 |
| PRKCH | M55284 |
| MADH1 | U59423 |
| KLF1 | U65404 |
| MME | J03779 |
| PTPRK | L77886 |
| IL1B | X04500 |
| YES1 | M15990 |
| ARPC2 | U50523 |
| IGFBP4 | M62403 |
| ITPR3 | U01062 |
| | M13929 |
| EFNB1 | U09303 |
| FHIT | U46922 |
| NME2 | X58965 |
| CCND2 | X68452 |
| MPB1 | M55914 |
| CDH2 | M34064 |
| IGFBP7 | L19182 |
| ALOX5 | J03600 |
| PTGDR | U31099 |
| PLXNC1 | AF030339 |
| EIF3S2 | U39067 |
| BLVRA | X93086 |
| HSPC022 | W68830 |
| | S67247 |
| MYLK | U48959 |
| SLC6A11 | S75989 |
| | X67098 |
| SERPINB1 | M93056 |
| LGALS1 | AI535946 |
| HRK | D83699 |
| | AL049313 |
| HBS1L | AB028961 |
| KIAA0437 | AB022660 |
| GDI2 | Y13286 |
| ITGA4 | X16983 |
| EEF1B2 | X60489 |
| MD-1 | AB020499 |
| POU4F1 | X64624 |
| TST | X59434 |
| PTPRF | Y00815 |
| ARHGEF4 | AB029035 |
| SCHIP-1 | AF070614 |
| ASMTL | AA669799 |
| DDR1 | L20817 |
| N33 | U42360 |
| CR2 | M26004 |
| AHNAK | M80899 |
| SCGF | AF020044 |
| EPB49 | U28389 |
| PSPHL | AJ001612 |
| MADH1 | U59912 |
| ITPR3 | U01062 |
| DPEP1 | J05257 |
| AKAP12 | U81607 |
| DBI | A1557240 |
| KIAA0736 | AB018279 |
| MAL | X76220 |
| S100A4 | W72186 |
| MDK | X55110 |
| CRK | D10656 |
| CAPG | M94345 |
| KCNH2 | U04270 |
| KIAA1069 | AB028992 |
| DKFZP564L0862 | AL080091 |
| KIAA0298 | AB002296 |
| DGKD | D73409 |
| DEPP | AB022718 |
| | AL049957 |
| CD8B1 | X13444 |
| EFNB1 | U09303 |
| | AI391564 |
| LDOC1 | AB019527 |
| EFNA1 | M57730 |
| CD44 | L05424 |
| PTPRC | Y00062 |
| PTPRC | Y00638 |
| PTPRC | Y00638 |
| TFPI | M59499 |
| TSPAN-5 | AF065389 |
| BCL11A | W27619 |
| | AJ001381 |
| KIAA1011 | AL080133 |
| FYB | U93049 |
| DKFZp761F2014 | AA149431 |
| FGFR1 | X66945 |
| | M63589 |
| PTPN6 | X62055 |

TABLE 58
Additional Genes selected by
T statistics for the Novel Risk Group

| Gene symbol | Accession Number |
|---|---|
| CHST2 | AB014679 |
| CLTC | D21260 |
| TUBA1 | X06956 |
| GNG11 | U31384 |
| PCDH9 | AI524125 |
| MDS019 | AA442560 |
| RAG2 | M94633 |
| ITGA6 | X53586 |
| UBE2E3 | AB017644 |
| CD34 | S53911 |
| CD34 | M81945 |
| FGFR1 | M34641 |
| ECM1 | U68186 |
| MADH1 | U59423 |
| FUT7 | AB012668 |
| PROML1 | AF027208 |
| CSNK2A1 | M55265 |
| FLNB | AF042166 |
| MADH1 | U59912 |
| LIG4 | X83441 |
| ZNF151 | Y09723 |
| CSF3R | M59818 |
| | AL080205 |
| STAU2 | AL079286 |
| AEBP1 | AF053944 |
| KIAA0320 | AB002318 |
| KIAA0746 | AB018289 |
| PTPRM | X58288 |
| IGFBP4 | M62403 |
| ZNF266 | AA868898 |
| PDLIM1 | U90878 |
| MTMR3 | AB002369 |
| TIMP1 | D11139 |
| TTC2 | W28595 |
| TM4SF2 | L10373 |
| PSA | AA978353 |
| HTR4 | Y12505 |
| MMS19L | AF007151 |
| | AI391564 |
| TJP2 | L27476 |
| BMP2 | M22489 |
| ARL7 | AB016811 |
| TLR1 | AL050262 |
| SMC2L1 | AF092563 |
| TGFBR2 | D50683 |
| TGFBR2 | D50683 |
| SPARC | J03040 |
| GPRK5 | L15388 |
| CDH2 | M34064 |
| KIAA0877 | AB020684 |
| ABLIM | D31883 |
| RNF3 | W25793 |
| CCBP2 | U94888 |
| CHN2 | U07223 |
| ITGA4 | X16983 |
| IQGAP2 | U51903 |
| FLJ22531 | W80358 |
| PIK3CD | U86453 |
| FXYD2 | H94881 |
| | W30677 |
| AMPD3 | U29926 |
| | D78577 |
| KIAA0125 | D50915 |
| FADS3 | AC004770 |
| DKFZP434C171 | AL080169 |
| EST00098 | AI885170 |
| BMP2 | M22489 |
| LILRB4 | AF072099 |
| KIAA0429 | AB007889 |
| DKFZP586G0522 | AL050289 |
| | U92818 |
| ATIC | D82348 |
| MONDOA | AB020674 |
| CNK1 | AF100153 |
| NGFR | M14764 |
| KIAA0540 | AB011112 |
| MYO10 | AB018342 |
| PIASX-BETA | AF077954 |
| ACVR1 | Z22534 |
| ARHGEF10 | AB002292 |
| PON2 | AF001601 |
| TST | X59434 |
| SPTBN1 | M96803 |
| ERCC2 | AA079018 |
| PRSC1 | D55696 |
| DKFZP434D174 | AL080150 |
| | AI184710 |
| CD8B1 | X13444 |
| | U79265 |
| DKFZp761F2014 | AA149431 |
| MEF2A | U49020 |
| JAG2 | AF029778 |
| ZNF143 | AF071771 |
| CASP1 | U13697 |
| HAP1 | AF040723 |
| FABGL | D82061 |
| ALDH1 | K03000 |
| RAD9 | U53174 |
| | AL109722 |
| CDC27 | AA166687 |
| B4GALT1 | D29805 |
| PTPRM | X58288 |
| AHR | L19872 |
| N33 | U42349 |
| IL12RB2 | U64198 |
| MTR | U73338 |
| KIAA0697 | AB014597 |
| CSNK2B | M30448 |
| | U15590 |
| | W28612 |
| HSU79253 | AF052186 |
| RBBP1 | S57153 |
| S100A11 | D38583 |
| TCF12 | M80627 |
| | AI971169 |
| EEF1E1 | N32257 |
| SAP18 | AW021542 |
| PVRL1 | AF060231 |
| | M13929 |
| MKP-L | AF038844 |
| | W26667 |
| CD79B | M89957 |
| KIAA0437 | AB022660 |
| | AF070633 |
| GCLM | L35546 |
| EDG6 | AJ000479 |
| MAL | X76220 |

TABLE 59
Additional Genes selected by
T statistics for the T-ALL Risk Group

| Gene symbol | Accession Number |
|---|---|
| SLP65 | AF068180 |
| CD3D | AA919102 |
| SH2D1A | AL023657 |
| CD79B | M89957 |
| CD3E | M23323 |
| CTGF | X78947 |
| PFTK1 | AB020641 |
| TRB | X00437 |
| CD24 | L33930 |
| CD22 | X52785 |
| TOP2B | X68060 |
| CD22 | X59350 |
| TCL1A | X82240 |
| BRAG | AB011170 |
| CD79A | U05259 |
| SCHIP-1 | AF070614 |
| MAL | X76220 |
| HLA-DQB1 | M16276 |
| PDE4B | L20971 |
| HLA-DQB1 | M60028 |
| CD19 | M28170 |
| KIAA0959 | AB023176 |
| LILRA2 | AF025531 |
| PTPN18 | X79568 |
| MEF2C | L08895 |
| PTP4A2 | U14603 |
| NPY | AI198311 |
| GAB1 | U43885 |
| lck | U23852 |
| TCF7 | X59871 |
| TERF2 | X93512 |
| ITM2A | AL021786 |
| MEF2C | S57212 |
| SLC9A3R1 | AF015926 |
| ENG | X72012 |
| DEPP | AB022718 |
| IL1B | X04500 |
| IL1B | M15330 |
| ECM1 | U68186 |
| HLA-DMA | X62744 |
| CRMP1 | D78012 |
| WFS1 | AF084481 |
| PRKCQ | L01087 |
| GNG7 | AB010414 |
| | X58398 |
| CDKN1A | U03106 |
| CD9 | M38690 |
| PTK2 | L13616 |
| TRB | M12886 |
| IF135 | L78833 |
| NUCB2 | X76732 |
| KIAA0942 | AB023159 |
| VATI | U18009 |
| ARL7 | AB016811 |
| USP20 | AB023220 |
| PLCG2 | X14034 |
| PRDX1 | X67951 |
| POU2AF1 | Z49194 |
| CMAH | D86324 |
| ALOX5 | J03600 |
| PTPN7 | M64322 |
| MEF2C | S57212 |
| KIAA0668 | AL021707 |
| LOC54103 | AL079277 |
| EFNB1 | U09303 |
| HELO1 | AL034374 |
| ADF | S65738 |
| KIAA0906 | AB020713 |
| IGFBP4 | U20982 |
| LDHB | X13794 |
| CTNNA1 | U03100 |
| ENO2 | X51956 |
| LAT | AJ223280 |
| PTPN7 | D11327 |
| | M16942 |
| CSRP2 | U57646 |
| GLA | U78027 |
| ADA | X02994 |
| RGS10 | AF045229 |
| KIAA0870 | AB020677 |
| CD3Z | J04132 |
| STATI2 | AF037989 |
| GSN | X04412 |
| INSR | X02160 |
| HLA-DNA | M31525 |
| CD72 | M54992 |
| EPHB6 | D83492 |
| MYLK | U48959 |
| HLA-DQA1 | AA868382 |
| LCK | M36881 |
| FHL1 | AF063002 |
| CRIM1 | AI651806 |
| AQP3 | N74607 |
| HLA-DQB1 | M81141 |
| GNG11 | U31384 |
| LARGE | AJ007583 |
| FOXO1A | AF032885 |
| NPR1 | X15357 |
| GAB1 | U43885 |
| PTPRE | X54134 |
| PDLIM1 | U90878 |
| NCF4 | AL008637 |
| ARHGEF4 | AB029035 |
| PTP4A2 | U14603 |
| CTNNA1 | AF102803 |
| SEPW1 | U67171 |
| CHI3L2 | U58515 |
| LILRA2 | U82277 |
| CD79A | U05259 |
| TCL1B | AB018563 |
| TCF4 | M74719 |
| TACTILE | M88282 |
| | AB002438 |
| TXN | AI653621 |
| ADE2H1 | X53793 |
| | AL049449 |
| GLUL | X59834 |
| ZFHX1B | AB011141 |
| P4HB | M22806 |
| IFITM1 | J04164 |
| KIAA0182 | D80004 |
| SH2D1A | AF100539 |
| GNA11 | M69013 |
| NCF4 | AL008637 |
| SLC2A5 | M55531 |
| KIAA0993 | AB023210 |
| HLA-DPB1 | M83664 |
| HLX1 | M60721 |
| CTNNA1 | D14705 |
| FADS3 | AC004770 |
| GATA3 | X58072 |
| GDI2 | Y13286 |
| TM4SF2 | L10373 |
| GNA15 | M63904 |
| BTG2 | U72649 |
| RAG1 | M29474 |
| MDK | X55110 |
| | X00457 |
| AKR1C3 | D17793 |
| SLA | D89077 |
| LDHA | X02152 |
| | AL049279 |
| PTPRC | Y00638 |
| BMP2 | M22489 |
| ERG | M17254 |
| ICSBP1 | M91196 |
| CCT2 | AF026166 |
| AKAP2 | AB023137 |
| | X58398 |
| KIAA0128 | D50918 |
| IGHM | X58529 |
| NOTCH3 | U97669 |
| JUP | M23410 |
| DKFZP586O1624 | AL039458 |
| MYO10 | AB018342 |
| CTNNA1 | L23805 |
| NOS2A | U31511 |
| | D00749 |
| | L29376 |
| ICB-1 | AF044896 |
| GNAI1 | AL049933 |
| S100A11 | D38583 |
| MAPKAPK3 | U09578 |
| ADA | M13792 |
| S100A13 | AI541308 |
| VDAC3 | AF038962 |
| | AL049265 |
| TRIM | AJ224878 |
| CTBP2 | AF016507 |
| F13A1 | M14539 |
| ZNF43 | HG620-HT620 |
| DKFZp761F2014 | AA149431 |
| KIAA0442 | AB007902 |
| CTNNA1 | U03100 |
| CD2 | M16336 |
| BMP2 | M22489 |
| HSPC022 | W68830 |
| ICAM3 | X69819 |
| NCF4 | X77094 |
| GS3955 | D87119 |
| CTSC | X87212 |
| GH1 | V00520 |
| ARPC2 | U50523 |
| HLA-DRB1 | M32578 |
| GAS1 | L13698 |
| LAMB2 | M55210 |
| EPHB4 | U07695 |
| COX8 | A1525665 |
| KIAA0618 | N29665 |
| KIAA0870 | AI808958 |
| PIK3CG | X83368 |
| IGHD | K02882 |
| IRF4 | U52682 |
| HSPCB | M16660 |
| CAPN3 | X85030 |
| CD6 | X60992 |
| WSX-1 | AI263885 |
| FXYD2 | H94881 |
| PTK2 | HG3075-HT3236 |
| FUCA1 | M29877 |
| FADS2 | AL050118 |
| KARS | D32053 |
| DSCR1 | U85267 |
| SOX4 | X70683 |
| TRD | X73617 |
| MHC2TA | U18259 |
| | AL049435 |
| MDK | M94250 |
| CALM1 | U12022 |
| PCLO | AB011131 |
| | AI391564 |
| FHIT | U46922 |
| MONDOA | AB020674 |
| TRG | M30894 |
| SPIB | X66079 |
| FLJ10097 | AL035494 |
| TAGLN2 | D21261 |
| LGALS9 | Z49107 |

TABLE 60
Additional Genes selected by
T statistics for the TEL-AML1 Risk
Group

| Gene symbol | Accession Number |
|---|---|
| ARHGEF4 | AB029035 |
| TNFRSF7 | M63928 |
| PCLO | AB011131 |
| TCFL5 | AB012124 |
| KCNN1 | U69883 |
| NME2 | X58965 |
| PTPRK | L77886 |
| | AL049313 |
| TERF2 | X93512 |
| GNG11 | U31384 |
| RAG1 | M29474 |
| | AL080190 |
| MADH1 | U59423 |
| | HG3523-HT4899 |
| MADH1 | U59912 |
| P114-RHO-GEF | AB011093 |
| | L29254 |
| MDK | M94250 |
| TERF2 | AF002999 |
| CRMP1 | D78012 |
| HLA-DOB | X03066 |
| NFKBIL1 | Y14768 |
| | AA216639 |
| | AL080059 |
| CBFA2T3 | AB010419 |
| MDK | X55110 |
| PIK3C3 | Z46973 |
| ALOX5 | J03600 |
| PTP4A3 | AF041434 |
| POU2AF1 | Z49194 |
| POU4F1 | L20433 |
| PRKCB1 | X07109 |
| GCAT | Z97630 |
| PHYH | AF023462 |
| SPTA1 | M61877 |
| IDI1 | X17025 |
| FYB | U93049 |
| ITPR1 | D26070 |
| GTT1 | AL041780 |
| FADS3 | AC004770 |
| CCT2 | AF026166 |
| ISG20 | U88964 |
| SCHIP-1 | AF070614 |
| DR6 | AF068868 |
| MYO10 | AB018342 |
| ZNF91 | L11672 |
| T-STAR | AF051321 |
| FUCA1 | M29877 |
| HLA-DQB1 | M60028 |
| | AB002438 |
| CTGF | X78947 |
| FKBP1A | M34539 |
| | AI391564 |
| RAB1 | AL050268 |
| INSR | X02160 |
| KIAA0540 | AB011112 |
| TM4SF2 | L10373 |
| CASP1 | M87507 |
| MT1L | AA224832 |
| MME | J03779 |
| | AI743299 |
| KARS | D32053 |
| CHN2 | U07223 |
| IQGAP2 | U51903 |
| KIAA0906 | AB020713 |
| STATI2 | AF037989 |
| HLA-DMA | X62744 |
| CD36L1 | Z22555 |
| PRKCB1 | X06318 |
| GS3955 | D87119 |
| ACTN1 | X15804 |
| FLJ20154 | AF070644 |
| KIAA0769 | AB018312 |
| SDC1 | Z48199 |
| SOX4 | X70683 |
| NRTN | U78110 |
| CTNND1 | AB002382 |
| FHIT | U46922 |
| FARP1 | AI701049 |
| FOXO1A | AF032885 |
| NPY | AI198311 |
| VDUP1 | S73591 |
| H2AFO | AI885852 |
| TACTILE | M88282 |
| SNL | U03057 |
| JUP | M23410 |
| NR3C2 | M16801 |
| PRPS2 | Y00971 |
| LILRA2 | AF025531 |
| RNAHP | H68340 |
| DPYSL2 | U97105 |
| ITGB2 | M15395 |
| PCDH9 | AI524125 |
| LAIR1 | AF013249 |
| CD79A | U05259 |
| NFKBIL1 | Y14768 |
| PCCA | S79219 |
| HLA-DMB | U15085 |
| SMARCA4 | D26156 |

### Example 2

To identify additional additional genes whose expression levels could be used as a diagnostic tool to identify ALL subgroups, leukemic blasts from 132 diagnostic samples were analyzed using higher density oligonucleotide arrays that allow the interrogation of a majority of the identified genes in the human genome.

A subset of the 327 diagnostic pediatric ALL samples described above were reanalyzed using these higher density microarrays. Case selection was based on providing a representation of the known prognostic ALL subtypes including t(9;22)[BCR-ABL], t(1;19)[E2A-PBX1], t(12;21)[TEL-AML1], rearrangement in the MLL gene on chromosome 11q23, and hyperdiploid karyotype with >50 chromosomes. Since the goal was to define expression profiles that could be used to accurately diagnose the known prognostic subtypes of ALL, we chose to over represent these subtypes compared to what is normally seen in a random population of childhood leukemia patients. A total of 132 samples met these criteria and had sufficient material remaining to be used for this analysis. The list of samples and subtype distribution of the cases used in this study are shown in Tables 61 and 52, respectively.

TABLE 61
Diagnostic ALL samples used for class prediction (n = 132)

| | | |
|---|---|---|
| BCR-ABL-#1 | Hyperdip >50-c18 | Pseudodip-#6 |
| BCR-ABL-#2 | Hyperdip >50-C21 | Pseudodip-C2-N |
| BCR-ABL-#3 | Hyperdip >50-C22 | Pseudodip-C3 |
| BCR-ABL-#4 | Hyperdip >50-C23 | Pseudodip-C5 |
| BCR-ABL-#5 | Hyperdip >50-C27-N | Pseudodip-C6 |
| BCR-ABL-#6 | Hyperdip >50-C32 | Pseudodip-C7 |
| BCR-ABL-#7 | Hyperdip >50-R4 | Pseudodip-C9 |
| BCR-ABL-#8 | Hyperdip47-50-C14-N | Pseudodip-C14 |
| BCR-ABL-#9 | Hyperdip47-50-C3-N | Pseudodip-C16-N |
| BCR-ABL-Hyperdip-#10 | Hypodip-#2 | Pseudodip-R1-N |
| BCR-ABL-C1 | Hypodip-2M#1 | T-ALL-#5 |
| BCR-ABL-R1 | Hypodip-C2 | T-ALL-#6 |
| BCR-ABL-R2 | Hypodip-C5 | T-ALL-#7 |
| BCR-ABL-R3 | MLL-#1 | T-ALL-#8 |
| BCR-ABL-Hyperdip-R5 | MLL-#2 | T-ALL-#10 |
| E2A-PBX1-#5 | MLL-#3 | T-ALL-C2 |
| E2A-PBX1-#6 | MLL-#4 | T-ALL-C6 |
| E2A-PBX1-#9 | MLL-#5 | T-ALL-C7 |
| E2A-PBX1-#10 | MLL-#6 | T-ALL-C11 |
| E2A-PBX1-#12 | MLL-#7 | T-ALL-C15 |
| E2A-PBX1-#13 | MLL-#8 | T-ALL-C19 |
| E2A-PBX1-2M#1 | MLL-2M#1 | T-ALL-C21 |
| E2A-PBX1-C2 | MLL-2M#2 | T-ALL-R5 |
| E2A-PBX1-C3 | MLL-C1 | T-ALL-R6 |
| E2A-PBX1-C4 | MLL-C2 | TEL-AML1-#6 |
| E2A-PBX1-C5 | MLL-C3 | TEL-AML1-#9 |
| E2A-PBX1-C6 | MLL-C4 | TEL-AML1-#10 |
| E2A-PBX1-C7 | MLL-C5 | TEL-AML1-#14 |
| E2A-PBX1-C9 | MLL-C6 | TEL-AML1-2M#1 |
| E2A-PBX1-C10 | MLL-R1 | TEL-AML1-2M#2 |
| E2A-PBX1-C11 | MLL-R2 | TEL-AML1-C4 |
| E2A-PBX1-C12 | MLL-R3 | TEL-AML1-C5 |
| E2A-PBX1-R1 | MLL-R4 | TEL-AML1-C6 |
| Hyperdip >50-#8 | Normal-C1-N | TEL-AML1 -C26 |
| Hyperdip >50-#12 | Normal-C2-N | TEL-AML1-C28 |
| Hyperdip >50-#14 | Normal-C3 -N | TEL-AML1-C30 |
| Hyperdip >50-C1 | Normal-C4-N | TEL-AML1-C31 |
| Hyperdip >50-C4 | Normal-C7-N | TEL-AML1-C32 |
| Hyperdip >50-C6 | Normal-C8 | TEL-AML1-C33 |
| Hyperdip >50-C8 | Normal-C9 | TEL-AML1-C34 |
| Hyperdip >50-C11 | Normal-C11-N | TEL-AML1-C37 |
| Hyperdip >50-C13 | Normal-R1 | TEL-AML1-C38 |
| Hyperdip >50-C15 | Normal-R2-N | TEL-AML1-C40 |
| Hyperdip >50-C16 | Pseudodip-#5 | TEL-AML1-R3 |

*Subtype Name-C# Dx Sample of patient in CCR
Subtype Name-R# Dx Sample of patient who developed a hematologic relapse
Subtype Name-# Dx Sample used for subgroup classification only
Subtype Name-2M# Dx Sample of patient who later developed 2nd AML
Subtype Name-N Dx Sample in novel group

TABLE 62
Subgroup distribution of ALL cases

| Subgroup | Train Set | Test Set |
|---|---|---|
| BCR-ABL | 11 | 4 |
| E2A-PBX1 | 13 | 5 |
| Hyperdiploid >50 | 13 | 4 |
| MLL | 15 | 5 |
| T-ALL | 12 | 2 |
| TEL-AML1 | 15 | 5 |
| Other | 21 | 7 |
| Total | 100 | 32 |

26,825 probe sets from combined Affymetrix® brand U133A and B microarrays (Affymetrix, Inc., Santa Clara, Calif.) showed variation in expression levels across the 132 diagnostic leukemia samples. In an initial analysis of these data, two complementary unsupervised clustering algorithms: two-dimensional hierarchical clustering and principle component analysis (PCA), were used to assess the major sub-groupings of the leukemia cases based solely on gene expression profiles. These unbiased clustering algorithms demonstrated that the pediatric ALL cases cluster primarily into seven major subtypes: T-ALL and 6 subtypes of B-cell lineage ALL corresponding to (1) rearrangement in the MLL gene on chromosome 11q23, (2) t(1;19)[E2A-PBX1], (3) hyperdiploid>50 chromosomes, (4) t(9;22)[BCR-ABL], (5) the novel subgroup, and (6) t(12;21)[TEL-AML1]. In addition, a heterogeneous group of B-lineage cases were identified that lacked any of the defined genetic lesions and failed to cluster into the novel subgroup. Several of these leukemia subtypes formed distinct branches when all differentially expressed genes were used in the two-dimensional hierarchical clustering algorithm (T-ALL, Hyperdiploid>50 chromosomes, and TEL-AML1), whereas other subtypes clustered in multiple branches, suggestive of gene expression differences within these subclasses. Using PCA, the distinct nature of the B-cell lineage subtypes is better appreciated when the T-ALL cases were removed from the analysis. A diagnostic accuracy of 100% was achieved for two of the leukemia subtypes (T-ALL and TEL-AML1), indicating the need to use supervised learning algorithms to achieve optimal diagnostic accuracy by gene expression profiling.

Statistical methods were used to identify probe sets that were the best discriminators of the individual leukemia subtypes. In order to identify the genes that provide the highest accuracy in diagnosing specific prognostic subtypes of leukemia, the decision tree format described elsewhere herein was used for the identification of leukemia subtypes. Briefly, we first defined whether a case is T- or B-cell in lineage. If the case is classified as T-cell, a diagnosis of T-ALL is made. If non-T, we then determine if the case can be classified into one of the known B-cell lineage risk groups, deciding sequentially if it is E2A-PBX1, TEL-AML1, BCR-ABL, rearranged MLL gene, and lastly hyperdiploid with >50 chromosomes. Cases not assigned to one of these classes are left unassigned. The use of this decision tree format directly influences the selection of genes, allowing the selection of discriminating genes for groups lower down the tree that might also be expressed by subtypes higher in the tree. Using a number of different supervised learning algorithms, it was found that a higher diagnostic accuracy is obtained using this decision tree format, as compared to a parallel format in which each class is identified against all others.

Discriminating genes were selected using a chi-square metric on the 100 cases in the training set. Genes were selected that discriminated between a class and all leukemia subtypes below it in the decision tree. The number of discriminating probe sets per leukemia subtype at a statistical significance level of p≦0.001 (as determined by a permutation test) were: T-ALL, 2063; E2A-PBX1, 1059; TEL-AML1, 805; BCR-ABL, 201; MLL chimeric genes, 726; and hyperdiploid with >50 chromosomes, 994. The lists of discriminating genes obtained using the top 100 ranked probe sets for the six prognostically important subgroups are contained in Tables 63-68. As multiple probe sets for the same gene are present on Affymetrix microarrays, the top 100 ranked probe sets represent between 75 and 92 distinct genes, depending on the leukemia subtype. As shown, distinct groups of either over or under expressed genes distinguish cases defined by E2A-PBX1, MLL gene rearrangement, T-ALL, hyperdiploid>50 chromosomes, BCR-ABL, and TEL-AML1.

The following tables contain a list of the top 100 probe sets for each diagnostic subtype, ranked by their chi-square value. Each table contains the Affymetrix® U133 series probe set number, a gene description, gene symbol, chromosomal location, and primary GenBank reference. Chi-square values were calculated utilizing only the samples in the train set in a differential diagnosis decision tree format. The calculation of the fold change was done in a parallel format using the total data set and comparing the mean signal value in the class versus the mean signal value in the non-class.

TABLE 63
Top 100 chi-square probe sets selected for BCR-ABL

| | | | | | | | Bcr | |
|---|---|---|---|---|---|---|---|---|
| | | | | | | Chi- | above/ | |
| | U133 probe | | Gene | Chromosomal | GenBank | square | below | Fold |
| | set | Gene description | symbol | location | Reference | value | mean | change |
| 1 | 241812_at | EST FLJ39877 | FLJ39877 | 2 | AV648669 | 47.4 | Above | 5.2 |
| 2 | 201876_at | Paraoxonase/ | PON2 | 7q21.3 | NM_000305.1 | 47.2 | Above | 18.7 |
| | | arylesterase 2 | | | | | | |
| 3 | 201028_s_at | Antigen identified | MIC2 | Xp22.32 | U82164.1 | 44.3 | Above | 2.6 |
| | | by monoclonal | | | | | | |
| | | antibodies 12E7, | | | | | | |
| | | F21 and O13 | | | | | | |
| 4 | 200953_s_at | Cyclin D2 | CCND2 | 12p13 | NM_001759.1 | 42.3 | Above | 3.5 |
| 5 | 202947_s_at | Glycophorin C | GYPC | 2q14-q21 | NM_002101.2 | 42.3 | Above | 3.1 |
| | | integral membrane | | | | | | |
| | | glycoprotein | | | | | | |
| 6 | 223449_at | Semaphorin 6A | SEMA6A | 5q23.1 | AF225425.1 | 42.3 | Above | 4.3 |
| 7 | 201029_s_at | Antigen identified | MIC2 | Xp22.32 | NM_002414.1 | 41.2 | Above | 2.4 |
| | | by monoclonal | | | | | | |
| | | antibodies 12E7, | | | | | | |
| | | F21 and O13 | | | | | | |
| 8 | 204429_s_at | Solute carrier | SLC2A5 | 1p36.2 | BE560461 | 41.2 | Above | 5 |
| | | family 2 | | | | | | |
| | | (facilitated | | | | | | |
| | | glucose/fructose | | | | | | |
| | | transporter), | | | | | | |
| | | member 5 | | | | | | |
| 9 | 210830_s_at | Paraoxonase | PON2 | 7q21.3 | AF001602.1 | 41.2 | Above | 23.6 |
| 10 | 215028_at | Semaphorin 6A | SEMA6A | 5 | AB002438.1 | 41.2 | Above | 4.5 |
| 11 | 220024_s_at | Periaxin | PRX | 19q13.13-q13.2 | NM_020956.1 | 41.2 | Above | 8.2 |
| 12 | 201906_s_at | HYA22 protein | HYA22 | 3p21.3 | NM_005808.1 | 41.1 | Above | 43.4 |
| 13 | 209365_s_at | Extracellular | ECM1 | 1q21 | U65932.1 | 41.1 | Above | 6 |
| | | matrix protein 1 | | | | | | |
| 14 | 238689_at | GPR110 G | GPR110 | 6 | BG426455 | 41.1 | Above | 10.9 |
| | | protein-coupled | | | | | | |
| | | receptor 110 | | | | | | |
| 15 | 222154_s_at | DKFZP564A2416 | DKFZP564A2416 | 2q33.1 | AK002064.1 | 40.4 | Above | 12.4 |
| | | unknown protein | | | | | | |
| | | with a histone H5 | | | | | | |
| | | signature. | | | | | | |
| 16 | 218084_x_at | FXYD domain- | FXYD5 | 19q12-q13.1 | NM_014164.2 | 38 | Above | 1.5 |
| | | containing ion | | | | | | |
| | | transport regulator 5 | | | | | | |
| 17 | 212242_at | Tubulin, alpha 1 | TUBA1 | 2q36.2 | AL565074 | 37 | Above | 3.2 |
| | | (testis specific) | | | | | | |
| 18 | 201445_at | Calponin 3, acidic | CNN3 | 1p22-p21 | NM_001839.1 | 36.3 | Above | 10.8 |
| 19 | 202771_at | KIAA0233 gene | KIAAO233 | 16q24.3 | NM_014745.1 | 36.3 | Above | 1.9 |
| | | product | | | | | | |
| 20 | 212298_at | Neuropilin 1 | NRP1 | 10p12 | BE620457 | 36.3 | Above | 13.8 |
| 21 | 212458_at | FLJ21897 | FLJ21897 | 2 | AW138902 | 36.3 | Above | 2.4 |
| 22 | 222488_s_at | Dynactin 4 | DCTN4 | 5q31-q32 | BE218028 | 36.3 | Above | 3.6 |
| 23 | 222762_x_at | LIM domains | LIMD1 | 3p21.3 | AU144259 | 36.3 | Above | 2.6 |
| | | containing 1 | | | | | | |
| 24 | 200951_s_at | Cyclin D2 | CCND2 | 12p13 | NM_001759.1 | 35.3 | Above | 12.7 |
| 25 | 204430_s_at | Solute carrier | SLC2A5 | 1p36.2 | NM_003039.1 | 35.3 | Above | 5.1 |
| | | family 2 | | | | | | |
| | | (facilitated | | | | | | |
| | | glucose/fructose | | | | | | |
| | | transporter), | | | | | | |
| | | member 5 | | | | | | |
| 26 | 205467_at | Caspase 10 | CASP10 | 2q33-q34 | NM_001230.1 | 35.3 | Above | 3.6 |
| 27 | 225660_at | Semaphorin 6A | SEMA6A | 5q23.1 | W92748 | 35.3 | Above | 3.3 |
| 28 | 225913_at | FLJ21140 | FLJ21140 | 15 | AK025943.1 | 35.3 | Above | 2.9 |
| | | (Ser/Thr protein | | | | | | |
| | | kinase) | | | | | | |
| 29 | 236489_at | EST | | 6 | AI282097 | 35.3 | Above | 16.7 |
| 30 | 240173_at | EST | | 4 | AI732969 | 35.3 | Above | 10.3 |
| 31 | 240499_at | EST | | 10 | AA482221 | 35.3 | Above | 1.3 |
| 32 | 201310_s_at | P311 protein. | P311 | 5q21.3 | NM_004772.1 | 35.2 | Below | 2.2 |
| | | Similar to | | | | | | |
| | | gastrin/cholecysto | | | | | | |
| | | kinin type B | | | | | | |
| | | receptor. | | | | | | |
| 33 | 215617_at | FLJ11754 | FLJ11754 | 2 | AU145711 | 35.2 | Above | 14.4 |
| 34 | 242579_at | EST | | 4 | AA935461 | 35.2 | Above | 10.2 |
| 35 | 202717_s_at | CDC16 cell | CDC16 | 13q34 | NM_003903.1 | 34.4 | Above | 1.1 |
| | | division cycle 16 | | | | | | |
| | | homolog | | | | | | |
| 36 | 205055_at | Integrin, alpha E | ITGAE | 17p13 | NM_002208.3 | 34.4 | Below | 2.1 |
| | | (antigen CD103, | | | | | | |
| | | human mucosal | | | | | | |
| | | lymphocyte | | | | | | |
| | | antigen 1) | | | | | | |
| 37 | 217967_s_at | Chromosome 1 | Clorf24 | 1q25 | AF288391.1 | 34.4 | Above | 3.2 |
| | | ORF 24 | | | | | | |
| 38 | 201656_at | Integrin, alpha 6 | ITGA6 | 2q31.1 | NM_000210.1 | 33.9 | Above | 2.8 |
| 39 | 207196_s_at | Nef-associated | NAF1 | 5q32-q33.1 | NM_006058.1 | 32.2 | Above | 1.4 |
| | | factor 1 | | | | | | |
| 40 | 219315_s_at | hypothetical | FLJ20898 | 16p13.12 | NM_024600.1 | 32.2 | Above | 5.3 |
| | | protein FLJ23058 | | | | | | |
| 41 | 202123_s_at | V-abl Abelson | ABL1 | 9q34.1 | NM_005157.2 | 31.4 | Above | 1.8 |
| | | murine leukemia | | | | | | |
| | | viral oncogene | | | | | | |
| | | homolog 1 | | | | | | |
| 42 | 219938_s_at | Pro-Ser-Thr | PSTPIP2 | 18q12 | NM_024430.1 | 31.2 | Above | 5 |
| | | phosphatase | | | | | | |
| | | interacting protein 2 | | | | | | |
| 43 | 228046_at | EST; DKFZp434P0235 | DKFZp434P0235 | 4 | AA741243 | 31.2 | Above | 1.1 |
| 44 | 64064_at | Immune | IAN4L1 | 7q36 | AI435089 | 30.9 | Above | 3.3 |
| | | associated | | | | | | |
| | | nucleotide 4 like 1 | | | | | | |
| 45 | 222729_at | F-box and WD-40 | FBXW7 | 4q31.23 | BE551877 | 30.5 | Above | 2.4 |
| | | domain protein 7 | | | | | | |
| | | (archipelago | | | | | | |
| | | homolog, | | | | | | |
| | | Drosophila) | | | | | | |
| 46 | 229975_at | EST | | 4 | AI826437 | 30.5 | Above | 9.1 |
| 47 | 200864_s_at | RAB11A | RAB11A | 15q21.3-q22.31 | NM_004663.1 | 29.7 | Above | 1.4 |
| 48 | 203089_s_at | Protease, serine, | PRSS25 | 2p12 | NM_013247.1 | 29.7 | Above | 1.7 |
| | | 25 | | | | | | |
| 49 | 205376_at | Inositol | INPP4B | 4q31.1 | NM_003866.1 | 29.7 | Above | 12.4 |
| | | polyphosphate-4- | | | | | | |
| | | phosphatase, type | | | | | | |
| | | II | | | | | | |
| 50 | 209229_s_at | KIAA1115 | KIAA1115 | 19q13.42 | BC002799.1 | 29.7 | Above | 1.3 |
| | | protein | | | | | | |
| 51 | 219871_at | Hypothetical | FLJ13197 | 4p14 | NM_024614.1 | 29.7 | Above | 14.5 |
| | | protein FLJ13197 | | | | | | |
| 52 | 222868_s_at | Interleukin 18 | IL18BP | 11q13 | AI521549 | 29.7 | Above | 7.1 |
| | | binding protein | | | | | | |
| 53 | 235988_at | GPR110 G | GPR110 | 6p12.3 | AA746038 | 29.7 | Above | 15.8 |
| | | protein-coupled | | | | | | |
| | | receptor 110 | | | | | | |
| 54 | 239273_s_at | Matrix | MMP28 | 17q11-q21.1 | AI927208 | 29.7 | Above | 90.5 |
| | | metalloproteinase | | | | | | |
| | | 28 | | | | | | |
| 55 | 206150_at | Tumor necrosis | TNFRSF7 | 12p13 | NM_001242.1 | 29.5 | Above | 3.2 |
| | | factor receptor | | | | | | |
| | | superfamily, | | | | | | |
| | | member 7 | | | | | | |
| 56 | 212203_x_at | Interferon induced | IFITM3 | 8q13.1 | BF338947 | 29.5 | Above | 2.3 |
| | | transmembrane | | | | | | |
| | | protein 3 | | | | | | |
| 57 | 217110_s_at | Mucin 4 | MUC4 | 3q29 | AJ242547.1 | 29.5 | Above | 47.5 |
| 58 | 223075_s_at | hypothetical | FLJ12783 | 9q34.13-q34.3 | AL136566.1 | 29.5 | Above | 3.9 |
| | | protein FLJ12783 | | | | | | |
| 59 | 229139_at | EST | | 8 | AI202201 | 29.5 | Above | 10.8 |
| 60 | 229367_s_at | Hypothetical | FLJ22690 | 7 | AW130536 | 29.5 | Above | 3.6 |
| | | proteins | | | | | | |
| | | FLJ22690. | | | | | | |
| 61 | 213093_at | FLJ30869 | FLJ30869 | Xq28 | AI471375 | 29.1 | Above | 2.5 |
| 62 | 216033_s_at | FYN oncogene | FYN | 6 | S74774.1 | 29.1 | Above | 2.7 |
| | | related to SRC | | | | | | |
| 63 | 202369_s_at | TRAM-like | KIAA0057 | 6p21.1-p12 | NM_012288.1 | 28.7 | Above | 3.3 |
| | | protein | | | | | | |
| 64 | 212592_at | immunoglobulin J | IGJ | 4q21 | AV733266 | 28.7 | Above | 7.9 |
| | | polypeptide, linker | | | | | | |
| | | protein for | | | | | | |
| | | immunoglobulin | | | | | | |
| | | alpha and mu | | | | | | |
| | | polypeptides | | | | | | |
| 65 | 219218_at | hypothetical | FLJ23058 | 17q25.3 | NM_024696.1 | 28.7 | Below | 6.2 |
| | | protein FLJ23058 | | | | | | |
| 66 | 242051_at | EST | | Y | AI695695 | 28.7 | Above | 2.2 |
| 67 | 200655_s_at | Calmodulin 1 | CALM1 | 14q24-q31 | NM_006888.1 | 28.5 | Above | 1.3 |
| | | (phosphorylase | | | | | | |
| | | kinase, delta) | | | | | | |
| 68 | 202794_at | Inositol | INPP1 | 2q32 | NM_002194.2 | 28.4 | Above | 1.6 |
| | | polyphosphate-1- | | | | | | |
| | | phosphatase | | | | | | |
| 69 | 218348_s_at | HSPC055 protein | HSPC055 | 16p13.3 | NM_014153.1 | 27.7 | Below | 1.1 |
| 70 | 205269_at | Lymphocyte | LCP2 | 5q33.1- | AI123251 | 26.9 | Above | 1.6 |
| | | cytosolic protein 2 | | qter | | | | |
| 71 | 238488_at | Ran binding | LOC51194 | 5q12.2 | BF511602 | 26.9 | Above | 2.7 |
| | | protein 11 | | | | | | |
| 72 | 202242_at | Transmembrane 4 | TM4SF2 | Xq11.4 | NM_004615.1 | 26.6 | Above | 1.7 |
| | | superfamily | | | | | | |
| | | member 2 | | | | | | |
| 73 | 218764_at | Hypothetical | MGC5363 | 14q22.1-q22.3 | NM_024064.1 | 26.6 | Above | 1.7 |
| | | protein MGC5363 | | q22.3 | | | | |
| 74 | 224811_at | FLJ30652 | FLJ30652 | 3 | BF112093 | 26.6 | Above | 1.5 |
| 75 | 225799_at | Hypothetical | MGC4677 | 2q12.3 | BF209337 | 26.6 | Above | 2.2 |
| | | protein MGC4677 | | | | | | |
| 76 | 228297_at | Calponin 3, acidic | CNN3 | 1p22-p21 | AI807004 | 26.6 | Above | 4.7 |
| 77 | 203508_at | Tumor necrosis | TNFRSF1B | 1p36.3-p36.2 | NM_001066.1 | 26 | Above | 2.6 |
| | | factor receptor | | | | | | |
| | | superfamily, | | | | | | |
| | | member 1B | | | | | | |
| 78 | 208071_s_at | Leukocyte- | LAIR1 | 19q13.4 | NM_021708.1 | 26 | Above | 2 |
| | | associated Ig-like | | | | | | |
| | | receptor 1 | | | | | | |
| 79 | 209321_s_at | Adenylate cyclase | ADCY3 | 2p24-p22 | AF033861.1 | 26 | Above | 2.1 |
| | | 3. | | | | | | |
| 80 | 226345_at | DKFZp434O1317 | DKFZp434O1317 | 10 | AW270158 | 26 | Below | 1.4 |
| 81 | 200863_s_at | RAB11A, member | RAB11A | 15q21.3-q22.31 | AI215102 | 25.8 | Above | 1.4 |
| | | RAS oncogene | | | | | | |
| | | family | | | | | | |
| 82 | 205270_s_at | Lymphocyte | LCP2 | 5q33.1- | NM_005565.2 | 25.8 | Above | 1.6 |
| | | cytosolic protein 2 | | qter | | | | |
| 83 | 208881_x_at | Isopentenyl- | IDI1 | 10p15.3 | BC005247.1 | 25.8 | Below | 1.7 |
| | | diphosphate delta | | | | | | |
| | | isomerase | | | | | | |
| 84 | 212862_at | CDP- | CDS2 | 20p13 | AL568982 | 25.8 | Above | 1.8 |
| | | diacylglycerol | | | | | | |
| | | synthase | | | | | | |
| | | (phosphatidate | | | | | | |
| | | cytidylyltransferase) 2 | | | | | | |
| 85 | 213385_at | Chimerin 2 | CHN2 | 7 | AK026415.1 | 25.8 | Above | 3 |
| 86 | 218013_x_at | Dynactin 4 | DCTN4 | 5q31-q32 | NM_016221.1 | 25.8 | Above | 3.6 |
| 87 | 218966_at | Myosin 5C | MYO5C | 15q21 | NM_018728.1 | 25.8 | Above | 1.8 |
| 88 | 200742_s_at | Ceroid- | CLN2 | 11p15 | BG231932 | 25 | Above | 1.5 |
| | | lipofuscinosis, | | | | | | |
| | | neuronal 2, late | | | | | | |
| | | infantile (Jansky- | | | | | | |
| | | Bielschowsky | | | | | | |
| | | disease). A | | | | | | |
| | | pepstatin- | | | | | | |
| | | insensitive | | | | | | |
| | | lysosomal | | | | | | |
| | | peptidase. | | | | | | |
| 89 | 203217_s_at | Sialyltransferase 9 | SIAT9 | 2p11.2 | NM_003896.1 | 25 | Above | 1.8 |
| 90 | 205259_at | Nuclear receptor | NR3C2 | 4q31.1 | NM_000901.1 | 25 | Above | 1.9 |
| | | subfamily 3, | | | | | | |
| | | group C, member 2 | | | | | | |
| 91 | 220684_at | T-box 21 | TBX21 | 17q21.2 | NM_013351.1 | 25 | Above | 3.3 |
| 92 | 225244_at | IMAGE3451454: | IMAGE34 | 1q42.13 | AA019893 | 25 | Above | 2 |
| | | GRASP protein | 51454 | | | | | |
| 93 | 239519_at | EST | | 10 | AA927670 | 25 | Above | 18.2 |
| 94 | 203005_at | Lymphotoxin beta | LTBR | 12p13 | NM_002342.1 | 24.3 | Above | 10 |
| | | receptor (TNFR | | | | | | |
| | | superfamily, | | | | | | |
| | | member 3) | | | | | | |
| 95 | 200665_s_at | Secreted protein, | SPARC | 5q31.3-q32 | NM_003118.1 | 24.3 | Above | 9.8 |
| | | acidic, cysteine- | | | | | | |
| | | rich (osteonectin) | | | | | | |
| 96 | 204004_at | PRKC, apoptosis, | PAWR | 12q21 | AI336206 | 24.3 | Above | 3 |
| | | WT1, regulator | | | | | | |
| 97 | 204576_s_at | KIAA0643 | | 16p12.3 | AA207013 | 24.3 | Above | 2 |
| | | protein | KIAA0643 | | | | | |
| 98 | 214255_at | ATPase, Class V, | ATP10C | 15q11-q13 | AB011138.1 | 24.3 | Above | 9.9 |
| | | type 10C | | | | | | |
| 99 | 216985_s_at | Syntaxin 3A | STX3A | 11q12.3 | AJ002077.1 | 24.3 | Above | 12 |
| 100 | 48106_at | FLJ20489 | FLJ20489 | 12p11.1 | H14241 | 24.3 | Above | 2.8 |

TABLE 64
Top 100 chi-square probe sets selected for E2A-PBX1

| | | | | | | | E2A | |
|---|---|---|---|---|---|---|---|---|
| | | | | | | | above/ | |
| | | | | Chromosomal | GenBank | Chi-square | below | Fold |
| | U133 probe set | Gene Description | Symbol | Location | reference | value | mean | change |
| 1 | 201579_at | FAT tumor | FAT | 4q34-q35 | NM_005245.1 | 88.0 | Above | 9.9 |
| | | suppressor | | | | | | |
| | | homolog 1 | | | | | | |
| | | (Drosophila) | | | | | | |
| 2 | 201695_s_at | nucleoside | NP | 14q13.1 | NM_000270.1 | 88.0 | Above | 3.8 |
| | | phosphorylase | | | | | | |
| 3 | 204674_at | lymphoid- | LRMP | 12p12.3 | NM_006152.1 | 88.0 | Above | 5.8 |
| | | restricted | | | | | | |
| | | membrane protein | | | | | | |
| 4 | 205253_at | pre-B-cell | PBX1 | 1q23 | NM_002585.1 | 88.0 | Above | 3549.2 |
| | | leukemia | | | | | | |
| | | transcription | | | | | | |
| | | factor 1 | | | | | | |
| 5 | 212148_at | pre-B-cell | PBX1 | 1q23 | BF967998 | 88.0 | Above | 5283.5 |
| | | leukemia | | | | | | |
| | | transcription | | | | | | |
| | | factor 1, splice | | | | | | |
| | | variant | | | | | | |
| 6 | 212151_at | pre-B-cell | PBX1 | 1q23 | BF967998 | 88.0 | Above | 7472.2 |
| | | leukemia | | | | | | |
| | | transcription | | | | | | |
| | | factor 1, splice | | | | | | |
| | | variant | | | | | | |
| 7 | 212371_at | DKFZp586C1019 | DKFZp58 | 1 | AL049397.1 | 88.0 | Above | 2.5 |
| | | | 6C1019 | | | | | |
| 8 | 219155_at | retinal | RDGBBB | 17q24.2 | NM_012417.1 | 88.0 | Above | 2.7 |
| | | degeneration | | | | | | |
| | | beta | | | | | | |
| 9 | 225483_at | hypothetical | MGC10485 | 11q25 | AI971602 | 88.0 | Above | 7.7 |
| | | protein | | | | | | |
| | | MGC10485 | | | | | | |
| 10 | 227439_at | E2a-Pbx1- | EB-1 | 12 | AW005572 | 88.0 | Above | 269.8 |
| | | associated protein | | | | | | |
| 11 | 227949_at | Q9H4T4 like | H17739 | 20q13.32 | AL357503 | 88.0 | Above | 59.3 |
| 12 | 230306_at | hypothetical | MGC10485 | 11q25 | AA514326 | 88.0 | Above | 19.2 |
| | | protein | | | | | | |
| | | MGC10485 | | | | | | |
| 13 | 231095_at | retinal | RDGBBB | 17q24.2 | AW193811 | 88.0 | Above | 25.6 |
| | | degeneration | | | | | | |
| | | beta | | | | | | |
| 14 | 203372_s_at | STAT induced | SOCS2 | 12q | AB004903.1 | 80.6 | Below | 23.4 |
| | | STAT inhibitor-2 | | | | | | |
| 15 | 206028_s_at | c-mer protooncogene | MERTK | 2q14.1 | NM_006343.1 | 80.6 | Above | 23.7 |
| | | tyrosine | | | | | | |
| | | kinase | | | | | | |
| 16 | 206181_at | signaling | SLAM | 1q22-q23 | NM_003037.1 | 80.6 | Above | 6.3 |
| | | lymphocytic | | | | | | |
| | | activation | | | | | | |
| | | molecule | | | | | | |
| 17 | 208788_at | homolog of yeast | HELO1 | 6p21.1-p12.1 | AL136939.1 | 80.6 | Above | 2.2 |
| | | long chain | | | | | | |
| | | polyunsaturated | | | | | | |
| | | fatty acid | | | | | | |
| | | elongation | | | | | | |
| | | enzyme 2 | | | | | | |
| 18 | 209760_at | KIAA0922 | KIAA0922 | 4q31.23 | AL136932.1 | 80.6 | Above | 2.9 |
| | | protein | | | | | | |
| 19 | 35974_at | lymphoid- | LRMP | 12p12.3 | U10485 | 80.6 | Above | 6.2 |
| | | restricted | | | | | | |
| | | membrane protein | | | | | | |
| 20 | 38340_at | huntingtin | HIP12 | 12q24 | AB014555 | 80.6 | Above | 3.8 |
| | | interacting protein | | | | | | |
| | | 12 | | | | | | |
| 21 | 208644_at | ADP- | ADPRT | 1q41-q42 | M32721.1 | 80.2 | Above | 3.0 |
| | | ribosyltransferase | | | | | | |
| | | (NAD+; poly | | | | | | |
| | | (ADP-ribose) | | | | | | |
| | | polymerase) | | | | | | |
| 22 | 212789_at | KIAA0056 | KIAA0056 | 11q25 | AI796581 | 80.2 | Above | 3.9 |
| | | protein | | | | | | |
| 23 | 221113_s_at | wingless-type | WNT16 | 7q31 | NM_016087.1 | 80.2 | Above | 2547.6 |
| | | MMTV | | | | | | |
| | | integration site | | | | | | |
| | | family, member | | | | | | |
| | | 16 | | | | | | |
| 24 | 224022_x_at | wingless-type | WNT16 | 7q31 | AF169963.1 | 80.2 | Above | 569.1 |
| | | MMTV | | | | | | |
| | | integration site | | | | | | |
| | | family, member | | | | | | |
| | | 16 | | | | | | |
| 25 | 231040_at | EST | | 9 | AW512988 | 80.2 | Above | 16.4 |
| 26 | 232289_at | FLJ14167 | FLJ14167 | 17 | BF237871 | 80.2 | Above | 144.1 |
| 27 | 235666_at | EST | FLJ20489 | 10 | AA903473 | 80.2 | Above | 654.6 |
| 28 | 203373_at | STAT induced | SOCS2 | 12q | NM_003877.1 | 74.2 | Below | 24.8 |
| | | STAT inhibitor-2 | | | | | | |
| 29 | 210785_s_at | basement | ICB-1 | 1p35.3 | AB035482.1 | 74.2 | Below | 4.1 |
| | | membrane- | | | | | | |
| | | induced gene | | | | | | |
| 30 | 224733_at | chemokine-like | CKLFSF3 | 16q23.1 | AL574900 | 74.2 | Below | 41.7 |
| | | factor super | | | | | | |
| | | family 3 | | | | | | |
| 31 | 225235_at | hypothetical | MGC14859 | 5q35.3 | AW007710 | 74.2 | Above | 3.6 |
| | | protein | | | | | | |
| | | MGC14859 | | | | | | |
| 32 | 204114_at | nidogen 2 | NID2 | 14q21-q22 | NM_007361.1 | 73.1 | Above | 15.1 |
| | | (osteonidogen) | | | | | | |
| 33 | 211913_s_at | c-mer protooncogene | MERTK | 2q14.1 | L08961.1 | 72.8 | Above | 37.7 |
| | | tyrosine | | | | | | |
| | | kinase | | | | | | |
| 34 | 219551_at | uncharacterized | BM040 | 3q21.1 | NM_018456.1 | 72.8 | Above | 3.0 |
| | | bone marrow | | | | | | |
| | | protein BM040 | | | | | | |
| 35 | 223693_s_at | hypothetical | FLJ10324 | 7p22 | AL136731.1 | 72.8 | Above | 65.6 |
| | | protein FLJ10324 | | | | | | |
| 36 | 200600_at | moesin | MSN | Xq11.2-q12 | NM_002444.1 | 72.5 | Below | 2.2 |
| 37 | 213909_at | FLJ12280 | FLJ12280 | 3 | AU147799 | 72.5 | Above | 12.5 |
| 38 | 221669_s_at | acyl-Coenzyme A | ACAD8 | 11q25 | BC001964.1 | 72.5 | Above | 2.6 |
| | | dehydrogenase | | | | | | |
| | | family, member 8 | | | | | | |
| 39 | 235911_at | ESTs, Weakly | | 3 | AI885815 | 72.5 | Above | 36.6 |
| | | similar to PIHUB6 | | | | | | |
| | | salivary proline- | | | | | | |
| | | rich protein | | | | | | |
| | | precursor PRB1 | | | | | | |
| | | (large allele) | | | | | | |
| 40 | 243533_x_at | ESTs | | | H09663 | 72.5 | Above | 23.2 |
| 41 | 202615_at | DKFZp686D0521 | DKFZp686D0521 | 9 | BF222895 | 68.6 | Below | 6.2 |
| 42 | 204774_at | ecotropic viral | EVI2A | 17q11.2 | NM_014210.1 | 68.6 | Below | 3.0 |
| | | integration site 2A | | | | | | |
| 43 | 218283_at | synovial sarcoma | SS18L2 | 3p21 | NM_016305.1 | 68.6 | Above | 1.6 |
| | | translocation gene | | | | | | |
| | | on chromosome | | | | | | |
| | | 18-like 2 | | | | | | |
| 44 | 209130_at | synaptosomal- | SNAP23 | 15q14 | BC003686.1 | 67.8 | Below | 1.9 |
| | | associated protein, | | | | | | |
| | | 23 kDa | | | | | | |
| 45 | 228580_at | serine protease | HTRA3 | 4p16.1 | AI828007 | 66.6 | Above | 3.8 |
| | | HTRA3 | | | | | | |
| 46 | 202796_at | synaptopodin | KIAA1029 | 5q33.1 | NM_007286.1 | 66.5 | Above | 52.3 |
| 47 | 218640_s_at | phafin 2 | FLJ13187 | 8q21.3 | NM_024613.1 | 66.5 | Above | 3.1 |
| 48 | 235099_at | ESTs, Weakly | | 3 | AW080832 | 66.5 | Above | 6.7 |
| | | similar to | | | | | | |
| | | PLLP_HUMAN | | | | | | |
| | | Plasmolipin | | | | | | |
| | | [H. sapiens] | | | | | | |
| 49 | 201889_at | family with | FAM3C | 7q22.1-q31.1 | NM_014888.1 | 65.3 | Above | 4.6 |
| | | sequence | | | | | | |
| | | similarity 3, | | | | | | |
| | | member C | | | | | | |
| 50 | 202106_at | golgi autoantigen, | GOLGA3 | 12q24.33 | NM_005895.1 | 65.3 | Above | 3.3 |
| | | golgin subfamily | | | | | | |
| | | a, 3 | | | | | | |
| 51 | 202208_s_at | ADP-ribosylation | ARL7 | 2q37.2 | BC001051.1 | 65.3 | Above | 3.2 |
| | | factor-like 7 | | | | | | |
| 52 | 205173_x_at | CD58 antigen, | CD58 | 1p13 | NM_001779.1 | 65.3 | Above | 2.4 |
| | | (lymphocyte | | | | | | |
| | | function- | | | | | | |
| | | associated antigen | | | | | | |
| | | 3) | | | | | | |
| 53 | 211744_s_at | CD58 antigen, | CD58 | 1p13 | BC005930.1 | 65.3 | Above | 2.5 |
| | | (lymphocyte | | | | | | |
| | | function- | | | | | | |
| | | associated antigen | | | | | | |
| | | 3) | | | | | | |
| 54 | 212552_at | hippocalcin-like 1 | HPCAL1 | 2p25.1 | BE617588 | 65.3 | Below | 2.6 |
| 55 | 213358_at | KIAA0802 | KIAA0802 | 18p11.21 | AB018345.1 | 65.3 | Above | 12.7 |
| | | protein | | | | | | |
| 56 | 222699_s_at | phafin 2 | FLJ13187 | 8q21.3 | BF439250 | 65.3 | Above | 3.5 |
| 57 | 225618_at | EST | | 17 | AI769587 | 65.3 | Below | 5.3 |
| 58 | 238778_at | DKFZp451L157 | DKFZp451L157 | 10 | AI244661 | 65.3 | Above | 23.5 |
| 59 | 239427_at | ESTs | | 1 | AA131524 | 65.3 | Above | 13.7 |
| 60 | 47069_at | Rho GTPase | ARHGAP8 | 22q13.31 | AA533284 | 65.3 | Above | 3.3 |
| | | activating protein 8 | | | | | | |
| 61 | 205769_at | solute carrier | SLC27A2 | 15q21.2 | NM_003645.1 | 65.1 | Above | 56.0 |
| | | family 27 (fatty | | | | | | |
| | | acid transporter), | | | | | | |
| | | member 2 | | | | | | |
| 62 | 210786_s_at | Friend leukemia | FLI1 | 11q24.1-q24.3 | M93255.1 | 65.1 | Above | 2.2 |
| | | virus integration 1 | | | | | | |
| 63 | 212985_at | DKFZp434E033 | DKFZp434E033 | 4 | BF115739 | 65.1 | Above | 7.1 |
| 64 | 227441_s_at | E2a-Pbx1- | EB-1 | 12 | AW005572 | 65.1 | Above | 1139.4 |
| | | associated protein | | | | | | |
| 65 | 234261_at | DKFZp761M10121 | DKFZp761M10121 | 12 | AL137313.1 | 65.1 | Above | 960.8 |
| 66 | 244565_at | ESTs | | 10 | AI685824 | 65.1 | Above | 7.6 |
| 67 | 202181_at | KIAA0247 gene | KIAA0247 | 14q24.1 | NM_014734.1 | 63.7 | Above | 1.8 |
| | | product | | | | | | |
| 68 | 202207_at | ADP-ribosylation | ARL7 | 2q37.2 | NM_005737.2 | 63.7 | Above | 3.2 |
| | | factor-like 7 | | | | | | |
| 69 | 207571_x_at | basement | ICB-1 | 1p35.3 | NM_004848.1 | 63.7 | Below | 4.4 |
| | | membrane- | | | | | | |
| | | induced gene | | | | | | |
| 70 | 209558_s_at | huntingtin | HIP12 | 12q24 | AB013384.1 | 61.1 | Above | 23.8 |
| | | interacting protein | | | | | | |
| | | 12 | | | | | | |
| 71 | 213005_s_at | KIAA0172 | KIAA0172 | 9p24.3 | D79994.1 | 61.1 | Above | 8.3 |
| | | protein | | | | | | |
| 72 | 236854_at | cDNA | DKFZp667F0617 | 20 | AA743694 | 61.1 | Above | 12.6 |
| | | DKFZp667F0617 | | | | | | |
| 73 | 226233_at | tubulin-specific | TBCE | 1q42.3 | BG112197 | 60.0 | Above | 2.6 |
| | | chaperone e | | | | | | |
| 74 | 203435_s_at | membrane | MME | 3q25.1-q25.2 | NM_007287.1 | 59.9 | Below | 2.2 |
| | | metallo- | | | | | | |
| | | endopeptidase | | | | | | |
| | | (neutral | | | | | | |
| | | endopeptidase, | | | | | | |
| | | enkephalinase, | | | | | | |
| | | CALLA, CD10) | | | | | | |
| 75 | 202478_at | GS3955 protein | GS3955 | 2p25.1 | NM_021643.1 | 59.3 | Above | 4.0 |
| 76 | 202479_s_at | GS3955 protein | GS3955 | 2p25.1 | BC002637.1 | 59.3 | Above | 3.3 |
| 77 | 203999_at | synaptotagmin I | SYT1 | 12cen-q21 | NM_005639.1 | 59.3 | Above | 3.9 |
| 78 | 212149_at | KIAA0143 | KIAA0143 | 8q24.12 | AA805651 | 59.3 | Below | 13.5 |
| | | protein | | | | | | |
| 79 | 212873_at | minor | HA-1 | 19p13.3 | BE349017 | 59.3 | Below | 2.9 |
| | | histocompatibility | | | | | | |
| | | antigen HA-1 | | | | | | |
| 80 | 218346_s_at | p53 regulated | PA26 | 6q21 | NM_014454.1 | 59.3 | Below | 4.7 |
| | | PA26 nuclear | | | | | | |
| | | protein | | | | | | |
| 81 | 224856_at | FK506 binding | FKBP5 | 6p21.3-21.2 | AL122066.1 | 59.3 | Below | 5.5 |
| | | protein 5 | | | | | | |
| 82 | 200811_at | cold inducible | CIRBP | 19p13.3 | NM_001280.1 | 59.1 | Below | 5.8 |
| | | RNA binding | | | | | | |
| | | protein | | | | | | |
| 83 | 201722_s_at | UDP-N-acetyl- | GALNT1 | 18q12.1 | NM_020474.2 | 59.1 | Below | 1.8 |
| | | alpha-D- | | | | | | |
| | | galactosamine: polypeptide | | | | | | |
| | | N-acetylgalactosaminyltransferase 1 | | | | | | |
| | | (GalNAc-T1) | | | | | | |
| 84 | 223711_s_at | HSPC144 protein | HSPC144 | 11q25 | AF182413.1 | 59.1 | Above | 2.0 |
| 85 | 233273_at | cDNA FLJ12010 | FLJ12010 | 1 | AU146834 | 59.1 | Above | 30.6 |
| | | fis | | | | | | |
| 86 | 201460_at | mitogen-activated | MAPKAPK2 | 1q32 | AI141802 | 57.9 | Above | 2.1 |
| | | protein kinase- | | | | | | |
| | | activated protein | | | | | | |
| | | kinase 2 | | | | | | |
| 87 | 202421_at | immunoglobulin | IGSF3 | 1p13 | AB007935.1 | 57.9 | Above | 4.4 |
| | | superfamily, | | | | | | |
| | | member 3 | | | | | | |
| 88 | 217983_s_at | ribonuclease 6 | RNASE6PL | 6q27 | NM_003730.2 | 57.9 | Below | 3.4 |
| | | precursor | | | | | | |
| 89 | 218087_s_at | sorbin and SH3 | SORBS1 | 10q23.3-q24.1 | NM_015385.1 | 57.9 | Above | 25.1 |
| | | domain containing 1 | | | | | | |
| 90 | 218491_s_at | HSPC144 protein | HSPC144 | 11q25 | NM_014174.1 | 57.9 | Above | 1.4 |
| 91 | 201825_s_at | CGI-49 protein | LOC51097 | 1q44 | AL572542 | 57.8 | Above | 2.2 |
| 92 | 202206_at | ADP-ribosylation | ARL7 | 2q37.2 | NM_005737.2 | 57.8 | Above | 3.9 |
| | | factor-like 7 | | | | | | |
| 93 | 218683_at | polypyrimidine | PTBP2 | 1p22.11-P21.3 | NM_021190.1 | 57.8 | Above | 1.8 |
| | | tract binding | | | | | | |
| | | protein 2 | | | | | | |
| 94 | 226590_at | cDNA clone | | 9 | AA031404 | 57.8 | Above | 3.1 |
| | | EUROIMAGE | | | | | | |
| | | 1517766 | | | | | | |
| 95 | 227440_at | E2a-Pbx1- | EB-1 | 12 | AW005572 | 57.8 | Above | 1168.9 |
| | | associated protein | | | | | | |
| 96 | 229770_at | hypothetical | FLJ31978 | 12q24.33 | AI041543 | 57.8 | Above | 51.8 |
| | | protein FLJ31978 | | | | | | |
| 97 | 40148_at | amyloid beta (A4) | APBB2 | 4p14 | U62325 | 57.8 | Above | 6.2 |
| | | precursor protein- | | | | | | |
| | | binding, family B, | | | | | | |
| | | member 2 (Fe65- | | | | | | |
| | | like) | | | | | | |
| 98 | 212959_s_at | MGC4170 protein | MGC4170 | 12q23.1 | AK001821.1 | 57.2 | Below | 3.0 |
| 99 | 203143_s_at | KIAA0040 gene | KIAA0040 | 1q24-25 | T79953 | 56.3 | Above | 2.4 |
| | | product | | | | | | |
| 100 | 209683_at | hypothetical | DKFZP566A1524 | 2p24.2 | AA243659 | 56.3 | Below | 10.0 |
| | | protein | | | | | | |
| | | DKFZp566A1524 | | | | | | |

TABLE 65
Top 100 chi-square probe sets selected for Hyperdiploid >50

| | | | | | | | HD | |
|---|---|---|---|---|---|---|---|---|
| | | | | | | Chi- | above/ | |
| | U133 probe | | | Chromosomal | | square | below | Fold |
| | set | Gene description | Symbol | Location | GenBank Ref | value | mean | change |
| 1 | 200600_at | Moesin | MSN | Xq11.2-q12 | NM_002444.1 | 34.0 | Above | 1.9 |
| | | (membrane- | | | | | | |
| | | organizing | | | | | | |
| | | extensio spike | | | | | | |
| | | protein) | | | | | | |
| 2 | 200737_at | Phosphoglycerate | PGK1 | Xq13 | NM_000291.1 | 34.0 | Above | 1.8 |
| | | kinase 1 | | | | | | |
| 3 | 200980_s_at | Pyruvate | PDHA1 | Xp22.2-p22.1 | NM_000284.1 | 34.0 | Above | 1.7 |
| | | dehydrogenase | | | | | | |
| | | (lipoamide) alpha 1 | | | | | | |
| 4 | 201136_at | Proteolipid protein | PLP2 | Xp11.23 | NM_002668.1 | 34.0 | Above | 3.3 |
| | | 2 (colonic | | | | | | |
| | | epithelium- | | | | | | |
| | | enriched) | | | | | | |
| 5 | 201807_at | Vacuolar protein | VPS26 | 10q21.1 | NM_004896.1 | 34.0 | Above | 1.7 |
| | | sorting 26 (yeast) | | | | | | |
| 6 | 202214_s_at | Cullin 4B | CUL4B | Xq23 | NM_003588.1 | 34.0 | Above | 1.9 |
| 7 | 202557_at | Stress 70 protein | STCH | 21q11 | AI718418 | 34.0 | Above | 2.0 |
| | | chaperone, | | | | | | |
| | | microsome | | | | | | |
| | | associated, 60 kD | | | | | | |
| 8 | 202593_s_at | membrane | MIR16 | 16p12-p11.2 | NM_016641.1 | 34.0 | Below | 1.6 |
| | | interacting protein | | | | | | |
| | | of RGS16 | | | | | | |
| 9 | 203680_at | Protein kinase, | PRKAR2B | 7q22-q31.1 | NM_002736.1 | 34.0 | Above | 3.3 |
| | | cAMP-dependent, | | | | | | |
| | | regulatory, type II, | | | | | | |
| | | beta | | | | | | |
| 10 | 204194_at | BTB and CNC | BACH1 | 21q22.11 | NM_001186.1 | 34.0 | Above | 1.8 |
| | | homology 1, basic | | | | | | |
| | | leucine zipper | | | | | | |
| | | transcription | | | | | | |
| | | factor 1 | | | | | | |
| 11 | 205324_s_at | FtsJ homolog 1 | FTSJ1 | Xp11.23 | NM_012280.1 | 34.0 | Above | 2.1 |
| | | (E. coli) | | | | | | |
| 12 | 208598_s_at | Upstream | UREB1 | Xp11.22 | NM_005703.2 | 34.0 | Above | 1.6 |
| | | regulatory element | | | | | | |
| | | binding protein 1 | | | | | | |
| 13 | 208861_s_at | Alpha | ATRX | Xq13.1-q21.1 | U72937.2 | 34.0 | Above | 1.7 |
| | | thalassemia/menta | | | | | | |
| | | 1 retardation | | | | | | |
| | | syndrome X- | | | | | | |
| | | linked (RAD54 | | | | | | |
| | | homolog, S. | | | | | | |
| | | cerevisiae) | | | | | | |
| 14 | 211342_x_at | trinucleotide | TNRC11 | Xq13 | BC004354.1 | 34.0 | Above | 1.8 |
| | | repeat containing | | | | | | |
| | | 11 (THR- | | | | | | |
| | | associated protein, | | | | | | |
| | | 230 kDa subunit) | | | | | | |
| 15 | 216071_x_at | Trinucleotide | TNRC11 | Xq13 | AF132033 | 34.0 | Above | 1.8 |
| | | repeat containing | | | | | | |
| | | 11 | | | | | | |
| 16 | 218573_at | APR-1 | MAGEH1 | Xp11.22 | NM_014061.1 | 34.0 | Above | 3.0 |
| | | protein/melanoma- | | | | | | |
| | | associated | | | | | | |
| | | antigen | | | | | | |
| 17 | 219485_s_at | proteasome | PSMD10 | Xq22.3 | NM_002814.1 | 34.0 | Above | 2.4 |
| | | (prosome, | | | | | | |
| | | macropain) 26S | | | | | | |
| | | subunit, non- | | | | | | |
| | | ATPase, 10 | | | | | | |
| 18 | 200655_s_at | Calmodulin 1 | CALM1 | 14q24-q31 | NM_006888.1 | 30.1 | Above | 1.7 |
| | | (phosphorylase | | | | | | |
| | | kinase, delta) | | | | | | |
| 19 | 200738_s_at | Phosphoglycerate | PGK1 | Xq13 | NM_000291.1 | 30.1 | Above | 1.8 |
| | | kinase 1 | | | | | | |
| 20 | 200944_s_at | High-mobility | HMG14 | 21q22.2 | NM_004965.1 | 30.1 | Above | 1.7 |
| | | group (nonhistone | | | | | | |
| | | chromosomal) | | | | | | |
| | | protein 14; | | | | | | |
| | | member of the | | | | | | |
| | | HMG 14/17 | | | | | | |
| | | family | | | | | | |
| 21 | 201092_at | Retinoblastoma | RBBP7 | Xp22.31 | NM_002893.2 | 30.1 | Above | 1.6 |
| | | binding protein | | | | | | |
| | | 7/RbAp46 | | | | | | |
| 22 | 201100_s_at | Ubiquitin specific | USP9X | Xp11.4 | NM_004652.2 | 30.1 | Above | 1.7 |
| | | protease 9 | | | | | | |
| 23 | 201688_s_at | Tumor protein | TPD52 | 8q21 | BE974098 | 30.1 | Below | 4.1 |
| | | D52 | | | | | | |
| 24 | 201899_s_at | Ubiquitin- | UBE2A | Xq24-q25 | NM_003336.1 | 30.1 | Above | 1.8 |
| | | conjugating | | | | | | |
| | | enzyme E2A | | | | | | |
| | | (RAD6 homolog) | | | | | | |
| 25 | 202325_s_at | ATP synthase, H+ | ATP5J | 21q21.1 | NM_001685.1 | 30.1 | Above | 1.6 |
| | | transporting, | | | | | | |
| | | mitochondrial F0 | | | | | | |
| | | complex, subunit | | | | | | |
| | | F6 | | | | | | |
| 26 | 202829_s_at | Synaptobrevin- | SYBL1 | Xq28 | NM_005638.1 | 30.1 | Above | 1.5 |
| | | like 1 | | | | | | |
| 27 | 202854_at | Hypoxanthine | HPRT1 | Xq26.1 | NM_000194.1 | 30.1 | Above | 1.4 |
| | | phosphoribosyltransferase | | | | | | |
| | | 1 (Lesch- | | | | | | |
| | | Nyhan syndrome) | | | | | | |
| 28 | 206846_s_at | Histone | HDAC6 | Xp11.23 | NM_006044.2 | 30.1 | Above | 1.5 |
| | | deacetylase 6 | | | | | | |
| 29 | 209370_s_at | SH3-domain | SH3BP2 | 4p16.3 | AB000462.1 | 30.1 | Above | 3.1 |
| | | binding protein 2 | | | | | | |
| 30 | 209565_at | zinc finger protein | ZNF183 | Xq25-q26 | BC000832.1 | 30.1 | Above | 2.2 |
| | | 183 | | | | | | |
| 31 | 212846_at | KIAA0179 | KIAA0179 | 21q22.3 | D80001.1 | 30.1 | Above | 2.0 |
| | | protein. | | | | | | |
| 32 | 217356_s_at | Phosphoglycerate | PGK1 | Xq13 | S81916.1 | 30.1 | Above | 1.8 |
| | | kinase | | | | | | |
| 33 | 218163_at | MCT-1 protein | MCT-1 | Xq22-24 | NM_014060.1 | 30.1 | Above | 1.8 |
| 34 | 218386_x_at | Ubiquitin specific | USP16 | 21q22.11 | NM_006447.1 | 30.1 | Above | 1.7 |
| | | protease 16; de- | | | | | | |
| | | ubiquitinates | | | | | | |
| | | histone H2A; | | | | | | |
| | | ubiquitous | | | | | | |
| | | expression. | | | | | | |
| 35 | 218402_s_at | Hermansky- | HPS4 | | NM_022081.1 | 30.1 | Below | 3.4 |
| | | Pudlak syndrome 4 | | | | | | |
| 36 | 218495_at | Ubiquitously- | UXT | Xp11.23-p11.22 | NM_004182.1 | 30.1 | Above | 1.5 |
| | | expressed | | | | | | |
| | | transcript | | | | | | |
| 37 | 218499_at | Mst3 and SOK1- | MST4 | Xq26.1 | NM_016542.1 | 30.1 | Above | 2.5 |
| | | related | | | | | | |
| | | kinase/STE20-like | | | | | | |
| | | kinase; contains a | | | | | | |
| | | Ser/Thr protein | | | | | | |
| | | kinase domain | | | | | | |
| 38 | 218757_s_at | Similar to yeast | UPF3B | Xq25-q26 | NM_023010.1 | 30.1 | Above | 2.3 |
| | | Upf3, variant B | | | | | | |
| 39 | 219038_at | Hypothetical | FLJ11565 | Xq22.2 | NM_024657.1 | 30.1 | Above | 6.9 |
| | | protein FLJ11565 | | | | | | |
| 40 | 229967_at | Chemokine-like | CKLFSF2 | 16q23.1 | AA778552 | 30.1 | Above | 4.3 |
| | | factor super | | | | | | |
| | | family 2. | | | | | | |
| 41 | 242794_at | EST | | 4q31.1 | AI569476 | 30.1 | Above | 3.2 |
| 42 | 201132_at | Heterogeneous | HNRPH2 | Xq22 | NM_019597.1 | 30.0 | Above | 2.0 |
| | | nuclear | | | | | | |
| | | ribonucleoprotein | | | | | | |
| | | H2 (H') | | | | | | |
| 43 | 201312_s_at | SH3 domain | SH3BGRL | Xq13.3 | NM_003022.1 | 30.0 | Above | 1.6 |
| | | binding glutamic | | | | | | |
| | | acid-rich protein | | | | | | |
| | | like | | | | | | |
| 44 | 201894_s_at | Decorin; | DCN | 12q13.2 | NM_001920.1 | 30.0 | Above | 1.5 |
| | | glycoprotein that | | | | | | |
| | | binds to type I | | | | | | |
| | | collagen fibrils & | | | | | | |
| | | plays a role in | | | | | | |
| | | matrix assembly. | | | | | | |
| 45 | 201923_at | Peroxiredoxin 4 | PRDX4 | Xp22.13 | NM_006406.1 | 30.0 | Above | 1.9 |
| 46 | 202371_at | Hypothetical | FLJ21174 | Xq22.1 | NM_024863.1 | 30.0 | Above | 3.6 |
| | | protein FLJ21174 | | | | | | |
| 47 | 203126_at | Inositol (myo)-1 (or | IMPA2 | 18p11.2 | NM_014214.1 | 30.0 | Above | 4.1 |
| | | 4)- | | | | | | |
| | | monophosphatase 2 | | | | | | |
| 48 | 204219_s_at | proteasome | PSMC1 | 19p13.3 | NM_002802.1 | 30.0 | Above | 1.3 |
| | | (prosome, | | | | | | |
| | | macropain) 26S | | | | | | |
| | | subunit, ATPase, 1 | | | | | | |
| 49 | 204835_at | polymerase (DNA | POLA | Xp22.1-p21.3 | NM_016937.1 | 30.0 | Above | 2.0 |
| | | directed), alpha | | | | | | |
| 50 | 212071_s_at | Spectrin, beta, | SPTBN1 | 2p21 | BE968833 | 30.0 | Below | 1.7 |
| | | non-erythrocytic 1 | | | | | | |
| 51 | 212419_at | EST | | 10q22.3 | AL049949.1 | 30.0 | Above | 13.1 |
| 52 | 212718_at | Hypothetical | MGC5378 | 14q32.2 | BG110231 | 30.0 | Above | 1.5 |
| | | protein MGC5370 | | | | | | |
| 53 | 213502_x_at | Homo sapiens | FLJ32313 | 22q11.23 | X03529 | 30.0 | Below | 1.8 |
| | | cDNA FLJ32313 | | | | | | |
| | | fis, clone | | | | | | |
| | | PROST2003232, | | | | | | |
| | | weakly similar to | | | | | | |
| | | BETA- | | | | | | |
| | | GLUCURONIDA | | | | | | |
| | | SE PRECURSOR | | | | | | |
| | | (EC 3.2.1.31) | | | | | | |
| 54 | 214051_at | Thymosin, beta | TMSNB | Xq21.33-q22.3 | BF677486 | 30.0 | Above | 3.1 |
| 55 | 226039_at | Mannosyl (alpha- | MGAT4A | 2q11.2 | AW006441 | 30.0 | Above | 3.0 |
| | | 1,3)-glycoprotein | | | | | | |
| | | beta-1,4-N- | | | | | | |
| | | acetylglucosaminyltransferase | | | | | | |
| 56 | 227279_at | hypothetical | MGC15737 | Xq22.1 | AA847654 | 30.0 | Above | 5.6 |
| | | protein | | | | | | |
| | | MGC15737 | | | | | | |
| 57 | 200642_at | Superoxide | SOD1 | 21q22.11 | NM_000454.1 | 26.7 | Above | 2.3 |
| | | dismutase 1, | | | | | | |
| | | soluble | | | | | | |
| 58 | 200799_at | Heat shock 70 kD | HSPA1A | 6p21.3 | NM_005345.3 | 26.7 | Above | 2.7 |
| | | protein 1A | | | | | | |
| 59 | 200943_at | High-mobility | HMG14 | 21q22.2 | NM_004965.1 | 26.7 | Above | 1.6 |
| | | group (nonhistone | | | | | | |
| | | chromosomal) | | | | | | |
| | | protein 14; | | | | | | |
| | | member of the | | | | | | |
| | | HMG 14/17 | | | | | | |
| | | family | | | | | | |
| 60 | 201018_at | Eukaryotic | EIF1A | Xp22.12 | BE542684 | 26.7 | Above | 1.8 |
| | | translation | | | | | | |
| | | initiation factor | | | | | | |
| | | 1A | | | | | | |
| 61 | 201311_s_at | SH3 domain | SH3BGRL | Xq13.3 | AL515318 | 26.7 | Above | 1.6 |
| | | binding glutamic | | | | | | |
| | | acid-rich protein | | | | | | |
| | | like | | | | | | |
| 62 | 201443_s_at | ATPase, H+ | ATP6IP2 | Xq21 | AF248966.1 | 26.7 | Above | 1.9 |
| | | transporting, | | | | | | |
| | | lysosomal | | | | | | |
| | | interacting protein 2 | | | | | | |
| 63 | 201472_at | Von Hippel- | VBP1 | Xq28 | NM_003372.2 | 26.7 | Above | 1.7 |
| | | Lindau binding | | | | | | |
| | | protein 1 | | | | | | |
| 64 | 201689_s_at | Tumor protein | TPD52 | 8q21 | BE974098 | 26.7 | Below | 4.3 |
| | | D52 | | | | | | |
| 65 | 202602_s_at | HIV TAT specific | HTATSF1 | Xq26.1-q27.2 | NM_014500.1 | 26.7 | Above | 1.5 |
| | | factor 1 | | | | | | |
| 66 | 203041_s_at | Lysosomal- | LAMP2 | Xq24 | J04183.1 | 26.7 | Above | 3.1 |
| | | associated | | | | | | |
| | | membrane protein 2 | | | | | | |
| 67 | 203102_s_at | Mannosyl (alpha- | MGAT2 | 14q21 | NM_002408.2 | 26.7 | Above | 1.6 |
| | | 1,6-)-glycoprotein | | | | | | |
| | | beta-1,2-N- | | | | | | |
| | | acetylglucosaminyltransferase | | | | | | |
| 68 | 203744_at | High-mobility | HMG4 | Xq28 | NM_005342.1 | 26.7 | Above | 1.9 |
| | | group (nonhistone | | | | | | |
| | | chromosomal) | | | | | | |
| | | protein 4 | | | | | | |
| 69 | 205518_s_at | Cytidine | CMAH | 6p22-p23 | NM_003570.1 | 26.7 | Below | 2.9 |
| | | monophosphate- | | | | | | |
| | | N- | | | | | | |
| | | acetylneuraminic | | | | | | |
| | | acid hydroxylase | | | | | | |
| | | (CMP-N- | | | | | | |
| | | acetylneuraminate | | | | | | |
| | | monooxygenase) | | | | | | |
| 70 | 208683_at | Calpain 2, (m/II) | CAPN2 | 1q41-q42 | M23254.1 | 26.7 | Above | 2.2 |
| | | large subunit; | | | | | | |
| | | calcium- | | | | | | |
| | | dependent Cys | | | | | | |
| | | protease. | | | | | | |
| 71 | 209440_at | Phosphoribosyl | PRPS1 | Xq21-q27 | BC001605.1 | 26.7 | Above | 1.4 |
| | | pyrophosphate | | | | | | |
| | | synthetase 1; | | | | | | |
| | | purine | | | | | | |
| | | biosynthesis. | | | | | | |
| 72 | 210786_s_at | Friend leukemia | FLI1 | 11q24.1-q24.3 | M93255.1 | 26.7 | Below | 2.5 |
| | | virus integration 1 | | | | | | |
| 73 | 212070_at | G protein-coupled | GPR56 | 16q13 | AL554008 | 26.7 | Above | 2.4 |
| | | receptor 56 | | | | | | |
| 74 | 213334_x_at | Three prime repair | TREX2 | Xq28 | BE676218 | 26.7 | Above | 1.7 |
| | | exonuclease 2 | | | | | | |
| 75 | 215117_at | Recombination | RAG2 | 11p13 | AW058148 | 26.7 | Below | 27.2 |
| | | activating gene 2; | | | | | | |
| | | V(D)J | | | | | | |
| | | recombinase. | | | | | | |
| 76 | 218694_at | ALEX1 protein | ALEX1 | Xq21.33-q22.2 | NM_016608.1 | 26.7 | Above | 2.8 |
| 77 | 222741_s_at | hypothetical | FLJ11101 | 6p21.1 | AI761426 | 26.7 | Above | 1.5 |
| | | protein FLJ11101 | | | | | | |
| 78 | 223082_at | SH3-domain | SH3KBP1 | Xp22.1-p21.3 | AF230904.1 | 26.7 | Above | 2.0 |
| | | kinase binding | | | | | | |
| | | protein 1 | | | | | | |
| 79 | 225105_at | clone MGC: 23936 | | 12q23.3 | BF969397 | 26.7 | Above | 2.1 |
| | | IMAGE: 3838595, | | | | | | |
| | | mRNA, complete | | | | | | |
| | | cds | | | | | | |
| 80 | 225406_at | Twisted | TSG | 18p11.3 | AA195009 | 26.7 | Above | 1.9 |
| | | gastrulation | | | | | | |
| 81 | 225553_at | Homo sapiens | | 14q22.2 | AL042817 | 26.7 | Above | 1.6 |
| | | cDNA FLJ12874 | | | | | | |
| | | fis | | | | | | |
| 82 | 226199_at | Hypothetical | MGC23937 | Xq13.1 | AL563795 | 26.7 | Above | 2.1 |
| | | protein | | | | | | |
| | | MGC23937 | | | | | | |
| 83 | 226875_at | Hypothetical | FLJ32122 | Xq24 | AI742838 | 26.7 | Above | 2.3 |
| | | protein FLJ32122 | | | | | | |
| 84 | 232974_at | cDNA FLJ12417 | | Xp22.31 | AU148256 | 26.7 | Above | 3.1 |
| | | fis | | | | | | |
| 85 | 46323_at | SCAN-1 Ca++- | SHAPY | 17q25.3 | AL120741 | 26.7 | Above | 1.7 |
| | | dependent ER | | | | | | |
| | | nucleoside | | | | | | |
| | | diphosphatase/apy | | | | | | |
| | | rase | | | | | | |
| 86 | 203694_s_at | DEAD/H (Asp- | DDX16 | 6p21.3 | NM_003587.2 | 26.3 | Above | 1.3 |
| | | Glu-Ala-Asp/His) | | | | | | |
| | | box polypeptide | | | | | | |
| | | 16 | | | | | | |
| 87 | 200658_s_at | Prohibitin | PHB | 17q21 | AL560017 | 26.3 | Above | 2.0 |
| 88 | 201898_s_at | ubiquitin- | UBE2A | Xq24-q25 | AI126625 | 26.3 | Above | 1.6 |
| | | conjugating | | | | | | |
| | | enzyme E2A | | | | | | |
| | | (RAD6 homolog) | | | | | | |
| 89 | 203556_at | KIAA0854 | KIAA0854 | 8q24.13 | NM_014943.1 | 26.3 | Below | 1.6 |
| | | protein | | | | | | |
| 90 | 203745_at | Holocytochrome c | HCCS | Xp22.3 | AI801013 | 26.3 | Above | 2.1 |
| | | synthase | | | | | | |
| | | (cytochrome c | | | | | | |
| | | heme-lyase) | | | | | | |
| 91 | 203909_at | Solute carrier | SLC9A6 | Xq26.3 | NM_006359.1 | 26.3 | Above | 1.9 |
| | | family 9 | | | | | | |
| | | (sodium/hydrogen | | | | | | |
| | | exchanger), | | | | | | |
| | | isoform 6 | | | | | | |
| 92 | 204446_s_at | Arachidonate 5- | ALOX5 | 10q11.2 | NM_000698.1 | 26.3 | Above | 4.2 |
| | | lipoxygenase | | | | | | |
| 93 | 205191_at | Retinitis | RP2 | Xp11.4-p11.21 | NM_006915.1 | 26.3 | Above | 2.1 |
| | | pigmentosa 2 (X- | | | | | | |
| | | linked recessive) | | | | | | |
| 94 | 206874_s_at | Ste20-related | SLK | 10q25.1 | AL138761 | 26.3 | Above | 1.6 |
| | | serine/threonine | | | | | | |
| | | kinase | | | | | | |
| 95 | 208073_x_at | Tetratricopeptide | TTC3 | 21q22.2 | NM_003316.1 | 26.3 | Above | 1.9 |
| | | repeat domain 3 | | | | | | |
| 96 | 209056_s_at | CDC5 cell | CDC5L | 6p21 | AW268817 | 26.3 | Above | 1.4 |
| | | division cycle 5- | | | | | | |
| | | like (S. pombe) | | | | | | |
| 97 | 210645_s_at | Tetratricopeptide | TTC3 | 21q22.2 | D83077.1 | 26.3 | Above | 2.2 |
| | | repeat domain 3 | | | | | | |
| 98 | 215773_x_at | ADP- | ADPRTL2 | 14q11.2-q12 | AJ236912.1 | 26.3 | Above | 1.6 |
| | | ribosyltransferase | | | | | | |
| | | (NAD+; | | | | | | |
| | | poly(ADP-ribose) | | | | | | |
| | | polymerase)-like 2 | | | | | | |
| 99 | 215884_s_at | Ubiquilin 2 | UBQLN2 | Xp11.23-p11.1 | AK001029.1 | 26.3 | Above | 1.9 |
| 100 | 217954_s_at | PHD finger | PHF3 | 6 | NM_015153.1 | 26.3 | Above | 1.5 |
| | | protein 3 | | | | | | |

TABLE 66
Top 100 chi-square probe sets selected for MLL

| | | | | | | | MLL | |
|---|---|---|---|---|---|---|---|---|
| | | | | | | Chi- | above/ | |
| | U133 probe | | | Chromosomal | | square | below | Fold |
| | set | Description | Symbol | Location | GenBank Ref | value | mean | change |
| 1 | 202603_at | a disintegrin and | ADAM10 | 15q22 | N51370 | 44.6 | Above | 1.8 |
| | | metalloproteinase | | | | | | |
| | | domain 10 | | | | | | |
| 2 | 219463_at | chromosome 20 | C20orf103 | 20p12 | NM_012261.1 | 44.6 | Above | 24.7 |
| | | open reading | | | | | | |
| | | frame 103 | | | | | | |
| 3 | 224772_at | neuron navigator 1 | NAV1 | | AB032977.1 | 44.6 | Below | 3.8 |
| 4 | 204069_at | Meis1, myeloid | MEIS1 | 2p14-p13 | NM_002398.1 | 44.4 | Above | 73.7 |
| | | ecotropic viral | | | | | | |
| | | integration site 1 | | | | | | |
| | | homolog | | | | | | |
| 5 | 218966_at | myosin 5C | MYO5C | 15q21 | NM_018728.1 | 44.4 | Below | 4.5 |
| 6 | 226939_at | cDNA FLJ37247 | FLJ37247 | | AI202327 | 44.4 | Above | 6.9 |
| | | fis | | | | | | |
| 7 | 204446_s_at | arachidonate 5- | ALOX5 | 10q11.2 | NM_000698.1 | 40.7 | Below | 66.8 |
| | | lipoxygenase | | | | | | |
| 8 | 206492_at | fragile histidine | FHIT | 3p14.2 | NM_002012.1 | 40.7 | Below | 36.6 |
| | | triad gene | | | | | | |
| 9 | 212588_at | protein tyrosine | PTPRC | 1q31-q32 | AI809341 | 40.7 | Above | 2.3 |
| | | phosphatase, | | | | | | |
| | | receptor type, C | | | | | | |
| 10 | 215925_s_at | CD72 antigen | CD72 | 9p11.2 | AF283777.2 | 40.7 | Above | 3.0 |
| | | (ligand for CD5) | | | | | | |
| 11 | 211733_x_at | sterol carrier | SCP2 | 1p32 | BC005911.1 | 40.1 | Above | 1.5 |
| | | protein 2 | | | | | | |
| 12 | 212386_at | cDNA FLJ11918 | FLJ11918 | | AK021980.1 | 40.1 | Below | 3.1 |
| | | fis | | | | | | |
| 13 | 218764_at | Protein Kinase C | PRKCH | 14q22.1-q22.3 | NM_024064.1 | 40.1 | Below | 7.6 |
| | | eta isoform. | | | | | | |
| 14 | 218847_at | IGF-II mRNA- | IMP-2 | 3q28 | NM_006548.1 | 40.1 | Above | 23.2 |
| | | binding protein 2 | | | | | | |
| 15 | 222409_at | coronin, actin | CORO1C | 12q24.1 | AL162070.1 | 40.1 | Above | 4.8 |
| | | binding protein, | | | | | | |
| | | 1C | | | | | | |
| 16 | 242172_at | ESTs | | | N50406 | 40.1 | Above | 33.6 |
| 17 | 201153_s_at | muscleblind-like | MBNL | 3q25 | NM_021038.1 | 40.0 | Above | 2.1 |
| | | (Drosophila) | | | | | | |
| 18 | 210487_at | deoxynucleotidyltransferase, | DNTT | 10q23-q24 | M11722.1 | 40.0 | Below | 2.9 |
| | | terminal | | | | | | |
| 19 | 219686_at | gene for | HSA250839 | 4p16.2 | NM_018401.1 | 40.0 | Below | 28.3 |
| | | serine/threonine | | | | | | |
| | | protein kinase | | | | | | |
| 20 | 226981_at | Homo sapiens, | | | AW002079 | 37.4 | Below | 1.0 |
| | | clone | | | | | | |
| | | IMAGE: 4401491, | | | | | | |
| | | mRNA | | | | | | |
| 21 | 203375_s_at | tripeptidyl | TPP2 | 13q32-q33 | NM_003291.1 | 37.2 | Above | 1.6 |
| | | peptidase II | | | | | | |
| 22 | 221676_s_at | coronin, actin | CORO1C | 12q24.1 | BC002342.1 | 37.2 | Above | 3.5 |
| | | binding protein, | | | | | | |
| | | 1C | | | | | | |
| 23 | 201152_s_at | muscleblind-like | MBNL | 3q25 | NM_021038.1 | 36.2 | Above | 2.2 |
| | | (Drosophila) | | | | | | |
| 24 | 221773_at | ELK3, ETS- | ELK3 | 12q23 | AW575374 | 36.2 | Below | 8.2 |
| | | domain protein | | | | | | |
| | | (SRF accessory | | | | | | |
| | | protein 2) | | | | | | |
| 25 | 201162_at | insulin-like | IGFBP7 | 4q12 | NM_001553.1 | 36.0 | Above | 4.3 |
| | | growth factor | | | | | | |
| | | binding protein 7 | | | | | | |
| 26 | 201163_s_at | insulin-like | IGFBP7 | 4q12 | NM_001553.1 | 36.0 | Above | 4.0 |
| | | growth factor | | | | | | |
| | | binding protein 7 | | | | | | |
| 27 | 203836_s_at | mitogen-activated | MAP3K5 | 6q22.33 | D84476.1 | 36.0 | Above | 13.9 |
| | | protein kinase | | | | | | |
| | | kinase kinase 5 | | | | | | |
| 28 | 203837_at | mitogen-activated | MAP3K5 | 6q22.33 | NM_005923.2 | 36.0 | Above | 4.2 |
| | | protein kinase | | | | | | |
| | | kinase kinase 5 | | | | | | |
| 29 | 213891_s_at | cDNA FLJ11918 | FLJ11918 | | AI927067 | 36.0 | Below | 3.2 |
| | | fis | | | | | | |
| 30 | 214895_s_at | a disintegrin and | ADAM10 | 15q22 | AU135154 | 36.0 | Above | 1.9 |
| | | metalloproteinase | | | | | | |
| | | domain 10 | | | | | | |
| 31 | 226415_at | KIAA1576 | KIAA1576 | 16q22.1 | AA156723 | 36.0 | Above | 40.7 |
| | | protein | | | | | | |
| 32 | 235879_at | ESTs | | | AI697540 | 36.0 | Above | 3.8 |
| 33 | 212387_at | cDNA FLJ11918 | FLJ11918 | | AK021980.1 | 35.8 | Below | 3.3 |
| | | fis | | | | | | |
| 34 | 218988_at | bladder cancer | BLOV1 | 12q15 | NM_018656.1 | 35.8 | Below | 16.3 |
| | | overexpressed | | | | | | |
| | | protein | | | | | | |
| 35 | 228555_at | EST; by BLAT | CAMK2D | | AA029441 | 35.8 | Above | 3.1 |
| | | calcium/calmodulin- | | | | | | |
| | | dependent | | | | | | |
| | | Protine Kinase | | | | | | |
| | | type II Delta chain | | | | | | |
| | | (CAMK GROUP | | | | | | |
| | | I) | | | | | | |
| 36 | 202975_s_at | Rho-related BTB | RHOBTB3 | 5q21.2 | N21138 | 35.3 | Above | 5.5 |
| | | domain containing 3 | | | | | | |
| 37 | 201105_at | lectin, galactoside- | LGALS1 | 22q13.1 | NM_002305.2 | 34.5 | Above | 14.5 |
| | | binding, soluble, 1 | | | | | | |
| | | (galectin 1) | | | | | | |
| 38 | 203434_s_at | membrane | MME | 3q25.1-q25.2 | AI433463 | 34.1 | Below | 31.2 |
| | | metallo- | | | | | | |
| | | endopeptidase | | | | | | |
| | | (neutral | | | | | | |
| | | endopeptidase, | | | | | | |
| | | enkephalinase, | | | | | | |
| | | CALLA, CD10) | | | | | | |
| 39 | 212135_s_at | calcium | ATP2B4 | | AW517686 | 34.1 | Below | 2.4 |
| | | transporting | | | | | | |
| | | ATPase plasma | | | | | | |
| | | membrane | | | | | | |
| | | protein. | | | | | | |
| 40 | 212136_at | calcium | ATP2B4 | | AW517686 | 34.1 | Below | 2.1 |
| | | transporting | | | | | | |
| | | ATPase plasma | | | | | | |
| | | membrane | | | | | | |
| | | protein. | | | | | | |
| 41 | 230179_at | cDNA | DKFZp547P158 | | N52572 | 34.1 | Below | 6.4 |
| | | DKFZp547P158 | | | | | | |
| 42 | 218217_at | likely homolog of | RISC | 17q23.2 | NM_021626.1 | 32.8 | Above | 3.4 |
| | | rat and mouse | | | | | | |
| | | retinoid-inducible | | | | | | |
| | | serine | | | | | | |
| | | carboxypeptidase | | | | | | |
| 43 | 225841_at | hypothetical | FLJ30525 | 1p13.2 | BE502436 | 32.8 | Above | 1.8 |
| | | protein FLJ30525 | | | | | | |
| 44 | 226668_at | Homo sapiens, | | | W80623 | 32.8 | Above | 2.4 |
| | | similar to WD | | | | | | |
| | | domain, G-beta | | | | | | |
| | | repeat containing | | | | | | |
| | | protein | | | | | | |
| 45 | 200989_at | hypoxia-inducible | HIF1A | 14q21-q24 | NM_001530.1 | 32.2 | Below | 1.8 |
| | | factor 1, alpha | | | | | | |
| | | subunit (basic | | | | | | |
| | | helix-loop-helix | | | | | | |
| | | transcription | | | | | | |
| | | factor) | | | | | | |
| 46 | 201151_s_at | muscleblind-like | MBNL | 3q25 | NM_021038.1 | 32.2 | Above | 2.6 |
| | | (Drosophila) | | | | | | |
| 47 | 201563_at | sorbitol | SORD | 15q15.3 | L29008.1 | 32.2 | Above | 1.8 |
| | | dehydrogenase | | | | | | |
| 48 | 203753_at | transcription | TCF4 | 18q21.1 | NM_003199.1 | 32.2 | Below | 2.9 |
| | | factor 4 | | | | | | |
| 49 | 205668_at | lymphocyte | LY75 | 2q24 | NM_002349.1 | 32.2 | Above | 2.1 |
| | | antigen 75 | | | | | | |
| 50 | 206471_s_at | plexin C1 | PLXNC1 | 12q23.3 | NM_005761.1 | 32.2 | Above | 7.7 |
| 51 | 211302_s_at | phosphodiesterase | PDE4B | 1p31 | L20966.1 | 32.2 | Below | 3.0 |
| | | 4B, cAMP- | | | | | | |
| | | specific | | | | | | |
| 52 | 212012_at | Melanoma | D2S448 | 2pter- | AF200348.1 | 32.2 | Below | 2.4 |
| | | associated gene | | p25.1 | | | | |
| 53 | 212063_at | CD44 antigen | CD44 | 11p13 | BE903880 | 32.2 | Above | 3.1 |
| 54 | 213241_at | PLEXIN c1 | PLXNC1 | | AF035307.1 | 32.2 | Above | 2.5 |
| 55 | 214651_s_at | homeo box A9 | HOXA9 | 7p15-p14 | U41813.1 | 32.2 | Above | 28.5 |
| 56 | 218140_x_at | APMCF1 protein | APMCF1 | 3q22.2 | NM_021203.1 | 32.2 | Above | 1.4 |
| 57 | 219988_s_at | hypothetical | FLJ10597 | 1p34.1 | NM_018150.1 | 32.2 | Above | 1.9 |
| | | protein FLJ10597 | | | | | | |
| 58 | 223046_at | egl nine homolog | EGLN1 | 1q42.1 | NM_022051.1 | 32.2 | Below | 4.2 |
| | | 1 (C. elegans) | | | | | | |
| 59 | 224150_s_at | p10-binding | BITE | 3q22-q23 | AF289495.1 | 32.2 | Above | 2.1 |
| | | protein | | | | | | |
| 60 | 224933_s_at | hypothetical | DKFZp761F0118 | 10q22.1 | AB037801.1 | 32.2 | Above | 1.9 |
| | | protein | | | | | | |
| | | DKFZp761F0118 | | | | | | |
| 61 | 201078_at | transmembrane 9 | TM9SF2 | 13q32.3 | NM_004800.1 | 32.0 | Above | 1.5 |
| | | superfamily | | | | | | |
| | | member 2 | | | | | | |
| 62 | 205550_s_at | brain and | BRE | 2p23.3 | NM_004899.1 | 32.0 | Above | 2.0 |
| | | reproductive | | | | | | |
| | | organ-expressed | | | | | | |
| | | (TNFRSF1A | | | | | | |
| | | modulator) | | | | | | |
| 63 | 212382_at | cDNA FLJ11918 | FLJ11918 | | AK021980.1 | 32.0 | Below | 2.7 |
| | | fis | | | | | | |
| 64 | 225019_at | calcium/calmodulin- | CAMK2D | 4q25 | AA777512 | 32.0 | Above | 3.6 |
| | | dependent | | | | | | |
| | | protein kinase | | | | | | |
| | | (CaM kinase) II | | | | | | |
| | | delta | | | | | | |
| 65 | 225202_at | Rho-related BTB | RHOBTB3 | 5q21.2 | BE620739 | 32.0 | Above | 5.5 |
| | | domain containing 3 | | | | | | |
| 66 | 228855_at | nudix (nucleoside | NUDT7 | | AI927964 | 32.0 | Above | 5.6 |
| | | diphosphate | | | | | | |
| | | linked moiety X)- | | | | | | |
| | | type motif 7 | | | | | | |
| 67 | 231899_at | KIAA1726 | KIAA1726 | 11q23.1 | AB051513.1 | 32.0 | Above | 33.0 |
| | | protein | | | | | | |
| 68 | 52164_at | chromosome 11 | C11orf24 | 11q13 | AA065185 | 32.0 | Above | 2.3 |
| | | open reading | | | | | | |
| | | frame 24 | | | | | | |
| 69 | 212660_at | KIAA0239 | KIAA0239 | 5q31.1 | AI735639 | 31.7 | Below | 1.7 |
| | | protein | | | | | | |
| 70 | 213513_x_at | actin related | ARPC2 | 2q36.1 | BG034239 | 31.7 | Above | 1.3 |
| | | protein 2/3 | | | | | | |
| | | complex, subunit | | | | | | |
| | | 2, 34 kDa | | | | | | |
| 71 | 222603_at | hypothetical | FLJ23309 | 9p24 | AL136980 | 31.7 | Above | 3.6 |
| | | protein FLJ23309 | | | | | | |
| 72 | 238558_at | ESTs | | | AI445833 | 31.7 | Above | 3.8 |
| 73 | 202391_at | brain abundant, | BASP1 | 5p15.1-p14 | NM_006317.1 | 31.3 | Above | 2.1 |
| | | membrane | | | | | | |
| | | attached signal | | | | | | |
| | | protein 1 | | | | | | |
| 74 | 202604_x_at | a disintegrin and | ADAM10 | 15q22 | NM_001110.1 | 31.3 | Above | 1.8 |
| | | metalloproteinase | | | | | | |
| | | domain 10 | | | | | | |
| 75 | 203435_s_at | membrane | MME | 3q25.1-q25.2 | NM_007287.1 | 31.3 | Below | 54.8 |
| | | metallo- | | | | | | |
| | | endopeptidase | | | | | | |
| | | (neutral | | | | | | |
| | | endopeptidase, | | | | | | |
| | | enkephalinase, | | | | | | |
| | | CALLA, CD10) | | | | | | |
| 76 | 204445_s_at | arachidonate 5- | ALOX5 | 10q11.2 | AI361850 | 31.3 | Below | 687.0 |
| | | lipoxygenase | | | | | | |
| 77 | 209705_at | likely ortholog of | M96 | 1p22.1 | AF073293.1 | 31.3 | Below | 1.5 |
| | | mouse metal | | | | | | |
| | | response element | | | | | | |
| | | binding | | | | | | |
| | | transcription | | | | | | |
| | | factor 2 | | | | | | |
| 78 | 214366_s_at | arachidonate 5- | ALOX5 | 10q11.2 | AA995910 | 31.3 | Below | 54.7 |
| | | lipoxygenase | | | | | | |
| 79 | 215000_s_at | fasciculation and | FEZ2 | 2p21 | AL117593.1 | 31.3 | Above | 1.7 |
| | | elongation protein | | | | | | |
| | | zeta 2 (zygin II) | | | | | | |
| 80 | 220643_s_at | Fas apoptotic | FAIM | 3q23 | NM_018147.1 | 31.3 | Above | 2.9 |
| | | inhibitory | | | | | | |
| | | molecule | | | | | | |
| 81 | 226459_at | Homo sapiens | | | AW575754 | 31.3 | Above | 1.6 |
| | | gastric cancer- | | | | | | |
| | | related protein | | | | | | |
| | | GCYS-20 (gcys- | | | | | | |
| | | 20) mRNA, | | | | | | |
| | | complete cds; | | | | | | |
| | | homology with | | | | | | |
| | | mouse epidermal | | | | | | |
| | | growth factor | | | | | | |
| | | receptor pathway | | | | | | |
| | | substrate 8 | | | | | | |
| 82 | 238712_at | ESTs | | | BF801735 | 31.3 | Above | 2.7 |
| 83 | 229686_at | cDNA FLJ35637 | FLJ35637 | | AI436587 | 31.0 | Below | 1.5 |
| | | fis | | | | | | |
| 84 | 222620_s_at | hypothetical | DNAJL1 | 10p11.23 | BF591419 | 29.8 | Above | 2.4 |
| | | protein similar to | | | | | | |
| | | mouse Dnajl1 | | | | | | |
| 85 | 224516_s_at | hypothetical | HSPC195 | 5q31.3 | BC006428.1 | 29.8 | Above | 2.7 |
| | | protein HSPC195 | | | | | | |
| 86 | 203217_s_at | sialyltransferase 9 | SIAT9 | 2p11.2 | NM_003896.1 | 28.8 | Below | 2.1 |
| | | (CMP- | | | | | | |
| | | NeuAc: lactosylceramide | | | | | | |
| | | alpha-2,3- | | | | | | |
| | | sialyltransferase; | | | | | | |
| | | GM3 synthase) | | | | | | |
| 87 | 204030_s_at | schwannomin | SCHIP1 | 3q25.32 | NM_014575.1 | 28.8 | Below | 17.6 |
| | | interacting protein 1 | | | | | | |
| 88 | 209191_at | tubulin beta-5 | TUBB-5 | | BC002654.1 | 28.8 | Above | 6.4 |
| 89 | 213541_s_at | v-ets | ERG | 21q22.3 | AI351043 | 28.8 | Below | 2.8 |
| | | erythroblastosis | | | | | | |
| | | virus E26 | | | | | | |
| | | oncogene like | | | | | | |
| | | (avian) | | | | | | |
| 90 | 213773_x_at | Williams Beuren | WBSCR20A | 7q11.23 | AW248552 | 28.8 | Above | 1.3 |
| | | syndrome | | | | | | |
| | | chromosome | | | | | | |
| | | region 20A | | | | | | |
| 91 | 219243_at | immunity | HIMAP4 | 7q35 | NM_018326.1 | 28.8 | Below | 13.4 |
| | | associated protein 4 | | | | | | |
| 92 | 219256_s_at | hypothetical | FLJ20356 | 4p16.1 | NM_018986.1 | 28.8 | Below | 2.6 |
| | | protein FLJ20356 | | | | | | |
| 93 | 223358_s_at | phosphodiesterase | PDE7A | 8q13 | AW269834 | 28.8 | Above | 1.5 |
| | | 7A | | | | | | |
| 94 | 224796_at | development and | DDEF1 | 8q24.1-q24.2 | W03103 | 28.8 | Below | 1.8 |
| | | differentiation | | | | | | |
| | | enhancing factor 1 | | | | | | |
| 95 | 203076_s_at | MAD, mothers | MADH2 | 18q21.1 | U65019.1 | 28.7 | Below | 2.0 |
| | | against | | | | | | |
| | | decapentaplegic | | | | | | |
| | | homolog 2 | | | | | | |
| | | (Drosophila) | | | | | | |
| 96 | 212385_at | cDNA FLJ11918 | FLJ11918 | | AK021980.1 | 28.7 | Below | 3.2 |
| | | fis | | | | | | |
| 97 | 216026_s_at | polymerase (DNA | POLE | 12q24.3 | AL080203.1 | 28.7 | Below | 3.0 |
| | | directed), epsilon | | | | | | |
| 98 | 217118_s_at | KIAA0930 | KIAA0930 | 22q13.31 | AK025608.1 | 28.7 | Above | 1.9 |
| | | protein | | | | | | |
| 99 | 219821_s_at | hypothetical | FLJ20330 | 6pter- | NM_018988.1 | 28.7 | Below | 5.5 |
| | | protein FLJ20330 | | p22.1 | | | | |
| 100 | 201875_s_at | hypothetical | FLJ21047 | 1q23.2 | NM_024569.1 | 28.5 | Above | 2.0 |
| | | protein FLJ21047 | | | | | | |

TABLE 67
Top 100 chi-square probe sets selected for T-ALL

| | | | | | | | T-ALL | |
|---|---|---|---|---|---|---|---|---|
| | | | | | | | above/ | |
| | U133 probe | | | Chromosomal | | Chi- | below | Fold |
| | set | Gene Description | Symbol | Location | GenBank Ref | square | mean | change |
| 1 | 201137_s_at | major | HLA- | 6p21.3 | NM_002121.1 | 100.0 | Below | 21.0 |
| | | histocompatibility | DPB1 | | | | | |
| | | complex, class II, | | | | | | |
| | | DP beta 1 | | | | | | |
| 2 | 202113_s_at | sorting nexin 2 | SNX2 | 5q23 | AF043453.1 | 100.0 | Below | 4.2 |
| 3 | 202114_at | sorting nexin 2 | SNX2 | 5q23 | NM_003100.1 | 100.0 | Below | 4.6 |
| 4 | 203675_at | nucleobindin 2 | NUCB2 | 11p15.1-p14 | NM_005013.1 | 100.0 | Above | 3.6 |
| 5 | 204670_x_at | major | HLA- | 6p21.3 | NM_002125.1 | 100.0 | Below | 13.4 |
| | | histocompatibility | DRB3 | | | | | |
| | | complex, class II, | | | | | | |
| | | DR beta 3 | | | | | | |
| 6 | 205297_s_at | CD79B antigen | CD79B | 17q23 | NM_000626.1 | 100.0 | Below | 23.3 |
| | | (immunoglobulin- | | | | | | |
| | | associated beta) | | | | | | |
| 7 | 205456_at | CD3E antigen, | CD3E | 11q23 | NM_000733.1 | 100.0 | Above | 20.7 |
| | | epsilon | | | | | | |
| | | polypeptide (TiT3 | | | | | | |
| | | complex) | | | | | | |
| 8 | 206398_s_at | CD19 antigen | CD19 | 16p11.2 | NM_001770.1 | 100.0 | Below | 5693.6 |
| 9 | 208306_x_at | major | HLA- | 6p21.3 | NM_021983.2 | 100.0 | Below | 8.3 |
| | | histocompatibility | DRB4 | | | | | |
| | | complex, class II, | | | | | | |
| | | DR beta 4 | | | | | | |
| 10 | 208894_at | major | HLA- | 6p21.3 | M60334.1 | 100.0 | Below | 20.9 |
| | | histocompatibility | DRA | | | | | |
| | | complex, class II, | | | | | | |
| | | DR alpha | | | | | | |
| 11 | 209312_x_at | major | HLA- | 6p21.3 | U65585.1 | 100.0 | Below | 12.6 |
| | | histocompatibility | DRB1 | | | | | |
| | | complex, class II, | | | | | | |
| | | DR beta 1 | | | | | | |
| 12 | 209619_at | CD74 antigen | CD74 | 5q32 | K01144.1 | 100.0 | Below | 15.1 |
| | | (invariant | | | | | | |
| | | polypeptide of | | | | | | |
| | | major | | | | | | |
| | | histocompatibility | | | | | | |
| | | complex, class II | | | | | | |
| | | antigen- | | | | | | |
| | | associated) | | | | | | |
| 13 | 210116_at | SH2 domain | SH2D1A | Xq25-q26 | AF072930.1 | 100.0 | Above | 150.7 |
| | | protein 1A, | | | | | | |
| | | Duncan's disease | | | | | | |
| | | (lymphoproliferative | | | | | | |
| | | syndrome) | | | | | | |
| 14 | 210982_s_at | major | HLA- | 6p21.3 | M60333.1 | 100.0 | Below | 23.4 |
| | | histocompatibility | DRA | | | | | |
| | | complex, class II, | | | | | | |
| | | DR alpha | | | | | | |
| 15 | 211990_at | major | HLA- | 6p21.3 | M27487.1 | 100.0 | Below | 19.6 |
| | | histocompatibility | DPA1 | | | | | |
| | | complex, class II, | | | | | | |
| | | DP alpha 1 | | | | | | |
| 16 | 211991_s_at | major | HLA- | 6p21.3 | M27487.1 | 100.0 | Below | 24.5 |
| | | histocompatibility | DPA1 | | | | | |
| | | complex, class II, | | | | | | |
| | | DP alpha 1 | | | | | | |
| 17 | 213539_at | CD3D antigen, | CD3D | 11q23 | NM_000732.1 | 100.0 | Above | 35.7 |
| | | delta polypeptide | | | | | | |
| | | (TiT3 complex) | | | | | | |
| 18 | 214049_x_at | CD7 antigen (p41) | CD7 | 17q25.2-q25.3 | AI829961 | 100.0 | Above | 312.2 |
| 19 | 214551_s_at | CD7 antigen (p41) | CD7 | 17q25.2-q25.3 | NM_006137.2 | 100.0 | Above | 228.1 |
| 20 | 217147_s_at | T-cell receptor | TRIM | 3q13 | AJ240085.1 | 100.0 | Above | 42.6 |
| | | interacting | | | | | | |
| | | molecule | | | | | | |
| 21 | 217478_s_at | MHC, class IIa, | HLA- | | X76775 | 100.0 | Below | 11.9 |
| | | HLA-DMA | DMA | | | | | |
| 22 | 221969_at | paired box gene 5 | PAX5 | 9p13 | BF510692 | 100.0 | Below | 3922.0 |
| | | (B-cell lineage | | | | | | |
| | | specific activator | | | | | | |
| | | protein) | | | | | | |
| 23 | 227646_at | early B-cell factor | EBF | 5q34 | BG435302 | 100.0 | Below | 85.0 |
| 24 | 229487_at | cDNA FLJ39389 | FLJ39389 | 5 | W73890 | 100.0 | Below | 7685.7 |
| | | fis | | | | | | |
| 25 | 229838_at | cDNA FLJ39156 | FLJ39156 | | AI377271 | 100.0 | Above | 12.7 |
| | | fis | | | | | | |
| 26 | 232204_at | early B-cell factor | EBF | 5q34 | AF208502.1 | 100.0 | Below | 7129.1 |
| 27 | 203965_at | ubiquitin specific | USP20 | 9q34.12-q34.13 | NM_006676.1 | 91.3 | Above | 9.0 |
| | | protease 20 | | | | | | |
| 28 | 204891_s_at | lymphocyte- | LCK | 1p34.3 | NM_005356.1 | 91.3 | Above | 13.8 |
| | | specific protein | | | | | | |
| | | tyrosine kinase | | | | | | |
| 29 | 205255_x_at | transcription | TCF7 | 5q31.1 | NM_003202.1 | 91.3 | Above | 8.4 |
| | | factor 7 (T-cell | | | | | | |
| | | specific, HMG- | | | | | | |
| | | box) | | | | | | |
| 30 | 207655_s_at | B-cell linker | BLNK | 10q23.2-q23.33 | NM_013314.1 | 91.3 | Below | 103.2 |
| 31 | 209771_x_at | CD24 antigen | CD24 | 6q21 | AA761181 | 91.3 | Below | 40.1 |
| | | (small cell lung | | | | | | |
| | | carcinoma cluster | | | | | | |
| | | 4 antigen) | | | | | | |
| 32 | 211796_s_at | T cell receptor | TRB | 7q34 | AF043179.1 | 91.3 | Above | 20.7 |
| | | beta locus | | | | | | |
| 33 | 213792_s_at | insulin receptor | INSR | 19p13.3-p13.2 | AA485908 | 91.3 | Below | 8.0 |
| 34 | 215193_x_at | major | HLA- | 6p21.3 | AJ297586.1 | 91.3 | Below | 12.1 |
| | | histocompatibility | DRB3 | | | | | |
| | | complex, class II, | | | | | | |
| | | DR beta 3 | | | | | | |
| 35 | 216379_x_at | KIAA1919 | KIAA1919 | 6q22.1 | AK000168.1 | 91.3 | Below | 44.0 |
| | | protein | | | | | | |
| 36 | 219191_s_at | bridging integrator 2 | BIN2 | 12q13 | NM_016293.1 | 91.3 | Above | 271.0 |
| 37 | 219563_at | hypothetical | FLJ21276 | 14q32.2 | NM_024633.1 | 91.3 | Below | 5.8 |
| | | protein FLJ21276 | | | | | | |
| 38 | 219724_s_at | KIAA0748 gene | KIAA0748 | 12q12 | NM_014796.1 | 91.3 | Above | 11.6 |
| | | product | | | | | | |
| 39 | 221750_at | 3-hydroxy-3- | HMGCS1 | 5p14-p13 | BG035985 | 91.3 | Above | 3.4 |
| | | methylglutaryl- | | | | | | |
| | | Coenzyme A | | | | | | |
| | | synthase 1 | | | | | | |
| | | (soluble) | | | | | | |
| 40 | 226157_at | cDNA FLJ39131 | FLJ39131 | 3 | AI569747 | 91.3 | Above | 4.4 |
| | | fis | | | | | | |
| 41 | 226496_at | hypothetical | FLJ22611 | 9p11.1 | BG291039 | 91.3 | Below | 7.6 |
| | | protein FLJ22611 | | | | | | |
| 42 | 266_s_at | CD24 antigen | CD24 | 6q21 | L33930 | 91.3 | Below | 69.7 |
| | | (small cell lung | | | | | | |
| | | carcinoma cluster | | | | | | |
| | | 4 antigen) | | | | | | |
| 43 | 39318_at | T-cell | TCL1A | 14q32.1 | X82240 | 91.3 | Below | 367.4 |
| | | leukemia/lymphoma | | | | | | |
| | | 1A | | | | | | |
| 44 | 204214_s_at | RAB32, member | RAB32 | 6q24.3 | NM_006834.1 | 90.6 | Above | 127.9 |
| | | RAS oncogene | | | | | | |
| | | family | | | | | | |
| 45 | 204777_s_at | mal, T-cell | MAL | 2cen-q13 | NM_002371.2 | 90.6 | Above | 96.8 |
| | | differentiation | | | | | | |
| | | protein | | | | | | |
| 46 | 204890_s_at | lymphocyte- | LCK | 1p34.3 | U07236.1 | 90.6 | Above | 18.6 |
| | | specific protein | | | | | | |
| | | tyrosine kinase | | | | | | |
| 47 | 205049_s_at | CD79A antigen | CD79A | 19q13.2 | NM_001783.1 | 90.6 | Below | 11.4 |
| | | (immunoglobulin- | | | | | | |
| | | associated alpha) | | | | | | |
| 48 | 205254_x_at | transcription | TCF7 | 5q31.1 | AW027359 | 90.6 | Above | 352.0 |
| | | factor 7 (T-cell | | | | | | |
| | | specific, HMG- | | | | | | |
| | | box) | | | | | | |
| 49 | 205504_at | Bruton | BTK | Xq21.33-q22 | NM_000061.1 | 90.6 | Below | 6.6 |
| | | agammaglobuline | | | | | | |
| | | mia tyrosine | | | | | | |
| | | kinase | | | | | | |
| 50 | 210915_x_at | T cell receptor | TRB | 7q34 | M15564.1 | 90.6 | Above | 15.9 |
| | | beta locus | | | | | | |
| 51 | 211211_x_at | SH2 domain | SH2D1A | Xq25-q26 | AF100542.1 | 90.6 | Above | 1963.5 |
| | | protein 1A, | | | | | | |
| | | Duncan's disease | | | | | | |
| | | (lymphoproliferative | | | | | | |
| | | syndrome) | | | | | | |
| 52 | 213830_at | T cell receptor | TRD | 14q11.2 | AW007751 | 90.6 | Above | 7411.2 |
| | | delta locus | | | | | | |
| 53 | 216191_s_at | T cell receptor | TRD | 14q11.2 | X72501.1 | 90.6 | Above | 253.7 |
| | | delta locus | | | | | | |
| 54 | 217143_s_at | T cell receptor | TRD | 14q11.2 | X06557.1 | 90.6 | Above | 151.9 |
| | | delta locus | | | | | | |
| 55 | 219528_s_at | B-cell | BCL11B | 14q32.31-q32.32 | NM_022898.1 | 90.6 | Above | 11.6 |
| | | CLL/lymphoma | | | | | | |
| | | 11B (zinc finger | | | | | | |
| | | protein) | | | | | | |
| 56 | 220418_at | ubiquitin | UBASH3A | 21q22.3 | NM_018961.1 | 90.6 | Above | 759.3 |
| | | associated and | | | | | | |
| | | SH3 domain | | | | | | |
| | | containing, A | | | | | | |
| 57 | 222895_s_at | B-cell | BCL11B | 14q32.31-q32.32 | AA918317 | 90.6 | Above | 11.7 |
| | | CLL/lymphoma | | | | | | |
| | | 11B (zinc finger | | | | | | |
| | | protein) | | | | | | |
| 58 | 223553_s_at | hypothetical | FLJ22570 | 5q35.3 | BC004564.1 | 90.6 | Below | 6.1 |
| | | protein FLJ22570 | | | | | | |
| 59 | 225090_at | HRD1 protein | HRD1 | 11q12 | AA844682 | 90.6 | Below | 3.6 |
| 60 | 226459_at | Homo sapiens | | | AW575754 | 90.6 | Below | 10.7 |
| | | gastric cancer- | | | | | | |
| | | related protein | | | | | | |
| | | GCYS-20 (gcys- | | | | | | |
| | | 20) mRNA, | | | | | | |
| | | complete cds | | | | | | |
| 61 | 228314_at | cDNA FLJ37485 | FLJ37485 | | BE877357 | 90.6 | Below | 4.7 |
| | | fis | | | | | | |
| 62 | 201384_s_at | membrane | M17S2 | 17q21.1 | NM_005899.1 | 83.8 | Above | 3.3 |
| | | component, | | | | | | |
| | | chromosome 17, | | | | | | |
| | | surface marker 2 | | | | | | |
| | | (ovarian | | | | | | |
| | | carcinoma antigen | | | | | | |
| | | CA125) | | | | | | |
| 63 | 202540_s_at | 3-hydroxy-3- | HMGCR | 5q13.3-q14 | NM_000859.1 | 83.8 | Above | 4.4 |
| | | methylglutaryl- | | | | | | |
| | | Coenzyme A | | | | | | |
| | | reductase | | | | | | |
| 64 | 203198_at | cyclin-dependent | CDK9 | 9q34.1 | NM_001261.1 | 83.8 | Below | 4.8 |
| | | kinase 9 (CDC2- | | | | | | |
| | | related kinase) | | | | | | |
| 65 | 203932_at | major | HLA- | 6p21.3 | NM_002118.1 | 83.8 | Below | 7.9 |
| | | histocompatibility | DMB | | | | | |
| | | complex, class II, | | | | | | |
| | | DM beta | | | | | | |
| 66 | 204613_at | phospholipase C, | PLCG2 | 16q24.1 | NM_002661.1 | 83.8 | Below | 3.9 |
| | | gamma 2 | | | | | | |
| | | (phosphatidylinositol- | | | | | | |
| | | specific) | | | | | | |
| 67 | 205267_at | POU domain, | POU2AF1 | 11q23.1 | NM_006235.1 | 83.8 | Below | 11.2 |
| | | class 2, | | | | | | |
| | | associating factor 1 | | | | | | |
| 68 | 208650_s_at | CD24 antigen | CD24 | 6q21 | BG327863 | 83.8 | Below | 74.7 |
| | | (small cell lung | | | | | | |
| | | carcinoma cluster | | | | | | |
| | | 4 antigen) | | | | | | |
| 69 | 208651_x_at | CD24 antigen | CD24 | 6q21 | M58664.1 | 83.8 | Below | 52.7 |
| | | (small cell lung | | | | | | |
| | | carcinoma cluster | | | | | | |
| | | 4 antigen) | | | | | | |
| 70 | 209995_s_at | T-cell | TCL1A | 14q32.1 | BC003574.1 | 83.8 | Below | 20166.2 |
| | | leukemia/lymphoma 1A | | | | | | |
| 71 | 210038_at | protein kinase C, | PRKCQ | 10p15 | AL137145 | 83.8 | Above | 12.7 |
| | | theta | | | | | | |
| 72 | 211126_s_at | cysteine and | CSRP2 | 12q21.1 | U46006.1 | 83.8 | Below | 18.0 |
| | | glycine-rich | | | | | | |
| | | protein 2 | | | | | | |
| 73 | 220068_at | pre-B lymphocyte | VPREB3 | 22q11.23 | NM_013378.1 | 83.8 | Below | 6559.8 |
| | | gene 3 | | | | | | |
| 74 | 226245_at | cDNA | DKFZp451C132 | | U55984 | 83.8 | Above | 8.7 |
| | | DKFZp451C132 | | | | | | |
| 75 | 202615_at | cDNA | DKFZp686D0521 | | BF222895 | 82.2 | Above | 3.1 |
| | | DKFZp686D0521 | | | | | | |
| 76 | 224861_at | cDNA FLJ31057 | FLJ31057 | | BF477658 | 82.2 | Above | 3.5 |
| | | fis | | | | | | |
| 77 | 201194_at | selenoprotein W, 1 | SEPW1 | 19q13.3 | NM_003009.1 | 82.0 | Above | 3.8 |
| 78 | 201349_at | solute carrier | SLC9A3R1 | 17q25.2 | NM_004252.1 | 82.0 | Above | 2.9 |
| | | family 9 | | | | | | |
| | | (sodium/hydrogen | | | | | | |
| | | exchanger), | | | | | | |
| | | isoform 3 | | | | | | |
| | | regulatory factor 1 | | | | | | |
| 79 | 202539_s_at | 3-hydroxy-3- | HMGCR | 5q13.3-q14 | AL518627 | 82.0 | Above | 3.5 |
| | | methylglutaryl- | | | | | | |
| | | Coenzyme A | | | | | | |
| | | reductase | | | | | | |
| 80 | 203588_s_at | transcription | TFDP2 | 3q23 | BG034328 | 82.0 | Above | 17.5 |
| | | factor Dp-2 (E2F | | | | | | |
| | | dimerization | | | | | | |
| | | partner 2) | | | | | | |
| 81 | 204852_s_at | protein tyrosine | PTPN7 | 1q32.1 | NM_002832.1 | 82.0 | Above | 9.5 |
| | | phosphatase, non- | | | | | | |
| | | receptor type 7 | | | | | | |
| 82 | 207434_s_at | FXYD domain | FXYD2 | 11q23 | NM_021603.1 | 82.0 | Above | 14.6 |
| | | containing ion | | | | | | |
| | | transport regulator 2 | | | | | | |
| 83 | 208872_s_at | DNA segment, | D5S346 | 5q22-q23 | AA814140 | 82.0 | Below | 2.6 |
| | | single copy probe | | | | | | |
| | | LNS-CAI/LNS- | | | | | | |
| | | CAII | | | | | | |
| 84 | 209200_at | MADS box | MEF2C | 5q14 | N22468 | 82.0 | Below | 7.5 |
| | | transcription | | | | | | |
| | | enhancer factor 2, | | | | | | |
| | | polypeptide C | | | | | | |
| | | (myocyte | | | | | | |
| | | enhancer factor | | | | | | |
| | | 2C) | | | | | | |
| 85 | 212795_at | KIAA1033 | KIAA1033 | 12q24.11 | AL137753.1 | 82.0 | Below | 2.4 |
| | | protein | | | | | | |
| 86 | 212827_at | immunoglobulin | IGHM | 14q32.33 | X17115.1 | 82.0 | Below | 13.1 |
| | | heavy constant mu | | | | | | |
| 87 | 213193_x_at | T cell receptor | TRB | 7q34 | AL559122 | 82.0 | Above | 10.9 |
| | | beta locus | | | | | | |
| 88 | 221002_s_at | tetraspanin similar | DC- | 10q23.2 | NM_030927.1 | 82.0 | Below | 2.1 |
| | | to TM4SF9 | TM4F2 | | | | | |
| 89 | 225314_at | hypothetical | MGC45416 | 4p12 | BG291649 | 82.0 | Above | 5.5 |
| | | protein | | | | | | |
| | | MGC45416 | | | | | | |
| 90 | 227432_s_at | insulin receptor | INSR | 19p13.3-p13.2 | AI215106 | 82.0 | Below | 6.0 |
| 91 | 203332_s_at | inositol | INPP5D | 2q36-q37 | NM_005541.1 | 81.5 | Below | 2.2 |
| | | polyphosphate-5- | | | | | | |
| | | phosphatase, | | | | | | |
| | | 145 kDa | | | | | | |
| 92 | 203589_s_at | transcription | TFDP2 | 3q23 | NM_006286.1 | 81.5 | Above | 35.1 |
| | | factor Dp-2 (E2F | | | | | | |
| | | dimerization | | | | | | |
| | | partner 2) | | | | | | |
| 93 | 205674_x_at | FXYD domain | FXYD2 | 11q23 | NM_001680.2 | 81.5 | Above | 12.2 |
| | | containing ion | | | | | | |
| | | transport regulator 2 | | | | | | |
| 94 | 209881_s_at | Linker for | LAT | 16q13 | AF036905.1 | 81.5 | Above | 1823.4 |
| | | activation of T | | | | | | |
| | | cells | | | | | | |
| 95 | 211005_at | Linker for | LAT | 16q13 | AF036906.1 | 81.5 | Above | 67.8 |
| | | activation of T | | | | | | |
| | | cells | | | | | | |
| 96 | 211075_s_at | CD47 | CD47 | | Z25521.1 | 81.5 | Above | 2.1 |
| 97 | 211210_x_at | SH2 domain | SH2D1A | Xq25-q26 | AF100539.1 | 81.5 | Above | 300.2 |
| | | protein 1A, | | | | | | |
| | | Duncan's disease | | | | | | |
| | | (lymphoproliferative | | | | | | |
| | | syndrome) | | | | | | |
| 98 | 213601_at | slit homolog 1 | SLITI | 10q23.3-q24 | AB011537.2 | 81.5 | Above | 1752.1 |
| | | (Drosophila) | | | | | | |
| 99 | 213857_s_at | CD47 antigen | CD47 | 3q13.1-q13.2 | BG230614 | 81.5 | Above | 2.2 |
| | | (Rh-related | | | | | | |
| | | antigen, integrin- | | | | | | |
| | | associated signal | | | | | | |
| | | transducer) | | | | | | |
| 100 | 214924_s_at | KIAA1042 | KIAA1042 | 3p25.3-p24.1 | AK000754.1 | 81.5 | Below | 2.3 |
| | | protein | | | | | | |

TABLE 68
Top 100 chi-square probe sets selected for TEL-AML1

| | | | | | | | TEL- | |
|---|---|---|---|---|---|---|---|---|
| | | | | | | | AML | |
| | | | | | | Chi- | above/ | |
| | U133 probe | Gene | | Chromosomal | | square | below | Fold |
| | set | Description | Symbol | Location | GenBank Ref | value | mean | change |
| 1 | 224722_at | KIAA1323 | KIAA1323 | 18q11.1 | W80418 | 75 | Above | 7.6 |
| 2 | 227377_at | FLJ12722 | FLJ12722 | 17q21.32 | AK022784.1 | 75 | Above | 2446.3 |
| 3 | 237206_at | EST | | 17p12 | AI452798 | 75 | Above | 23.7 |
| 4 | 241505_at | EST | | | BF513468 | 75 | Above | 13.4 |
| 5 | 203184_at | Fibrillin 2 | FBN2 | 5q23.2 | NM_001999.2 | 69.1 | Above | 14.4 |
| | | (congenital | | | | | | |
| | | contractural | | | | | | |
| | | arachnodactyly) | | | | | | |
| 6 | 205109_s_at | Rho guanine | ARHGEF4 | 2q22 | NM_015320.1 | 69.1 | Above | 148.1 |
| | | nucleotide | | | | | | |
| | | exchange factor | | | | | | |
| | | (GEF) 4 | | | | | | |
| 7 | 210650_s_at | Piccolo | PCLO | 7q21.11 | BC001304.1 | 69.1 | Above | 101.2 |
| 8 | 213558_at | Piccolo | PCLO | 7q21.11 | AB011131.1 | 69.1 | Above | 77.5 |
| 9 | 220451_s_at | Livin IAP | BIRC7 | 20q13.3 | NM_022161.1 | 69.1 | Above | 25.4 |
| | | (inhibitor of | | | | | | |
| | | apoptosis) | | | | | | |
| 10 | 224720_at | KIAA1323 | KIAA1323 | 18q11.1 | W80418 | 69.1 | Above | 4.3 |
| 11 | 235694_at | IMAGE: 4661943 | | 20q13.33 | N49233 | 69.1 | Above | 9.3 |
| | | Unknown EST | | | | | | |
| 12 | 202808_at | Hypothetical | FLJ20154 | 10q24.32 | AK000161.1 | 68.9 | Above | 3.7 |
| | | protein FLJ20154 | | | | | | |
| 13 | 206032_at | Desmocollin 3 | DSC3 | 18q12.1 | AI797281 | 68.9 | Above | 54.1 |
| 14 | 206033_s_at | Desmocollin 3 | DSC3 | 18q12.1 | NM_001941.2 | 68.9 | Above | 357.1 |
| 15 | 209228_x_at | Putative prostate | N33 | 8p22 | U42349.1 | 68.9 | Above | 20.8 |
| | | cancer tumor | | | | | | |
| | | suppressor gene | | | | | | |
| | | N33 | | | | | | |
| 16 | 224725_at | KIAA1323 | KIAA1323 | 18q11.1 | W80418 | 68.9 | Above | 3.6 |
| 17 | 203910_at | PTPL1-associated | PARG1 | 1p22.1 | NM_004815.1 | 64 | Above | 7.1 |
| | | RhoGAP | | | | | | |
| 18 | 204849_at | Transcription | TCFL5 | 20q13.33 | NM_006602.1 | 64 | Above | 8.9 |
| | | factor-like 5 | | | | | | |
| | | (helix-loop-helix | | | | | | |
| | | domain) | | | | | | |
| 19 | 206231_at | Potassium | KCNN1 | 19p13.1 | NM_002248.2 | 64 | Above | 72.7 |
| | | intermediate/small | | | | | | |
| | | conductance | | | | | | |
| | | calcium-activated | | | | | | |
| | | channel, | | | | | | |
| | | subfamily N, | | | | | | |
| | | member 1 | | | | | | |
| 20 | 208056_s_at | Core-binding | CBFA2T3 | 16q24 | NM_005187.2 | 63 | Above | 2.5 |
| | | factor, runt | | | | | | |
| | | domain, alpha | | | | | | |
| | | subunit 2; | | | | | | |
| | | translocated to, 3 | | | | | | |
| 21 | 211222_s_at | Huntingtin- | HAP1 | 17q21.2 | AF040723.1 | 63 | Above | 80.8 |
| | | associated protein | | | | | | |
| | | 1 (neuroan 1, | | | | | | |
| | | HAP-1) | | | | | | |
| 22 | 223468_s_at | hypothetical | RGM | 15q26.1 | AL136826.1 | 63 | Above | 10.6 |
| | | protein from | | | | | | |
| | | EUROIMAGE | | | | | | |
| | | 363668 RGM: | | | | | | |
| | | likely ortholog of | | | | | | |
| | | chicken repulsive | | | | | | |
| | | guidance molecule | | | | | | |
| 23 | 227266_s_at | FYN-binding | FYB | 5p13.1 | BF679849 | 63 | Above | 3.1 |
| | | protein | | | | | | |
| 24 | 228158_at | Lymphocyte- | | 2p11.1 | AI623211 | 63 | Above | 7.9 |
| | | specific protein 1 | | | | | | |
| 25 | 37986_at | EPO receptor | EPOR | 19p13.2 | M60459 | 63 | Above | 15.5 |
| 26 | 203464_s_at | Epsin 2 | EPN2 | 17p11.1 | NM_014964.1 | 62.9 | Above | 43.3 |
| 27 | 213317_at | chloride | CLIC5 | 6p21.1 | AL049313.1 | 62.9 | Above | 99.3 |
| | | intracellular | | | | | | |
| | | channel 5 | | | | | | |
| 28 | 213423_x_at | Putative prostate | N33 | 8p22 | AI884858 | 62.9 | Above | 15.7 |
| | | cancer tumor | | | | | | |
| | | suppressor | | | | | | |
| 29 | 226817_at | Desmocollin 2 | DSC2 | 18q12.1 | AU154691 | 62.9 | Above | 48.3 |
| 30 | 227862_at | ESTs | | 1p35.1 | AA037766 | 62.9 | Above | 14.7 |
| 31 | 229339_at | EST | | 17p12 | AI093327 | 62.9 | Above | 31.1 |
| 32 | 211795_s_at | FYN binding | FYB | 5p13.1 | AF198052.1 | 59.4 | Above | 4.1 |
| | | protein | | | | | | |
| 33 | 218627_at | Hypothetical | FLJ11259 | 12q23.1 | NM_018370.1 | 57.9 | Above | 4.6 |
| | | protein FLJ11259 | | | | | | |
| 34 | 221748_s_at | Homo sapiens | TNS | 2q35 | AL046979 | 57.9 | Above | 6.6 |
| | | cDNA FLJ32766 | | | | | | |
| | | fis | | | | | | |
| 35 | 200709_at | FK506 binding | FKBP1A | 20p13 | NM_000801.1 | 57.1 | Above | 1.8 |
| | | protein 1A (12 kD) | | | | | | |
| 36 | 204615_x_at | Isopentenyl- | IDI1 | 10p15.3 | NM_004508.1 | 57.1 | Above | 2.6 |
| | | diphosphate delta | | | | | | |
| | | isomerase | | | | | | |
| 37 | 208881_x_at | Isopentenyl- | IDI1 | 10p15.3 | BC005247.1 | 57.1 | Above | 2.6 |
| | | diphosphate delta | | | | | | |
| | | isomerase | | | | | | |
| 38 | 213301_x_at | Transcriptional | TIF1 | 7q34 | AL538264 | 57.1 | Above | 2.0 |
| | | intermediary | | | | | | |
| | | factor 1 | | | | | | |
| 39 | 221747_at | Tensin | TNS | 2q35 | AL046979 | 57.1 | Above | 49.2 |
| 40 | 224726_at | KIAA1323 | KIAA1323 | 18q11.1 | W80418 | 57.1 | Above | 26.1 |
| 41 | 231455_at | ESTs | | 2p25.2 | AA768888 | 57.1 | Above | 7.7 |
| 42 | 232750_at | Homo sapiens | FLJ13750 | 2q35 | AU158570 | 57.1 | Above | 35.0 |
| | | cDNA FLJ13750 | | | | | | |
| 43 | 209685_s_at | Protein kinase C, | PRKCB1 | 16p11.2 | M13975.1 | 53.6 | Above | 1.9 |
| | | beta 1 | | | | | | |
| 44 | 204404_at | EST like | SLC12A2 | 5q23.3 | NM_001046.1 | 53.4 | Above | 2.0 |
| | | Na+/K+/Cl− | | | | | | |
| | | transporter with | | | | | | |
| | | AA permease | | | | | | |
| | | domain, memb 2 | | | | | | |
| 45 | 239673_at | ESTs | | 4q31.23 | AW080999 | 53.4 | Above | 9.0 |
| 46 | 240950_s_at | Homo sapiens | FLJ32658 | 19q13.33 | AA400740 | 53.4 | Above | 9.9 |
| | | cDNA FLJ32658 | | | | | | |
| 47 | 204297_at | Phosphoinositide- | PIK3C3 | 18q12.3 | NM_002647.1 | 52.5 | Above | 4.5 |
| | | 3-kinase, class 3 | | | | | | |
| 48 | 206591_at | Recombination | RAG1 | 11p13 | NM_000448.1 | 52.1 | Above | 5.4 |
| | | activating gene 1 | | | | | | |
| 49 | 209962_at | Erythropoietin | EPOR | 19p13.2 | M34986.1 | 52.1 | Above | 17.0 |
| | | receptor | | | | | | |
| 50 | 209963_s_at | Erythropoietin | EPOR | 19p13.2 | M34986.1 | 52.1 | Above | 7.6 |
| | | receptor | | | | | | |
| 51 | 210186_s_at | FK506 binding | FKBP1A | 20p13 | BC005147.1 | 52.1 | Above | 1.8 |
| | | protein 1A (12 kD) | | | | | | |
| 52 | 219866_at | Chloride | CLIC5 | 6p21.1 | NM_016929.1 | 52.1 | Above | 60.3 |
| | | intracellular | | | | | | |
| | | channel 5 | | | | | | |
| 53 | 203474_at | IQ motif | IQGAP2 | 5q13.2 | NM_006633.1 | 51.6 | Below | 2.8 |
| | | containing | | | | | | |
| | | GTPase activating | | | | | | |
| | | protein 2 | | | | | | |
| 54 | 210058_at | Mitogen-activated | MAPK13 | 6p21.1 | BC000433.1 | 51.6 | Above | 2.3 |
| | | protein kinase 13 | | | | | | |
| 55 | 211891_s_at | Rho guanine | ARHGEF4 | 2q22 | AB042199.1 | 51.6 | Above | 452.6 |
| | | nucleotide | | | | | | |
| | | exchange factor | | | | | | |
| | | (GEF) 4 | | | | | | |
| 56 | 214214_s_at | Complement | C1QBP | 17p13.3 | AU151801 | 51.6 | Below | 2.0 |
| | | component 1, q | | | | | | |
| | | subcomponent | | | | | | |
| | | binding protein | | | | | | |
| 57 | 218152_at | High-mobility | HMG20A | 15q24 | NM_018200.1 | 51.6 | Above | 1.7 |
| | | group 20A | | | | | | |
| 58 | 234983_at | ESTs | FLJ21415 | 12q24.22 | BE893995 | 51.6 | Above | 2.4 |
| 59 | 240446_at | KIAA1323 | KIAA1323 | 18q11.2 | AI798164 | 51.6 | Above | 102.2 |
| 60 | 244107_at | ESTs | | 18q12.1 | AW189097 | 51.6 | Above | 518.9 |
| 61 | 205794_s_at | Neuro-oncological | NOVA1 | 14q12 | NM_002515.1 | 51.4 | Above | 40.4 |
| | | ventral antigen 1 | | | | | | |
| 62 | 217628_at | chloride | CLIC5 | 6p21.1 | BF032808 | 51.4 | Above | 87.4 |
| | | intracellular | | | | | | |
| | | channel 5 | | | | | | |
| 63 | 218804_at | Hypothetical | FLJ10261 | 11q13.3 | NM_018043.1 | 51.4 | Above | 41.6 |
| | | protein FLJ10261 | | | | | | |
| 64 | 230698_at | EST | | 7q11.22 | AW072102 | 51.4 | Above | 8.7 |
| 65 | 225129_at | cDNA FLJ37548 | FLJ37548 | 16q13 | AW170571 | 49.4 | Above | 3.0 |
| | | fis | | | | | | |
| 66 | 201266_at | Thioredoxin | TXNRD1 | 12q23-q24.1 | NM_003330.1 | 48.2 | Above | 1.7 |
| | | reductase 1 | | | | | | |
| 67 | 203611_at | Telomeric repeat | TERF2 | 16q22.1 | NM_005652.1 | 48.2 | Above | 5.3 |
| | | binding factor 2 | | | | | | |
| 68 | 213017_at | Lung alpha/beta | LABH3 | 18q11.1 | AL534702 | 48.2 | Above | 4.0 |
| | | hydrolase 3 | | | | | | |
| 69 | 236430_at | hypothetical | MGC23911 | 16q22.1 | AA708152 | 48.2 | Above | 16.8 |
| | | protein | | | | | | |
| | | MGC23911 | | | | | | |
| 70 | 209035_at | Midkine (neurite | MDK | 11p11.2 | M69148.1 | 47.7 | Above | 4.6 |
| | | growth-promoting | | | | | | |
| | | factor 2). | | | | | | |
| 71 | 209193_at | Pim-1 oncogene | PIM1 | 6p21.2 | M24779.1 | 47.7 | Above | 2.0 |
| 72 | 218625_at | Neuritin 1 | NRN1 | 6p24.1 | NM_016588.1 | 47.7 | Above | 5.1 |
| 73 | 226038_at | Hypothetical | FLJ23749 | 8p23.1 | BF680438 | 47.7 | Above | 5.2 |
| | | protein FLJ23749 | | | | | | |
| 74 | 232227_at | EST | | 9q34.3 | AV736391 | 47.7 | Above | 14.7 |
| 75 | 204160_s_at | Ectonucleotide | ENPP4 | 6p12.3 | AW194947 | 46.5 | Above | 7.2 |
| | | pyrophosphatase/phosphodiesterase | | | | | | |
| | | 4 (putative | | | | | | |
| | | function) | | | | | | |
| 76 | 206233_at | UDP- | B4GALT6 | 18q11 | AF097159.1 | 46.5 | Above | 2.6 |
| | | Gal: betaGlcNAc | | | | | | |
| | | beta 1,4- | | | | | | |
| | | galactosyltransferase, | | | | | | |
| | | polypeptide 6 | | | | | | |
| 77 | 218813_s_at | SH3-domain | | 9q34.11 | NM_020145.1 | 46.5 | Above | 6.2 |
| | | GRB2-like | SH3GLB2 | | | | | |
| | | endophilin B2 | | | | | | |
| 78 | 227111_at | Homo sapiens | FLJ31099 | 9q33 | BG179317 | 46.5 | Above | 2.7 |
| | | cDNA FLJ31099 | | | | | | |
| | | fis, clone | | | | | | |
| | | IMR321000230 | | | | | | |
| 79 | 202382_s_at | Glucosamine-6- | GNPI | 5q21 | NM_005471.1 | 46.2 | Above | 5.6 |
| | | phosphate | | | | | | |
| | | isomerase | | | | | | |
| 80 | 202838_at | Fucosidase, alpha- | FUCA1 | 1p34 | NM_000147.1 | 46.2 | Above | 4.8 |
| | | L-1, tissue | | | | | | |
| 81 | 225731_at | Hypothetical | KIAA1223 | 4q26 | AB033049.1 | 46.2 | Above | 2.8 |
| | | protein | | | | | | |
| | | KIAA1223 | | | | | | |
| 82 | 225835_at | FLJ21409 | SLC12A2 | 5q23.2 | AK025062.1 | 46.2 | Above | 3.6 |
| 83 | 229790_at | Telomeric repeat | TERF2 | 16q22.1 | AW006832 | 46.2 | Above | 7.4 |
| | | binding factor 2 | | | | | | |
| 84 | 230069_at | Hypothetical | FLJ12876 | 5q35.3 | BF593817 | 46.2 | Above | 9.4 |
| | | protein FLJ12876 | | | | | | |
| 85 | 235872_at | ESTs | | | BE408975 | 46.2 | Above | 17.7 |
| 86 | 239300_at | EST | | 18q12.3 | AI632214 | 46.2 | Above | 3.0 |
| 87 | 241940_at | EST | | 18q11.2 | BF477544 | 46.2 | Above | 2.9 |
| 88 | 203370_s_at | Enigma (LIM | ENIGMA | 5q35.3 | NM_005451.2 | 45.9 | Above | 8.1 |
| | | domain protein) | | | | | | |
| 89 | 215149_at | LOC149153: | LOC149153 | 1p36.32 | AF052109.1 | 45.9 | Above | 9.2 |
| 90 | 217901_at | Desmoglein 2 | DSG2 | 18q12.1 | BF031829 | 45.9 | Above | 6.7 |
| | | desmosomal | | | | | | |
| | | cadherin | | | | | | |
| 91 | 235333_at | UDP- | BA4GALT6 | 18q12.1 | BG503479 | 45.9 | Above | 2.0 |
| | | Gal: betaGlcNAc | | | | | | |
| | | beta 1,4- | | | | | | |
| | | galactosyltransferase, | | | | | | |
| | | polypeptide 6 | | | | | | |
| 92 | 242881_x_at | EST | | | BG285837 | 45.9 | Above | 11.8 |
| 93 | 200783_s_at | Stathmin | STMN1 | 1p35.1 | NM_005563.2 | 45.8 | Above | 1.5 |
| | | 1/oncoprotein 18 | | | | | | |
| | | leukemia- | | | | | | |
| | | associated | | | | | | |
| | | phosphoprotein | | | | | | |
| 94 | 201334_s_at | Rho guanine | ARHGEF12 | 11q23.3 | NM_015313.1 | 45.8 | Above | 6.1 |
| | | nucleotide | | | | | | |
| | | exchange factor | | | | | | |
| | | (GEF) 12 | | | | | | |
| 95 | 203038_at | Protein tyrosine | PTPRK | 6q22.33 | NM_002844.1 | 45.8 | Above | 9.1 |
| | | phosphatase, | | | | | | |
| | | receptor type, K | | | | | | |
| 96 | 209735_at | ATP-binding | ABCG2 | 4q22 | AF098951.2 | 45.8 | Above | 4.5 |
| | | cassette, sub- | | | | | | |
| | | family G | | | | | | |
| | | (WHITE), | | | | | | |
| | | member 2 | | | | | | |
| 97 | 212063_at | Unactive | P23 | 12q12 | BE903880 | 45.8 | Below | 7.4 |
| | | progesterone | | | | | | |
| | | receptor, 23 kD | | | | | | |
| 98 | 212399_s_at | Hypothetical | KIAA0121 | 3p25.2 | D50911.2 | 45.8 | Above | 1.8 |
| | | protein | | | | | | |
| | | KIAA0121 | | | | | | |
| 99 | 212438_at | Putative nucleic | RY1 | 2p13.1 | BG252325 | 45.2 | Above | 1.7 |
| | | acid binding | | | | | | |
| | | protein RY-1 | | | | | | |
| 100 | 214761_at | OLF-1/early B- | OAZ | 16q12 | AW149417 | 45.2 | Above | 2.1 |
| | | cell factor | | | | | | |
| | | associated zinc | | | | | | |
| | | finger protein | | | | | | |

Biologic Insights from the New Class Defining Genes

Interestingly, the overall quantitative pattern of expression of discriminating genes varied significantly between leukemia subtypes (Table 69). Within the B-cell lineage leukemia subtypes, E2A-PBX1, TEL-AML1, BCR-ABL, and Hyperdiploid>50 chromosomes were characterized primarily by genes that were overexpressed, where as almost 40% of the discriminating genes that characterized MLL fusion gene expressing leukemias were underexpressed. More remarkably, the discriminating genes for the leukemia subtypes defined by chimeric transcription factors were markedly overexpressed, with an average fold increase of 112 and 48 for E2A-PBX1 and TEL-AML1, respectively. By contrast, the discriminating genes for BCR-ABL and MLL fusion gene expressing leukemias showed an average fold increases of only 6.8. and 8.6, respectively, whereas the discriminating genes for hyperdiploid>50 chromosomes had an average fold-increase of only 2.6 fold. These data suggest that the quantitative global changes in a cell's expression profile vary markedly depending on the genetic lesion(s) that underlie the initiation of the leukemic process.

TABLE 69
Summary of fold change by diagnostic
subgroup (by gene)

| | Mean fold | |
|---|---|---|
| Subgroup | change | Range |
| BCR-ABL | 6.8 | 1.1-90.5 |
| E2A-PBX1 | 112.0 | 1.6-5435 |
| Hyperdiploid >50 | 2.6 | 1.3-27.2 |
| MLL rearrangement | 8.6 | 1.0-75 |
| T-ALL | 387 | 2.1-7685 |
| TEL-AML1 | 48.3 | 1.5-2446 |

Tables 70-74 show genes whose expression is limited to a single B-cell lineage class, and therefore function not only as class discriminators in the decision tree format, but are also class discriminators in a parallel format in which a class is distinguished against all others. Thus, these genes have the potential of serving as unique class specific diagnostic or therapeutic targets. In addition, these genes may provide unique insights into the underlying biology of the different leukemia subtypes. For example, BCR-ABL expressing ALLs are characterized by the over expression of Dynactin 4, which encodes a RING finger containing protein that is part of the 20S dynactin multisubunit complex involved in movement, intracellular transport and division through its interaction with the cytoplasmic microtubule-based motor dynein; PSTPIP2, which encodes a proline/serine/threonine phosphatase-interacting protein that is also involved in controlling the organization of the cytoskeleton, and is tyrosine phosphorylated following activation of receptor tyrosine kinases (Karki et al. (2000) J. Biol. Chem.275:4834-4839); and several novel ESTs.

TABLE 70
Genes highly Correlated with BCR-ABL

| GenBank Reference | Gene Description |
|---|---|
| AK002064 | DKFZP564A2416 histone H5 signature |
| BE218028 | Dynactin 4 |
| NM_024600 | FLJ20898 |
| NM_024430 | Pro-Ser-Thr phsphatase interac. protein 2 |
| AV648669 | FLJ39877 |

E2A-PBX1 expressing leukemias are characterized by the expression of PBX1, the receptor tyrosine kinase gene C-MERTK, and the FAT tumor suppressor, which encodes a member of the cadherin repeat domain containing family of transmembrane proteins (see Table 64). Among the discriminating genes were two genes, EB-1 and Wnt16 that had previously been shown to be over expressed in this leukemia subtype (Wu et al. (1998) J. Biol. Chem. 273:30487-30496; and Fu et al. (1999) Oncogene 10 18:4920-4929). In addition, the retinal degeneration B beta gene (McWhirter et al. (1999) Proc. Natl. Acad. Sci. U S A. 96:11464-11469), and a number of novel ESTs were identified as being uniquely over expressed in this leukemia subtype, whereas the SOCS2 negative regulators of cytokine signaling was found to be under expressed (Fullwood and Hsuan (1999) J. Biol. Chem. 274:31553-31558).²⁶

TABLE 71
Genes highly Correlated with E2A-PBX1

| GenBank Reference | Gene Description |
|---|---|
| NM_012417 | retinal degeneration B beta |
| AI971602 | MGC10485 |
| AW005572 | EB-1 |
| AL357503 | Q9H4T4 like |
| NM_016087 | Wnt16 |

Hyperdiploid leukemias with >50 chromosomes were characterized by the over expression of MST4, which encodes a novel serine/threonine kinase (Horvat and Medrano (2001) Genomics 72:209-212); SH3BP2, which encodes a SH3-domain containing binding protein (Lin et al. (2001) Oncogene 20:6559-6569) histone deacetylase 6, which encodes a protein involved in transcriptional repression; the retinoblastoma binding protein 7 gene, which encodes a protein found in many functional histone deacetylase complexes (Bell et al. (1997) Genomics 44:163-170), and TNRC11 a trinucleotide repeat containing gene that is also known as HOPA or TRAP230 and is part of the thyroid hormone receptor-associated protein (TRAP) complex (Huang et al (1991) Nature 350:160-162; and Ito et al. (1999) Mol Cell. 3:361-370.

TABLE 72
Genes highly Correlated with Hyperdiploid >50

| GenBank Reference | Gene Description |
|---|---|
| NM_002893 | Retinoblastoma binding protein 7 |
| AB000462 | SH3-domain binding protein 2 |
| NM_006044 | Histone deacetylase 6 |
| BC004354 | trinucleotide repeat containing 11 |
| NM_016542 | Mst3 and SOK1-related kinase |

Cases with MLL gene rearrangements were characterized by the over expression of HOXA9 and Meis1 (see Table 66). Included in the up-regulated genes was a novel transcript from chromosome 20 that was over expressed almost 25 fold. This transcript is predicted to encode a protein of 280 amino acids that shows a low level of homology to a lysosome-associated membrane glycoprotein (LAMP). Also specifically over expressed in this leukemia subtype is a gene encoding an insulin growth factor (IGF) II RNA binding protein, that has been shown to repress the translation of the IGF-II growth factor (Armstrong et al (2002). Nat. Genet. 30:41-47). Among the down regulated genes was neuron navigator 1 (Nielsen et al. (1999) Mol Cell Biol. 19:1262-1270), which encodes an 1874 amino acid protein and is involved in direction guidance of migratory cells, and a member of the TCF/LEF family of transcription factors, TCF-4. TCF-4 functions downstream of β-catenin in the Wnt-mediated signaling cascade and has been shown to be essential for the maintenance of intestinal crypt stem cells (Maes et al. (2002) Genomics 80:21-30).

TABLE 73
Genes highly Correlated with MLL

| GenBank Reference | Gene Description |
|---|---|
| NM_012261 | C20orf103 |
| AI202327 | FLJ37247 |
| NM_006548 | IGF-II mRNA-binding protein 2 |
| NM_018401 | gene for serine/threonin protein kinase |
| NM_018728 | myosin 5C |
| AB032977 | neuron navigator 1 |

Genes that were discriminators of TEL-AML1 leukemias included a gene localized to chromosome 18q11.1 that encodes a 795 amino acid protein that has 8 ankyrin repeat domains and a C-terminal RING finger domain. This combination of domains is identified in only a limited number of mammalian proteins, most notably BARD1, a regulator of the BRCA1 tumor suppressor (Korinek et al. (1998) Nat Genet.19:379-383). Other genes overexpressed in the subtype include desmocollin (Irminger-Finger and Leung (2002) Int. J. Biochem. Cell Biol. 34:582-587), FLJ12722 a novel protein of unknown function, and a member of the IAP family of apoptosis inhibitors, BIRC7, which is overexpressed 25 fold (Whittock et al. (2000) Biochem Biophys Res Commun. 276:454-460).

TABLE 74
Genes highly Correlated with TEL-AML1

| GenBank Reference | Gene Description |
|---|---|
| W80418 | KIAA1323 |
| AK022784 | FLJ12722 |
| NM_0022161 | BIRC7 |
| A1452798 | FLJ39434 |
| A1797281 | Desmocollin 3 |

Expression Profiling Accurately Identifies the Prognostic Subtypes of ALL

To assess the accuracy of identifying prognostically important ALL genetic subtypes by expression profiling, the class discriminating genes identified using a chi-squared metric were used in an ANN-based supervised learning algorithm. Class assignment utilized the decision tree differential diagnostic format described elsewhere herein, and required that the node value for assignment exceeded a statistically defined confidence level. Using this approach resulted in exceptionally accurate class prediction in a randomly selected training set that consisted of three-fourths of the total cases (100 cases). When this classification model was then applied to a blinded test set consisting of the remaining 32 samples, an overall accuracy of 97% was achieved for class assignment. To control for over-fitting of the data, 10 additional rounds of this analysis were performed in which for each round new training and test sets were developed, genes reselected using the new training set, and then their performance assessed on the new test set. This resulted in an average accuracy of class assignment in the blinded test sets of 97.2%, with a range from 93.8% to 100%. Although the number of genes required for optimal class assignment varied between classes, the best overall diagnostic accuracy was achieved using the top 50 genes per class. A similar level of accuracy was achieved using a variety of other supervised learning algorithms, including κ-NN and SVM.

Interestingly, of the rare misclassification errors, two were cases of BCR-ABL expressing ALL that by gene expression analysis was classified as hyperdiploid>50 chromosomes. The karyotype of these cases showed the presence of both the Philadelphia chromosome and a hyperdiploid karyotype consisting of >50 chromosomes—including trisomy of chromosomes X and 21 (data not shown). The expression profile thus correctly identified the presence of the hyperdiploid>50 chromosomes class; however, since each case is assigned to only a single class, the algorithm failed to correctly identify the presence of BCR-ABL. Nevertheless, the data presented demonstrates the exceptional accuracy of this single platform for the diagnosis of the prognostically important subtypes of ALL.

Overview of Experimental Procedure

A. Gene Expression Profiling

The preparation of mononuclear cell suspensions from diagnostic bone marrow aspirates, extraction of total RNA, and preparation of hybridization solutions was performed as described for Example 1. Individual hybridization solutions from our previous study had been stored at −80° C. since initial hybridization (approximately 1 year). These solutions were thawed and hybridized to Affymetrix® HG-U133A and HG-U133B oligonucleotide microarrays (Affymetrix Inc., Santa Clara, Calif.) according to Affymetrix protocols. In two cases where the original hybridization solutions were no longer available, replicate viably frozen mononuclear cell preparations from the diagnostic bone marrow aspirate were obtained, RNA isolated, cDNA and cRNA synthesized, labeled, fragmented and hybridized as described for Example 1.

After sample hybridization, arrays were then stained with phycoerythrin-conjugated streptavidin (Molecular Probes, Eugene, Oreg.). Antibody amplification was performed with biotinylated anti-streptavidin (Vector Laboratories, Burlingame, Calif.), followed by staining with phycoerythrin-conjugated streptavidin (Molecular Probes). Arrays were scanned using a laser confocal scanner (Agilent, Palo Alto, Calif.) and then analyzed with Affymetrix® Microarray suite 5.0 (MAS 5.0). Detection values (present, marginal or absent) were determined by default parameters, and signal values were scaled by global methods to a target value of 500. Microarray scan images were visually inspected for apparent defects, and Affymetrix internal controls were utilized to monitor the success of hybridization, washing, and staining procedures. Minimal quality control parameters for inclusion in the study included greater than 10% present calls and a GAPDH 3′/5′ ratio of ≦3. The arrays included in this study had an average % present call of 35.9% for the A chip and 21.0% for the B chip (combined average of 28.5%).

B. Statistical Analysis

The dataset was separated into a train set (100) and test set (32). The identification of subtype discriminating genes was performed using the training set. Moreover, both gene discovery and subsequent class predictions were performed using a differential diagnosis decision tree format. In this format, classification was performed in a sequential order starting with T-ALL and proceeding in order E2A-PBX1, TEL-AML1, BCR-ABL, MLL rearrangement, and Hyperdiploid>50 chromosomes. Unassigned cases were classified as other. Samples classified into the class under diagnosis were removed prior to proceeding to the next level in the decision tree. In addition, prior to analysis a variation filter was applied to remove any probe set that showed minimal variation across the dataset, and thus contributed minimally, if at all, to the discrimination of leukemia subtypes. Specifically, probe sets were eliminated from further analysis if the number of cases with a present call was less than ½ the number of samples comprising the leukemia subgroup under analysis, had a signal value<100 in all samples in the dataset, or had a maximal signal value in the dataset—minimal signal value in the dataset that was less than 100. In addition, all signal values with absent or marginal calls were reset to 1, while probe sets with a present “P” call and a signal<100 had the signal reset to 100. The values for signals from the Affymetrix® control sets were removed prior to analysis.

Unsupervised hierarchical clustering and principal component analysis (PCA) were performed using GeneMaths software (version 1.5, Applied Maths, Belgium). Data reduction to define the genes most useful in class distinction was primarily performed using a chi-square metric. In this procedure, an entropy-based discretization method was first applied to identify genes whose expression across the dataset showed differentiation between class and non-class.¹⁷ The assigned descretized value for the gene was then used in a chi-square calculation to determine if the association with a class was more than would be expected by random chance. The stronger the association with the class, the larger the chi-square value calculated. For the genes that couldn't be discretized, their chi-squared values were set to zero. To evaluate the statistical significance of the discriminating genes, we used a permutation test in which for each class, case labels were randomly reassigned to generate new groups of identical size. The label permutated data was discretized again and the chi-square values were recalculated. The permutation test was repeated for a total of 1000 times. The true chi-square values for each probe set were then compared to the values generated from the 1000 permutations to determine how many times a chi-square value for a probe set in a randomly labeled group was greater than that obtained for the true class distinction. A p value was calculated as the number of times the chi-square value exceeded the true value in the 1000 permutations.

The discriminating genes selected were then used in supervised learning algorithms to build classifiers that could identify the specific genetic subgroup. Algorithms used included k-Nearest Neighbors (k-NN), Support Vector Machine (SVM), and an artificial neural network (ANN). See, Example 1, Witten and Frank (1999) Data mining: Practical machine learning tools and techniques with Java implementation. Morgan Kaufinan; Platt (1998) Fast training of support vector machines using sequential minimal optimization in Advances in kernel methods—support vector learning Schlkopf B, Burges C, and Smola A, eds. MIT Press; and Cover and Hart (1967) IEEE Transactions on Information Theory 13:21-27. Performance of each model was initially assessed by three-fold cross validation on a randomly selected stratified training set. True error rates of the best performing classifiers were then determined using the remaining one-fourth of the samples as a blinded test group. Class assignment required that a sample's calculated node value exceed a statistically determined confidence level in order for it to be assigned to a class. Details of the supervised learning algorithms and their use are described below.

Detailed Experimental Procedures

A. Patient Dataset

132 cases of pediatric ALL were selected from the original 327 diagnostic bone marrow aspirates described in Example 1 to reanalyze on the higher density U133A and B microarrays. The selection of cases was based on having sufficient numbers of each subtype to build accurate class predictions, rather than reflecting the actual frequency of these groups in the pediatric population.

B. Hybridization of Microarrays

The hybridization solutions according to Example 1 were thawed at 45° C., then microcentrifuged for 5 minutes to remove any insoluble material from the mixture. The hybridization solutions were added to U133A chips and allowed to hybridize for 16 hours at 45° C. At the end of the incubation period, the hybridization solution was removed from each U133A chip and refrozen. Subsequently, the hybridizations were thawed and hybridized to the U133B chip.

A non-stringent wash buffer (6×SSPE, 0.01% Tween 20) was added to each chip cassette after the hybridization solution was removed and the cassette allowed to equilibrate to room temperature. The microarray cassettes were then placed on the fluidics station and the antibody amplification protocol performed. The arrays were washed at 25° C. with the non-stringent buffer followed by a more stringent wash at 50° C. with 100 mM MES, 0.1M NaCl₂, 0.01% Tween 20. The arrays were then stained with Streptavidin Phycoerythrin (SAPE, Molecular Probes, Eugene, Oreg.) for 10 minutes at 25° C. Following another non-stringent wash, the arrays were hybridized for 10 minutes at 25° C. with an antibody solution (100 mM MES, 1 M [Na⁺], 0.05% Tween 20, 2 mg/ml BSA, 0.1 mg/ml goat IgG, and 3 □g/ml biotinylated antibody). This solution was removed and the cassettes restained with the SAPE solution.

Arrays were scanned on a laser confocal scanner (Agilent, Palo Alto, Calif.) and then analyzed with Affymetrix® Microarray Suite 5.0 (MAS 5.0). Detection values (present, marginal or absent) were determined by default parameters, and signal values were scaled by global methods to a target value of 500. After completing the scans, the arrays were visually inspected for defects and Affymetrix internal controls were utilized to monitor the success of hybridization, washing, and staining procedures.

C. Statistical Methods

The chi-square metric and the kNN and ANN supervised learning algorithms were performed as described for Example 1. The SVM supervised learning algorithm that was used in this study is available as part of the software package Rv 1.6.0. See, Ribeiro, and Brown. The ISBA Bulletin, 8(1):12-16, and www.r-project.org.

To determine the performance of each model using ANN, a confidence threshold was built for each diagnostic subtype utilizing a modification of the method described by Khan et al. (2001) Nat. Med. 7:673-679. Models were built based on a decision tree format where each level of the decision tree contains only two possible distinctions—class and non-class (for example, T verses non-T). At each level, using only samples in the training set, 3 ANN models were built by 3-fold cross validation. The training set samples were then shuffled and 3 additional ANN models were built. This model building process was repeated for a total of 100 times at each step of the decision tree. Then an empirical probability distribution for the ANN output node value was built only for subtype under study, for example, T-ALL at the first step of the decision tree. Only nodal values greater than 0.5 for each subtype were included. For each individual sample in the training set, the 100 validation subtype node values were averaged and compared to threshold. Individual samples were assigned to the subtype under study only when its average subtype nodal value was greater than the 95% confidence threshold. For samples in the test set, subtype nodal values are averaged from all models generated in the 3-fold cross validation. A sample is assigned to the class under study when the average subtype nodal value is greater than the 95% confidence level defined on the training set. A sample not assigned to the subtype will progress to the next level of the decision tree, where the entire process is repeate.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method of assigning a subject affected by leukemia to a leukemia risk group, said method comprising:
a) providing a subject expression profile of a sample from said subject affected by leukemia;
b) providing a plurality of reference expression profiles, each associated with a leukemia risk group selected from the group consisting of T-ALL, E2A-PBX1, TEL-AML1, BCR-ABL, MLL, Hyperdiploid>50, and Novel, wherein the subject expression profile and each reference expression profile comprise one or more values representing the expression level of a gene having differential expression in at least one leukemia risk group; and
c) selecting the reference expression profile most similar to the subject expression profile to thereby assign said subject affected by leukemia to a leukemia risk group.

2. The method of claim 1 wherein the subject expression profile and the reference expression profile associated with the T-ALL risk group comprise values selected from the group consisting of:
a) values representing the expression levels of at least 20 genes selected from the genes shown in Table 7;
b) a value representing the expression level of the gene shown in Table 14;
c) values representing the expression levels of at least 20 genes selected from the genes shown in Table 21;
d) values representing the expression levels of at least 20 genes selected from the genes shown in Table 28;
e) values representing the expression levels of at least 20 genes selected from the genes shown in Table 35;
f) values representing the expression levels of at least 20 genes selected from the genes shown in Table 59; and
g) values representing the expression levels of at least 20 genes selected from the genes shown in Table 67.

3. The method of claim 1 wherein the subject expression profile and the reference expression profile associated with the E2A-PBX1 risk group comprise values selected from the group consisting of:
a) values representing the expression levels of at least 20 genes selected from the genes shown in Table 3;
b) a value representing the expression level of the gene shown in Table 10;
c) values representing the expression levels of at least 20 genes selected from the genes shown in Table 17;
d) values representing the expression levels of at least 20 genes selected from the genes shown in Table 24;
e) values representing the expression levels of at least 20 genes selected from the genes shown in Table 31;
f) values representing the expression levels of at least 20 genes selected from the genes shown in Table 55;
g) values representing the expression levels of at least 20 genes selected from the genes shown in Table 64; and
h) values representing the expression levels of at least one of the genes shown in Table 71.

4. The method of claim 1 wherein the subject expression profile and the reference expression profile associated with the TEL-AML1 risk group comprise values selected from the group consisting of:
a) values representing the expression levels of at least 20 genes selected from the genes shown in Table 8;
b) values representing the expression levels of the genes shown in Table 15;
c) values representing the expression levels of at least 20 genes selected from the genes shown in Table 22;
d) values representing the expression levels of at least 20 genes selected from the genes shown in Table 29;
e) values representing the expression levels of at least 20 genes selected from the genes shown in Table 36;
f) values representing the expression levels of at least 20 genes selected from the genes shown in Table 55;
g) values representing the expression levels of at least 20 genes selected from the genes shown in Table 68; and
h) values representing the expression levels of at least one of the genes shown in Table 74.

5. The method of claim 1 wherein the subject expression profile and the reference expression profile associated with the BCR-ABL risk group comprise values selected from the group consisting of:
a) values representing the expression level of at least 20 genes selected from the genes shown in Table 2;
b) values representing the expression levels of the genes shown in Table 9;
c) values representing the expression level of at least 20 genes selected from the genes shown in Table 16;
d) values representing the expression levels of at least 20 genes selected from the genes shown in Table 23;
e) values representing the expression levels of at least 20 gene selected from the genes shown in Table 30;
f) values representing the expression levels of at least 20 genes selected from the genes shown in Table 54;
g) values representing the expression levels of at least 20 genes selected from the genes shown in Table 63; and
h) values representing the expression levels of at least one of the genes shown in Table 70.

6. The method of claim 1 wherein the subject expression profile and the reference expression profile associated with the MLL risk group comprise values selected from the group consisting of:
a) values representing the expression levels of at least 20 genes selected from the genes shown in Table 5;
b) values representing the expression levels of the genes shown in Table 12;
c) values representing the expression level of at least 20 genes selected from the genes shown in Table 19;
d) values representing the expression levels of at least 20 genes selected from the genes shown in Table 26;
e) values representing the expression levels of at least 20 genes selected from the genes shown in Table 33;
f) values representing the expression levels of at least 20 genes selected from the genes shown in Table 57;
f) values representing the expression levels of at least 20 genes selected from the genes shown in Table 66; and
g) values representing the expression levels of at least one of the genes shown in Table 73.

7. The method of claim 1 wherein the subject expression profile and the reference expression profile associated with the Hyperdiploid>50 risk group comprise values selected from the group consisting of:
a) values representing the expression levels of at least 20 genes selected from the genes shown in Table 4;
b) values representing the expression levels of the genes shown in Table 11;
c) values representing the expression levels of at least 20 genes selected from the genes shown in Table 18;
d) values representing the expression levels of at least 20 genes selected from the genes shown in Table 25;
e) values representing the expression levels of at least 20 genes selected from the genes shown in Table 32;
f) values representing the expression levels of at least 20 genes selected from the genes shown in Table 56;
g) values representing the expression levels of at least 20 genes selected from the genes shown in Table 65; and
h) values representing the expression levels of at least one of the genes shown in Table 72.

8. The method of claim 1 wherein the subject expression profile and the reference expression profile associated with the Novel risk group comprise values selected from the group consisting of:
a) values representing the expression level of at least 20 genes selected from the genes shown in Table 6;
b) values representing the expression level of the genes shown in Table 13;
c) values representing the expression levels of at least 20 genes selected from the genes shown in Table 20;
d) values representing the expression levels of at least 20 genes selected from the genes shown in Table 27;
e) values representing the expression levels of at least 20 genes selected from the genes shown in Table 34; and
f) values representing the expression levels of at least 20 genes selected from the genes shown in Table 58.

9. The method of claim 1, wherein said sample from said subject affected by ALL comprises leukemic blasts.

10. The method of claim 9, wherein said sample from said subject affected by ALL comprises at least 35% leukemic blasts.

11. The method of claim 10, wherein said sample from said subject affected by ALL comprises at least 75% leukemic blasts.

12. The method of claim 9 wherein said sample comprises leukemic blasts derived from peripheral blood.

13. The method of claim 9 wherein said sample comprises blast cells derived from bone marrow.

14. A method of predicting whether a subject affected by leukemia has an increased risk of relapse, said method comprising the steps of:
a) assigning the subject affected by leukemia to a leukemia risk group selected from the group consisting of T-ALL, Hyperdiploid>50, TEL-AML1, MLL, E2A-PBX1, BCR-ABL, and Novel;
b) providing a subject expression profile of a sample from said subject affected by leukemia;
c) providing a reference expression profile associated with the occurrence of relapse in the leukemia risk group to which the subject affected by leukemia is assigned, wherein the subject expression profile and the reference expression profile comprise one or more values representing the expression level of a gene having differential expression in subjects affected by leukemia who will relapse after conventional therapy; and
d) determining whether the subject expression profile shares sufficient similarity to the reference expression profile associated with relapse in the leukemia risk group to which the subject affected by leukemia is assigned to thereby determine whether the subject affected by leukemia has an increased risk of relapse.

15. The method of claim 14, wherein the step of assigning the subject affected by leukemia to a leukemia risk group is performed according to the method of claim 1.

16. The method of claim 14, wherein said subject affected by leukemia is assigned to the T-ALL risk group and said subject expression profile and said reference expression profile comprise values representing the expression levels of at least 8 genes selected from the genes shown in Table 44.

17. The method of claim 14, wherein said subject affected by leukemia is assigned to the Hyperdiploid>50 risk group and said subject expression profile and said reference expression profile comprise values representing the expression levels of at least 5 genes selected from the genes shown in Table 45.

18. The method of claim 14, wherein said subject affected by leukemia is assigned to the TEL-AML1 risk group and said subject expression profile and said reference expression profile comprise values representing the expression levels of at least 3 genes selected from the genes shown in Table 46.

19. The method of claim 14, wherein said subject affected by leukemia is assigned to the MLL risk group and said subject expression profile and said reference expression profile comprise values representing the expression levels of at least 5 genes selected from the genes shown in Table 47.

20. The method of claim 14, wherein said subject affected by leukemia is not assigned to the T-ALL, Hyperdiploid>50, TEL-AML1, MLL, E2A-PBX1, or BCR-ABL risk group and said subject expression profile and said reference expression profile comprise values representing the expression levels of at least 4 genes selected from the genes shown in Table 48.

21. A method of predicting whether a subject affected by TEL-AML1 has an increased risk of developing secondary AML, said method comprising:
a) providing a subject expression profile of a sample from said subject affected by TEL-AML1;
b) providing a reference expression profile associated with the occurrence of secondary AML in subjects affected by TEL-AML1 wherein the subject expression profile and the reference expression profile comprise one or more values representing the expression level of a gene having differential expression in subjects affected by TEL-AML1 who will develop secondary AML; and
c) determining whether the subject expression profile shares sufficient similarity to the reference expression profile associated with the occurrence of secondary AML to thereby determine whether the subject affected by TEL-AML1 has an increased risk of developing secondary AML.

22. A method of choosing a therapy for a subject affected by leukemia, said method comprising:
a) providing a subject expression profile of a sample from said subject affected by leukemia;
b) providing a plurality of reference expression profiles, each associated with a leukemia risk group selected from the group consisting of T-ALL, E2A-PBX1, TEL-AML1, BCR-ABL, MLL, Hyperdiploid>50, and Novel, wherein the subject expression profile and each reference expression profile comprise one or more values representing the expression of level of a gene having differential expression in at least one leukemia risk group; and
c) selecting the reference expression profile most similar to the subject expression profile to thereby choose a therapy for the subject affected by leukemia.

23. A method of choosing a therapy for a subject affected by leukemia, said b method comprising the steps of:
a) assigning the subject affected by leukemia to a leukemia risk group selected from the group consisting of T-ALL, Hyperdiploid>50, TEL-AML1, MLL, E2A-PBX1, BCR-ABL, and Novel;
b) providing a subject expression profile of a sample from said subject affected by ALL;
c) providing a reference expression profile associated with the occurrence of relapse in the leukemia risk group to which the subject affected by
leukemia is assigned, wherein the subject expression profile and the reference expression profile comprise one or more values representing the expression level of a gene having differential expression in subjects who will relapse after conventional therapy; and
d) determining whether the subject expression profile shares sufficient similarity to the reference expression profile associated with relapse in the leukemia risk group to which the subject affected by ALL is assigned to thereby chose a therapy for said subject affected by ALL.

24. The method of claim 23, wherein the step of assigning the subject affected by leukemia to a leukemia risk group is performed according to the method of claim 1.

25. The method of claim 23, wherein said subject affected by leukemia is assigned to the T-ALL risk group and said subject expression profile and said reference expression profile comprise values representing the expression levels of at least 8 genes selected from the genes shown in Table 44.

26. The method of claim 23, wherein said subject affected by leukemia is assigned to the Hyperdiploid>50 risk group and said subject expression profile and said reference expression profile comprise values representing the expression levels of at least 5 genes selected from the genes shown in Table 45.

27. The method of claim 23, wherein said subject affected by leukemia is assigned to the TEL-AML1 risk group and said subject expression profile and said reference expression profile comprise values representing the expression levels of at least 3 genes selected from the genes shown in Table 46.

28. The method of claim 23, wherein said subject affected by leukemia is assigned to the MLL risk group and said subject expression profile and said reference expression profile comprise values representing the expression levels of at least 5 genes selected from the genes shown in Table 47.

29. The method of claim 23, wherein said subject affected by leukemia is not assigned to the T-ALL, hyperdiploid>50, TEL-AML1, MLL, E2A-PBX1, or BCR-ABL risk group and said subject expression profile and said reference expression profile comprise values representing the expression levels of at least 4 genes selected from the genes shown in Table 48.

30. A method of choosing a therapy for a subject affected by TEL-AML1, said method comprising:
a) providing a subject expression profile of a sample from said subject affected by TEL-AML1;
b) providing a reference expression profile associated with the occurrence of secondary AML in subjects affected by TEL-AML1 wherein the subject expression profile and the reference expression profile comprise one or more values representing the expression level of a gene having differential expression in subjects affected by TEL-AML1 who will develop secondary AML; and
c) determining whether the subject expression profile shares sufficient similarity to the reference expression profile associated with the occurrence of secondary AML to thereby chose a therapy for the subject affected by TEL-AML1.

31. The method of claim 30, wherein said subject expression profile and said reference expression profile comprise values representing the expression levels of at least 7 genes selected from the genes shown in Table 48.

32. A method to aid in the determination of a prognosis for a subject affected ? by leukemia, said method comprising:
a) providing a subject expression profile of a sample from said subject affected by leukemia;
b) providing a plurality of reference expression profiles, each associated with a leukemia risk group selected from the group consisting of T-ALL, E2A-PBX1, TEL-AML1, BCR-ABL, MLL, Hyperdiploid>50, and Novel, wherein the subject expression profile and each reference expression profile comprise one or more values representing the expression of level of a gene having differential expression in at least one leukemia risk group; and
c) selecting the reference expression profile most similar to the subject expression profile to thereby determine the prognosis for the subject affected by leukemia.

33. A method to aid in the determination of the prognosis for a subject affected by leukemia, said method comprising the steps of:
a) assigning the subject affected by leukemia to a leukemia risk group selected from the group consisting of T-ALL, Hyperdiploid>50, TEL-AML1, MLL, E2A-PBX1, BCR-ABL, or Novel risk group;
b) providing a subject expression profile of a sample from said subject affected by leukemia;
c) providing a reference expression profile associated with the occurrence of relapse in the leukemia risk group to which the subject affected by leukemia is assigned, wherein the subject expression profile and the reference expression profile comprise one or more values representing the expression level of a gene having differential expression in subjects who will relapse after conventional therapy; and
d) determining whether the subject expression profile shares sufficient similarity to the reference expression profile associated with relapse in the Leukemia risk group to which the subject affected by leukemia is assigned to thereby determine the prognosis for the subject affected by leukemia.

34. A method to aid in the determination of the prognosis for a subject affected by TEL-AML1, said method comprising:
a) providing a subject expression profile of a sample from said subject affected by TEL-AML1;
b) providing a reference expression profile associated with the occurrence of secondary AML in subjects affected by TEL-AML1 wherein the subject expression profile and the reference expression profile comprise one or more values representing the expression level of a gene having differential expression in subjects affected by TEL-AML1 who will develop secondary AML after conventional therapy; and
c) determining whether the subject expression profile shares sufficient similarity to the reference expression profile associated with the occurrence of secondary AML to thereby determine the prognosis for the subject affected by TEL-AML1.

35. A method of assigning a subject affected by ALL to an ALL risk group selected from the group consisting of T-ALL, E2A-PBX1, TEL-AML1, BCR-ABL, MLL, Hyperdiploid>50, and Novel, said method comprising:
a) providing a subject expression profile of a sample from said affected by ALL;
b) providing a reference expression profile associated with the T-ALL risk group wherein the subject expression profile and the reference expression profile comprises one or more values representing the expression level of a gene having differential expression in the T-ALL risk group;
c) determining whether the subject expression profile shares statistically significant similarity to the reference expression profile associated with the T-ALL risk group to thereby determine whether the subject affected by ALL is in the T-ALL risk group;
d) if the subject affected by ALL is not in the T-ALL risk group, providing a reference expression profile associated with the E2A-PBX1 risk group wherein the subject expression profile and the reference expression profile comprises one or more values representing the expression level of a gene having differential expression in the E2A-PBX1 risk group;
e) determining whether the subject expression profile shares statistically significant similarity to the reference expression profile associated with the E2A-PBX1 risk group to thereby determine whether the subject affected by ALL is in the E2A-PBX1 risk group;
f) if the subject affected by ALL is not in the E2A-PBX risk group, providing a reference expression profile associated with the TEL-AML1 risk group wherein the subject expression profile and each reference expression profile comprises one ore more valued representing the expression level of a gene having differential expression in the TEL-AML1 risk group;
g) determining whether the subject expression profile shares statistically significant similarity to the reference expression profile associated with the TEL-AML1 risk group to thereby determine whether the subject affected by ALL is in the TEL-AML1 risk group;
h) if the subject affected by ALL is not in the Tel-AML1 risk group, providing a reference expression profile associated with the BCR-ABL risk group wherein the subject expression profile and each reference expression profile comprises one or more values representing the expression level of a gene having differential expression in the BCR-ABL risk group;
i) determining whether the subject expression profile shares statistically significant similarity to the reference expression profile associated with the BCR-ABL risk group to thereby determine whether the subject affected by ALL is in the BCR-ABL risk group;
j) if the subject affected by ALL is not in the BCR-ABL risk group, providing a reference expression profile associated with the MLL risk group wherein the subject expression profile and each reference expression profile comprises one or more values representing the expression level of a gene having differential expression in the MLL risk group;
k) determining whether the subject expression profile shares statistically significant similarity to the reference expression profile associated with the MLL risk group to thereby determine whether the subject affected by ALL is in the MLL risk group;
l) if the subject affected by ALL is not in the MLL risk group, providing a reference expression profile associated with the Hyperdiploid>50 risk group wherein the subject expression profile and each reference expression profile comprises one or more values representing the expression level of a gene having differential expression in the Hyperdiploid>50 risk group;
m) determining whether the subject expression profile shares statistically significant similarity to the reference expression profile associated with the Hyperdiploid 50 risk group to thereby determine whether the subject affected by ALL is in the Hyperdiploid>50 risk group;
n) if the subject affected by ALL is not in the Hyperdiploid>50 risk group, providing a reference expression profile associated with the Novel risk group wherein the subject expression profile and each reference expression profile comprises one or more values representing the expression level of a gene having differential expression in the Novel risk group; and
o) determining whether the subject expression profile shares statistically significant similarity to the reference expression profile associated with the Novel risk group to thereby determine whether the subject affected by ALL is in the Novel risk group.

36. An array for use in a method of assigining a subject affected by leukemia to a leukemia risk group comprising a substrate having a plurality of addresses, wherein each address has disposed thereon a capture probe that can specifically bind a nucleic acid molecule selected from the group consisting of:
a) a nucleic acid molecule that is differentially expressed in at least one leukemia risk group selected from the group consisting of T-ALL, E2A-PBX1, TEL-AML1, BCR-ABL, MLL, Hyperdiploid>50, and Novel;
b) a nucleic acid molecule that is differentially expressed in subjects affected by leukemia who will relapse after conventional therapy; and
c) a nucleic acid molecule that is differentially expressed in subjects affected by leukemia who will develop secondary AML after conventional therapy.

37. The array of claim 36, wherein each nucleic acid molecule that is differentially expressed in at least one leukemia risk group is selected from the group consisting of the genes shown in Tables 2-36, 63-68, and 70-74.

38. The array of claim 36, wherein each nucleic acid molecule that is differentially expressed in subjects affected by leukemia who will relapse after conventional therapy is selected from the group consisting of the genes shown in Tables 44-48.

39. The array of claim 36, wherein each nucleic acid molecule that is differentially expressed in subjects affected by leukemia who will develop secondary AML after conventional therapy is selected from the group consisting of the genes shown in Table 52.

40. The array of claim 36, wherein the substrate has greater than 20 addresses.

41. The array of claim 40, wherein the substrate has greater than 40 addresses.

42. The array of claim 41, wherein the substrate has greater than 68 addresses.

43. The array of claim 36, wherein the substrate has no more than 500 addresses.

44. A kit for assigning a subject affected by ALL to a leukemia risk group, said kit comprising:
a) an array comprising a substrate having a plurality of addresses, wherein each address has disposed thereon a capture probe that can specifically bind a nucleic acid molecule that is differentially expressed in at least one leukemia risk group selected from the group consisting of T-ALL, E2A-PBX1, TEL-AML1, BCR-ABL, MLL, Hyperdiploid>50, and Novel; and
b) a computer-readable medium having a plurality of digitally-encoded expression profiles wherein each profile of the plurality has a plurality of values, each value representing the expression of a nucleic acid molecule detected by the array.

45. A kit for assigning a subject affected by ALL to a leukemia risk group, said kit comprising:
a) an array according to claim 37; and
b) a computer-readable medium having a plurality of digitally-encoded expression profiles wherein each profile of the plurality has a plurality of values, each value representing the expression of a nucleic acid molecule detected by the array.

46. A kit for predicting whether a subject affected by leukemia has an increased risk of relapse, said kit comprising:
a) an array comprising a substrate having a plurality of addresses, wherein each address has disposed thereon a capture probe that can specifically bind a nucleic acid molecule that is differentially expressed in subjects affected by leukemia who will relapse following conventional therapy; and
b) a computer-readable medium having a plurality of digitally-encoded expression profiles wherein each profile of the plurality has a plurality of values, each value representing the expression of a nucleic acid molecule detected by the array.

47. A kit for predicting whether a subject affected by leukemia has an increased risk of relapse, said kit comprising:
a) an array accrding to claim 38; and
b) a computer-readable medium having a plurality of digitally-encoded expression profiles wherein each profile of the plurality has a plurality of values, each value representing the expression of a nucleic acid molecule detected by the array.

48. A kit for predicting whether a subject affected by TEL-AML1 has an increased risk of relapse, said kit comprising:
a) an array comprising a substrate having a plurality of addresses, wherein each address has disposed thereon a capture probe that can specifically bind a nucleic acid molecule that is differentially expressed in subjects affected by TEL-AML1 who will relapse after conventional therapy; and
b) a computer-readable medium having a plurality of digitally-encoded expression profiles wherein each profile of the plurality has a plurality of values, each value representing the expression of a nucleic acid molecule detected by the array.

49. A kit for predicting whether a subject affected by TEL-AML1 has an increased risk of relapse, said kit comprising:
a) an array according to claim 39; and
b) a computer-readable medium having a plurality of digitally-encoded expression profiles wherein each profile of the plurality has a plurality of values, each value representing the expression of a nucleic acid molecule detected by the array.

50. A kit to aid in choosing therapy for a subject affected by leukemia, said kit comprising:
a) an array comprising a substrate having a plurality of addresses, wherein each address has disposed thereon a capture probe that can specifically bind a nucleic acid molecule that is differentially expressed in at least one leukemia risk group selected from the group consisting of T-ALL, E2A-PBX1, TEL-AML1, BCR-ABL, MLL, Hyperdiploid>50, and Novel; and
b) a computer-readable medium having a plurality of digitally-encoded expression profiles wherein each profile of the plurality has a plurality of values, each value representing the expression of a nucleic acid molecule detected by the array.

51. A kit to aid in choosing therapy for a subject affected by leukemia, said kit comprising:
a) an array according to claim 37; and
b) a computer-readable medium having a plurality of digitally-encoded expression profiles wherein each profile of the plurality has a plurality of values, each value representing the expression of a nucleic acid molecule detected by the array.

52. A computer-readable medium comprising a plurality of digitally-encoded expression profiles wherein each profile of the plurality has a plurality of values, each value representing the expression of a gene that is differentially expressed in at least one leukemia risk group selected from the group consisting of T-ALL, E2A-PBX1, TEL-AML1, BCR-ABL, MLL, Hyperdiploid>50, and Novel.

53. The computer readable medium of claim 52, wherein the expression profiles comprise values selected from the group consisting of:
a) values representing the expression levels of at least 7 genes selected from the genes show in Tables 2-8, 16-36, 54-60, and 63-68;
b) a value representing the expression level of the gene shown in Table 10;
c) a value representing the expression level of the gene shown in Table 14;
d) values representing the expression levels of the genes shown in Tables 9, 11, 12, 13, and 15; and
e) values representing the expression level of at least one gene showin in Tables 70, 71, 72, 73, and 74.

54. A computer-readable medium comprising a plurality of digitally-encoded expression profiles wherein each profile of the plurality has a plurality of values, each value representing the expression of a gene that is differentially expressed in subjects affected by leukemia who will relapse following conventional therapy.

55. The computer readable medium of claim 54, wherein the expression profiles comprise values selected from the group consisting of;
a) values representing the expression levels at least 8 genes selected from the genes show in Table 44.
b) values representing the expression levels of at least 5 genes selected from the genes shown in Table 45;
c) values representing the expression levels of at least 3 genes selected from the genes shown in Table 46;
d) values representing the expression levels of at least 5 genes selected from the genes shown in Table 47; and
e) values representing the expression levels of at least 4 genes selected from the genes shown in Table 48.

56. A computer-readable medium comprising a plurality of digitally-encoded expression profiles wherein each profile of the plurality has a plurality of values, each value representing the expression of a gene that is differentially expressed in subjects affected by leukemia who will develop secondary AML.

57. The computer readable medium of claim 56, wherein the expression profiles comprise values selected from values representing the expression levels of at least 7 genes selected from the genes show in Table 52.

58. The method of claim 1 wherein the subject expression profile and the reference expression profile associated with the T-ALL risk group comprise values selected from the group consisting of:
a) values representing the expression levels of at least 20 genes selected from the genes shown in Table 7;
b) a value representing the expression level of the gene shown in Table 14;
c) values representing the expression levels of at least 20 genes selected from the genes shown in Table 21;
d) values representing the expression levels of at least 20 genes selected from the genes shown in Table 28;
e) values representing the expression levels of at least 20 genes selected from the genes shown in Table 35; and
f) values representing the expression levels of at least 20 genes selected from the genes shown in Table 59.

59. The method of claim 1 wherein the subject expression profile and the reference expression profile associated with the E2A-PBX1 risk group comprise values selected from the group consisting of:
a) values representing the expression levels of at least 20 genes selected from the genes shown in Table 3;
b) a value representing the expression level of the gene shown in Table 10;
c) values representing the expression levels of at least 20 genes selected from the genes shown in Table 17;
d) values representing the expression levels of at least 20 genes selected from the genes shown in Table 24;
e) values representing the expression levels of at least 20 genes selected from the genes shown in Table 31;
f) values representing the expression levels of at least 20 genes selected from the genes shown in Table 55;
g) values representing the expression levels of at least 20 genes selected from the genes shown in Table 64; and
h) values representing the expression levels of at least one of the genes shown in Table 71.

60. The method of claim 1 wherein the subject expression profile and the reference expression profile associated with the TEL-AML1 risk group comprise values selected from the group consisting of:
a) values representing the expression levels of at least 20 genes selected from the genes shown in Table 8;
b) values representing the expression levels of the genes shown in Table 15;
c) values representing the expression levels of at least 20 genes selected from the genes shown in Table 22;
d) values representing the expression levels of at least 20 genes selected from the genes shown in Table 29;
e) values representing the expression levels of at least 20 genes selected from the genes shown in Table 36; and
f) values representing the expression levels of at least 20 genes selected from the genes shown in Table 55.

61. The method of claim 1 wherein the subject expression profile and the reference expression profile associated with the BCR-ABL risk group comprise values selected from the group consisting of:
a) values representing the expression level of at least 20 genes selected from the genes shown in Table 2;
b) values representing the expression levels of the genes shown in Table 9;
c) values representing the expression level of at least 20 genes selected from the genes shown in Table 16;
d) values representing the expression levels of at least 20 genes selected from the genes shown in Table 23;
e) values representing the expression levels of at least 20 gene selected from the genes shown in Table 30; and
f) values representing the expression levels of at least 20 genes selected from the genes shown in Table 54.

62. The method of claim 1 wherein the subject expression profile and the reference expression profile associated with the MLL risk group comprise values selected from the group consisting of:
a) values representing the expression levels of at least 20 genes selected from the genes shown in Table 5;
b) values representing the expression levels of the genes shown in Table 12;
c) values representing the expression level of at least 20 genes selected from the genes shown in Table 19;
d) values representing the expression levels of at least 20 genes selected from the genes shown in Table 26;
e) values representing the expression levels of at least 20 genes selected from the genes shown in Table 33; and
f) values representing the expression levels of at least 20 genes selected from the genes shown in Table 57.

63. The method of claim 1 wherein the subject expression profile and the reference expression profile associated with the Hyperdiploid>50 risk group comprise values selected from the group consisting of:
a) values representing the expression levels of at least 20 genes selected from the genes shown in Table 4;
b) values representing the expression levels of the genes shown in Table 11;
c) values representing the expression levels of at least 20 genes selected from the genes shown in Table 18;
d) values representing the expression levels of at least 20 genes selected from the genes shown in Table 25;
e) values representing the expression levels of at least 20 genes selected from the genes shown in Table 32; and
f) values representing the expression levels of at least 20 genes selected from the genes shown in Table 56.

64. The array of claim 36, wherein each nucleic acid molecule that is differentially expressed in at least one leukemia risk group is selected from the group consisting of the genes shown in Tables 2-36.
